SEQUENCE LISTING (33 sequences)

SEQ ID NO: 1 - DNA, 19 bp, synthetic construct
  misc_feature 1..19: note = Description of Artificial Sequence: Synthetic Polynucleotide
  source 1..19: mol_type = other DNA
aagtctctcagctggtaca

SEQ ID NO: 2 - DNA, 32306 bp, Homo sapiens
  source 1..32306: mol_type = other DNA
atgtggggagctttccttctctatgtttccatgaagatgggaggtgagtctcaatctaatagtaaatgctgctaggaattttcaaaacaatttcctttcagctaaattattgcaaattttgacatttgtaatgagagtatttcctgaatatgcattttcctaacgtggtgctaattgtcctcctgttactattgctgctgctgttactgcaaccatttatttcagtctaagaaattctcccatcaatggcagttcttttgtgaccacatggaagcatcatttaaaaaattattccaatagtttttggaggaaacatcatttttaataatgatggggcttctgggggtgctgccctagtaacaatcatgtatcttgtcataggcactgcaggacaaagccttgagcagccctctgaagtgacagctgtggaaggagccattgtccagataaactgcacgtaccagacatctgggttttatgggctgtcctggtaccagcaacatgatggcggagcacccacatttctttcttacaatgctctggatggtttggaggagacaggtcgtttttcttcattccttagtcgctctgatagttatggttacctccttctacaggagctccagatgaaagactctgcctcttacttctgcgctgtgagagacacagtgactatgaggcctccttaactgtgccaaaattcaaaagacaatcagtggagtacaggtgggcttgagaagttctagaacttcctgagtgtatctttgcttaccgtctaattttaaacttgtttagaatatgtagcatagggctctgagacggaaccccattattttctgaaccattgattgctaaagagaaggataaactattttctctctctctttcttctttagtttcttcttttcctcttcctcctgcttatttatgcctccagtatcctgtggttccacgtatggaagatggaatttacttttggaggaagctttttatacaagggaaggatcccttggtgagtgctttagtttatgcagtagtaagttacattaatattcatgtatccttgggaggctctgaaatttgtgaagtaaatgcaaattcctcagcttgagatagtatgatttcatttaattatgtcaaatatatcctcatttttgtatttacctgagtgataatgttgatattatttagcattttttggtggatttatccaacatattggagagtgtgggggctgtggacttagagggtgaagagaagatcatggagagctcttctctgtgaaacagaagagtgaggtcatgcgatgaggtgggggtcaagtgtggaaggaattggtcagtagggaattcctgtaaaagcactggataatggacagaacagtaatataaaatattcagtttaaagattttcttttactgaattgtaatttttgtgtgctatcctaaatgtttttgaagtttgttaaacattacagcctacatagtttgtccttagagggcaatgcagaatttcttccccataggtgaattattttttggcttagtacatttatgtatgtatgtatgtatgtatgtatgtatgtatgtatgtatgtatgatggagtctcgctctgtcacccaggctggagtgcagtggcccaatctcggctcactgcaagctccgcctcctggattcacgccattctcctgcctcagcctcctgagtagctgggcctacaggcatcggccaccacgcctggctaattttttctatttttagtagaaagggagttttaccgtattagccaggatggtcttggtctcctgacctcatgatccaccagcctcggcctcccaaagtgctgggattacaggcgtgagccactgcgccgacctctattagctaatttttaatgacttattttggggtaaacagaataagagggacttggtggtagggagtagaaaggtagagtggatggttttcactcaacaaatattttactgaatacttatatgtgctaggcactctttcaattgctatggtgcatcactaaataaaacatacctgcactgaggttgcttatgttcttgtgaagagagagagtaagtagatgttttagcagactttgaaagcacagggaaaatacatttgaccctcagaactcagcttcattttctttaataggaagagagctgagatgtatgtaagcaatgctgatgtcctgtgcttgttataattttataatataaattaatcatcagctgaataagggctgtataacattggatagttataagactgcaagtcaagttatccaaagaatctcagtctttcaaattattcaaatgtagctgtgtctatagtgggactctatataaaatcagaaagatactgaacgtttgaatggagaatctaacactgaggcaaatgacctttaacatgtgagttatttgtaaaaatgaaaaaaatttcgagtgcttcattgaaatcaagaaggagataaagtctaaaaaactttcagcaaggagtgaatggtcaatggaaatgaactatacaaaatcaattatcttaaatttgaagacgtgtaagttactgatattctccttaatcagtatgctatctttttatctttttaatttagtgaccatgaagagtatccgtaagaacttttataggttcattttgaccttgttttcatatctattttctcctaaagtcttaatttttttttttgagatggagtctcactctgtcgcctaggctggagtgcagaggtgctatcttggctcactgcaacctctgcctcccaggttcaagtgattctcatgcctcagcctcccaagtagctgggattacaggtatgccaccatgcctggctaacttttgtatttttagtagagatggggtttcaccatgttggccaggctgatctcgaactcctgacctcaagtgatccacccaccttggcctcccaaagtgttggaactataggcatgagccaccatgcccagccaagccttaagtttttagataacatgtttttctgccacagagattttcaagaagaaagagtaccattctttgttactttctctctctgctagtagagtttctctttaacccagcaacttctttcatttccacctagaaggtggttgaatgctagatgttggagcaagccattaccagaggataggagacaaggtttttcgaattattgtagatcaaactgggaacccagctctcttgtcatgttcaaagagcagtagctgtgtttggacttcggagttagatatttctaagttcaaatccaagcttcacagggtgttcagataaaatctcacagaatgaaagaaacataggaaggattggaaggaatttgacatgtatcaaatctaactatcttctacaatgtttgagtaaagctctgatttttgagcatctctttgaccttgttttcttgagtagttggggatgagtccagcttcaatgtctcttgccttgagatcttacctttggagctttttatatccctcagttcgggcaagatagaacatcccattgtccagttattttctagtcctgtggtatctgtggagtttccagagccacactggtgctcggatgtctggagcatgttctcactgtggaaattaacctcgctttagttctagttcctaaggaagataaccaagagaacacaggtggaagaagcttgcatcaagaagactacactctatcttggaggtgaactgttcactattacatacataactcaggaaaacttagatacctctcttgtttcttttggcttaatggtggtaaataatttaggtgttactgagccctaggttattcaattgtttcttgtggtttctctactcccatatctttgtacatagttcctatgcagagaaaactctccgcaaattatcatgttttgagtaggctatctattgctttttggaaaatcagcaccatccatgtgccttaagagtgatttgcacatgtactcaggggtacagggaagtgaaggaggtcagttggaagaaatacaaaaaccaagatcttcattaagaagagtttaaaaattttgaaggaatagttggatctttttctaagaaaaatccttaagagatttgataatatgaatttggatgggaaataaggagtgtgctgtgtaccactggatttaggacatgagcgctggaggcccagaatgcacacataacccttctcaggcaacccttgaggctgtggtttggatactgaaccaagggtaaaggcacagacaaaagctgacattttgagggatcagtttatgaggtggagtaaaactctaagctggactctgccagtacccttttgaaaccctgtcttttttcctaaatgaggaatctcaattccagatagaaaatgaggacgaatggctcacgcctgtaatcccagcactttgggaggccgagatgggcagataacaaggtcaggagtttgagaccagcctggccaatatggtgaaaccccgtttctactaaaaacacagaaattagctgggcgtggtggcgtgcacctgtaatcccacgtacttgggaggttgaggctggagaatcgcttgaacctgggaggtggaggttgcagtgagctgagatggtgccactacactccagcctgggcaacagagtgagactctatctaaaaaaaaaaaaaaaagaaaatgaggacgaaacacagccttggttgaatgttctgccaggcagtgttctgtgtggcttttgagcaagtgcaagcacataattttcctatttgtcatctttctgaatctcaagcaagtgcttcctgcctggaagacattagttcacatatttgactcacgtatactctgaattttaagtgtattttgactctgcctctacagtggttaccacatgcttatccattatttattattgagttttatatgcatttgtacttttcaactgacccatatccagccattggtcatacatgagctttcccttggctctgttctcctgggccaattctgggcatggacttttaaactcaccttcccattttgacccagccattcctcagttatcccctcagctgaaacttgatttctgtgaatgcacttagtcactgagtgtggggagggaggtgacttgaaaagcaggtgctacatctcataattaaattatgtaatcctggtacttcagaatctcctggtctctgttgttacatttccttatgaacttctgaggggattaggaagtacttcagaacttcctttcctagtaggcatatgtttcttttttttttttttttttgagatggagtcttgctctgttgcccagactggagtgcagtggtgcgatctcggctcactgcaagctctgcctcctgggttcccgccattctcctacctcagcctcctgagtagctgggactacaggcacctgccaccatgcctggctaattttttgtatttttagtagagacggggtttcaccgtgttagccaggatgctctcaatctcctgacctcgtgatatgcccaccttggcctcccaaagtgctgggattacaggggtgagccactgcacctggccaggtaggcatacctttaaataatactactgattaagagaggcctgtgcatgttttgtggctaataagtaggcagaactagaataaataagatttacacttactggtctagacaacttaccttgggaattgcaacctaggtttggggacttgaccttccaagtctgtggactgacttgcaaactaaccatacttttcggcttgctaaccaagtcttgatctggaaagtaagatctccccgaggactgtcagatctacttgggcaatacttttcagtctgcattagaatcacattttaaaattccctatgcttaggtcccatcccaggtcaatttaatcagaatttctggtgatggagccagaactatttaaataaaagtctcccaagtgattctaaatgaaacaaagttggaaaactacagtctcaaaagataaattggactaaacctctagagttgaaaactcacccaaatttattgtacaagggtcaaaaagtggctgagtgccccatctgggatggtgaggcagatgaggggttgctggttactaatattggcattccttcctttcattgtgagttgctcttataacttgcacctgctgctctctagcaacatgagcctgaggtccaatgttaaactgaggcatgtgacacctgtgctcagatcagtgggcacccagggaccattaaagacttttctttcttacactctttgcctttgtgttccttggagaagaacttcaaaagagcaagcccttcctcagggccatcatggtcatctaaggtttagtacacattttagtaatgacacattaaagataacttaaatatacattttttgagggataaatatcaacatgaggaatagaggactcttggccaggtgcggtggctcatacctgtaatcccagcgctttgggaggctgaggcaggaggattgctatgggttcaagatcagcctgggccaatagtgagattcctatctctactaaaaaaaaaaaaaaaaaagaacagaaggctcttaattacctgtccaagctcttcagacaatcaagaccagtccttgtggagattaaaaatttcagaaactaaaagtttgttatagcttggtcattgctctcatgaagcagcataacttggtcctcctagtgctctgtgaatgtgaaaatgaaaactctctttgtctgtgcttctgttgctgttgctgacgtactctccacttgtctggtttattgataattcactctccagtattacactccatcctctggagggaatgatggatcccttcatcacaaaatgttttctgtatttgcactcttctatgattccctgctcatcccttaagacaaacccttctttaagtcccggatgataacaagagtgatgattgcagcactctgtatttattaagtgtctaccatgtttcagaaacttcacattagttatcttattcaatgctcttaataccttgtattggtccattttcatagtgctataaggaactagctgagactgtataatttatgaagaaaagaggtttaattaattcatagttccacaggcttaacaggaagcatgactgggaggcctcaggaaacttacaatcatggtggaaggtgaaggggaatgacacatgtcttaccatggtggagcaggagagagagacagtgaagggggaagtgccacacacttttaaaccatcagatctcattagtatcagttagtatcagtcagttttcacactgctacacttactacctaagactgtgcaatttatgaagaaaagaggtgtaactgactcacagttccacaagcttaacaagaagcatgactgggaggcctcaagaaacttacaatcatggcggaaggtgaaggggaagcaacacatcttaccatggtggagcaggagagagagagccaagagggaatgccacaagcttttaaaccatcagatctcatgagaactcacacactatcacaagaacagcaagggggaaatctgccttcatgattcactcgcctcccaccaggcccctcctccagtttgacatgagatttggccagagacgcaaatgcaaaccatattatgtctcctttgaagtaagtcttattgcccttattatatacgtaaagtaatcaaggtttagaaaggatagttaattttcccaaggacacatagctggttgatggcggaaccaagattcttctaacagactctaaaagttgtactctttccttttcataccaatatatttttaaaaatgggtagatatgctgaaagtttatcaggttttatcattaaaacctggggtcactgaagtaaagctatctttcactaactatctgtaaattgatagtcaactatcctgggaagttaagtgacccgccacccttaatatttagcatgaatcaatttaattagttcctttagtttcttcagacaaatggaagttatcttaaaggggatggaataaaggaagaaagaagcagttgtgcaaaaggaggtggaaataggtctcagaaaggatgctgttgaaatgaaggggcccacttttttttaaatgaaattgctttttaattcagttaagagaataaaagagatgaaatatgcagttatattttcttttatacttacctacataattacctttaccagcactttttttgtgtgtgtgcatagattcaaacggtattgtgtggtgtcacttgctttctgcctgaagaacttcctttattatttcttgtaaagcaatcttctactaacaaatttttccctgcttttgtttatcttgaatgtctttatttagcctttatttaaaactagcctattttttccagagatttaaaagctcactttatatttgctttctttaaccttttattttaggtttgggggtacatgtgcaggtttgttacataggtaaactcgtataacaggggtttgttgtacacattatttcatcacccaggttaaggccagtacccaatagttatcttttctgctcctctccctccctccactctccaccctcaagtagaccctagtgtctagtgtctgttgttcccttctttgtgttcatgagttctcatcatttagctcccacttataagtaagaacatatgatatttggttttccgttcctggttcctccagcatggatggagctggaggctattatccttagcaaactaacccaggaacagaaacatacatctcattcttttttatggctgcatagtattccatggtgtttatgtcccacattttctttatccaatctgtcattgataggcattttggttgattctatgtctttgctattgtgaatagtgctgcaatcaatgcacatttgcttgcatgtgtctttatggtagaatgatttgtattcctctgggtatatgcccagtaatgggattgctattaaatgtaaatggtaattcttcttttagctctttgaaggatcgccatactgctttccacaatggttgaattaatttacactcccacgaacagtgtatgtgttcccttttctccataacctcaccaacatctattattttttgcctttttcataatagccatactggctcaagtgagatggtatctcattttggttttgatttgcatttctttaacgatcagtgatactgagcttttttttgtttgcttgttggatgcatgtgtgtcttcttttgaaaagtgtctgttcatgtgggatccaggtttcatacagcaaacagaagcaagcacaggtgcagcctcatgtttgtttacattgattcagagtttgagctggtttcaccggtttgactcacagttttctttgttgaggctaggtgagttcctgctaacgactctgtaccacacacacacaaatcactcagtatcactcaagattagttctgttttaatgatgaacttagcattcatacatgtaatcctaagagtagttatagtttgtggatcaactcacatatgaccttaagatctcatgtcttctgacaagagcaatatggcacattggcttaagtgccgcatagaagacttgctagaccaatcagtgtcttctagattctattagaaatgctattttttaaatgtccagttggaaattttcaatggcttgtttcaagattaacaaattattgtggactctgtactgccttgcttgtcgtctcatactatgtgtaacatgtattatatgtatgcataacatataatatccttcctgatattatggtgtttctgtatagcacagtgtgattcttatatccacattaatatatttttacctttacgaattttgaatctttccatgcatttgtttaaattttactgttaaaatgggccaacatgataccacacatccaaagaagtgaaaaatatgccagcgaatgaaagaatacatttaccttgtttttggttattcaccaaggaactgtccatgcttgcttttaggttgccatgtagttgaatcttctgtcttcagtttcccttaaatgttctcttttttttttcttttttcaggtgagaccaggacaggtaatcggcagaactgacaataatttcttcagtttaagatacatttcaagagacttttgcagtgcggtttaaattcagcaacaaatggccgggcgcggtggctcacgcctgtaatcccagtactttgggagatggaggcgggtggatcacgaggtcaggagatcgagaccatcctggctaaaacggtgaaaccccatctctactaaaaatacaaaaaattagccgggcgtggtggcgggtgcctgtagtcccagctactcgggaggctgaggcaggagaatggcgtgaacccgggaggcggagcttgcagtgagccgagatcgcgccacggcactccagcctgggcggcagggcgacactccatctcaaaaaaaaaaaaaaaaaaaaaattcagcagcaaatattgatttctgtgtctgctcgtagttattaggcaatagaggcagacaaacacaaatgaatgctttatgtcctttgagggctcacaatccatttgaagtggaaagagagataaattccttttttttttagacttttttttttttttttttttagatgaagtctggtactgtcacctaggctggagtgcagtggtgcaatctccgctcactgcaacctctacctcctgggttcaagcaattatcctgcctcagcctcccaagtagttgggattacaggtgcatgccactatgcctggctaactttttatatttttagtagagatgggggtttcactatgttggccaggctggtcttgaactcctgacttcgtgatctgcccacctcggcctctcaaagtgctgggattacaggcgtgagccactgcgcccggtaggagataaattcttgactgtaatacgtgacagactggggcaaggtctatactaaagaagccatggcaataccagggaagaggagttaagtcctgcctgggaaaaacagatgaggaagtatttaatatggatctgaaggaagcataaattgtcacttggtcaatatggagagggaggacttatattccaggcagagaagagagcttgagcaaaagagtacaggcaagtatagagcatgctcacggaacggcaataaatcagtgagggtgggagagggaagcagaggaaatggcttaagagaacatgtatgtgaagccaagattgcattggattttgaattccagattaaggagcctggccttattcattctaccttcgccgaagagtcctacagattctgtgtttcagagggtaattttgattgtggtctagatgaggaagaactagtcaagaattaagagagacagtctacactgaatcagtttgagctggtttcatagttttgaggagacagaatctgatgtgttagcacatcacgagggcgagggacaggaagaaaacacaaataatccagaattttcaagttcaagtgcttggaaatattcatgccatgcacaagcaaagaaatggaatttagaggggtctaggattaggagcaaagacaatgaaacaaggggttatggagccccctttttactcttttcaaattttctggcatgatatactttctaagacattgacacattctcatcttattcaacctttggtaaggcattttctcatctcacaagatagaggtgatagaaataatgtatccatttatttctaaaagattatgtaaaacaacctgaaccagtttaattaggggaaaagattttgggtcgaaataattcagaattttctgccaaagaaaatgaaaataaattcactttaaatccttagcagtttattgggcatttgtcacatgcagcagtagctgaagagagtggcagtgataatgatgcagatgtagttacttattttcattcagtccttcaacctgctgttatcctcttcagggcagacttcacttcctggttcctcagtgtatagatgagggggttcagtaatggagtgacaacagtgtaaaacacagccgctgccccatccagggggtctttggagccagccctaaggtagatgaaaatacaggggacatagtagactgtgaccacgattaggtgggagccacaggtggagaaggcccggcgcctcccatcagtggtgcgtatcttcaggatggcatttactatgttggcatacgagagcagaattaacatgaagcaactggcggccactaccccgacgtccacaaaggtcacaagctcattgacagttgtgtcagcacaggccagtctcaatactgcggggatgtcacagataaagtaatccacctgattgggcccacagtagggcaggcggaaggtcaaggtggcctggatagacccatgcatggagccggcgacccaagctccagccacaaggactgtgcataacctcccattcatgagcactgggtagcgcaggggctgacatattgccaggtacctgtcataggccatcaaggtgtagaggaagcactgggtgctgcccaggaagtgaaagaaatacagttgagccacacagccaccaaacgggatagccttgatggaaggagtaaaatccaaaataagcagaggaacggtgactgaggagagccacatgtccaggaatgagagcactcccagaagaatgtacatggggcgagcacagagcttcgggtcagcccacatggtgagcagaatgagcaggttccccagctgagtgaggatgtaaatgatgaagaagaccaggaagaggaggcttcttagatttggggggtgagacaaacccagaagaatgaaatctgtcaccacggcatccagcgatgtgtttttggtctttcccatgtctttttgtagtctgctaagatgaatggtgacgtttgacaagagaaacacagcacaatgatgttgtaagtgaatgcttttgtcggatgattgatgcctaggaattatgagataaacaagatagagtcaaattgttttttaaaagaggaggtggtgtcaccattagttatttaggtttacttaaaaggagaagtcctccagaggctggttagtttcctagatgggaggaaactcttgtccattgagagatactgaaagccaggatttgccagatgaaggaagagggacacatgagagtgatagagaagtaccgaaaattccaaccaaaaagtgagttcgccaaggaggggctaaagttgatgcagagcagtgtggaacaaacaccctataatgaccacatctactttttcttcacctactatcattgtcccctttttcctcagctaaaatctcatctccttaacatatcatgcaaaaatattcctcatctggcccttatttatatcgctattgtaccacttgtaattccttcttattgttgggaggcatgacactcttcaagcttctcacagtagtgtgctctaacttgggacttacgcacacactatcttttctgctcccttcacataggtagtgcccacttttccttcaggattcagtttgagtgtcttcaccttcaagaaggctttccggatccctgaggacaatgcttatacttttctgagcacgtacattatgttgcattgtctaatttctgtttttctctcttaataatcatcccgtctagcatgctaaaagtgataaataccgtaattattttctgaatgactgagatagatagaatggatgttgccaaatccccctgactgaatacattgtttctcttgatcttttttgtctgacccctgtgctcacatggctgggacttttaggtcaagggaccacctcattcaccatgtaatatgggtataatttcattataaaaaactccaagaaacatttatgcctgtttttgctccaggatatcatttagatttatttgttgatttatatggttcattggtttgggattgggttttttttttggtatgaggtcacagaggaatttttattgtttgttataaaatcattgattaatgcaggacttgatctttagtgtgcttgtgtgtaatatctatgtattgcagtctatcaaacagcaactctctctaaagacatttacagattttcaagtatctgaacaagatgataaaatagagggatggaatgtgtttgtttcactctttttgttaaataggttgcttgataaagacttttcgctctatatatggtttgagatgcatttatctttcatttaactacaaaatatctttgtaatgcaaatgactactttcataaagcaattaaaatgacaatagattattataaataacatgtgaatattaataagtttaatctctatcggaaattctccaaagagattttatcttaagtattctgtgaaataacttaattcatatgttaacattcaagagttattagtagtaataataataatactgaatatatctggctcttcgaagtctggaaaatgttaaacttgtcctttgcccaccaagaacgaagaaatatcagatttttattgcctgagtttcaggaggacactttgatttatttgaaaatggatgggaaaactaaatcttatttttgtggttagaatatagagaaaagaggctgggcgtggtggctcacgtctgtaatcccagcactttgggaggccgaggcaggcagatcatgaggtcaagagatggagaccaccctgaccaacatggtgaaaccctgtctctactaaaaatacaaaaaaattagctgggtgtggtgtcgggcgcctgtagtcccagctactcgggaggctgaggcaggagaatctcttgaacccgggaggcggaagttgcagtgagtggagatcgcaccactgcactccagcctggtgacagagagagactctgtctcaaaaagaaaaaaaaaaaatgaatatagagcaaagatagaaagagttgagattaaaatggtgcataaaatactaattatggccaggcgcggtggctcatgcctgtaatcccagcactttgggaggccaaggtgggtggatcacgaggtcaggagattgagaccatcctggctaacacggtgaaaccccgtctctactaaaaatacaaaaattagctgggtgtggtggtgggcgcctgtagtcccagctactcgggatgctgaggcaggagaatggcgtgaacccgggaggcggaggttgcagtgagccaagattgcgccactgcactccagcctgggtcacagagcgagactccatctcttaaaaaaatatatatacatatacatatatatatatatatatacttattatgagctaaaagtaattttcttttctttctttttttttgtgtgattgagtcttgctctgttgcccaggctggagtacagtggcctgatctcggctcactgcaacctctgcctcccagattcaagtgattctcctgcctcagcctcccaagtagctgcgactacaggtgcgcgccaccacgcccagctaatttttgtatttttggtagagagggagtttcaccatattggccaggctggtcttgaactcctgacctcatgatccacccaccttggcctcccaaagtgctgggattataggcgtgagccaccgcgcccagctgagctaaagtaattttctttgccaatacaggaatataaactagtaggcttctacttaggcaaataatatattcatgaaatgttttatgaaaatccatttttatatttaaaatcagatagaactgtaccactggggcaattatttgaaggacttccatgatgcatactttcgtatagtgaaaagtttatccctaatttatctactttgccggtctgattttcccccttttgttgtgcctaagtctggtttcatttctcccaggctccctgttgaaagaaagctctttttacccctctaggttctgggttccctgcctgggaggaggcaggaaactgagatgggttatagctggtcggtttgaggacaggatacctttccaggcagacttggtaaagaacagtggttttagcttggtgtttggtattcagaggctgcagaggaagatagcttagacttccagaaggtagatttttggagaaacaaagagtgtgtttacagaaggattggctttcttggcaagagaggtgaacagtctgggaccaccttagaggagacaaaagaaaggccgggcatggtggctcatgcctgtaatcccagcactttgggaggatgaggctggagaatcgcttgggcccagaagtttgagaccagcctaggcaacatagtgggaccctgtctgtaaaaaaaattttaaaaattagccaggtataatggtgggcacctgtagtcctaatttctcaggaggctgagacaggaggatcactggagcctggaaggtcaaggctgcagtgagccatgatggtgccactgtactccttcctgggtgacaggttgagaccttgtctaaaaaaaaaaaaaaaaaaaagacaaatgtagaggccgtatttgtgtatcctcttttccccctcccttatcttccagatttgtgtcattttattttaggtctgttcctcttttttagcttttgaaacttatgatatttaagataaggaaagttaaatacaattttagtatatctaccctagctaattagataaccaaaatattctaaaattccccaagttttagactgtattggatattctccaattagataggtcagaatttaaataatcaaaacacatgaaaataacaaaaagttaaagttcaatataaaaagcatctgaaaataagagcccagtgacatcagagaataaacagcatatttttacaaagcaattatcctggaaactctaagaagagactttgggaatggggtgaaagagtagcactgagaaggtaaggacagggagggtcaagaacaccttcaagggacctctatggcattatgggatcacaggataagctgctttctatgcccctactctgccccaaatcaagctagtttgatatgtgaacacatttcttgcagtgtatttagagaggtgccgaggagggcaagcctctatccagcaatttgaacctagatttctacagtgacctttgggatctggactactatgcaacggttatgaaatcttgaagatgaggagtgaaggctggtccagcattgcgttggacacattatgatcaaaggactcctgtttacccttgtgacaaaactcaacccaatctgcttccaaaaagaagtttcaccttctgtcatcctttattcagagttagacatcctggtagagctagggcagagtagaggaaatggatagaatttgaggtggcctagacatattttatcgaattcacaagagtacattctttcctattaccgggggttaaaaaaaacgaacttgctttagaaaaagtacttagtgccctcttcattggagaaggggaaggttatgaatccagcctttctttgtgtttgcacaatcactgaagaatattcttcaagacccctatgactttatcactgcccaactctgccctttatcactgccctaataaaaagagaatatgaaaagaaaatagtgttgtaatgcttttttttgagtcattttctcaacacaaaaagagtttccccaagtttgaaaagaaggaacttaattctagcccgtttgggaaagagttgaggaaacgcatggagaaaagagacggattaaattaacactcggacaaaataacagaaagtcttaaagttccaaaattagtctgcagccttattttgaacatagctggtgtcaggcctgctttttccatccttttagactcacttgtatagagctgcacaattgtggaggaggctggctgcccttgagtgcaacggtccagcggatggagagatggtcttcagagattcagtctagcctactataaagggctcccacatctgtttcggaaagcctttataatttttttttaaggagtttggcccagcaaataagtgttcagaatctttgagaacgcctgtctttggggttcttggacaagtgtgacttctgtatatattcagtcatattttatatcagtttccatcagctgttgcttttttggattaaaagaaaaaaagacactgccaagagatttaaagaaaaattctaacctcgagggaaaccctactactctgaagtatgtccctaatgagatctttcctcaccaaatcctcattattttgttttgagagagagagctggaaatgtttccagtaggatccctggataaggaactgtggggaataaccttgtttgcatgttatttgaatgacctctaatttcttagaggtgttatgaagtctcaactgatccccttccctccccaaatactcatcaggattccaccatcatcttcaggtttctgttcaggcatcacctaatcagagaggtttctgttgactctctcctttcccttccctattttatttttctctcggcacttgccaccatttgacatactgtatttacttgtttgtttactgcttgtctcccttcactagaatgtgggttttgcttagctttgaatccccagcactactagcatagtgcctggcatgtaataatttctcaatatgtacttctgaataaattaatagttgaaactgccacacatgttaaatgctgtgggcagaaaaaaaaagctgtggtttcaaatataaatctaatgtttaaaattacctggagtagatctgacatcaaattgatcctcgagtgacctgcatataccagggcaagtagcttcggaaatctatttattcattttttcatatttaatgttgggcagggcacagtggctcatgcctgtaatcccaacagtttgggaggctgaggcaggaggatcgcttgcagcctggagttcaagagcagcctgtacaatatagcgagacctcatctcaacaataaaatacaaaaaattagctgggtgcctgtagtcctagctacatgagaggcagaggcagaaggagcgcttgagcccagaaggttgagcctgcagtgaaccatgatcccgccactgcactccagcatgggcgactgagagagaccctctctcaaaaaaaaaaaaaaaaaaattcatgttaatctaaataacagtgggaaattgactacaaatgaagatatgaagcctggagtgtgataaaagcttcaagagtttcaagaacttcccctgtaagatgatggagctcgacaaagactggatgatttctaagtttcttttaattctaaagtaagaaaatgttaggcgtgtgtgtgtgtgtgtgtgtgtgtgtgtgtgtgtgtgtgtgaagttgcactgctacctgttgccacaagatgacagtatcatctttattgaaacaaagtacagttgctatatggagtaaatagaaataaatccagagagttcatgagggatataaattactttggtaaagatattatatgtgaggcattaattagtttgggaaaactatgtatcaaaaagagttaacttccttttgtaacacatgggaaatagtttgaaatatagttttcttcctccctccgtcttttccttcctgttttacacctagtcttcctccttccttttttattctttctttttctttcttccacattctcagtttattatgtaacaaatttcaagtcaaaggaaacagagatatataagaaaaagttaacattttcaccatttcttttaatttcactcatctgaagaaactaatttttttttttttttgagggagttttgctcttgtcacccaggctggagtgcaatggtgtgatgtcagctcactgcaatctccacctcccaggttcaagtgattctcctgcctcagactcccgaggagctgggattacaggcatgcgccaccatgcccggctaattttgtattcttaatagagatggggtttcaccgtgttggtcaggctggcctcaaacgcctgaccttaggtgatccacctgcctcagcttcccaaaatgttgggattacaggcgtaagccactgtgcccggcagtaactaatgttaatagcttttttctcatttcttcctgtatattcgtacatgtacatacacctacagacatacaaattatgtttcttttgaactttaaattaatatctgatcatctgttgggagacaaatttagatggttgaaaactctctgtttcttccttattgtcacaaacaccaccaccaccactaccaccaacacacacacacacacacacacacacacacacacacacacacaccccacctctaattgcaaccttgtttccatttcttcctgtcttgagaagagaaacacttatggggaaattgaatactttgtctttttttttttttttttttttttttttgagacggagtctcactttgtcgcccaggctggagtgcagtggcgcgatctctgctcactgcaaactccgcctcccgggttcccgccattctcctgcctcagcctctcgagtagctgggactacaggcgcccgccacagcgcccagctaatttttttgtattttttggtagagacggggtttcaccgtgttagccaggatggtctcgatctcctgacctcgtgatccgcccacctcggcctcccaaagcgctgggattaaaggcgtgagccaccgcgcccggcctactttgtcttatgttattcccatttgccgtctctgttccttatacattatgctttttcaactttaccagaatcacttggattaaaacccgtggatttctcagtaggaaatgttcatgtgaagacacttctgtagtaacagaacctacagctgctcctgtagaaggaagttgaaagtcatcccttcaagaaaggggctcctccccttgtaattctactgggttttgcatccagactgagtttccttccctcacccacatgaagtgtctaccttctgcagactccaatggctcaggaactgggaatgcagtgccaggctcgtggtatcctgcagcagatgtggggagttttccttctttatgtttccatgaagatgggaggtaagtctcaatctaatagcaaatgctgctgggagttttcaaaacgattttctttcagctaaatgattgccatttctgacactcgtaacaaaagtgtttcttaatagatattttctttatgcggtgctaattgttattggatatcaaataataacgactttgctgccatttatttctaagaatctctccttaatggcaattcttttgtgaccacatggagacatcgtttttaatgataatgggtttccaggagcgctgccctagtaacagtcatgtatcttgtcttaggcactacaggacaaaacattgaccagcccactgagatgacagctacggaaggtgccattgtccagatcaactgcacgtaccagacatctgggttcaacgggctgttctggtaccagcaacatgctggcgaagcacctacatttctgtcttacaatgttctggatggtttggaggagaaaggtcgtttttcttcattccttagtcggtctaaagggtacagttacctccttttgaaggagctccagatgaaagactctgcctcttacctctgtgctgtgagagacacggtgactatgaggcctctttagctgcaccaaaattcaaaaggcaaccacagcagcgagaagctgtatttcctgagtgtatgcctgctgtgagttaagactggggactttggaaccagactgcctggatttgagtttgctactctctagttgggtggtcttaagtcagtcactatttttttttcttttggtttatttccttatccagaaagtgagaataatgagtatctacttcacaagcctgttgtgagttatgactctctgtatcatctatatctctatctatagctatatctagtgttactcagagaagtagtggcacacagtaaactttatttaagtttagttatttcaattctagtcttgaattttcactaagtattacgtggtaggacctctgagactggaactcagtatttctctacccttggtttgtggaaggtgaaatcctgaccactatttgtatcttgtggatctaaagctccacctaatgatggagtttgtcgttcaggtggattgttgctttggaaggatcactgtggtgatactcattttctttggggggactccgaactttggaaatccaaatgcaaatgcttcagagtgagatgttaagtagcctgattaattacgaaaggctatcttcattatgtgcatcctctataaataaatgtaatcttaacatattttggaggtttataactcagactagaagtgaagtttaggggcttcagacttggggagcatgaacataatcaggggagtctatagaagatgggaaaaagaatgaacataaacaatttggtggcaaatctgggtgaggcaggagagccagtaggagattcctatgacaagatgcagtcagtaggcattaagcacaataaaaatttacagtaaatgttaagattttcatttcctgctttataattcccttatgttctcttcaaatcttttaatgaagtctgttaaatgctacaacttgctgtcagctaggtttagagagtacacgattgctgtgtggctacctccacatgagataaagccgatcagagtgcagaagctcagattgcaggttattgggacatcaaaagtgagggctgctccaagttaacatagttatatggccagtcatatgttacatatatatatattttttgagtcaaagattaagcaaaattctctttgggtgagataggagtgctttttcattctaatttataaaatgaacatttgaggtggaatttgaacagacatagtcacactggaaagtaatcttgacaacatgaatccaggccactgaaatgttcatgttccttttgtctaataattcatttttttgagctctcatttaaataaatgcaggctagaaatgaggctttgtttttttccagtttacaagacaaccaaagaagccatagaaatgatatttaagggctttggacttggtgagtatgaattgttcatatagttatcaattagggaaaacaatactatgcaaatgatatggtgaatgctatgatcaatggaatattttaaagttgtattagtttctattgctgcataacaaattaccacaatacccatatattagcttgtagttctgtaggtcataagtccaagcaagttcagctgagttccatgcttagggcataaccatagtaaagtcgaggcattggctgggtgagtactcaagtgctcacacagagttcagagtcctcttccaagttaattctttaaatttttttctattttatatatatatatatatatatatatttttagacggtgtctcgctctgtcacacaggctggagtgcagtggcgtgatctcggttcactgcaagctctgcctccctggttcatgccattcttctgcctcagcctcccgagtagctgggaatacaggtacccgccaccatgcttggctaattttttgtatttttagtagagacagggtttcaccatgttagccaggagggtcttgatttccctgaccttgggatctgcttgccttggcctcccaaagtgctgagattacaggtgtgagccaccgtgcccggcctattttttatatattttttaaaatttagagacaaggtgtttctctgttgcctggggtagagggcagtggcatgatcataggtcactgtgggatcaaattcctgggctcaggaaattctctcacctcagcctcccaagtagctagggctataggcacatactaccatacctggctaattaatatatatatttttgtagagacagggtcctgctctgttgcctaggctggttttgaactcttgagctcaagcagtcctctggactcagcctcctgagttgtgattacaggtgctttttttttttcttttttttttttcggagatggagtcttgctctgttgctgaggctggagtgcagtggtaggatttcagctcactgtaacctctgcctcctgggttcaagcgattcttctgcctcggcctcccaagtagctgggattacaggtacctgccaccatgcctggctaattttcctatttttagtagagacaaagtttcatcttgttggccaggctggtctcgaactcctgacctcaagtgatccgctcgcctcggcctcccaaagtgctgggattactagtgtgagccaccatgcccggccagttcttattattgtcagaattctagtccatgtagatgtgggactaaagttcccatattcttaatgtttgtgggctgggggcatttctgagctccttaaggcaacctgcactctttctcatgtgagtcccaccatcttcaaacgagcaatagcatgtgaagttcatctcatgcttggaatctttctggcttaatcttccaaagttcatgtgtttttaaggtctcatgtcaaccggtacatagatatctcccttttgaaaagtcaactgtgggcctggtgtggtggctcacacctgtaatcccagcactttgagaggccgaggcgggtggatcacctgaggtcaggagtttgagaccagcctgaccaacgtgaaaccctgtctttctactaaaaatacaaaaattagctgggcgtggtggtgggcgcctgtaatcccaactacttgggaggctgaggcaggagaattgcttgaacccggaaggcggaggtttcagtgagccgagatcttgccattgcagtccagtctgggcaacagaacgagactccatctcaaaaaaaaaaaaaaaaaaaaaagtcaactgtttgttcagcgcggtgcctcacgcctgtaatcctaactctttgggatgccgagttgggcggatcacttgaggtcaggatcaaaaccagcctggccaacatggtgaaaccccatctctactaaaaatacaaaaaagttagctgagcatggtggtgggcacctgtaatcccagctactagggaggctgaggcaggagaattgcttgaacccaggaggctgaggttgcagtgagccgagattgcaccactgcactccagtctgggggacagaggtgattctgtctcaaaaaataaaataaaataaaataaaacaaaacaaaacaaacaaaaagtcaactgtgccatagaacccaacacaatcaccagagtaatattgcataacattcaagattctgaggattagggcagggaatcttgggtctattcactgaaattttacctaccacatagcaaataaaggatgtccattaagactgcataaaaatggtgaactgcttatgatataacaatgaatcaagaaaaccagtgtgcataattgaatatgcggaataatcacagctgtgtaaaaacttttgggcatagaaaaacagggaaaggaattacaatcttccctctttactgcatttcccttactcaaagtcttagtatgggtagattttcttctctctctctccctctagtctcttggctaggattggagggtgcacttccaagatggcttgctcacatagctgttgttggcaaaagtccccagattcttatcacatggctgtagtgtgttgacagagagagagacagagagagagagagagagagagagagagagagagccatcttttattgtccagtctcagaagtgttataccatcacttctgctgttttttttcccccagcttctaacatttttctccttttacatataacaaagtagaagaaataatataggattcaaactgcaaaagtagttaatcagtagaatgtatacacacacaaaatatccccataagaagacaggcttatttacaatgaagaaacacatgtacatttaagatagttctcatataaaacatttaaaaaataaaacatatcccacaatgggcaccactgtttacagcaatattaaaggggagaaaacaacactcatttaatggtgagcattaaatgtaaaggtacagcatcagtggaataaattcacaaaactatattacagcttgttaatccatttaagaattgttccaaatatatcatatatttggccctcagaggttcattcctacagatttcaactactgtaaatttctagctactgagaggtagaaaatggttattatcatgattataatgattattagaaacacacacacacacacacacacacacacacacacaaactcaccaaggagttacaaaatctatggagaccagaggccaactatcatcacctcatgtctgctctcaccaacagccttgcatttttcagagaactatctaaaaaaatagtcagacaccagtgtgatcactatctcctatgccaaacctattgggaatgaaacaagcagtattaccataaatgagaacattgaggcttacctttcaaggcttattctggtctcagaaattaattagatacgattattttctattgaaatgaattttagctaacataaaagactagtcctttactagctggtcttagggtaaaggtcgtacacatgagtcttaacctactctctgtcatagctaaagtcagctgaaaatccaaaattaaaagtatgccaaaaatcctaagtacaatcttttggaagtctgctgtgaaaaagcctttaaggatgtactaacttgaagtctaagtgcaaggtcataagagctctaacgctgttagatcttcctcgtttgggagaaaggatcaatgttccctacaaaacgctaagttttgcataaaggtgaaacctacatatactagggtatagaattaatttaattttgttccatcaggatcctttttccatggtaaaaaagcagaatgaggaatgaaggaatgaatcttttttttttttttttttttgagacggagtctcgctctgtcgcccaggctggagtgcagtggcgcgatcagggctcactgtaagctccgcctcccgggttcacgccattctcctgcctcagcctcccgagtagctgggactacaggtgcccgccaccacgtccagctaattttttttgtatttttagtagagacggggtttcacagtgttagccaggatggtctcgatctcctaacctgtgatccgcccgcctcggcctcccaaagtgctgggattacagccatgaaccactgcgcccggccgaggaataaatcttaattggcctcttcatcagaagaggtaaacttggtctctgtattcatgaagctagacagttttttaagcaggcagtggaagcagacagtagaacaagattcctgtagccacagaccactacctggtatcaaactaaagcaaggatcaacttaaacaattatccaaagtcattttaactgtactaaagggtaaagttcaccactgtattattttaaaggaagttgtttaatgtaagaattccattaaccctgggtgagaaaaatcaaaacaagtcatactctctaatgagttatccatgtaagaccaccaataaaaatgcttttaactgtatttgactcgaagacacttacttggtaagtgtgaatttctttcttttttttttcttttatcccattggatttagactattgaataattaagggtagagtgagggagaacaaagagctacttctttaacattttagtatctggatagtatagcagaaactgttcctagggacaatgtatatgccattaatcacattgaataaagtggatgaattattcatttttcttttctttttgttcgagaagcttgtttatctccctttggcctttccagtgtggtttgaaatgattccgtgttggtttagttgaggaagcaacagtaagaaagtcactgtcaggtagctgagaagcaggttgtggacttgctgtcccatccaaacaactgcagcgagataaatgatcatggccatgaaacacatttcaggcttttcttccttcagtgcgaagatttgtttccaccagtccatagctctgcattgagtttttagtagattgctgcaaatttcatggaatttttgctgttgattgctggtgcatttattggagccaaaaatgttaggtgctagaatgggaacaaggtagtcagccaagcaccaaaacacaacaaaagaggaaacagcagacagaacagatggatctagatagtagataattgaagcaccaaagaaaccaagtccatgatggcaggtgaaaaccaagctctttcccataataagactttatccaccatcaacatcacttttccccattcttgcagctattgttctaaacttgcagtctctgcagccagcaaattggtgccgtggttacctccctctgccatcatccctgaagtgctgtctcagcaagcacaaaccaccctccaactcagccttcctcctgagatccacagggcgattctggccacttctgctgtattctgttggtcacacagacccacccttatacagtgtagaaggcgattgcacagtggtgtgaataccagagatgatggagatcattgaggtcatcttgttgactggcttccatttaccaactcctgtcagccattggttgaaggatattaattttattaattaccagggatattaattttctagtattcagggtttactatgtacatgggcaaagaaagcgccagttgccagaaagatcccctcaggcacagaggtgctgacagttgaaagttgggccgtccatggaaaatgtaaacgggttatgcagaggagggtattgatagtgtcagtcaacccttccactgctttgataccctcttacccaacattatatttgctttgtactgtcattgattcctcaaggtggtggctgattacagtttcttaaaaaaaaaaaaaaaaaagacctaaaacagaggatttagccagataagtagagttgttgctgcagttggtcccaagaccatcactgttatctattgtctccctcttctatcactcattctaggctccctcattcagcattcctgctggtctcagtttcttgctctttggtgactcagaccttcatccctgaagggcccaagcccctggttactgtacccttttcagaccttggttgatgcaattacctcttcactgctatcaccaagcatggaaacaccaagacatgttccaatacatctctgagttccattggtacttctctctatccccgttacatagtagcaactctatatcctcatgatagtagtcaattacctctgccactatagttttgccaaatttggggagttttcaggcattatttctttgaatactttttcagtcccacttctttcctcttaaactccaatgatatgaacttagatctcttgttattatcccataggtctctgaaactctgctcatttttctttcagtctattttctccctgtttttcaaactgagtaaattctattgatctgtttaaagtttacagattcagttctctggcatctcccctttatattaaatctatccagagagctttttaatttcagttattgtatttttcagttctctggactcagctgctgcctcaaattcctgggcttaagaaatcctccttcctcagcctcccgagtagttggaactacaggcactcaccaccacgcccagctgattttcaattttttgtagagatgggatctcactatattgcccaggctggtctcaaactcctgggctcaagtgatcctcctacctcagcctcccaaagtgatgggattataggcatgagccaccatgcctggactcacttattgaagcatttttgtgagagctgttttaaagtccttttccaataatttgagcatctgattcgtcttcgtattgaatcagttgattgtcttttctcatttatgttctgatttcctaattcttggaatgaaaagtaatttttgattgtagcctggacattatgggttttttattaggaaacctggatcttatttaatcttctttttagcaggtagttctcctgttgagatgtagtgtaaagtttggtgaatatatatgttcagtttcctgctgggtgccactggcaccaccccaccaaaaggggaccattaagtcacattgccttattgcctctgaatggagatgatcagctttctgctgggccagctgggagaggggaaaggagagggccaactagacttacctcctgcctggttggtatggcaggagtggaggcttagctcaccagtggtccttgttgacaagggagtgggaacaggaactctgattagccctactttccattgctttgttcaattacatggccactggatgggtgtggaggttcagctcctcactgggcattgctgacattggggtgggggaagctgagtgtttactgtctcacctcatagcacctcattctacctcattgttgctaggtagagatggaggctcagctcattgttggacctcactgacagtatcttgctgggagactggagcaccacctgcttctgccatctggggaatggaagatcagctccctgtttggcctgctgacactaccaagggggaaaccagagtgcacagtggcttctattgggaaggaaggtggggtggaagatcaacttccttgctcactgactgagacctgggggtgtggtttatttgttggtgtttagctggagcaggaagggtattgccagaaagatttcctgctgttaggccaccgcctttttccttgtcctttggctagaaggaatgggctttccttggagcttttgttatctatgcatattggtgtctccaggttggaggtttctgtaacatcctgtccagaaatttttttgataaatattagggtggtatattatttttccttttttaaaaattctaatgtagttattgatatgattggtttaaatctaccattttgtgatttgttttctattttccccatttcttctgtgttcattttctttcctttttggtctagattgagtttttttgtatgtttgtttttgggttttattttagtatttaattttatttcatcttcactataatcttattagttatattactttatgttagctgttgctctagaatttagattatacatctttaatatagtataacttcaagtgatattatatcatttcctgtatgatataagaaactttcaaacttctgtttttctcctcccaggctttttgttattactttaatatattttacttctacatgttataaatttcacaatatatttttgctttatatagtcaaaatatttaaaaagcgatttaaaaactgattataaaacactttgatatttttgccttacttataaagattaccttacccagacaacctgtaaagtgtggagaatatagatttatagatattgctttgaacatctgcccaagcagtagaagaggcaaattaggaagtgtttagcaagatgcttctggaagctaagttagttcgaggctgtatatcaggcatcctgggtacccactgactggtaccaatttacttagtgttcatgggtgaggtttcacttgtccatggactttgaatccctggtgtgtatgcagtggttattacagtggctctacaaactaatgtcatgctaattgccaaaggtttggaactcttccatatttgaaggcttctggattttttgtccacttattaaatggctaaatgaatgagactaataacaggaagggcattggaatggaaaacttttaataatacctccgagtattcattgatggcaagttcagttatgtcatgtgatgatcacttcaaaatcctgtgtcagataggtaaaaattccaaagctttcaagcctaaaatttaccctgtgcagaattttgagtttcatgattttatatcctaggcttctataatagggtatcagaggcgaatggaatcattagaggagatacagagtagatggtaagtgtgtgatatatattgagaacatttattttaaacaaataagatagatagtttttatgtataaacaaatgttactgattatacattgaagaatattgctttggactttagaagacagcctaagcttaatagagctcctgggttgattgatttgattttatgatgctgagttttgctatcctcttcctgcctcacctttataatgtgatcatgattattgttacttatttaaggtgggtgctgaggtgaggagaaaggagataatag

SEQ ID NO: 3 - DNA, 32306 bp, Homo sapiens
  source 1..32306: mol_type = other DNA
gatcttggctcgctgcaagctccgcctcctgggttcacgccattctcccacctcagcctcccgagtagctgggactacaggcgcccaccaccacgcccggctaattttgttttagtatttttagtagggacagggtttcaccatgttagccaggacggtctcgagctcctgacctcgtgatgcgcccgcctcggcctcccaaaatgctgggattacagtcgtgagccaccgcgcccagcccagcaaaggctacctttttaagatgtatggtaataaaaaggagatgcggtagtagctcgatataaatagaattaggtaaaagtattatcagaacagagtactctggagcataaatgtagcttgaaaaagcagagaaggtatagggaagaatcgatggaataaattctacagaggcagaagtgcttggaatctggagcactgtgggaggatgagctaagagtagggagagaagaatggtgaaggtgaagattgatgacgactttgacgacagtgagcaatcaattttaagcacttattgtgcacaagccgaacattatgtaccttattgccactaactaggtactattatctccattctatagatgataaaatgttaagtaacttgtctgagtttgcacagctattaagtacaagagttgggatttcaacccatgtctacctgacttgggagcctaaattcctaaccactcgcctctgctttatcatcatagtctttaacaccactgctatgtgaactcctatgactacatgcttgctgtctcgcacagaataaccaaacagtagtctccacacttcaggtaatctatcctttgttcaacccagattctcttgcatttgtaaccttctgacctccccggcaaggtacccatttgcatttgcacatccccacactaacatgcagtgaaatagtctcttagccactctcttcagtgctcctcagagaggtgacaatcatcagtaactgtgaccccattgagagtgtggtttctgaccacgctcaagattatttgctgcaattgaagcagttagtagttgcttattcttttcttttccttgtttgcagatacctatgaaaatggaaattattttttggcaaactttatagcagctcaggtaagactaatttactacattttacaaatcttttcccaggtgtctacagattaaacattacagttaactgcgtaaatgtaatatcccacatcttaataggaggttcttcttgcctttacttttataatccaggtctgttgtcattaaagtaaggaagaaacaatccttactttggcagttttccagtatactatctccatgaaaagaaacttggaattcatacaatgctggggaaaaaatggggccttaaatgtgtaacatgttttccaaagatgagaaatttactagctgataaattttgaaagaaaaatgctatataagctacttattatgcaataatgcataaaagacgacctcaaaaccttagtggtatctaacaataagcacttacttagctcatgagcttgcagggctcagaggatgtgggtctggtcttggctgatctttgctagctcacatacatctccagttagtcttgggtctattgggctgtttagataatcttggctgtattcacatatctggggggtcagctgactctcatatgatttatcatggccttgtgggaaggtctaggcacggcttcttcataattatggaagagggctaaaataaaaataaaagtggagatcccttagttctttataatttttttgtatcgagttgactaatttcccattaccaaagcaaaatcacatggtcaagcccagagacagaggagggacagaatgcctcaactggagtgggagggcattgcaaaactgcatggaaaggggtatggatttgtaagggttgaatatttcataacagtgatgcaatcaatgcaccttgcatattaaatatcccattggtaatctacataattttaattagagtatgaggagaatcattctaggtcaaactggattctgcttctctatttttacctagtcactttaaccaagaagtaatatagcacaaatctattttttttctataacagtgattcacatatttgaagatgtctctcagttatttcttttgtcttcttagaattatacatcaccaggctaacttcatattttctctatttcctgcttatagtggtttccaggtctgatcattctgtttagactccagttaacttagatttcttttaaaacatcagaaccgagagcatattttagagctgtctatccagtatgatagccactagccacatgtggctattgaacacttgaaatgtgaccagcctaatttgaaatattcaaaagttagcttaaaaaattcaaaaatgatttgtgggcttcctgggcaagatggccaaataggaacagctctggcctgcagctcccagtgagatcaatgcagaaggcaggtgatttctgcatttccaactgagatacctggttcatctcactgggactgattagacagtgggtgaagtccacagagggcgaggagaagcagggtgtggtgtcacctcccccaggaagcacaaggggtcagggtactccccccgtagccaaaggaagccacgagggacagtgctatctggcccagatactacacttttcccatggtctttgcaacccatagagcaggagattccctcaggtgcctacaccaccaggaccctgggtttcaagcacaaaactgagtggccgtttgggcagataccaaactagcttcaggagtttttttttttcataccctagtggtgcctggaacaccagtgagacagaaccattcactcccctggaaagccagggagccgagtgcccttgctcagcggatcccaaccccacagagcccagcaagctaagatccacaggcttgaaatactcgccgccagcacagctgtctgaagttgacgtgggaagctcgagtttggtggagggaggggcatccgccattactgaggcttgagtaggcggttttcccctcacagtgtaaacaaagccaccaggaagttcgaactgggcagagcccaccacagctccacaaagccgctgtagcaagactgcctttctagattcctcctgtctgggcagggcatctctgaaaaaaaggcagcagccccagttaggggcttgtagataaaactcccatctctctgggacagacacctgggggaaggggctgctgtggatgcagcttcagcagacttaaaccttcctgcctgccagctctgaagagagcagtggatctcccagcacagtgctggagctctgctaagggacagactgcctcctcaagtgggtccctgacccccgtgcctcctgacagggagatacctcccagcaggggtcaacagacacctcatacaggagagctccagctggaatctggtgggtgcccctctgggatgaagcttccagaggaaggagcaggcagcaatcttaactgttctgcagtctccactggtgatacccaggcaaacatggtctggagtggacctccagcaaactccagcagacctgcagaagaggggcctgactattagaaggaaaactaacaaacggaaagcaatagcatcaacatcaacaaaaaggatgaccatgcaaaaaatccatccgaaggtcaccaacagcaaagaccaaaggtagataaatccacgaagatgaggaaaacccagtgcaaaaagcctaaagattccaaaaaccagaatgccccttctcctccaaaggatcacaactcctcaccagcaagggaacaaaactggagagagaacgaatttgatgaactgacagaaataggcttcagaaggtgggtaataacaagctcctccgagctaaaggagcatgttctaacccaatgcaaggaagctaagaaccttgataaaaggttagaggaattgctaactagaataaccagtttagagagaacataaatgacctgatggagctgaaaaatatagcatgagaacttcgtgaagcaaacacaagtatcaatcactgaatagatcaagcaaaagaaaggatatcagagattgaacagcaacttaatgaaataaagcgtgaagacaaaattagagaaaaaagaatgaaaaggaacaaacaaagcctccaagaaatatgggactatgtgaaaagaccaaacctacatttgattggtgtagctgaaagtgacgaggagaatggaaccaagttgtaaagcacacttcaaggtattatccaggagaattttccaacctagcaagacaggccaacattcaaattcaggaaatacagagaacaccacaaaaatattcctcaagaagagcaaccccacgacacataattgtcagattcaccaaggttgaaatgaaggaaaaaatgttaaggtcagccagagaggaaggttgggttacccacaaagggaagcccatcagactaacagcagatctgtctgcagaaacctcacaagccagaagagaatgggggccaatattcaacattcttaaagaaaagaattttcaacccagaatttcaaatccagccaaactaagcttcataagtgaaggggaaataaaatcctttacaagcaatccatttacaagcaaatgctgagggatttggtcaccactaggcctgccttacaagagctcctgaaggaagcactaaatatggaaaaggaaaaatgagtatcagctcctgcaaaaataaaccaaaatgtaaagaccattgacactatgaagaaactgcatcaactaatgggcaaaataaccagctagcatcataatgacaggatcaaattcacaaatagcagtattaaccttaaatgtaaatgggctaaatgctctgattaaaaggcacagactggcaaattggataaagagtcaagacccactggtgtgctgtattcagaagacccatctcatgtgcaaagacacactaggctcaaaataaagggatggaggaagatttgcccatcaaatgggaagtaaaaagaaaaagcaggggttgcaattttcatctctgataaaacagattttaaaccaacaaatatcaaaaaagacaaagaaggccattacataatgttaagagctaactaccataaatatatatgcacccaatacagaagcacccacattcataaggcaagtccttagagacctacaaagagacttagactctcacacaataatagtgggagattttaacacccactgtcaatattagacagaccaacaagacaggaaattagcaaggatattcagaacttgaactcagctctggaccaagcagatgtaatagacatctacagaactctccaccctaaatcaacagaatatacattcttctcagcaccaatagcacttattcgaaaatcaaccacataattggaagtaatacgctcctcagaaaatgaaaaagaacagaaatcataacaaagagtctctcagaccgcagtgcaatcaaattagaactcaggattaagaaactcactcaaaaccacacaactacagggaaactgaacaacctgctcctggatgactactgagtaaataatgaaattaaggcagaaataaataagttctttgaaaccaacaaaaacaaagacacaacatatcaaaatctctgggagttttcagagggaaatttatagcactaaatgcccacaggagaaaacaggaaggatctaaaatcaacaccctaacatcatagttaaaagagctagagaagcaagggcaaaaaagttcaaaagctagcagaagacaaaaaataactaagatcagagcagaactgaaggagagagagacacgaaatacccttcaaaaaaatcagtgactccaggagctggttttttgaaatgattaacagaatagttagaccgctagccagactaataaagaaaaaaagagagaagaatcaaatagacacaataaaaaatggtagagaggagatcaccgctgatcccacagaaatacaaactaccatcagagaatactataaacatataaacacctctatgcaaataaactagaaaatctagaagaaattgataaattcctggacacatacaccctctgaaggtaaaaccagggagaagttgaatccctgagtagaccaataacaaggtctgaaattgaggcactaaattaagagcctaccaaccaaaacagcccaggaccagacagattcacagccgaattctaccagaagtacaaagaggaactggtaccgttcctcctgaaactattccaaacaagagggactctgccctaactcattttatgaggtcagcatcatcctgataccaacacctggcagagacacaataaaaaaagaaaatttcaggccagtatccctgatgaacatcaatgcaaaagtcctccataaaatactggcaaaccgaatccagcagcacatcaaaaagccatgatcaagttggcttcatccttgggatgcaaggctggttcaacatatgcaagtcaataattgcaatccatcacataaacagaaccaatgacaaaaaccacatgattatctcaatagattcagaaaaggcctttgataaaattcaacaccctttcatgctaaaaacactcaataaactgggtattgatggaacaaatataaaaataatatgagctatttatgacaaaccaacagctaatatcatattgaataggaaaaagctggaagctttccctttgaaaaccggcacaagacaaggatgccctctctcaccactcctattcagcatagtattggaagttctggccagggcaatcaggcaagagaaagaaataaagcttattcaaataggaagagaggaagccaaattatctctatttgcagatgatgtgattgtatatttagaaaaccccattgtctcagccgaaaatctcattaaggtgataagcaacttcagcaaagtctcaggatacaaaatcaatgtgcaaaaatcacaagcatttcgatacaccaataatagacagccaaatcatgagcaaactcccactcacaattgctacaaagagaagaagatacctaggaatacaacttacaagggatgtgaaggacctcttcaaggagaactacaaaccactactcaaggatataagagaggacacaagcaaatggaaaaacattccatgctcatggataggaagaatcagtatcgtgaaaatggtcatacttcccaatgcaaatcaaaaccacaatgagatatcatctcatgccagttagaatggccattatcaaaaagtcaggaaacagcagatgctggagaggatgtggagaaataggaatgcttttacactgtcagtgggagtgtaaattagttcaaccattgtggaagacagtgtggtgattcctcaaggatctagaaccagaaataccatttcacccagcaatcccattactgggtatatacccaaagggttataaatcattctgctataaagaaacaggcacaagtctgtttactgcagcactgttcacaatagcaaagacttagaaccaacccaaatgcccatcagtgttagaccagataaagaaaatgtggcacatatataccatggaatagtatgcagccataaaaaagaatgagttcatgcccttttcaggctcatggatgaagctggaaaccatcacactcagcaaactaacacaggaacagaaaaccaaacactgcatgttgtcactcatatgtgggagttgaataatgagaacatatgggcacagagaggggaacttcacacaccagggcctgttgggggtggagggcaaggagacagatagcattaggagaaatccctaatgtagatgacgggttgatgggttgatggatgcagcaaaccaccatggcacatgtatacctatgtaacaaacctgcatgttctgcacatgtatcccagaacttaaagtataattaaaaaaaaaaaaaaacatttggctgagcgtggtggctcatgcctgtaatgccagcactttgggaagccaaggcaggtggatcacctgaggtcgggagtttgagaccagcctgaccaacatggagaaaaccctgtctctactcaaaatccaaaaattaggcgtggtggcgcatgcctgtaatccccactacttgggaggctgaggcgggagaatcgcttgaacctgggaggcagaggttgtggtgagccgagatcgcgccattgcactccagcctgggtagcaagagcgaaactctgtctcaaaaaaaaaaaaaaaaaacctcatgaataactttatattgaaaaatgttgtaataataacattttggctatatcatgtaaataaaatatattttgaaattaatgtaccctttttcttttcactttttaaaaaaatgtgactactagaaaatattcaattacatatatagtttcccttatatttctataagatggcatagacttccatgttgcctactacctctttttagctagagtctatattttttctgctgttattacattgtgttattttttaacacatgtatcagatgtttggcacatataaatctcatggtcagctaagaccttcaaattattctcatagacatgattatcaggtcatattgttccatcctgcatttggcctctctgaaactaactgctagactttgtatactaattaaatttgtgtattctctgcccttaagagtagttgggttgaagactctacatattttttcctctcaaatattattttaatgaatattattttaatgatctggaagtcctgaattttagttctatttctactgctattattcaaattcggagagacactgtttaatctgtgttttttaaaaaattgtaaaacattagattgccctttaataacatctaacttcaaaattccatttttctgataaatgaattcttatttctgtgatgtttgatagattatttccttaagaataaggggcctgaaatgaaagaaagatcgcaactttccttaggggataactattatcagcggtttcctggttggtaaaccagcttgtgggggtgggagatgatttgtaacatttgctaatttctgtagtgtagcttctcccaccatcttaaccaatttcaagcatcagctggcttgcaaaattctaggaaatttaaaaatcaactctctcaaatcaataagaactagctttagtacacccatttattttgagctgtatattttttccttgattctgcctgcccagtagcaaaagtcttgttcatccttcatgatgccaattgaacatgtttcttttttctcttcctactgcagccatcataattgagaaatgcatagcttccctctgctacaactatctccttgattattgcatgtaatcttcaaaatctttccttacatacctaactggcctcacgtctctgtttgctcagcaccagctatggttgtccttgcactcctccatgaatgtggctttgttcaatgtgtccctgtacccctatttcctgtctcataataaatccttcccatgattaccaggagttccgaagttacatgtccttcctaagttctctcttgaccctcttcctcccatgtaatttttcctttctcttcacatccatgaaaaatatccactatgattttcatttgggccttcatcatgtgccacctcataatttatgtcatattattttaacccttgtattgatacttaaacaattcatgatttggttaacttcctatgtttcttgcacactgtcctcagtctagggactgtttgccacacttctttgttcactctaaattaacttgtgcagtactatgtacaggggcaccttgcgcttccaactttacctatgttatctagccttgtcgctttggactactcacttcattttccagagcctcagtttccttatctctaagatagcaataatgatattttcttttcaaaatgattataagaatacttatgatatagggaaaataactttcataatgctggtacaataaacttatttgtggcattacccaatttattatagcaggtactcaggaagtatgtcttcatgaagtgtgtggagagtagaattatagtaaccagaggctgggaaggggtgggggccataaagagaggttggttagtgggaacaaagagacagttaaatagcaggagtgagttcttgtgtttgctaacatactagggtgatgatagttaacaacaatttattgtatatttcaaaatagctagaagagaatatttgaaatgtttccaacacgaagaaatgatgaatgtggcttggtgtggtggctcacacctgtaatcccatcactttgggaggccgaggcaggaggatcacgtggtcagggatttgagaccagcctggccaacatagtgaaaccccgtctctattaaaaatacaaaaattagccgggtatggtggcacacacctatagtcccagctgcttaggaggctgaggcaggagaatcgcttaaacccaggaggcggaggttgcagtgagcccagaccacgccattgcactccagcctgggtgacagagtgagactccatctcaaaacaaacaaacaaacaaacaaatgttgactatttgaggtcatggatatcctaaataccctgatttgatcactacacattgtatacatgtatcaaaatatcacatgttctccataaatatgtataattattatgtatcaataaaaatttaaatacataaatagaaatagcaaactaatataacaaaagtaaatttaggtttgttaatattttaatatggaccatagtctatcaacaatatgttatgtattcttttctctttataaattctttatagaacatcctgcaaatccagaccatacacaataagtatttgagtgactcgaatccactaaaagttgggtatattagaatctggggtgtgaatacctatgtgacacaagtcagtttcacctatgacaaccggcaatttatggagacaaatttcaagagtgaaccttataatcaggtaggtctgaaaggaatattagcatatcacaagtaaattttatcattctgcaaaattatcaccaataattttgctaacaattaaaattatcattgttaaattttcatgtattagatggttataaatacgtgcaatagatatgttcctgaaaagttgcaaatgttgaatcatattgtaaaatatgcaacattaatggtctttccagtatggcctaggggtcagtgatcactggcctgatgagatcctctccatgtatttaccaaagagactgtgctatgttttgataggttagtcttccacattagcaaaataaaacttatttctgctgcagaggctaaaacaaaggaaagggtgattccctaaagctgttgaaggttgcccccatgcttcctgcagttattcatgtccctggtcaaattgatggatcttataggtagacgaaaattggcagtaaactgtggtaatcttcgaatgaatacatcccacctgtggtataggataaaaggttgataaaacagggggacataaatagcaggccctactagaggattctagaatcaactaaaccagaattttggaagatgaaataccaatagttaccaaaggcaaccacaaagaaaaaagtatctcacttttagaacataagacaacataaatacggaaccagcaacccaaaagggatggtttagaaaagtgaaaatggataaccatagttcttgttttctaaaacgtgaaatgagtgccctgatggtatttggactttggaaaatggcccctctgagcactcaaatagatcttctccctctaggctcttctgggtatttaccttaaagcattttacctggactattgagttcaaaaccactctttgcaagtaattgcttctagttaagattgtgcagaaaatccttgaataccttctttttaaaaactctatggcatatctgatgcctattatgtgccagacaccatactgtttgaaatgaggaaaacatgaaaccaacctgctgtcaagaggactaaaacccaatggcaaagcaaagagattttaataaatgttggttttaatcacagctacaaatatttgtaaacaataaggttctgactaaatgaagtgaaaagaaaagatttccagcatagggattatgcacacagagcatctctctgtatttatctgaccaaaatcaacatgacctaacccaaaaaggaatgtttatatcctattactggaaaggaatgaatccatcattttttgttaaattgtaatttaagaggtgagctacatgtgaggtttaagaagttctaaaatacccatttatatctcttcttttctggcagatactaactattcaattgactgacaagactatcaacctggaaaagttaactgaggttacttggattgatggtggtcctgtacttcctactccaactaagacaagtaccatcccaatgagttctcatccttctccatctactaccaatgccaccagttctgagacaatcaccagttctgccagtgcaaatactaccactggcactactgatactgttcctatcacaaccacatctttcccaagtactactagtgttacaactaatactactgttcctgatacaacttctcctttccctacaagtactactaatgctagcactaatgctactgttcctatcacaaccacacctttcccaacaagtactattggtgttacaactaatgctactgttcccaatacaactgcccctttcccaacaaatgctagtactgctagcactaatgctactgttcctatcacaaccacatgttttgcaacaagtactattggtgttacaactaatgctactgttcccgatacaactgcccctttcccaacaaatactactactgctagcactaatgctactattcctatcacaaccacaccttttgcaacaagtactattagtgttacaactagtactactgttcctgatacaactgctcctttccctacaagtactactagtgctagcactaatgctacccctgttcctatcacaaccacactttttgcaacaagtactattggtgttacaactggtactactgttcctgatacaactgctcctttccctacaagtactactagtactagcactagtgctactgttcctattacaaccacaccttcccctacaaatactgctgatgctaacactagtaatactgttcctaataccactatgccttctcctacaagtagtactactgtgagtactattgctaccgttcccatttcagtgactccttctctgacaagtactgctgatgccaccattagtactactgtacttattgccactacttcttctctaacaggtactactgatgttagcactagtactactattaataatataagtactcctgttcaaacaaatactactaatgctagcactagtactaatgttgctaatataactgctacctctcatacaagtactgatgatactgttcctaataatactgttccagttacagctattccttctcttgcaaatactggtgttgacactactagcaacagtttttccattatgaccacttctttctctgaaagtactaatgctatgaacactactgttattatggcaactacttctcctacaagtactgatgttgctagcacaaataatgatgcttctatgacaaattttcttttagctacaatgtctgctggtaatataactagtaatagtatttccataacaactacttcttttggtaatagtgttccttttgtgactactccttctccaagtactgatgctactactacaagtaataatactaatcctggcatgactacttattaccagacttctcctaccattcctacccatactcttacttctattcctagctctattacttctattttgagcatgtttccaacaagtaatacattcactactgataaaattactaattttactacccctacaaatgcaaacaccattattttcaacactcttgatacaaaaagtaccatggtaatagatgctacggtcactactaccagcaccaaagataataccatgagtccagatacaacagttacttccatagacaaattcaccacacacatcacacagttcgctactccccattctgctactactacaacactggccttaagccacacctcattagctcctacaaatctttctaatctaggcaccatggatattactgatgcagataactccagcagtgttacaggtaacacgacacacatttctgtttcaaatctcacaacagcctcagtcacaataacagccactggtctagattcacaaactccccatatggtaataaattctgtggctacttatttacctattactgcaactagtgctaccacagatactacaaatattacaaaatatgctttaaatactaccactcctgatagtacagtacatacctctgctactgcacctacttatattgcaaatgccataaatgctactcaagttccatgattactactcaaggcaggatagttacctcagataacgcctacaaacaactattacagatatagaaaatcagttacgagacactctatctatcttatgctacttaagttttcacggatattagtactctagccataaaagacacagctactccaaacactctcgtcattgcagacatgtttaggaaggtttacaaaccttataggtttcaccaaagaagctgtgggtacttattttgcaaccatagtatgtgctcttattcttttaaattatagttattactcctataacctcatcagttaaactacagatgttttatatatcctatatatccttgctacagatttcacaggtaattttggtatctcagataacaccacattaggctctgtaaatactattgccttagatttagttactgaaatagcccaacttgtttcttgggtcacatgacgtaccataactgagagtactggtgatacccccagcactcaaatggtttctataccaacagttattgatattacaggagagtatgtaattagtaatgctaaaaaaatgcactttattatcctatggacttttccaaatgccatagctaccaatagagtcatttgcattacacatactaatagtattatttcttctgaggagatcctagctgtagctacagatatagaaaattctaccattgaagatcttgtataaccttacttcagccactgaaataatttaaattataaatattacatgtgggtttgactatcacagaaaataaaatgattatagatcctaaaaacataaattcctgaactttgcaaccattaattcataggtactactaatactcttactacagattttataagtacttccacttatagacagaagagcattctcagaaaattagaattaatctaaattatgagatagtcttaaagcctcactgttaccaccaatgactggctctgactctagtataaatcagaaattatccagacagaggtgagaatgtaaatatacagcatgaggaagacagcctccacattggaatcagagttcataatcttatttgcattttcttctattaatacaagcttttcccttataatttcactgttcagactccccttcattgtgttagggttttcagtctattcctggtttttcatatccaaattcatcatccatccttgatccattgttttcttagatcactcaaacccccatgagtagacccatggtcattaccaaatctggtcatttgggtactattgtaggtttaaccctagggcaatattctcgaagtttagttcagggatccatggggttccccaagactttctggagcacacagcttcttcaaatctactttcataatgatgctaagttatttgttttttcagtctcattgtctcattagtatatagtggcactgtccagatgctctatgctgtattgtgtcataaaagatgaatatagaaaaagaaaaaagaatctagctgtattctattaagctagacattaaacagatttacaaaatgtaaaataatgtcattcttctaatttttttttgctttgggaaatacagttatttttcattaaaaacatatttatgttatcacaaagtgaatatgttattttttatgaatgaaaaataaatatttaaaaaatattttacaaatgactcaattttgatttctaatgcagtaaatattagcagatataacccacataaataaagcacagtgccctcaccaattttgaatgtaaaggggttctgagactaaagagtttgagaacaacttcctttaagtgggcagaaacatcgtatttccactctcatgtcctggtcaagtatattatcaacttccccagcatgtaggtactccagcatctgtggcttactcaacatgaatatctttccactgtctacaaacacatctacccagctttttgatctgtcagcccagctgtactattgtccagacaatttgatggtactccagatgaggggcacagattcatcagtcagcaacatttgcttttggaatctgcccacaagctacagtcctctgaacaaagttgatagaccttattcttgtgtatacatgactcttgaccttcccaactcaaaacaaaactcaaaattctatgggcttggtcagactccctcacttgtatctttactgagggagacttagtgcatttgtcttcttggcatcacaaactctgatacaggacaaagtgctctgtgtgatagggcaccatgagattctgctgccaggccaccatacattgctcattcactgaccaacaacttcattaaagcagctgggatttggaaaactgaagatgaatctgcttgaacaagcatagttctgaagccaccaaagtccaacctcttaagtacgtaaatggaacataattgaaagcatggaattttgaaaaggtaagaaatactttgaatatacaaaagtcttgactttggtcagatgtttgaaaagacttgagaattgaaactcaggagtcaatgcacaagtagtggtcctggaaccaggttcagaaaacatgagggcaatgttgacatgctaattctaccattgacaacactagcaaaacctgtgcatggactctgatttataaggtctagagaaacttctaagctccataatgactggccccgctgggtcacccacatgtgtgatattaattctgcactaatgattataacttatgttctaagtataaagaaatttacttttctacacttctggtacttgacacattcctacctttaccattactgtggacatcattatttttacacctaaaatttctagttattaatttaaacttcagtttctagtattccaagcatcgctatgtaaacactattcatgttgactatttaagagatgtcacacatactgcatgattaattttccagtgaggaatatagatgacagatgttttggattaacaggaaactcactgggagtctaaaggtatttaggaatacttcaaggagtaagggaagctgggaggtaccctgtagcacaaaaaagagtcaagaaaagctttcaggtggggaatgcactggacaaagagactgactttacatggtgtgattaggagatataaaatagtccactcttttctacagcaggagcattgtgagaagagacaagatttaaagtttatcaagttccatcaccaaacttccgtgtatatgagattagacattcaaatgagggttgaaagagcattcataccagtaaaggaacttacagggctagagaatgctttggtttccacatccctttccccatgggagagtggagtggtgctctgtgtgaagggcactgcagaagaagtgctcgatggagattaagagtgcctgtgaggagctgggactggcatctcactcccagctgggcactcgctgaactgagaaactcttgcctctcttccccatcagatgatacagtggagcatggagcatgggcaaggtttcagaaacctcatcatgcatctcccagagtgtggaagccaatgaaatcacaaagaatggcgctagagtcttgcccagagaattccaaaagtggcagagattggagaccctgcagtagtggatgcagagccagggaagtagtggtgcatcctggactcaacagtgttcaatggacaaggaagctgaagctcctcaggtgggtgtggggagatggctgggctagggctgtcatgaagcaatctgtccagtggagaagggactctagtgatgagaggtcgtggatggacttccaagagtggaaactaagtttgtagtggggagaggtcaaaataggtcatgcagattgcatctcacctcatgttagatgctgcacaaggggcctctgaaaaactcactaatgtgcccatgggaagaaatcagcactggggtcctctcacactgagggtacctgcggatgccttgccatagcatccatcacatgggaacctttgtcttttcctttaatttccatctggctgtgaaggatacccagaaacagtcaaggaagaggagaaggagaatatggaagtgtaaaagcatagaccgtaatccccttcctcactgctggaggtggtctctgcggaaggagaaaacttgacatggatgtgaaaatgacataccgatttagatgatacaaattcttgaaaacttccaaatgaccagaaagtgatgaggtctgttgacgggataagaaaagaatttcaactcagcattgaggaagaggggctaagattaagaaaaaatgaaaagcttgagaatgctgaaaagacacacactgcctcatttcacattgctcttacctgttagaatgatgggtgaacattcatccttaactttgtaatgctttataaatctcttattccaaggaaaagtggagaatggcttggagtacaaaagttagatctgggccccaaaggacgtttgaggttttctcaccttagccaatctctggtggcttcagagaaggtggctttggaaattttttactatgtaattgagaaaagtttggctcaagagaagaagtaaaggggtctgtacaacctatgggtcttgttacttaaaaacaacttcttatatttgcacaataatccatagtttgcaaagggcctcacataaatgttttattttgttccactcctgctgtgaggcagagagggaagaaatctttacactttttttctcagaggcagaaaggaagacttagaaaggttaaatgacttcttcaagaacacagagtcttaaaacatagtagagcttggccttctgattcacttcctattacccgtctgcttcagggtcccatctcctgaggaaagtccatctttgccatgatgtgatgaactaaaataaaataaacaaaaacccacttctagaaccaaataagtcttgtaaaagagtcctgatattattcctatctatttacgtgacctggatcaacttgcttgaattatctgagccttatttaattccttcattggtaaaatggaaggattgagagagtgcctaggaaaatatctagtgtagcaagaacccataatttggtattctttttggcatatcaaaacatcttgtaaccttgaatatatacaattttcatttgtcaattatacctcaataaagctggaagaaaaaaaaagagctgactactctattccttactgctaatacaccaagatgatacgtctggcttcctaggtcctctctcccaaattcttccctttttgataataattttaaaatgaacaataaatagataagttgtggtgaggttgccaatgtgttagaaacattgagtggaacttacgaaaaggaaccccttatgaaagttgtaagattcaaaatggagtcacttgtgccaaaccctggcaaaatagagttggggaaggtcctgaagagagggctctcacacaatttgtctgataacaggaactatcacaggacatttttccaaactgcagctttttacatgagtcacgccaagacagctagctgcctacacaagaatacttgcctgacacactgtctcacaaacctaatccctgcaagggagctacagtaacttccaggttacaaatcccacctagcaactatagactgttgccaatcacaactcgccagctcttgtaagacactgctagtgccaatgaactttctttcaaaacgacttgcataaccttctctttccccaataaaactctaatgtttttctttgttctctggatatagcagaggccaccctgttgtgcctgtatcccaaattgcagttctgttttcacattttaattccaaataaaagttctgtgctttgggaacatctctacattttttcttggaagttgacaccctttaactctgcctttgctttccttcttaccctcttttgctgggtggaaatgtggagtcagacagtagcaatagtgagttggctctagatgcccacaacaccttggagggggagctggagagcagctctgcatgcagaaagtaggtgtgggcggagtagctctgtcatgtgcctccacacaggagtgaagcttgacatggaacatggtcaccatatgccatttgcgcctccctctaattcaatgacttctcaaaaaaaccaccattttaatatatttattattcatttttaaatttccttttacttaaattactaatgcattggcagtttttgtagatgaagattcttatttattgactgtctacaaaaataaaattgtaatctttcttaataagaggaaaacccaaatttttgagggtgagctatgcaaagagcctcttcagggtgcatgtgtttggacatagaggtcgcagtagagggagaattgacagaccttgtggtcctaaatcttcccctgggaggttaattgctccttagtggtcacttactgataaatggcacctccatatgctgacatcaggcagaagtggagtgctgatgcaggacaggcaagcccccaaattggggctcagccagacagggttcttggctttccccaggaaagaattcaagggcaagtaggtggtgctaaacagaaacttttactgaagtggcagtgcatagcagcagcagaggcagtgcttcctgtggaacagggctatgctgtaggcagtgcacccagagtagcagctcagaggcagttctgcagcctcattaatacctacttttaatcacatgtaaattaaggagcaggttattcagaaatttctagaaaaggggtggtaacttccaggtcattgtcatggaaagggttgtaacttctggctgttaccatggcaatgccatggcaatggtaaactgggaagaggtcgagtgacatggcattggtggatgtgtctcagggagagatgcttttgcctcttcactatttcagctagtcttcaaactggtctggagttcaagccttacctcagtgctgcttccaatactctgtccaatactgattgttatctttctcttattttagagtgcagttgagataaggtggaaccactgaatgttttaggaaagcatactagacaggaaacaagagtataagaattatgatcattttctctgtaaggtaaaatccatcctttcttcctgagttctcagagaaaggatgtctgagctatgtctgcctgtgaattagtccatttttctctttcttaggtcctaactttcttcttcctttccagagggcagtcccaacaagacgacaattagttatttgaataaaagggcagtttcttaaatctactaaagatttaatctactaaatttaatctactgaagaccctgtgctaaacattttaattctataggcatgtgaaatgacagaaaaacaaaactgttgttcagagagttaagcaactggtctgatactgtttatcaggttaagtgttggagaaacgattacaatgagatctgcctgcctgtaaatagcattataatttcaaacttcccctgaggaagaaccttggggttcctaaaagctgctttaggatgagggaaggtcaaactcttccttcccagttgtatcagaattgctttgttttttgtgttttctcttctgtatttttgcataagattttatttgaagaaagggcatcattgcttaaaaaaattgaggaaaaccaattctgaacaatgccatactttcgataatttctgctgtatgagttagacagtattttgtgggctagctcaggaacctaaccaacatgcattctaagtaggaagcatattacctataaatcagctatcagaacacaatctccatgtaatctaaatactttatgtcaggatacaaagatggccaaattgaagaaatccttaaagcaaatgttgggagatgaggagaagtgaatgaaagtctcttcctcatgatggcgggagatacctggttctcaggagagggaagactacagagcttgtctgggacttaggctgccttggcctctaggacttgccccatctgttgtgcaaaggatagactgaagaaacagctctactgtttttgaataacctctttactacgacctgttactctcctgtcatggcaacccagagaagcatgggatttttttttccattacatgatgttttccatttaatgggattataatatgaactatgcatttaaaggtagtattctccattttttccaagtctttgtttttttatttttattcatttatttaaaaatacaatttcaatttcattttagattcagggtgtgcatgtgcaggcttgttaccttcgtatatcatgtgatgctgaggtttggtgtatgactgatcccaccacacaagtagtgagcatagtacccaacagttttccaaatctcatccccctccttccctccccactctagcagtccccattgtttgttgttgccatctttaggcccatgaatagctaatgtttagctcccacttataagtgtgaacatgtggcatttgcttttctgttcctgcattaatttgtttagaattattgccaccagcttcatccatgctgctgcagaggacatgattacatcctttttatggcttagtattccacggtgtatatgtaccaattttctttataacaaggaatttttgaacttgtcatttgtggttgtgtagggaattaatcacattgtatggggaatgatgtcttaggcaaaagtcagcttgatagctatcatttacattattttgtggcattgtactaaatgtattacatgtgttatctaagtaaatatgctgaatacaagtccaggaatcttttcacttgattggacttggcttttacggaattttcccagacataagggaaaactttgtggcatagcatttaataatttctggctctgccatcagatgacaagggttcaaatctcacctctgctacttaggaacttgccatcttgtgataattatttaacttatataagcttcaatgtcctcatttataagatggaaataatgtagtatttctttatatttctagtagtactttttgttttttttgatacagagtttcgctctgtcacccaggctagagtgcagtggcaccatctcggctcactgtagcctctgtctccaaggttcaagcgcttctcctgcctcagcctcccgagtaactgggattacaggcatgcgccacacatccaactaatttttgtatttttagtagagatggggtttcaccaggttggacaggctggtctcgaactcctgacctcagatgatctgcctgcctcggcctcccaaagtgctgggattacaggcatgagccgccgcgcccggccagcagcttgctacttctgagtcctgagttcagctggctcagacgccagatcccaatacatatagcagactccattggacctgcactaagagcaccacctgtaggggctgttgcttcaggtggacactttttttcctttcttgcctcagttcccacatctggaggatgaggcaaacatctgaactacaataaagtacatggaaatgcattttcagtctgtaaaatgctgcacaaaggtttactattatttattattgttattctataattccctgagtgttctgatgtctattgcgggttgttttttaagttttgtttttgtttttgttttttttgtcttcctttttcttccccttggatcttctggaagcagagaagtttctctttagtgctggtgaaacaggagagttccctgattcccccctgcaggacatgtgacaagggtgtggcctgcttggtcgccctgcagctcagccccctgtagggggagcatgcagacaggcaggcgtggaggccagcatgagcgcttctgggctccggccccagggcagtgtctagggaccggtgtctgagactcccgaagcccaagtggacgtgtcttactaagctcttttagatattctgtctgcagatggcttgtgtgttaatcagcttagtggatcctctgccttatcccaagggcagtgggccagagtgacaaccttctgcatcccaagtccttgcccagtgtattggaagaatcagatcacatgtgggctcgaaggatgagtgcaagattttattgagtggtggaggtaaacgttttctctgtttcagtcagtcaacaaccgcatgtaattacttaattttaatacaaaactaggatcagagggtaggagctgcggtgtgaagagaactaagagccccttcaaatatatgggaatctaggttttctggagagctaggtactctgctaaaaagaaataaggaagaaataagaataagagatgaggaaggggggctgtgaccacaagagataaataagattatctctagccaatgactctcccatagtcagaggacgcctaaaagcagagttgaatgttgactctcagtctgctgaatacaaatccagggatcttttcacttgattggacttgatttttatggaattttcccaggcataagggaaaactctgtggcatagcatctcatgagatgaatggggagccagagaggggggatggagagggaaggtggtcttcccctgagttgggccacccatgggtgggagaaagactctcctccaaccgcccccagctgaactcccctagggatccagacctccttcttctctctttctctgcctcttgttctgtcattgctgttctgctggttccaacattcagcagcttgcttgcatgtgcccactaaggtctacggtttatatgggggcaggatggggggcatggcgggccaaaaggcaatttttagggtacaaaaacagaattgcctgtcctcacttagggccatgggtcttcagacttgagggtggggccttttccagggaaccaccctcttctacccagaatttccccctcctgtctgtatcactggtgctttataataaggctgctctgagtccttttttcaaattgaagttttgtgagggaaagctatctccagaggtgtctctagtgtgtctcaaactgtgattctgggtaaaattggctcttatttgagctggaagaaaatgtttcactgctaaattaaataatgctcatctttcctcactttataaaatagttaatggatacagttatcccaattttatgttatttttcaatgattttttttcttaaacctcttatgtttgctgctatttatttgttgccttgtagaagctctagctaagcacaaaatgtatgaacaaataacaggcgggaaagattttgaaacagtcagtagggtttctattgactgtattggtgtggttgaccattactttatatggcaaaactcacattttatacaagtaaaattttctttggtttgtgtgattaaattgaacatagtgtttcatcatgttctttttttttgagacagagtctcgctctgttgcccaggcaggagtgcagtggtgcaatctcagctcactgaaacctctgcctcccgggttcaagcaattctcctgcctcggcctcctgagtagctaggattacaggcatggctaatttttgtgtttttactagagacggggtttcaccatgttggtcaggctggtctcgaactcctgaccttgcaatccacctgccttggcctcccagagtgctgggattacaggcgagtaatcctgtatgattataccgccaccattcccggccatcatattcttctaagaacactgttatttgatggtgatccacagaataaatgtgaacattattgctgagagagactttggcttgtctcctgctctggcagtgagatgttgagttttacagtgtttttccaaagcagacttgtgtccagcctcagctgaggtgtacacaggatgctaattgtccctcaacttgtctgcacatcgttctacttatactcacttatgttttgtcaaaatatgatattaatattaaaatatttgttgaaataagttacaaacattttataatctatacaaataattcacatttgaagtagaatattattaagatataacatcttggtatttttcaatgtattctatttaacattcaatttttaatattcaaattattatcattcactactcaatctaaccccgtttataaagttgtataattttacaactgcagggaatattatggatgactttatacaactacttaattttactgctgtggaaagtgaggttcatagtgattaaatgattaagcaatttgcaaaagtattatggctttgtagtttattgatctttccaccacatggtccccagctctgtttaaaaatatttctgagaacttaaagaagtcagctcctcctctgcaccaggcactgacctgttgccaagagactcttgtccaagccaagtagtggtggccaagagagtcttgtccttggagaatatacattctattagggtaggctatcaataaataggtgaacaattgcataatgatgagatttcaggactctgctgccagtgtgaatgtgtgcatggacactggctacccacacctccccttcactttgccatggccaccagtgtgatcatgcacacagagggcagcaggccctgccacaggtgcaaatgcaagcacagacctcggcaaccctgtccccacctatgactcactgctggcatgagcatgtgcaggaatgtcacagccccgcttctgccagtatccccctgccccagccaacatgcatgcatcctgctgtgctgtgccacggctgccagcacaacccgtcggagtgttgtggccagtggaccaggaatacctcggccccctcagcacagtaggttcctagccttgaggggccagagaagaaagctggggtcctggaaccagctccccagggttagagcacacagctcaggagtgctaagctgagtcttgggacccaaaaatcttccagaaacaaccccagggaactgaacccaccttataccacagtcaaacccccaagggcatcaaagaaaataaaagcaagtaaacaaacaaaaaatcatccaaagtatagtgccttcaaagattgaagaaacatcagcccacacagtgagaaagaaccagtgcaagacgtctggcaactcaaaaagccagagtgtcttcttacctgcaaataactgcactagttcctcagtaatggtttttcgctaggctgaaatggctgaaatgtcagaaatagaattcagaatatgattaggaatgaagaacatcaatatccaggaggaagttgaaacccaatccaaggaatctaaggtatccaataaaacaatataggagataaaagacaaaatggccatgtaaagaaataaccaaactgatctgatagagctgaaaaactcgctttgagaattgcagaataccatcacaagtactaacagcagaactgaccaagctgaggaaagagtctcagagcttgaagaatggatctccaaaatagtcagaaaaaaaggaaaaacaataaagaagagtgaacaaatcctccaagaattatgggattatataaaatatgaaatctatgactcactggtgtccctggaagagaaggagaaaaagcaagcaacttggaaagcatattgagaatattgtccatgaaaatttccccaacctcactagacaggccaacattcaaattcaggaaatgcggagaacctctgtgagatattacacaagacaacaattcccaagacacataatcatcagattctccaacgtcgaaatggaagaaaaaatgttaaaggcagctacagagaagtgacaggtaatctaccaagggaaacccatcatgctaagagcagacatttcagcagaaactctacaaaccagaagatactgggggcctatagtcaagaactcttaaagaaaagaaattccaaccaaggatttcatatccagcaaaactaagctttgtatgcaagggagaaataagttccttttcagacaagcaaatgctaaggaaattacttaccaccagactgccttacaagaggtccttagggaagcgccaaatacagaaaggaaagaccattactggccaagaagaagacttaactatctaaagtatatatacactcaacattgaagcacccagattcataaaacaactacttgcttaatgatttattctttctatgaagcacatgatgatgccatcagctgtgaatcagagatgtagggaggtggttgttggaagcttgagaaagaaggtgaaacagccatcctggaaagcagaaacattaaaagacaagggacctacttacacaaggatcaaaagctgtgttgtaagacagtttgtcacaaacgcacgagtgctgtttcttctgcaacagtcagctacatgggtacaactgaaggaaaaaaaggtaggtagaaaatttggccagaattgagatattactatgcaaatatgacagaaagagagagggagttaataaaggtaagagtgtttgtaaggaagcagttatagttctggcccatggaatctggcctgggaaagaagggaagttagtacaataaaagactgagttcaagaaactgagaggctggtatgctggacagaaccactctgtgaatatcgatatttccaaaaagacacaggaggagtggtggaagaagagacagttgattgtagtcatcaagggatgaatggtgtgacttgcagaggacattggatgacagcagcaagcagctgtgttcttagtacaagcttttcaaaggagctcgggtttgagagaaggagagaaattggagaggggagcagggaggatgtcaatctacctccacttcttacaaagagatggtagaaactttcagagagtttgttgctttctaaaggtcttttattgtggaggggtttgagagaaggtgtgggatggggttagactggggctgtgcagagcagtgtgaggatgagggccttatgatggtgagttaccccagaggattatattgtggatggtgatttagtatatggggtagtgagcaagataggaatggtactctggttggagagtaagcagtgatttcccatggcttgagggttttctagatcacattaggcagaggaaaacctcagaaaaggtgatttcaatccagttgagggttgatttatttgaggaaaagatgcttaaagatctccaggggattactttcacccaatactagatgttttaccatgtgatcattacaagagtaatacaagctattctaatttcagattagactagacattatatttttctttctggcagattataatttatgattacaacaattcctgtgacctgtatgaagacacatatttctaatttcagcttgagagaaatttagtcttttattggatgatttaagtagatgattagtcagagggaaaattggtaatatagaggacagagtgtattataagatgcaacagaaacttttctccaatatatgcccagataaaagtacaccagagtaggaacagagtttagggcatatgtggtttaaaatttccacccataaaagagtttcgtccacaccacatctttccaatctcatcagcatgtgatttatacatcctcaaaaggttgcccattaaatttttgctcattacttattttaaaaacgtttttgtcggtactgagataaattctggcttcccagaggtctacctataagctttaatttttcatgttaaaccatctctctgatactgtttcgctttttagataatggctactattttcttaccagtcatccagattaaaactccttattcctctaaaacatctgtctcatgacatgtagctaaaaaagaatatatataatatatatatttatttgaattctaccatcttgtggttatttaagtagttgtctataaaatcctatatatgtattttgcagccttaagccatgcatgttattactcgttatgccagacagcttgacagctgaaattagtccaagggtccagggctctactatagcttggagatgggaagttctctgctcagagtgaaaattggtcaggaatgcttgcttgggaaaacgtgcaggtgttgtttagaaacaacatttttctaattttttttccaggttactctttctctcccttgtttccattttttctgatttccacatttttaatactgtttttatatagatcctgtgctggtttctttgctttaattaatatttcattttcaaataagctcaaacttggagaaaagttgcaagtatagtaacaaattccagtattctctttatccatttttcccagttatctcctgtaaattacaaattgttcactatatttactttatcacttttctctttttctctcctcctttcccccctctctatctcacaaacattcgacagtttgtatacaatgtgtttcgttatcacaaaaatactttagtatgtatttcctaaaaagacaagaacgtattctcacataactacaacatcatcatccaaaccaggaaattaacatgcatacactaccaccacacaatgcacagacaccaggcacatttcaccaattgcaccaataatgtccaatataggaagcacaaacaacagcaacacaaaagaaattagtctgcgagcacaattgcatgtcgcatttaattgtttcattgttttattttgcttgttttttactcccctctattctggaactattccttggtttctttttttctttcataattttgacatttgaagattactagtcagttatttgtagaatatccctcatacttgagggttacctaatatgtcctcgtgattgcactgaggttatgcatttttgagggaaataacacaggcacgatgttgagcccatctcagtgctccctatcaggagccacacaatgtccatctgtccattatcgctgatgttctgtggggttacttggttgaggtggtgtcttccaggtttctaaagctactattttgtctcaacataaataataaatatactgtgagaagatactttgaagttatgcagcatcccatttgttagttgtggcatccattgatgattcctgcctgaatcaattattactctcaaatttgctaaacagtgattttgtatctccttattccttttgcctttgttattttgcttgggaagaacttgtctttctttgctatttatttattgagtgatgtttggtaatagccacttcctgaggcctctaaacgtgccacttctctgagatcccacatattctcatgatctagcaactaaaattctaactacaggaaatcagggggcttccaggatttgtttccactgatgtccattttgactggtcagtgcctgtgctgtggcttaaatgtgtgatgttagaatgtgattcctgttccatccaggaagtagactggtgaagaacatttgaacttctaattcaggggtcctcagtcttaatagcatattggaaatgcctggagaattcaataacctacgaatgcctgggtggtagctcatcatcaggtgatactaatatgctgtcaagattgacagccactgggttagtgtagttgagagctttcctgttcattgtcagctaactaagtagtagcagggacttagatactgatggaaaccctagcaaacagggagaagaaactaaattattcgactcatgaaggtatggagagggagggaggaagagagaaagaggagaaggagaaggaggaggagaaataagagaaaagga

SEQ ID NO: 4 - DNA, 16439 bp, Homo sapiens
  source 1..16439: mol_type = other DNA
tgaactccccagtaggacccttcccacgtgcaaacagcattcagtgtgagcctctctatccccacttaccctcccttcattcctgcctccttcccctcaacgctcacctgatagacctggtcattcctcaacagagcttgtgtgtcggcagctagaagaaccagagagagacatcaatggcctagcagatgggactgtgagatccaccctcccacaccctcagaagtctgcatgagtgatatgaacacacaagttctttcaacagtcaaagttctaggagtctttaggagattgcaagagtaactcccagctgagactaaacctaccaccagccccattccttagctggtgcataagctcactggtacacacacacacacacacacacacacacacacaaacacactctcatgctctgctcttccactaacccccagacagccttccagtctcatgtccagcaaagcagaagactcccaaagcaaggagcagagtggcaatgacatcagtgacaatgatgccagccacggtggctggatccagctccacacagctctggcacactgtgggggaagggaggagagaggagaggttgagagcctttaagatcagggaaccatcctctgcctcctagggcaacccttagatctcttatgccaaaacccaaacttcaataagaccctgggagaaaggcctggtgatgggcttgccacaccttcctccaccctgcatcctaaggaatccctgggaagagccaagaagtaatctcagcttttgggaccttgaacaaggtggtgggccaaacctctcacccaaccaaggctgcagtaacatgacccctactgctctgtcttgctgagttaagctctctcttccactttgaaggtccccaaatctggcttgtaccattacaatagtgacctcactttgtttagagaacagatgggttcaccatatccctctctagccagaaagttctcacatccagaagccctatccattccaacccaaagggttcaggaagcacgtacttcgataatgaacttgcacggtagattctttgtccttgtatatatctgtcccattacacctatatattcctcgtgggtccaggatgcgttttcccaggtccagtcttgtaatgtctgagagcagtgttcccaccgttccctctacccatgtgatgctggtattgcaattcacaaacactctgtcctcaagttcctctataggtatcttgaaggggctcactaaaggggaaaaaatatcacagttggagacagctctttgatctgcaccaagccctttgttctgcggaagctcatacttaacagagaccattttcctggtccaggacagtttatggcttccatcaagagagacagaagtcacaagaaaaagccttcagaaagttccccaccaactgcaggggtcaagggggacatgaggatgccattcaagcagaggacaggtcttggggccttggtgcaaaagaggacccctcagagcaggattgacccaagcaccttcctggaaatgaatccagaccactgatgaggagtagggggagcacggaccactgaagcacctggaagatgtggaaagacagaagaacattcctcgattggaaatgtctgcattttttcttcaaggaaacatctaattccacttcccagccatctacaacactcccacttcagcttcttatcctcatccttcctcattgcccctgctccattgacaaccaagaaagcggggctctcaacactgaagcctttcccagggccagggatggctgtgggtggagaccagctggtttaccagcccctgaattatcagccaagtggtccagaacgggaccagggcaaatcccatgtacagttttccacccttggttagaaggaggagaacaggaaaaaaattttattgaatccatccctagagctcctcacaagtcaagtcttgtgggagacttttagggctggaggtgagtgcagcaacattccagatgcagtgagttcctctgacagcctgagcacatctccacaggccacagaggcactacagtctatgcctccaaacacagggaaaagtggaggctacattcattcatcctgggcttcacactaagtcccaaatttggatacaagagcatcttctagaaaaccctgaaacagctgttgctcacacttctgaagcaggttggaagtatatgcatgtatcctcagggagacacatgcacatcaaatgcttcacgtcctacagtcgcgtcctcttcagggatctgtctccagtggaaatcctgagtgccctagtgcagccaactattaggtgaccattggacccagtttgcttagtgttgaaggggttcctcggacatgggactttccattttaaaactgaaattggcaaactgagatgagttaaaatcctaccatgtaacaacccctcaaatcttccctccgtcctgctcaacctaaagttaacttctcttaaagcattcacataagtgctaggacatgcctccagggatgacataatcatggccaaacaaacaagagtcctgattccagaggccatcaggcctaaaaggagtagtgcaggaagctgtgctcccatggccagtcccagattcaggtacatacgtactgagctattttctgcagatctctggcctaaggccttctgagagacattctaggcccacatgcacccatggctggagtcagtcaaagccaagagcctgtttcccagactctatgctacatcctgcccctgccctcctgacacccctggggtgcctggtgaactgaagctagcaccgagaagcacttttttttttttttgagataaggtctcacaggttgcctaggttggagggcagtggcatgatcacagctcactgaagccttgaaatcccgggctcaagtgaccctcctgcctcagcctctcaaatagctgggactacagttgtgcaccaccatgcctggctaattcttttgttttttgtaaagatagagtctcatcatgttgctcagactggtctcaaactcctggcctcaaaggatcctcccacttcggcctcccaaagctctgggattactggtgtgagccaccgtgcctggcaagaaacactttcaagtgggcctcactcccatcagtaatgtccctctcaggtcccttcccccacccacctggagtagccttaccttgcgagagaagggtagccagtaccaggccagagagaaacgtgctatgttccatctcccagcggaactcatccagtagataaagccaggtcaccgaactatcagcctgggtgagagctgccctcccctagctgactcacaggtaccggaaagaggagagtgggggaggaactagaagatgtctgcttctctgctttctgccgttgatggtgggaaattttcagagtgggggtgggctaggggtagggtaggaaggaagcggtgtaaattgctctatctctgagcagggagctggcagagaatatggaaaaggtggcctgaactttagcgtccctattgacaatgcaaccacatttacacacaacctaaacactgccacatctcgaagccccttgagagaagccgtcggccccatagcgcaagccgtagcagctagatttctcatggaggctgatctttctcaggacccttcactaggcagccagggacaccagatctagcagcttcttgtcagtgggaggttgggctttagagaccccagccagagatttgaatcctgggtccaatactgcctacctgtggggcctgggccagccataaaatttttcagagtcttattccattagtaccattattaggattcaaacaagatatttgcatggtgcctcacgcatcatatgtgctcattaaggggtagttattaataataatataattgactgacaggcaatattgagcctcccggtgagacaaatggacctttttcccctgtggcctacgaggatctgaaactcttcacgctgctgcagttagactgtcacttacctggggacagagtcatgcctgtcttgctcactgctgtatcttgtgcctggcacataacgggagctctgcacatttttgttggctcactgactgactggctgagggagataggggcctgagatcctggacattcagtccgggctctggcccctgaaaatgtgctggcctgtcctcggaattgttccacctattgccttccaggcgcctctttcatgatctcaaaagaatagtgaaaccaggtgccgtgtctcacgcctgtaatcccaacactttgggaggctgaggcaggtggatcacaaggtcaggagttcgagaccagcctgaccaacaaggtgaaaccccgtctctactaaaaatacaaaaattagccgggcttgctggcacgcacctgtaatctcagctactcaggaggctgaggcaggagaatcgcttgaaccccggaggtggaggttgcagtgagctgagatagcgccactgcactccagcctgggcaacaaagtgagactctgtctcaaaaaagaaaaagaaaaaaaaagtgaaaaaaattcctgaatgaaggcctggactgaggtggctttccatttggaggtccagccccaagcatctgagagtccctcctaaattcattacctacagcaacaacaactagcaacaagtaacaactggctacgatcctaacaactaatgacagggacatttatctccctcatgaagaaacggtcccaggaccatctcccacccagcatccattgcggttccctgtgcaagatgagtctctgagtgggaatccagcactctctccctcttcttccccaccaccttcaccctccttaacggaaaaacaaaaggcatctgcacctgcagccctgctgaggcccctgctgctcacacttgcagcagagggtggaggctctgggttcttgccttctctcaaaggccccagccccaacagtgatgggtggagccagtctagctgctgcacaggctggctggctggctggctgctaagggctgctccacgcttttgccggaggacagagactgacatggaacaggggaagggcctggctgtcctcatcctggctatcattcttcttcaaggtaagggcctactaggggtctggaagcctggggaagggctcaagggaagagcccatcactagtgagacaggaatattggtatccctaaccttcagcctacctctgctgtcaccttagagttcaaagaagggcaaaatggaggctcttaactgttctctgctagagagaaacagtgtcccatggaggagaaggaatccttgtctctgaaaaatgcaaacagagtacttaaatggctgaagagaggaccctgttaccgccatcttagatttgaatgcagcccaaaagggcataggccaagaaactaaaaggaaaaagtatatgttccctacttcagagctgggggctagcagtcgacctaggaaatgtccattcactcagttgggcagttggccaattggtcaacaaagatgggtgagaccctaatatgtgatatgccatgaggaaataaaaatgaataaaacacttatgctctacagaactcatagtctcattggagagattagttttcatggtgccagactgagtgtacagtatagtatgggtgcctgcatgaatactccattggactatccaataggaagtggatgaactgggttgatgcctggagtctggacatcctgggaaagttaaaaccatggaatttgctgaagtcacacaagtactatgtgaggaagagggagctgaagatggaacagtgagggcaccaattttaaggggcagttagagaaaaaaaaacattccaggaaagagataaaaggagaatcaaagagctgaaataatcaaatgtatgcactaccttggaaaccaagagagtagagagcttctagaagaagagaattaccaagagtataaaatgttaaagagagcccactaatataagaatatatttaaaagtttaagtctgcgggatgtggcagaatatgcatcatgggtgatttagcacagtttcagcagggtattgtgagtggaagccaagtgaaaattggtagaaggtgaaggtcaggtgatgaggtcagcaagtgtagactattctttaaagacatttggatgaggagggagaaaagactgggtggtaggtagaggaggaagtagagtctaagaataaatgtttttgagggtgagagaaacttgagcacatttctaggctaaaggagaagatgatgagtagagcaaggtcctggagttgatggtggaaggacagagtcaagagtggggtcaagagcacagagaggaaggcaacgttaccctctaagaccaggagaaaggaagtgcaaacggcactggccacaaataggtttataaagggaaaggaagctaaaggagaaaggacaataacaacttccgtgttctaaaaaatccaggtgggaggcaatgccacctgctgaaactgagctgggtgatgaccggttgggagacttgaagaggacagtgagagtttagaatagaattatttctgagagaaatgcaaggaaaagtcaggtaaagacaacacaggttgaggagcaactctcaagcctgggtgaggtggctgcatatcatggaaagagcatttgagttcagcagtagaaaacctgagctctggatccagcttggacaccaatgagctatgtgtacttgaacaactcctccactgtctgggagttctcatgccagccaatccagaaatgtggcagggacgggcatggtggctcatgcctgtaatcccagcactttgggaggtcgaggcaggtggatcacctgaggtcaggagttcgagaccatcctggtcaacatggtgaaactctatctctactaaaaatattttaaaaattagtggggtgtggtggtgggtgcctgtaatcccagctactcaggaggctgaggcaggagaattgcttgaacccaggagatggaggttgcagtgagccaacacggtgccactgcactccagcctgggcgacagagtgagactccgtctcaaaaaacaaaacaaaaaaaaaagaaatgtggctatatgaactcttaagtcctaaaaggaaacctcactcagatatgcaacattaaaagatgtcaatcagccaggcacagtggctcatggctataatcccaacactttgggaggctagggagggaggatcatttgagcccaggagttcaagaccaacctgggcaaaatagggataccctgtgtctacaaaaactacgaaaattagccaggcatgttggcatgcacttatgccagctacttgggaggctgaagtgggaggattgcttgagcccaggagcttgaggctgcagtgagccatgatcacaccactgcacttcagcctgagtgacagagcaagaaaaaaaaaaaaagatgtctcaaaaaaaaaaaaaaaaactatgtcaatgatcagtgcaatctccttccctgaatccaatttagaatgaggggtctctgtcttctctcggtcttctgaccaggtggtcaggagaaaaactagttagaaacctctttaactgtttgtcttgccttttctcccatttgctcatttattcatttgcacattcattcaacaaataaagttcctaccatgttccagacactgcactggatactgttcaaaaataatcccgccctgcatgaggttctaagacagttcacttcttgtatttccattatactttgactcatgaccgcctctccctatcacacacactccacctctagccacactacctctcttgtccttttccaaagctgcatcttattcagctattctttctactcagaatgactttctctatctggtatattcttgcttactcttcaaggcccagacaaatgtcgtttcctctattaagctttccctgatcctcacccttggacaaaattaatagcttctacccttttttcatatgcagtcatataagcactaaaagataatgtatacattctattaaattaattgcatatatgttttaattttttcacttcattcataaactttggaagcagcagttgattcttcatatcagcactcttttctttcataatatctaattattttaggtcataatagtcctcgttaaatgtttggattaaatctaattgaattgaattcaagtgccagatctctgtaatatgtctaccatgcaactctactaccctaaggttttgtttgtctgtttgtttgtttttatttttttgagacagctttaacacctaggctggagtgcagtggtgcaatcttggctcactgcaacctccacctcccaggctcaaccattcctcccacctcctgagtagctgggactacaggtgcataccaccatgcccggctaatttttttgtattgtctgtagagacagggtttcgccatgttgtccaggctggtgtctaactcctgggctcaggcaatctgcccacctcagcatcccaaagtgctgggattacaggtgtgagccactgctgccagtcaactctgagttatgagacagatttgaggactaattgaaaagctaacttccctgggacatccagggtttttataataaaaggatcaccaaggctgaatattttataaggaactcagcctaaaggttttggatagtgcacacccactccttgccagggcattagctgctcaagaagcagagtgttctggactggataggcaatatccttacttacatatactacaatacaattctgatgctaaccccccaaattagtgtcagaccctacaggttaagagcatggttcccaaccagattgccctcactttaaatgccagctacaagttcagggtaccccagaccactcacatttctgacaaactgcctgcaatcttaaggattcccatgaccctcctcattgataatttgctagaataactcacagaatttaagaaagtgctgtacttcctatttcagttttattaaaaaataaaataaaatcagaaccagctaaatgaaaagggcaaggtctaggagggtcccgaacacagagcctccgtgccctctccccgtggaattagaacacatcgcccccagcactctcccacactctgccagcacattgatgggttcaccaaccaggaagctccaccaagtttttattgaggtatctttacataggcatgattgattgaatcattggccgcttgactgatctcaatctctaggatcccttcctggggctgataccactagtttcaaagctgcaatcctcttaccatatggttggtcttgaccagcaccatcctgagtcattgccatgcataaactcaggtatggtctaaggatccaccacagataacaaagccactcctgtcactcatgaaattccaacggttagaaacaccctcccaggataccaggacaaagatgagacaaattgtttattatcaccttgaattcaattgagtgatttagtctacaatccggaaaactaagtatagatactaccattttcatggatttggatctttcttcatcttggcctcaaataaccatggaaatacttcagggcatctgaacaactccatgcccagctaatactctatctctcttctgtctttacaggtactttggcccagtcaatcaaaggtaggagaaatggcttctttctatactcagactcagaatattgacggaaatttggcttcctacaacagtagtcctacaggagcgaacagtttagaatgaatgaaatgacggggatagagaggtgatgtctctattgtcaaccaaatcagtgacctgacataacctgttccgggcagcttgcctgtagctaagcatttaactggtctcttacaggagaagcaggaccctagtagctagggacacatctcaaactgtgacccatgaaccagtaggtattggcgtcacctgggatcctgacgttagtctctgtcaatccttctctttagttcatctattctacccaaagtgatctcatcatctggtatgctgttagcagtttcttacctgtatagtatcttccaaataacatgccccaaaatcccaaagttttacccctactaattacagcaatgtctcttttattcttcaccccctgacgcagatattggcgtcacccgagagcatgttagtaatgcagaatctcccctccccagaactactaaatagcacctgaaattttaacaagatccccatgtgattcatgtgcacatcaaagtttgagaaacactactctaatgatctcctggtatgcagaagcagggagaatttcagaggcaagatccttaatagaaccacggcttttctcatttcaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgagggatgtatcagtgtaaaggatcacagaacaagtcaaaaccactccaagtgtattacagaagtatgtaatcccctttggtctgtttgttgtgaaattaatcagtatttgctgttctggtgagctttttatctggggtgaaagtggaaatagatcctcaacagtaatattatcgcctgttctcttaatttcagcttgcctcttttaaaatactgtaagatacttccctcaccctattgaaaaactacagccagtcctgtaaaattttgtttacctttgggtgggctccatggattcaagcaattcagcactgagttgaatgaaggggttggggagacagaggtgatcataaggtgagcagcagtgagaactggagcgcagtggagcatgaagagtatctagtcttccttctgttccagatacttcctggtagttagactacatgggcttccccaggaatcctgggggacttaagagcatcaagatgcattgagttttggcgcaaggatcttcccttgccctgccacccacagaggaaaagcctgctgccctccacagcggcattattgcagacaggcaggagaaaacgaaccaggaaaaacaactttcgcaacctgaaggtttgtctctccttttccctacagtgtgtcagaactgcattgaactaaatgcagccaccatatctggctttctctttgctgaaatcgtcagcattttcgtccttgctgttggggtctacttcattgctggacaggatggagttcgccagtcgagaggtaaaagaatgctcttagatgagagatgggaccacctgagaccctcagctttcctcctaccattatgtacccaatggaagagactgagttggtgcttcttgctagtgtgcatagttgggtgaggctgtatttctctgagacaggagaaaggatacttggtgttatcacagcatgttcacctgtctcaagatacagctccctctgtgaaaaaaaaaaaaaacaaaaacaggcgcagtggctcacgcctgtaatcacaacactttgggaggccgaggcgggcagatcacgaggtcaggagatcgagaccatcctggctagcacggtgaaaccccgtctttactaaaaatacaaaaaattagccgggcgtggtggcgggtgcctgtagtcccagctactctggaggctgaggcaggagaatggcatgaacccgggaggcggagcttgcgtgagccgagatcgtgccactgcactccagcctgggcgacagagcaagactccgtctcaaaaaaaaaaaaaaaaaaaactattttaagtaaacccttacatacagaaaagtgaatcacaagcatacagctcaaaaaaaaaattcacagagcaaatacacccatgcagccagcacataatttaagaaagacaatattaccaatgccattgaagcccctcttgtggtcacttccagccactacccatcctcaagagtgagcattgtcctgacttctaacaacaaggattaactttgcttgtttttgtcctttatgtaaatggactcatatactaagtattcttgtgagtagcttctttcactcaacaacatgtttatgtgctttatccatactcttgtgtatatataaaactcttattgttatttagtattccattttgtgaatataacccaatttattatgcattctaccttgtgtggattctgtttcttttttgtgcagcttcagacaagcagactctgttgcccaatgaccagctctaccaggtaaggggatgaagaataaaagagacattgctgtaattagtgggggtaaatctttgggattgaggggcatgttatttggaagatcctatacaggtaagaaactgctaacagcataccagatgatgagatcagtttgggtagaataaatgaactaaagagaaggattgacagagactgaggtcaggatctagtgagcacaagttgaagaacacactgagagggacacacgaagaaactctcgacaggctgggcactcactatagacaggccatggctcatgcctgtaatcccagcactttgggaagccaaaacaggtgaatcacctgaggtcaggaattcgagacaagcctggccaacatgatgaaactctgtctctactaaaaatacaaaaaaaattaaccaggcgtggaggcgcgcgcctgtagtcccagctactcaggaggctgaggcaggaggaccacttgaacccaggaggtcgaggttgcagtgagctgcgattgtgccactgcactccagcctgggcaacagagaaagactccgtctcaaaaaaaaaaaagagagagagagaaaaagaaaaaagacagagcctccatctccttgtcctctttccatcctcaggaccatgaagtacccactccaaattctcacatataaaaaacattcaataaacatgcatcaaattaattaatagaggatggaaaaaatgacttatgactgtgctgtcctttccagcccctcaaggatcgagaagatgaccagtacagccaccttcaaggaaaccagttgaggaggaattgaactcaggactcagagtaggtgggttcttcaatgccaattctaataaaggacccttgcatcaactgccctcgcaattgcttctaagtctagctcccttccctaagcggctataagcatcagactctggggatcagggattgggacgtggtttggggtactcttttctaaaaattctggggccatactgattgtcttggcctaggtaaatatgaattttatgtatctgtaaatcctgtcagagcagggcctcaagccatagagatgctgaatattaatcttaacctacatttgaatttctcattatctacactattaacattttgggctaattaattatttgtgatgaggggctagcctgtgcattgtaggagttatggaagcatccctggcctctctccaccagatgctggtagattgtccagtgtgacaatcaaaaatgtgtccagacattaccaaatgtgtccaaacatcacctccagggcaaaatcacccttagttaagaaccactaacccatattaaccttccaatcaataaatcaatcagtcagaagttatgatttaattaatctatctgaagtttctatcaggaagacagggttgaaagcattatttgttttttttgaacaaattgcaatttttcttttttcagtccaggtgttctcctcctattcagttcccagaatcaaagcaatgcattttggaaagctcctagcagagagactttcagccctaaatctagactcaaggttcccagagatgacaaatggagaagaaaggccatcagagcaaatttgggggtttctcaaataaaataaaaataaaaacaaatactgtgtttcagaagcgccacctattggggaaaattgtaaaagaaaaatgaaaagatcaaataaccccctggatttgaatataattttttgtgttgtaatttttatttcgtttttgtataggttataattcacatggctcaaatattcagtgaaagctctccctccaccgccatcccctgctacccagtgaccctgttgccctcttcagagacaaattagtttctcttttttttttttttttttttttttttgagacagtctggctctgtcacccaggctgaaatgcagtggcaccatctcggctcactgcaacctctgcctcctgggttcaagcgattctcctgcctcagcctcccgggcagctgggattacaggcacacactaccacacctggctaatttttgtatttttagtagagacagggttttgctctgttggccaagctggtctcgaactcctgacctcaagtgatccgcccgcctcagcctcccaaagtgctgggattacaggtgtgagccaccatgcctggtcttaaaaccagtttcttatatatctctctggaggtattctaggcatatatgagcacattctcaagtacatattatcctcccttcccctatcttttagacaaatgatatcaaactatacatcttgtgagattattgcataccattatatgaagataccattatatcctttttaatgcaaccatattgtacaaatagactatgatttatttaacctgttatctatcagtggatatttaagttggtagttggttccaatcttttgctcttacaacaattctgcaatgactaacattgtataaatatcatttttaaaaataattgcattgaagcataatgtacatgccataaaatccacccatcttaagtgatttcacctgttctcagaaatttttagtaaatttaactaattgtacagccattaccataatccagctttaggacattttcttttttttcttttcttttcttttttttcttttttttttttttttgaagtggaatcttgctctgtggcccaggctggagtgcagtggcgcgatctcagctcactgcaacctccacctcctgggttcaagcgattctcttgccttggcctcccgagtagctgagactacaggcacatgccaccacgcccagctcattttttgtgtatttagtatttgtgtatctagtatttgtgtacttagtagagacagggtttcaccatgttggccaggctggtctccaattcctgacctcaggcgatccacccgccttgacctcccaaagtgctgggattacaggtgtgagccaccgcgccaggcccgtaactgtattttaatatagccattctatggatttaatatggtattttattatggccttaatttgcatttccctagatactaaccatgctgagtgtcctgtcttgtgtttattaaccattcatatatttttagtgaaatgtgtatcaaatcttttgcccatttttaagttgacttatttgtttgtcttcttactattgggttgcatatgtttttgatataagtcctttatcagatatatgatttggaaatattttctaccaatctgtggtttgtttttcttaatggtgtcttttgaagtgcaaaaggtttgaattttgaagtacattttattgattttttcttctatatattgtgcttttggtatcatgtctaataaatctttaccaaacccacagttacaaagattttctcctgtcttctttttatactttttacagctttatggttttagctctaacaataaatgtgattttgaacatacataagactatttgtaacaaacacaaataaattgaattgttgggcacttgtatttgcatttgtaattttattaactattgccaaatagcagttgtaaacacgtacacacacacaagtgtatatacacatatacatatatatacatatacatgtatgcatatatgcatatatacatatatgcatatatacacatatatacatatatacacatatacatatatgcatatatacacatatatacacatatacatatatgcatatatacacatatatacatatatacacatatacatatatgtatatatacacatatacgtatatgtgtatatatacacatatacgtatatgtatatatacatatatgtatatgtatatatacacatatacacatgtatatatacacatatacacatgtatatatacacatatgtatatatacacatatacacatatgtatatatacacatatacacatatatacacatatgtatatatacacatatacatatatgtatatatacacatatgtatatatacacatatacatatatgtatatatacacatatacatatatgtatatatacacatatacatatatgtatatatacacatatacacatatacacatacatatatgtgtatctacacatatacacatatacacat

SEQ ID NO: 5 - DNA, 10671 bp, Homo sapiens
  source 1..10671: mol_type = other DNA
caggaggtgggaggaatggttgagcctgggaggtggaggttgcagtgagccaagattgcaccactgcactccagcctaggtgttaaagctgtctcaaaaaaataaaaacaaacaaacagacaaacaaaaccttagggtagtagagttgcatggtagacatattacagagatctggcacttgaattcaattcaattagatttaatccaaacatttaacgaggactattatgacctaaaataattagatattatgaaagaaaagagtgctgatatgaagaatcaactgctgcttccaaagtttatgaatgaagtgaaaaaattaaaacatatatgcaattaatttaatagaatgtatacattatcttttagtgcttatatgactgcatatgaaaaaagggtagaagctattaattttgtccaagggtgaggatcagggaaagcttaatagaggaaacgacatttgtctgggccttgaagagtaagcaagaatataccagatagagaaagtcattctgagtagaaagaatagctgaataagatgcagctttggaaaaggacaagagaggtagtgtggctagaggtggagtgtgtgtgatagggagaggcggtcatgagtcaaagtataatggaaatacaagaagtgaactgtcttagaacctcatgcagggcgggattatttttgaacagtatccagtgcagtgtctggaacatggtaggaactttatttgttgaatgaatgtgcaaatgaataaatgagcaaatgggagaaaaggcaagacaaacagttaaagaggtttctaactagtttttctcctgaccacctggtcagaagaccgagagaagacagagacccctcattctaaattggattcagggaaggagattgcactgatcattgacatagtttttttttttttttttgagacatctttttttttttttcttgctctgtcactcaggctgaagtgcagtggtgtgatcatggctcactgcagcctcaagctcctgggctcaagcaatcctcccacttcagcctcccaagtagctggcataagtgcatgccaacatgcctggctaattttcgtagtttttgtagacacagggtatccctattttgcccaggttggtcttgaactcctgggctcaaatgatcctccctccctagcctcccaaagtgttgggattatagccatgagccactgtgcctggctgattgacatcttttaatgttgcatatctgagtgaggtttccttttaggacttaagagttcatatagccacatttcttttttttttgttttgttttttgagacggagtctcactctgtcgcccaggctggagtgcagtggcaccgtgttggctcactgcaacctccatctcctgggttcaagcaattctcctgcctcagcctcctgagtagctgggattacaggcacccaccaccacaccccactaatttttaaaatatttttagtagagatagagtttcaccatgttgaccaggatggtctcgaactcctgacctcaggtgatccacctgcctcgacctcccaaagtgctgggattacaggcatgagccaccatgcccgtccctgccacatttctggattggctggcatgagaactcccagacagtggaggagttgttcaagtacacatagctcattggtgtccaagctggatccagagctcaggttttctactgctgaactcaaatgctctttccatgatatgcagccacctcacccaggcttgagagttgctcctcaacctgtgttgtctttacctgacttttccttgcatttctctcagaaataattctattctaaactctcactgtcctcttcaagtctcccaaccggtcatcacccagctcagtttcagcaggtggcattgcctcccacctggattttttagaacacggaagttgttattgtcctttctcctttagcttcctttccctttataaacctatttgtggccagtgccgtttgcacttcctttctcctggtcttagagggtaacgttgccttcctctctgtgctcttgaccccactcttgactctgtccttccaccatcaactccaggaccttgctctactcatcatcttctcctttagcctagaaatgtgctcaagtttctctcaccctcaaaaacatttattcttagactctacttcctcctctacctaccacccagtcttttctccctcctcatccaaatgtctttaaagaatagtctacacttgctgacctcatcacctgaccttcaccttctaccaattttcacttggcttccactcacaataccctgctgaaactgtgctaaatcacccatgatgcatattctgccacatcccgcagacttaaacttttaaatatattcttatattagtgggctctctttaacattttatactcttggtaattctcttcttctagaagctctctactctcttggtttccaaggtagtgcatacatttgattatttcagctctttgattctccttttatctctttcctggaatgtttttttttctctaactgccccttaaaattggtgccctcactgttccatcttcagctccctcttcctcacatagtacttgtgtgacttcagcaaattccatggttttaactttcccaggatgtccagactccaggcatcaacccagttcatccacttcctattggatagtccaatggagtattcatgcaggcacccatactatactgtacactcagtctggcaccatgaaaactaatctctccaatgagactatgagttctgtagagcataagtgttttattcatttttatttcctcatggcatatcacatattagggtctcacccatctttgttgaccaattggccaactgcccaactgagtgaatggacatttcctaggtcgactgctagcccccagctctgaagtagggaacatatactttttccttttagtttcttggcctatgcccttttgggctgcattcaaatctaagatggcggtaacagggtcctctcttcagccatttaagtactctgtttgcatttttcagagacaaggattccttctcctccatgggacactgtttctctctagcagagaacagttaagagcctccattttgcccttctttgaactctaaggtgacagcagaggtaggctgaaggttagggataccaatattcctgtctcactagtgatgggctcttcccttgagcccttccccaggcttccagacccctagtaggcccttaccttgaagaagaatgatagccaggatgaggacagccaggcccttcccctgttccatgtcagtctctgtcctccggcaaaagcgtggagcagcccttagcagccagccagccagccagcctgtgcagcagctagactggctccacccatcactgttggggctggggcctttgagagaaggcaagaacccagagcctccaccctctgctgcaagtgtgagcagcaggggcctcagcagggctgcaggtgcagatgccttttgtttttccgttaaggagggtgaaggtggtggggaagaagagggagagagtgctggattcccactcagagactcatcttgcacagggaaccgcaatggatgctgggtgggagatggtcctgggaccgtttcttcatgagggagataaatgtccctgtcattagttgttaggatcgtagccagttgttacttgttgctagttgttgttgctgtaggtaatgaatttaggagggactctcagatgcttggggctggacctccaaatggaaagccacctcagtccaggccttcattcaggaatttttttcactttttttttctttttcttttttgagacagagtctcactttgttgcccaggctggagtgcagtggcgctatctcagctcactgcaacctccacctccggggttcaagcgattctcctgcctcagcctcctgagtagctgagattacaggtgcgtgccagcaagcccggctaatttttgtatttttagtagagacggggtttcaccttgttggtcaggctggtctcgaactcctgaccttgtgatccacctgcctcagcctcccaaagtgttgggattacaggcgtgagacacggcacctggtttcactattcttttgagatcatgaaagaggcgcctggaaggcaataggtggaacaattccgaggacaggccagcacattttcaggggccagagcccggactgaatgtccaggatctcaggcccctatctccctcagccagtcagtcagtgagccaacaaaaatgtgcagagctcccgttatgtgccaggcacaagatacagcagtgagcaagacaggcatgactctgtccccaggtaagtgacagtctaactgcagcagcgtgaagagtttcagatcctcgtaggccacaggggaaaaaggtccatttgtctcaccgggaggctcaatattgcctgtcagtcaattatattattattaataactaccccttaatgagcacatatgatgcgtgaggcaccatgcaaatatcttgtttgaatcctaataatggtactaatggaataagactctgaaaaattttatggctggcccaggccccacaggtaggcagtattggacccaggattcaaatctctggctggggtctctaaagcccaacctcccactgacaagaagctgctagatctggtgtccctggctgcctagtgaagggtcctgagaaagatcagcctccatgagaaatctagctgctacggcttgcgctatggggccgacggcttctctcaaggggcttcgagatgtggcagtgtttaggttgtgtgtaaatgtggttgcattgtcaatagggacgctaaagttcaggccaccttttccatattctctgccagctccctgctcagagatagagcaatttacaccgcttccttcctaccctacccctagcccacccccactctgaaaatttcccaccatcaacggcagaaagcagagaagcagacatcttctagttcctcccccactctcctctttccggtacctgtgagtcagctaggggagggcagctctcacccaggctgatagttcggtgacctggctttatctactggatgagttccgctgggagatggaacatagcacgtttctctctggcctggtactggctacccttctctcgcaaggtaaggctactccaggtgggtgggggaagggacctgagagggacattactgatgggagtgaggcccacttgaaagtgtttcttgccaggcacggtggctcacaccagtaatcccagagctttgggaggccgaagtgggaggatcctttgaggccaggagtttgagaccagtctgagcaacatgatgagactctatctttacaaaaaacaaaagaattagccaggcatggtggtgcacaactgtagtcccagctatttgagaggctgaggcaggagggtcacttgagcccgggatttcaaggcttcagtgagctgtgatcatgccactgccctccaacctaggcaacctgtgagaccttatctcaaaaaaaaaaaaaagtgcttctcggtgctagcttcagttcaccaggcaccccaggggtgtcaggagggcaggggcaggatgtagcatagagtctgggaaacaggctcttggctttgactgactccagccatgggtgcatgtgggcctagaatgtctctcagaaggccttaggccagagatctgcagaaaatagctcagtacgtatgtacctgaatctgggactggccatgggagcacagcttcctgcactactccttttaggcctgatggcctctggaatcaggactcttgtttgtttggccatgattatgtcatccctggaggcatgtcctagcacttatgtgaatgctttaagagaagttaactttaggttgagcaggacggagggaagatttgaggggttgttacatggtaggattttaactcatctcagtttgccaatttcagttttaaaatggaaagtcccatgtccgaggaaccccttcaacactaagcaaactgggtccaatggtcacctaatagttggctgcactagggcactcaggatttccactggagacagatccctgaagaggacgcgactgtaggacgtgaagcatttgatgtgcatgtgtctccctgaggatacatgcatatacttccaacctgcttcagaagtgtgagcaacagctgtttcagggttttctagaagatgctcttgtatccaaatttgggacttagtgtgaagcccaggatgaatgaatgtagcctccacttttccctgtgtttggaggcatagactgtagtgcctctgtggcctgtggagatgtgctcaggctgtcagaggaactcactgcatctggaatgttgctgcactcacctccagccctaaaagtctcccacaagacttgacttgtgaggagctctagggatggattcaataaaatttttttcctgttctcctccttctaaccaagggtggaaaactgtacatgggatttgccctggtcccgttctggaccacttggctgataattcaggggctggtaaaccagctggtctccacccacagccatccctggccctgggaaaggcttcagtgttgagagccccgctttcttggttgtcaatggagcaggggcaatgaggaaggatgaggataagaagctgaagtgggagtgttgtagatggctgggaagtggaattagatgtttccttgaagaaaaaatgcagacatttccaatcgaggaatgttcttctgtctttccacatcttccaggtgcttcagtggtccgtgctccccctactcctcatcagtggtctggattcatttccaggaaggtgcttgggtcaatcctgctctgaggggtcctcttttgcaccaaggccccaagacctgtcctctgcttgaatggcatcctcatgtcccccttgacccctgcagttggtggggaactttctgaaggctttttcttgtgacttctgtctctcttgatggaagccataaactgtcctggaccaggaaaatggtctctgttaagtatgagcttccgcagaacaaagggcttggtgcagatcaaagagctgtctccaactgtgatattttttcccctttagtgagccccttcaagatacctatagaggaacttgaggacagagtgtttgtgaattgcaataccagcatcacatgggtagagggaacggtgggaacactgctctcagacattacaagactggacctgggaaaacgcatcctggacccacgaggaatatataggtgtaatgggacagatatatacaaggacaaagaatctaccgtgcaagttcattatcgaagtacgtgcttcctgaaccctttgggttggaatggatagggcttctggatgtgagaactttctggctagagagggatatggtgaacccatctgttctctaaacaaagtgaggtcactattgtaatggtacaagccagatttggggaccttcaaagtggaagagagagcttaactcagcaagacagagcagtaggggtcatgttactgcagccttggttgggtgagaggtttggcccaccaccttgttcaaggtcccaaaagctgagattacttcttggctcttcccagggattccttaggatgcagggtggaggaaggtgtggcaagcccatcaccaggcctttctcccagggtcttattgaagtttgggttttggcataagagatctaagggttgccctaggaggcagaggatggttccctgatcttaaaggctctcaacctctcctctctcctcccttcccccacagtgtgccagagctgtgtggagctggatccagccaccgtggctggcatcattgtcactgatgtcattgccactctgctccttgctttgggagtcttctgctttgctggacatgagactggaaggctgtctgggggttagtggaagagcagagcatgagagtgtgtttgtgtgtgtgtgtgtgtgtgtgtgtgtgtgtgtaccagtgagcttatgcaccagctaaggaatggggctggtggtaggtttagtctcagctgggagttactcttgcaatctcctaaagactcctagaactttgactgttgaaagaacttgtgtgttcatatcactcatgcagacttctgagggtgtgggagggtggatctcacagtcccatctgctaggccattgatgtctctctctggttcttctagctgccgacacacaagctctgttgaggaatgaccaggtctatcaggtgagcgttgaggggaaggaggcaggaatgaagggagggtaagtggggatagagaggctcacactgaatgctgtttgcacgtgggaagggtcctactggggagttcattgctgggtgtgactggagaggtcaggcaggagctctcatcgtcagggcctgctggggcccttccttgaagagttctaaccctgcctcctgaccgttttctcttctcctctcttagcccctccgagatcgagatgatgctcagtacagccaccttggaggaaactgggctcggaacaagtgaacctgagactggtggcttctagaagcagccattaccaactgtaccttcccttcttgctcagccaataaatatatcctctttcactcagcaggtgcctgggcttcttaaggctcctgggcaaggcgtgggagttgtcctgacttgcttgggatctcgccctcctacctacctgtttcttccttcatctccttccttcctctgcctcacacagacagtgtgttgggcagtctctcgccacatcctggctgtctggtgtttctagcccacagggctccctgggtgaagggtctgcgccaccacgttcccacagctacttgttgactccagtccctatcacaacccttttagggttttggctgcctcatccagccgagggcaggctgctgtgttcaggagcccagccgcacagaagtttccatgatgtcatgaatgggagtggcagagacaacaaggggccctgggactagtttctcaccaagactcagagggtgtgtgtaacaaagtcccactgctgctaaggggatggcctcaactctacggccctgctgaccaggtgccactagctgtaaggaggtctcacctaagcccctttcgtatccctttcccctgtatcaaaggcaccattttagcttcctttcgccaggacctgtctctggcttgctcctgccttctgcatgataaatggggatcagaaacccttcaccaattaaagcacattatcccgctccatctacctggaaagcaggagtttgagacagggattttttttttttttgagatggagtcttgctctgttgctcaggctggagtgctgtggcatgatcttggcttactgcaacctctgtctcctggagtcaagtgattctcctgcctcagcctcctgagtagctgggattgcaagcatgtgtcaccaagttcagctaatttttgtatttttagtagagacagggtttcatcatgttggccaggctggtctcgaactcttgacctcaaatgatccactcactcagcctccaaaagtgctgggattagaggcatgagccactgcacctggctgagacagggatattttcaatagatgtcatcttctattacttctaatggtagtggttctaggctgggcacaatggctcatgcatgtaatcccagcactttaggaggcagaggcaagaggatcacttgagcctaggagttcaaaaccagcctgggcaacatagcaacatcccgtctcgaccaaaaaaaaaaacaaaatcagctcagtgcagtggtgggcacctgtagtcctagctatttgggaggctgaggtgggaggattgcatgaggctgggaattggaggctgcagtgagttatgattgtgccactgcactccagcctgggctacagagtgagaccctatcttaaaaaaaaaatagctatggttctgaaactttggaggcctgagggatttattaaaaatataaatgcagccaggcatggtggctcacacctgtaatcccagcactttgggaggtttaggcgggcagatcacaagatcaggagattgagatcatcctggccaacatggtgaaaccccgtttctactaaaaatacaaaaattagctgggcatggtggtgcacgcctgtaatcccagctactcgggaggctgaggcaggagaattgcttgaacccgggaggcggaggttgcagtgagtcaagattgcgccaccgcactccagcctggtgacagagcgagacttggtctcaaaataaaataaaataaaataaaataaaataaaataaaagtgctagagacgcactctaggcagggtgtttccagttgattctgaaacagcccaaattcagagtctcttgtttagagggagaatagaggccactttctctcttatgtcttttatgcctgttcctggatcccagagcaggggattccttcttttgttctctgattacacaaataatttaaagagcctctggaaagtttaatgactgagaatttcatagacctgggttcccaaaggagaaaagaattagtaaccacatagccagaaaattggatgattctgcccaccatcttctcaagatacattca

SEQ ID NO: 6 - DNA, 18596 bp, Homo sapiens
  source 1..18596: mol_type = other DNA
ctagagtagatacacaggaagaagttgagtaactgacaatgggacatcagctgaccattaaacttgatctgcccattataagctaagcagtgtcagaccctccaagtcacaagtttgggtgggcgcaacaatccactgtaagaaggaaggaatgcagtctggctgaggcatgaatagtgccagaggacacaagcagcaactgcaggagcacatggcctccatcatccaccgctactacactaataattcaccttcagttcaacctgtggctacctgatgggctattcttgaagtccagctgacagaggagaataaagcctgagcttccttgacagatgtcatcttagtatgtggatgcaagccaaaaatggaatgctgcagtcctgcagtcctgcattcccactagggaacgagcttgaaagagtagtaaagggaaccgggtgtggtggctcacacctataatcccagcacttttggaggccaaggcgggtggattgcctgagcttgggagttcaagaccacccttggcaacatggtgaaaccttgtctctactaaaatacaaaaaattagctgggagtgttagcacgtgactgtagtcccagctactcgggaggctgagacacaagcatcgcttgagcaccagaagcagaggttgcagtgagccgagatggtgccacagcactccagcctgggcgacagagtgagactgtgtctcaaaaaaaaaaaagaaaagaaaagaaaaagaaagaggagtaaagggaaatcctctcaaagggcagagcttctgccattgtacgtggttattcactttatgtggaaagagaagtggcctaatgttagaatatacagagatccatgggcagtggtgagtggcttgatttattgattaggggcctggaacaagtaagagtagaagattggggacagaaaagtctggggaagatgcacgtgtatgggcccacgggagtcgttaccagatgtgaagatctttgtgtcacatgtgaacaccaagcagagaacatccgtcgtgggagaagcaccacaacacaaagtagaagaaaatgacttggcccgttaaagtcaactagcctctgtccatggccatctcaagactcaaacagtggggttatgaatgtagtagctatggtggcaatggtaaatgctagacctcggcacaacagggtgagctgtcactcacaaaggttgatccagctactgccactgccaaaacagagttcagtgcagagcctccaatttggcatcatcgttgaaggaaaccaaccagtcatttcttgacaagaggattacattggacccttttcaaaccaaaatctggaaagggctcaattcattgacaggacaaatcaattcatttgacatactctgggtatgagtttgattctcttacctgcagtgcctgagctagccctactatccaagagttcacatactgtctgatccgtcagcattagatcttccatagcatcacaacagaccatgggacccactttaaaacaaggatgtgtccgtggacatatgaccatgggatccactggccctatcatataccacaacacccagaagctgccagcctgatggatccatggaatagtcttttcaagacatagctaaggtatcaacttagagatgacaccctgctaggattgggcactgtccttcaggatgtggtgtacatattaagccaatgactgttatatgatactatgtctccaaaaggtaaaacgcatgtgtgcaagagctaaaggttagaagtagggatattcatgcttaccattacttccagtgacccacttaggaaatctgtgcttcctgtcccgctaactctaggctctgagcttctaggggccatggtttccagagacagaacactttcaccacagcacccattaaactttaagctacaccactgccaagagaccagcatgccaaaaaaaaaaaaaaaatgatttgctcccccagcagagtgaatctttccatatctgatgataaggaggaggcagggctgcagggacaactcagatagaatccaagggctcccctggggcattttttagtgcaattttgatggtaaatgggcaagtgcagcagccacaacccgaaaagaccacgacggccaggggctcagactcctcaagctatgtcttccctgaggtcagcagaggtgctaactgaggctaggagcaatccagaaaagggaacaagcattgcctatcagatgcgccctggggcaactgtgctaatggggctgtaatttgtcccattaggaaactttccttttataagcttctccaaaaaaagagataaccagaatcccgaaaaggctgtttaaggacaggatgaaattatcacatgaagcgaatggctccaagcagtgcaagtgttaaactgcatggatgctgtggctgcacccaggtcatcactccagggctgctccctcccccaactgctgggattcttgtactcagaggactcacagttgagctctttcaaagacattgccttcagcagaagcagctgcctcacccaacttcgcagccccctcacccaacttcgcagcccctcaattcataatacctgtatttttcatggcccctttgctcaattcaggataactcaggataacttgggaaggtgatcccaggctaagaactccccatggctctggctgaggctcctgatatgtctcatcgcagttcacctctctctgccagatgctgcccctctctcccttacagatgatgctcctgtgcttcctcagagagccacctgcccaaatctccatctttttttcccagaaaacccgatggatagcaatgcacatttacagacacgaaggtaaccaccaggaaaaaaaaaaaaaagttgaaaaacttaaaagttgttttctctagggagaagtgtaggggtgaagcaaggtagaacaggggctattgttatacagcccttctatatgcattgtttttaacatgcacgtattacttccatcattaaaacttttttcattttggccaggcatggtggctcatgtctgtaatcccagcacttcaggaggccaaggtgggcagatcgcttgagctcaggagttcgagaccagcctgggcaacatggtgaaactctatctctaccaaaaatacaaaaattagttgggcgtggtggcaggcacatgcaattccagctacctgggaggatgagacaggaggatcccttgagcccaggagggggaggttgcagtgagccaagatcgcacgattgcattccagcctgagcaacaaagtgagacttcatctaaaaaaaaccccaactttttccatttgaatattataagaatttgatactgtcactcacctttttaaaactatttgaatacttcctacagccctaagaaaatccttaagaatccctaacatagcttgcaaatcccttcccatctggcccctgctgagtttcctatctcttttatttttttcatttttattttttgacagggtcttgctctattgcccaggctggagcatagtagcacaatcatagctgactgcagcctcaacctcctgagctcaagtgatcctcccacctcagcctcccgagtagctgggactacaggtgagcaccatcacatccggctaattttttgtatttttttgtagagatagggtttcaccatgttgctcaggctggtcttgagctcctggcctcaagcaatctgcctgctttggcctcccaaagtaccatatctggcgaatttcttatattatttcttacactcctcgttctccctgttacctgccgtgctggattttccatttctcttcctcatctctggacttcaaacatgctgctgcctctatctggaatgctttccactccacctggttcattgctgttcctccatcaaatttcagcttagatgtcacctgctcagggggaccttctcttccccatcccaccccagactatcttagatcccttctatcactcccacagcacctgaacgtcttttttcttagcatcaatctcctgaagttatctggggggttttttagagatgggatctcactatgttgcctaggctggagcgcagtggctattcacaggcacaatcacactcactgcagtctcgaacccctgggctcagtcagtcctcctgcctcagcctcctgagtagctgggaatacaggtgtacaccatcatgcggggcatggttattggttatgaggttatctgtttaatctccacatttccttttttatccaagaggactatttatcttgtttctcctgtattactagcacccagcatataactacagtacagtagacagaaaatatgtattaaataagtgaatttcatcaaaatgttttctagagtcccagctctgtaatctgcaacttagataacctgcctgagccttagtttgctcaagtataaaatggaaataaaaataaaaataatacttaccttagagggtcgttttgaggattaaataagatatatcagaaaagctcttagaacggtgcttggatatagtaagcatttgattaatgcatgcttaaacataaagaaaggttacatcaattcacactcataccagcagactttgacactatcactctttaccctgttcaacattgagtcttgtgtttttaaatatttttgtaaagaatataggtaaaaagtggcattttttctttggatttaattcttatggatttaagtcaacatgtattttcaagccaacaagttttgttaataagatggctgcaccctgctgctccatgccagatccaccacacagaaagcaaatgttcagtgcatctccctcttcctgtcagagcttatagaggaaggaagaccccgcaatgtggaggcatattgtattacaattacttttaatggcaaaaactgcagttacttttgtgccaacctactacatggtctggacagctaaatgtcatgtatttttcatggcccctccaggtattgtcagagtcctcttgtttggccttctaggaaggctgtgggacccagctttcttcaaccagtccaggtggaggcctctgccttgaacgtttccaagtgaggtaaaacccgcaggcccagaggcctctctacttcctgtgtggggttcagaaaccctcctcccctcccagcctcaggtgcctgcttcagaaaatggtgagtctctctcttataaagccctcctttttcatcctagcattgggaacaatggccccagggtccttatctctagcagatgttttgaaaaagtcatctgttttgctttttttccagaagtagtaagtctgctggcctccgccatcttagtaaagtaacagtcccatgaaacaaagatgcagtcgggcactcactggagagttctgggcctctgcctcttatcaggtgagtaggatggagtggaaagggtggtgtgtctccagaccgctggaaggcttacagccttacctggcactgcctagtggcaccaaggagcctcatttaccagatgtaaggaactgtttgtgctatgttagggtgagggattagagctggggactaaagaaaaagataggccacgggtgcctgggagagcgttcggggagcaggcaaagaagagcagttggggtgatcatagctattgtgagcagagaggtctcgctacctctaagtacgagctcattccaacttacccagccctccagaactaacccaaaagagactggaagagcgaagctccactccttgttttgaagagaccagatacttgcgtccaaactctgcacagggcatatatagcaattcactatctttgagaccataaaacgcctcgtaatttttagtccttttcaagtgaccaacaactttcagtttatttcatttttttgaagcaagatggattatgaattgataaataaccaagagcatttctgtatctcatatgagataaataataccaaaaaaagttgccatttattgtcagatactgtgtaaagaaaaaattatttagacgtgttaactggtttaatcctacttctgcctaggaaggaaggtgttatatcctctttttaaaattctttttaattttgactatataaactgataagtcctctctacttcacagattaagaaattgatactcaaaaaagttaaataacttgttttaaaccacatagtaagtgccgaagccaatctgtgagaccaggactgtttgtactctaaatggctgcaccacatgaggcaaaatggctcgtgatggttttatttcaaagacctagaaaacactatcacagctggtgctcccgtctcagacccacagcaacgatgtctccacttcctgcttcatcttgggtttctcacgtcttgaatgtgcacacaaatcacctggggatcttgttaaaaatgcagcttctgtttcaaaagacccagactggaaatggagattccgcatgtctagtacgctctcaagtttattaatctgctgctggtcctaggaacatattgagtagcgaggggcaggatgtgactcctgtaaggagtggccaggcattttcttagagacctgtgttataaagtatgcttttccttaaaaaaaagaagaaggaggaggaggagaagggccaggtgcagtggcttacgcctgtaagcccagcattttgggggaccaaagtgggaagatcacttgagctcaggagttcaagaccagcctgggcaaagtagtgaaaccccatttctacaaaaaattaaaaattagccaggcaaggtggcacacacctatagttccagctacttgggaggctgaggtggaaggatcacttgagcccaggaggtcgaggctgcagtaagccatgatcaagccactgcactccagcctggagtgtctcaaaatagataaataaataaataaataaataaataaataaataaataaaagagacagtatcaaagacccaatcacctctagacatctggcatcataggaatgtgcccagtctgctctggggataggaaagtggggatcctgtctccccctgtgtagaggtttcagtaaaagaaaggcctaggtgtgcagaaagctttcaggcaatgccagggaaactgatcattgtaatgaatccagggtattgctgagtgagggcatcctggagggcccggtggaaatgtggtcaggctcttcaatgcacaggccctagttgatgagtaatcagggtttcaaatatttccatctctgtctcaagcagaaaacaaatggaaaaactgaaccaccagaaaagcagagccagagatggaacaagaatcccagtgtttgtacccaaccaagagcgtgtttttcttccacagacaccaatgttcaaaatggaggcttgggggcaaaattcttttgctatgtctctagtcgtccaaaaaatggtcctaactttttctgactcctgcttgtcaaaaattgtgggctcatagttaatgctagatgcttccttcctctatttccccccaaatttcctgggaacccctggtcaataccagcagtaagttccactgttctagggtgtagaaatggctgtgacccagcagcaagagggaaggacatcagatgtcatcagtggtcatactgcaacacagccctttttctgtttaggaatgcaggtacccacaacatttactaacactttttttttcttatttattttctagttggcgtttgggggcaagatggtgagatatgctttctttctttcttttttatgaaatcaccccatcattctttgtagttatgaatggagctttctcttaggcctcccacagaacttccacagaggtcaggaaaaggagtttctgccatctacccctttgactttcctcacaagtctggagatatttctagcccagaagagggaagcaacagaggcaggaaataatgagtcttaaccatacaaaagaaaaattgagacttaaatgaagttgaaagcactaacagttttcatttgtttgcatttcatatttgatgtgagattctgcagaggagacgtagccagaatgcatgcacagggttactctggataagctgctggggcaacatttggatgtgtgttcagaatcacatgtctgaatactctgaatatatgtgtgtacatgtgtatttatgcaagtgcacatgcatatgagtgtgcccggcctgaacttactctctcaaccacagcggtagagtcaggagtgttccaacattggaagcccctctattcaatcagctcttccaaactgagtgaaccaatgttgtatttaatggcaaccatggctggacaccatggctcacacctgtaatcccagcactttgggaggccgaggtgggcagatcacttgaggccaggagttcgagaccagcctggccaacatggcgaaaccccgtctctactaaaaataccaaaatcagccagacatggtggtgtacgcctgtagtcctagctactcgagaagctgaggcaggagaatcgcttgaacctgggaggcagaggttgtagtgagccgagatcacaccactgcactctagcctgggtgacacatcgagactgtctcaaaaataaaataaagacaaccattatgccagcctagattccgccatgctgcctaatttgtagtgtccttaggagccatttttgtaaatagtcatcagataagatgtaaggcccataacagctttttctatgcagctgagggaattggaagatccattgtttcctaagagttgagggaagagtcccaacccacgggagcagggtctgatcttcattgccgatagaaacattactaatggcttcttactgtttccttttcaggtaatgaagaaatgggtaagaagatttccactctatctagcaaaagttttcaaatatggaatgaaatgctcatagagtacaatcacagtaacaaaccctgagaactaaaactattaaagggaaaatacaagtatctttcaatgggatccgtatgaaacttgcctgtatttgttgctagctgtcatgtcagattatagctgtgcatatatgtatctctgatcatacacatatggatgtgggttggagctaccatgtgtttttgtataagccatgaaatctttgaaggcagacagagacagtgtctcatttacctagcccagtgtctggcacatagtaggtgctcaatgaatattttttgaatgaataaatgaacaaacatatgaacacattgctaattacctcccctcaagaagctgatggtcttgtgtgagagacaaataattgaaaatatagtgagttgcatgttataatatgggtagatacagagtaaaatgaagtataaagaggggagtggtcaactctactgagtgtcgttgggaaaggttccctgggggaggtggtccttgagctgaattttaaaggataagcttatgttttagggaagaaaaatattttatgcagaagagataaagctgtatagtatgaggataagagtctaactgagctagatcagaatgtttgaatcttggctcaactctctacttgctgggtgtgtttgagtaatttacctaacttttctgtgccacagcatcatcatggtacaatggaaataatagtgctacctaacttgtagggttattatgaggaccaaatgagtaattcatttaaggcacttagaacattatctgacataaaaggcagtaggagggccgggcatggtggctcacacctgtaatcccagcactttgagaagccgaggtgggaggatcacctgaggtcaggagttcgagaccggcctggccaacatggtgaaactccatctctgctaaaaatacaaaaattagccaggcatggtggcaggtgcctgtaatctcagctactcaggaggctgaggtaggagaattgcttgaacctgggaggcggaggttggagtgagctgagattgtgccattgcactccagcctgggcgacagagcaagactctgtctcaaaacaaacaaacaaacaaacagacagtaggtgaattttagctattaatacatggaaagcatgctgactatagatgataagcattaaagtttactgagcatgtatgttttaggcattgctctaaatattttacttgaatttcctcatttaattcttccaacacccctactgtacagttaaggaaacaaagcctcaaataaatacagaaataaacaaaaataagtaaacaatccagtcctggggatataaatgcagatttaggccaagtgccatggttcatgcctataatcccaacactttgggaggccaaggcaggaggctcgcttgagctcagaaggttgaggctgcattgagcaaagattgtgccactgtactcgagcctctgtggcagagaaagaccctgtctctgaaaaaattaataaatagaaatttaaaaataaaaaaattttaatgcagatttatatgataccgaagttcattttctcaaccattatgaaatactgtttctggatatgtataaaatctttgtgagcacacatatctttttttaacttaactttcattttaaattcaggggtacatgtgcaggtttgttatataggtaaacttgtgccatgggggtttgttgtatagattatttcatcacccaggcattaagcctggtacctgttagttatttttcctgatcctctccctcctcccaccctccaccttctgagaggtcccagtgtgtgtcatttccctctgtgttcatgtgttctcatcatttagctcccacttctaaatgagaacatgtggtatttggttttctgtcactgtgttagtttgctaaagataatggcctccagtcccatccatgttcctgcaaaggacgtgatctcattcttttttatggctgcgtagtattccatggtgtatatgcagcacatttttttatccagtctaccactgacaggcatttaggttgattccatgtctttgctattgtgaatagtgcacaatgaacatacgtggagcacatttctgtctaagcacagacatctagacccttgtgtgagcatgagttaagtctaagctctgctactgaatttgtgccaataaaagttgtgagcaattttctttacatttttttcaaacaaacacacccagcagagtataatgtctatgtactttatttatgatttctagttcatttaacatgtctaagaaacatccgtgttgaaaaattatttataaattaaaataatataaactatctactgtccttatactcaactcccaattataagcaggtggaaaaacctggagaatgttttgtttacattctgtgcagtctttgtcagagggctgcctgagcaactgggtcagagtttagttctgctctgggagtagcaggacctcaagaaggaaaggaggaaaggaagtaactttttcttgagcacctgctatgtgtcattcactttcaccttcataatccatttaattttcgcaaaaactttgtgaggttggtgttttatctccatttccctgataaagaagttgaggttcagcaaagttaaatgacttgccctcagtcacacagactagggcagatccaggattcaaactcagggcttctgactcttgagtccagagctctgtccctgacagcagcagcactgcctctcctctcttccagctgttatgtccagactgtagcagaacccagtgttccagccacaagttttccaggaaataataaaggactcctagctccacctcccagggcaaaaatggctgctgtgggaaacacaggctggacctacgaatggcattagtggtttattagttgatttcagttgtccacactaataggcctccctctaacaaaaataattgagagctgattatgctcagatataatgtaaagtgaagccactttttattggaagaagcattccctcaaaacgtgtagagtatttcacattatttaaaggcaaatagagagaaaattatatggaataagaacaaagatgtttcttctctattatgagggactcagttctgagaaaggattttaaattgtaagaaataggtaagtccacgaatcagtgattcagtggtgtggagagctttatttctgagaaggccagtagcgctcccttctgacaagcaaatctaagacctggatgacagatgacttcctgcatttggttggttcttttgtcattcatatctatctgtaatacagttctggctaatttaagaggataagcttgaagacctctggaatttttcggctttaggactttaaggctttctgagcttcagtagatctagatctaggagctcatgctggtatattctgaatccgatgtatctgagttacatctatgagctacttaataaatatatctatgagctaaatctcataggctaagcatgaacctcacctccaagactcggggttcctaaatggatgagaccctctttgggaagtcttgtgggcagtgtctaattccactagaaaagttttacctacaatttaaacttaaaccatgatattttcttactgctgtttccttttttcattttcaggtggtattacacagacacgtgagtttattggtcttttatttatgccctgtctgaggatgcagattggtgggtagatgagaaggaactgattgagagagattaaccccaagaactgatatcttcccagcattgcattctcaactccattttagaaaggttccaaatagggacttctgtgggtttttctttacatccatcttacccttcccaagtccccatgtccctgcgtaaaccctaaagccacctctcaaaaggttctctagttcccttcaaggttctctagttcccttcattccacatatctcctcttccacaccctctagccagtagagctcccttctgacaagcaagtctaagatctagatgacagatgacttcctgcatttgggtggttcttttgtcactaatttgccttttctaaaattgtcctggtttcttctgccaatttcccttctttctccccagcatataaagtctccatctctggaaccacagtaatattgacatgccctcagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagaggtaatccaggtctccagaacaggtaccaccggctctttagggaggaccattcaaaagggcattctcagtgattttccctaacccagctcacagtgcccaggcgtctttgcgcttcctcccacactcaatcctgggactctctggtaccacacggcatcagtgttttctggaatatagattaaacaccaatatgaggcttctgggtaaccccagtctgtgcgagatctaaaatagcaactccctaagagacaggactgggtcatttgcaccgcatcacacccaggttcatagcacaccaacatgagtttatctaatgcttcctccagagataaatttttcagaaaggtttgcaaaaaacactcaaggccactatagtaaaatggcataagctaaggtataataataaaataataacaatacttaacatttattgagtgcttattaagtctcaagcactgtctgtacccaacacttatcaaggattctttttcatgtaatcctctcaacaactatatgggttaagtatcattttattcccatgagtaaagggatgaggaaacagagggtttgtgagttgaaaacacatttcacgcttctcacagctagtgagtaataaagctgggactcaaacccagggctgtttgactccagtgcctctacccacggccaccactctttgcttgtcaatgttgttctaaacatattgaagggggggctctgaccgtggcaagcgtgtgagtagtaaggggagaatggccttcatgcactccctcctcacctccagcgccttgtgttttccttgcttagtgatttcccctctccccaccccaccccccacagtgtgtgagaactgcatggagatggatgtgatgtcggtggccacaattgtcatagtggacatctgcatcactgggggcttgctgctgctggtttactactggagcaagaatagaaaggccaaggccaagcctgtgacacgaggagcgggtgctggcggcaggcaaaggggtaaggctgtggagtccagtcagaggagattcctgccaagggggacgaccagcctgggccagggtgggtggcaagtccacagctaggtcagaacagcttctctagagcttctatgcacagcttctattactgtgatgacaagatctcaacagacggtttcaaatctcacatcactcccctccttcccatcctagaaaagtgcaaaaaagtttatgaaagtgatgggcttcctcacatacctgtcaatgcctgcagtcatccgattccgcccctaagctgtgggaagagagactttgatatattagctcctgccttttcctttcccttcccctatggagagaaacaatgggaggatcttgagctgaggaaagtcacaaaatgatgagaagagtgtagggtccttagagatgaatgaaagaaaaaaaaagagaaaggacgtctgaacagaaaagggagcggtagaggagagaacaatggggtttgccattctctatctgggtctcactggcacagacagtgctgcaagattggttccctcatgggaatgaaatgtttcccctccttcctccgcaggacaaaacaaggagaggccaccacctgttcccaacccagactatgaggtaacgtgggatagaaatgggccaggacgctggaggggatgtccctccaggggggaaggaaacagatgggatggcccatcttgtctgccagatgcctcaaagcccctcactcagggcttccattacaaccctctatgtgccacctctgcgtccttcatggtaaaacaggactgtctcaaaggctgcatggcttccacaaccatggagaggtggaagcttgcaggagacatactcctctttctctggcttattcattgactgggatacagccatggagaatattatatatgcaaattctaacacaataaattctgggctgatattccaccagcatgcaccagtatagcgagttattgaaatattaaaattatataaatattatataaaagttattgaaatattaaaatactcattgggaaatagccccaaactttgctcaccccaacccacccttacacacacacatacacacacacacacacacacacacacacacacacacacacgtgaccagacatcccagtccctcccctaccgggctgcctcttgagttggggtaacaaagagttaatgcctggcatggcagaggatcaccaggattgttctagttgattggtatgtgtgcactcctagttgttaaatattttcactatcacacctggatatactcaacaaatatttgttgagccaaatactcaacaccagccaaacacgtagtatttactttagcttaagcgaattatttagccctgacagaagccctggaatgtgggtctttaagttcctatttttgagatgggaaagctgaggctcacggaaggaggtgaccagctcaagtctcctaccgtccatgccaaattagaattccagcctgcctcctgacttcaagtccaaagttcttcccacgcactaaagctagctcttcagtgtcctttcttaggaggtacttcctcccgcaccactgaccgccccctctctatttcacccccagcccatccggaaaggccagcgggacctgtattctggcctgaatcagagacgcatctgaccctctggagaacactgcctcccgctggcccaggtctcctctccagtccccctgcgactccctgtttcctgggctagtcttggaccccacgagagagaatcgttcctcagcctcatggtgaactcgcgccctccagcctgatcccccgctccctcctccctgccttctctgctggtacccagtcctaaaatattgctgcttcctcttcctttgaagcatcatcagtagtcacaccctcacagctggcctgccctcttgccaggatatttatttgtgctattcactcccttccctttggatgtaacttctccgttcagttccctccttttcttgcatgtaagttgtcccccatcccaaagtattccatctacttttctatcgccgtccccttttgcagccctctctggggatggactgggtaaatgttgacagaggccctgccccgttcacagatcctggccctgagccagccctgtgctcctccctcccccaacactccctaccaaccccctaatcccctactccctccaccccccctccactgtaggccactggatggtcatttgcatctccgtaaatgtgctctgctcctcagctgagagagaaaaaaataaactgtatttggctgcaagagttgctgtccctgttttctgagagaccctcagaggctgccttaactcccaagaccattcaggttctcactatctttgactaaattctcagctatgaccccagatgcgagcactctctgtttccacagctggtagggccgttacctgaagctctctttagagagggcataagggaataagaaaaaaaagagagagagagagtgaagggaggggaaagaaaagaaaaagagagagagaaagaaaaataaaaggaaagggaaagaagggagggattgaggaagggaagaaagaaagaggatgggagggagagaaagaaagaaagtttcaccaacttctcttggaggaaaacatcaaacgtggcagagaaagactgatgggtccctgatggagtggttggtagaaaatggacaagacagaaataggccttgaaagacagatgaaggtacggaagaggacggtggcggtggaagccgggcttggagatgggacacagatttccacaagctgccaggaaaagctgcgagccagggctggggaagtgaaggagggaggtgtctcaagcaggcacacccccaccctgaggcagccgcctgcagccagaggcgggctgtggttaagcagtgcaggatgtgggctgcactgctaagcgtggcttctgggagtgagggtgggagaggtacagcggcagctgggcggaggcccgtgtgagagcgctttgttctcagtctcccacagcacactctgcttgcagagagaggaggctggagcatccaggaactgtcctaatccagctcaaaccagtgggctctcttctctccctccctgcactaataattcaccctgctgatttatgagtgcgaccaccactaaaaaggccattcgtgaccagcagcctccacatactgctccccctcagccttgggctgtagctacagcttctatttgtttttggtcgctgttgaagagtcacttccttcactctaagcatcaacacttaggttcagaagatgcctccatgatgttgtccatcactagctgctggatggcagttttgtcccaagtatacttgattgtgccctgtcaaggggaggcatggctcagcgtaatacaggcaataacagctgacatgtgttgagcatttactcaatacatgcttcagaaattgtacgtagcactttacctgcattatctcatttaatctttatgttgacacaagaagttagatgtcatcctcattttacagacgaggatactgaggctcagagaggtcaaatcattgcccaagtcacagatggggtggggcagagctggaactagcagccaattttaatggtcagagcatctactcattactgactgtgatcataaagaatgttccccattgtcgcacactggggcctgtcgggggtgtgggggccaagggcaggaagagcattaggacaaatacccaatgcatgaggggcttaaaacctagacgatgggttgataggtgcagcagatcaccatggcacatgtatacctatgtaacaaacctgcacgttctgcatatgcatcccagaacttaaagtaaaatttaaaaataaaaattttaaaaaagaatgtgtcgggcacggtggcttacgcctgtaatcctagcactatgggaggccgaggcaggcggatcatgaggtcaggagttcgagaccagcctggccaacatagtgaaaccctgtctctactaaaaatacaaaaaattagccaggcatggtggtgggtgcctataatcccagctatttgggaggctgaggcaggagaatcacttgaacccaggaagcagaagttgcagcaagcggagatcgtgccattgcactccagcccaggcaacagtgcaagactccgtctcaaaaaaaaaaaaaaaaaaaaaaagaatgtttctcattgtcctgacttaaaagctctgtatttaattgtttatgcattccttcaatgagtgcttttctaatcactcatgtggctatcagc

SEQ ID NO: 7 - DNA, 32306 bp, Homo sapiens
  source 1..32306: mol_type = other DNA
tttctttaattactaatgatgtttagcatcttttcacttgcttattggccattcatttatcttcttttgggaattgtctattcaaactttgtgcccattttcttattgagttgattactttactattgagttaaaatagttctttacatattctggatataagtcctctgtctgatgtatgtgtttggacataatttctcctagtctatggcttctttttcattttcctactgctgtctttcaatgaacaaagcaaaaacttttaattttgataaagtccagtttaccaattttttcttttatggtttttactttttgtgttctatcacaaatatttttctcctatgttttattccagaaattttatagttttgggttttacatttaggtctatgaaccattttagataatttttgtgtatggtatgaaccgagggtcaattttttttccatgtggatatccagttgttctaccaccatttgttgaaaagactaccatttcctgctgaatttttcttgtacctttattgaaaaccaattgaccatatatgtttaagtctatagtaccctaagtactgtatgataattacaccccttttattgtttgatatctggcagggttatttcactctctttactcttctttgcaagaattttcctaactattcttgtgtttgttttagcattatttttgtattgagatataattcatataacataaaatgtgtcattttaaagtgcatacttcaatggtttttagtatattcactctgttgcacaccatcaccactgtccaattctagaacatgttcatcatctcaaaggaaccctcatagctattgacagtcccaattccccactctccccatcccctggcaactactaacctgctttctatctctatgaatttgtttattctggacattttatataaatggaatcatacactatgtaaccttttgtgcctggcttcttccgcttagcataatgttttcaagggtcatcccagttgcagcatgtaacagtgcttcattctttttgatggctgagtcatatttcattgtttggatacggcgcatttgcttatccattcctagttgtggccattcgaattgtttccagtttagggccactgtgaataatgctgctatgaacattcctttacaagtttttgtgtgagcctaggtttccgattctcttgggtatatatctaggaatagaattgctgggtcacatgatactatgtttaactttttgaggaactgccaaattatttttcacaagggcttcaccatttatataacccacaaaaatgtatgagaattccagtttctccacatccttatcaacacttgtattgtctgttttcttattttagctatcctagtgggtgagaagtggtatctcactgtcgttttgatttgtatttccctaatgactaatgatgttgtacatcttttcatgtgctaattggccatttgtatgtcttctttggagaaatgtctatttacatctttggccaatttttaattgggttatgttttttattgttgagtcataggggttatttatatattctgaatcctagacccttatcagtatatttacaaatgttttctttcattctgtgggttgggttgtcttttcactttcttcgttctttcttttgtctttctttgtcatcatatttcatgatttcttgtgaacaaagtattgtctagaggacaagcattgtaagtattaacatcggaatcagtgaaaacaatcagtatagatcaagtttttaagtgatactttctcagaaatgtatgtgtacaggtcattgaagattatctataagcaattggtcttggaaaagcataatttttgaagaactctcttgccctgtgagagtgatctaatatgaatgaataaaatatatttctgtagttgaactgaaaataggtacatctctgttgttgaattctaataattttattctctttaaaaatttacatagagtacaattaccagtgttcttttgaacgtgctattttctcaaaattcacatagctatcccaataacactcttaaagacttaagcatagtacttttataaagcctcttctttcaatatgtctcctaaagtccccttataaaatgtcttgttttaaaatagccgtctttggaatactttatgatgcagagttttatttatgtatgttggttgtttcaagccacttcacgtaatggtgattcgatgatcaatgtggcaagagtgaatataagacaatgtaagtatggaccactagttatgttgaaactcagaccctctgaggatgcaaaagagaaccagcaagtatgcatccttcaagctgagtaaaagctaaacaaacatacatcggatagaaacaaacaacaataaaatccttatggctttgttgatcactgtggtgtttaattgactctattttaagacttttctacacttagccctctctccctactcaaggtgcagcaaatcacttttttgttaataatgcatgtctggatcttatcttggctgtgtctgtattgcagtcaatggttcaagtcctcaaggccactgccagcaccctggcacatgggggccctctgctgtctttgttgtcctgttgactgctaaagaactcaggctaacaatgttactacccacattcatgagttacttgttgctggttgtcatctgttgcctttcaagtcttttctggaatctaattgttttttgtttgtttgtttgttttgttttgtttttggtctggaacccagaagacatttatctaataaaatattaggtgataatttcctaagaatttcaacagatttaaacattttgtctttaacatgggtgagttaaaatcacttacactatacaaaagttaagcatgtttccatcaccacttttcagataataaaacctctttcatttccgtgtccatggggaactatgcaccctaaggtcctccacacgtgggaggggagaatctgttcaggaactctctgaagtcagtattcatcaagaaatgggtatttgggatgatcacaccacatgagcatttctatgaagcggacattttcactccaggcttcaatgtccttgattcagtgagagaaaagtcaaaggcctgaaggttttccttgatcttttcaggattaaagcttttaggaatgacaaccaccccttgctggatgttgaaacgcaaaataacttgagctgtgatcttattgcacctttttagaagtgcatcctttaacgaaggcagagaagacatatttgtccaggttggattcctagtggtccccaaagggctatatgcaatcatgacaatgtcatgttgttggcgaaatttcaaaagttttggttgggtgtaatatgggtggcactcaacctcgttgctgactggcttgggtttgagtcctgggttgttctggatgagctccagctggcctgctgttaaaactggatactccgagggatttcaccaagccagcatctttacaagcttccgaagtctcccaagtggcacacagatttgacttgtgatataaccatttgccattctcacctatagggtagattttgtcttctggcttagcggccatgggtatttcagtgatgtaaagatccacacaatctagctggaggaccttgagcatcctctctaggctcgggcagaccagctctgggttgtgatttgtagcccacagttttccacagtagaagatatccttcctctgcacctttccttctgctattttccccctgatggcctccccagctgtgtcttcattttggtatatgtaggcttcatcagtgtgtcagcaccccagaccaatgacaaccttcactgatgaagactcagactaaatgtagagacagactacttgggctgtagtgacatctctacctaccacctacttgctgtgtggccttgagtaagtcatcggcccccttaggggtcaatttaggctctgagtaggtaccaagtctgatgatgggaatgctgtttccatcacttagaggtatgcggtgatttgcaggactcactttcttcatagtgtcctttgatgcacaaaaacttttaattgtgatgaagtccaatttatcaattttatgttggattgctgtgactttggattattggctattcttgtacattaatttttttcatatcaattttggaatcagcttaacttgttctaaaactttttgttggtattttcaatgaaatagattgattaattaagagagaatggtggtctccttaaaagagagcttcctatcaaagaagggtatgcctttccatttacccaagtcctttcggcgttcttcagcaatgttttaaagtttgtttcatatagatatattacaatttttagacgaagtttatgtctaggttttaatgttttattattgtaaattactctggtgtttaaatgttgacaactaacacaagtgtttaaaatactatgtgagctacagtatttcacacaatgatccaaattcaggccaagttcagatctgcaacctcagatctactccagctctttgctttatagataaggaagctgaagtctagagatgttgagtgactgactcaacgccacacagcaagtaggtggtaggcagagatgtcactaaagcccaaggaatctgtctctacatttgtcctggactctaatccagggttctgtcattttgccagccaccataaaatatttgacagtctaggaaaacagcaaatgttcttacatccatcgagaacttgtatttgcctagagatggactttaccaatcaagataggaattagataagaattattttttttaaaaaaaggagcattaaaacaatggtcaaatgagagaaaaagcaaaattcagacagacagatacatacacacaccccaaaccctcaaacctccagggcttcctgcctgtgaaccgaaagggggagtgcgaatttcttggccctgtcggcaggtgctttctcaaaggccccacagtcctccacttcctggggaggtagctgcagaataaaaccagcagagactccttttctcctaaccgtcccggccaccgctgcctcagcctctgcctcccagcctctttctgagggaaaggacaagatgaagtggaaggcgcttttcaccgcggccatcctgcaggcacagttgccgattacaggtagggccgacgtgtcgacggcagggaacttctagaaactctttgtgtatgtgtaggggagtgggtgtgggtagtggagggtggtgaggggctggggtctccttcaaatcctgccctcctcaaatcaatgggcaagtgagcagtgaggggtccggcccggtgctggcatctgctccgtcacagccgagttgggcatcagttcagagccagctgctagaatacacagctcctccaggtacagatttttcgagtgataagagaggaaggaagaaggaagcaagagagcaaccgaggcaggcccagggtagagaaacggagagggagtgtttcacagaagctggacaggatcggaaaatgtcagccaggccatgtttcgtgctgcgacgttctagtcccttcagcttctttcaccctctggctggctccgtcactcagcccctttctttcttcctttgccaacaaagaagattttgttggtcagtcaaggtcatggcctgaaacactccaatgggcccgggagtaagtggggtaaccaaggaggagaggggcacaggcaggaagcaccgggtggcagcctccacagcgccttcgccacctcatggccaagcgtccgtgtctaggctcagccttgtcatggctgtccccacacccacgcctgggactgccgggtgacgcaccatcccaggctccgtgactcaccccagcctgccccttaggcggccatctagcccagccagagggcccggcattgacagaacacggggcagatgagacgatgtgcctggttaagtcaccaccacgagcagcgcccagccaacaagagtgcttgctcctcacctcattcccgatcgtgggagcccagctttgctgcaagcctttagcttccaccatgttttctagcccaggtaatgaggaagaaaaccagtgaaaagaggacaggacaaactgctcagggggatcagcctcgtggcatcttcctgagaagtggggcaggctgaatttcaatctaaagaaacagggaaggtgaagcaagcaaatgtcctcggcccacctgatcgctcctggtaactcacctgggtgtgggaatttgtttaggggccctgttgggagggcaggagaggccatgggcacttctgctcccaccagggagccaggcacagcccgggccagcgcctcgggggattctgaggcccgtgcagctgcaggggtttctgtcaggtcacagctgttttctcagaaaagccttgtggggtctaagtcacacacacagtgtggcagagtgagggtgcctgagcttgctcagagcagcccatccttgtgtgctggggacaaggggacccagaacgctggaatttagtgacttcaggggagacagccaggggcaatagctccagcctctgagcttcaggcttgagacaccacaccagggagctcatataacctttcagggtctttacctgcagtttcctcactggtccatttgtgttcggcttggagagaccaaggtaggggcaaagtgaagttagaaagaaaaatgaaagattgttgggtggcggtggctgccaagcgtggtaaacagaaccccacccagcagacaagatgggggccgtgcctcatttccacaaatagggggcaggaggagaaaactgcacattggctggattttttgcttctcagtgccacttactttgcctctatggagactgatattttagctattttagataaagctaatagacaaaagtgtttcaaaatatcctagcccagaaacgaggtacggtaaggcaaagcgcgactcagagaggccttgacaccgagggctgcgatggtgcttttgctgcctgaggggacacctccacagtctccccggaggaaaggccgcacatagaatttcagtcaggtcttctctcttttcttgctcagttaagtgacccattgcatacatcccttttaaaatgtctactttttactaagctgttgcatttgcatttctttggaaatgcccagattattaggatcaaagcaaatgatgacctggtctgtccacatgaggaggcctccctgagagagaatctaccaagacagtcaccagggcttgaggccagaagatcctaagatggcgtgcctagtggcatgggcacacttgtgcctgcagagccctcagtgcctctaactccctactctgcagtagccaagaatgcacccagcggtcagtgtggtgtttgtcttttatcagtgaggagatggagctctgacatgcagccttggggaatgcaaagcttccaggcttgtgagtggctctgcttttaggaaggtggctggacgtggtagaaagagccaggctaatttcaggtggtgtcccagtgctgccgcttcctagctgtgcaaccttaggcaagttacttaacctctctgggcctcagttttctcacctctaaagaaagacttgtaataccttctttgcagagatgtgacaatgagaagtaatgtaatgtgtccaatacatagtagacagccaataagtaaacattgtatttactattactattattacatctcggactgtcttcttgttctcaaaacaggagatggaaacccatggctgggattcttcatttgctttcaatatgttataaagcagcagtgggcattttctccatctgttgcaatccaactcatccctcttctgatttctgggcacacccataccagcacagaaacataacaatgattgcagaggtaggcccgatagtgaaaaagcagcaaagctctggctggtcataggatgactgttacatccaccattttcattttttaaagatacttactctgtaggggaatattctcccgaagtaaaaacatgtctgaaaaaatatatgcaagatgggggctgtgccccatttccacaaatagggggcagggggattggctggatttttttacttctcagtgccacttactttgcctctgtggagactgacattttagctatcttagataaggctcatagacaaaagtttttcaaagtattactttgaaaggaatcagaaacaaaatccacccagtcatcactggtccgggtggccacctagattctgaagccaaagggctattgtgggtgcacagcgactgagtatctaaatgcaaacttcttgaacactaaatctttgctaaaattgctgggaggtgaatgtgaggcaagcttttaagtgataccagtacaaatttttcatcaatggcattttcaccatcagattgtttcgccctccaatcgcctagctctgctcatccaaatgagctatttcatttttcaattttagcgtttctcaggcttactagcctagagatcacatctgcttctgctaagcatcccccagaaacggtcctggggagctctggcttaggtgaataaccactgctgtatttaattttaaattctttgtatttttgcttttgttctttaaaaaaatacatgtaattattgttggatatatacttttccagtcaaaagttacccagcattgtgtcttgtgctttacttagaagggtccagttgccctgagggaactctcttagattagagttgtccaaacacgcactttgggaatcgtttctcccaagccttatttatctaaggcttaggaccttatataggtcagataattgatttaataaataccggaaatagttcaaaataaatttcttagctctgcttcggccaaggctaatttttattaacactaaagtgatggtgttcgtcactttgaacaaacattcaatatggttaaaaaaaaaaattaaaccggtcattagacctgttaatcaaaacaaaatgatcgctttagtcagcgcatgtacatagtatgtcttccggtctaaaaaacaactttaagttccctgaaaacaaagacgcccctgtcacgctcacagctctacccagcacttagcatggtacctggagcagagtcaatgtgcaataattatcaataagaattgttgaatgcactctgggaggccgagacgggtggatcacctgaggtcaggaatttgagaccagcctggccaatgtggtgaaaccccatctccactaaaaaatgcaaaaaattaactgggcatggtggcacgtgcctgtagtcccaactacttgggaggctgaggcaggagaatcccttgaacccgggaggcggaggttgcagtgagctgagatcgtaccactgcactccagcctgggcgacagagcaagattccatctcaaaaaaataaataaaataaaataaaataagaattgttgaatgaattaaaaggtgagtgaatgaaggaaatctagatgttttcatacgattaaattaactttaatggtttatttggtgggtatattgaattggcattgtttggacctttcctctagagaacgtagtattaaacaatgttattttaaaaacaaaacaaaaaccagaaattaaaccagtggaggggacagggcagtttgagtgattagaattatttgaaatgagttgtaaggatggtttaagataaaaccctgccattattaaagttttctggttggaccatatgaaatttccactatttgtttatttcagatctacaaaaaggataatttcatatgattcaactaacattataactaaaaataaagaaatatgctcattttttctgattaggctttaacaccggaggaaagcggaaaataagcaaggctaacactgttggagattgttaactaatgaactaaaaataaactaatgaaaggcatcaggtagcaaaagactggattatagtataaaccacagtatccaatcacccagtttcctcaaaatatagaaagaaaaaaaagcaagatacttactattagtctggtttgatccaggttactaaataaatatttcaggtagccaacatttttctgttctcttagtttgagatattttagtaaagatataaaaatcactgacggaagttaaaatttaattaaatttttttcaaaattcctaccaagccagcccaataatattgcattctcacttctttatattccaatccaacattttttacaagtgtaaaaaattttactatctttttatagttaacataaaaacataagtcatatcataatcaaaacataaaacatatcattaaaaagtttcaagtgctttttttcttaatattttattgtaagcattgttttcatgtttagcctttgtgattgtttttaattgctatgtaagcttccatagcatccatgcatcgtattttcttcactatttccttattgttgtacatttggactatttctgatttttgttattttagctaatcctacaagaaaatctttacatatgtagcttttgttactttcttgggcaaatccaggaatgacatttgaaagaataggttaaagtaaaacagcattttgcaggatgctctagacttctgttgaaaacaatcccatcaaggaaagaaaatgtttctctaactctctgttaccatctccaagcctttgtgtctcctctggagcaggcactatggtggaaggagaggaggctctggagccagaagtcctgagccgccttcccctcccgacctcagagccctgttcttcctctgtgagatggcggcaatggtgcctcttcacgcaggggactgtggagtggagatctgattcggaagtgcctaaacacaaaaggcacttggaaagccagaaccattagcatgtgtgcctcagtttcccaatctgcaaaatagaatatcattttcccaatgcaataatgggggaacactggactgacatgctttttttcttcctctgtcattacattgcagtgtatacttgggcagaaatttctctctaggctttagttttagcatctgtgagataaattatgagaggattgaacatcccttttagctacaaaatgagttgtgtgtggtcttgaaattacaaagttttaaagaacatttaatgactcagcccccgatccttaccccaccccacaggtattggctctcagaattcagagcctgaacttccttcttccttgcctctccaaatactttttaaatctgggctctcaccttaccacaatatttttaacattctaagtgggagcctctgctgtgaaaaatccaaggacaaagtccatcccttctgcttgtgcgtcggctgcagatttattgggggccgtgcaaggacttcggttggtgagaacagacttgaagagaaagcaaattactgcccgaagcagccaaattgcagtggtgaaaatgttaggcgtttgcactgtccactttgcttgcctgttttgatttgtgctactcagcaactaattttcaggctcaaggagaaaaaaataaatagaaagcattagaacaaagacaagcaggacccttgccagtccagaccgccgttctctgagtgcagccatccctttacagttgcaaaatgagtttccatgcctttgtttactggatcctcataaccatcctatggcaaatagcagaaaaagcagcaggatctatctcttctcgtaaatgaagaaagtgaggctcgtaggtctggtgacttgcctacgagtagaaccaggcctcaaacatggtttctcggggccctcgtacagggtctgcaccttgaacagtgtgcagccgctcgcccaggtccgtacaccatcatatacccaaagactggattctgaagcctacttctaaaggtattccccttactaagcacttactcttttcctctcaatttttacagtaaaaaataattattttgatgctgaatattctcatttaatatacattttctgctgagtatgaatattaggttattaatatagaactatatgtttttctagaacgtattatgatggttctaaatggagaacttacattttctccaaagctgataaatccatagtttccattttactctcagaaaatgcatctattctggaacttccttagagggatgcgcctcaatttgcagttcaacccactcctgtgtgtgcaggtatgcacaccgctcaccaccccactgtatgcagaggttataagtaaaatgtaaattaaatcatatttgagaggttttgccctgaataaattgcctgtaattaatctgcttggtaagagtggatctccatatggtgtgaaaggttttatgtgtatgagattctctaaaagaacagcacaccgtcacgcacagactctgaattgagatttgttttttcactaactcctccaggactgggagagttatctttgaatgacaaatgtctataatctattcacaattatttacgtcatattttagcctggggttcacctgcaggctcggtgcaggtagtgcaaatccttgtgctgttgactggagcaaagcaaaagatggttttctggtgctcagaacaaatgcatgaagcattaaacagctggagatgtgaatatctgagactcttagtgggccacccccaaagctggctgagaggagggttgggaagaggggaagtattcaagtgctgcagcaggatgtctgtgatgggctgtaggtgctgttgcctttgccaagccatggcaggtggggaggagggctcagaatggaaaccaacatcaccatttaagattaacaaacccctacccccctcttttgctgtagatactagagtctactgtgtcttcttgtattttgctaattccccaccatgcaaaattgtactggctccaagacagaatgtgaatgacagccctcagttagacctgccctttcatcatctgggctgggcatgatgggagagaagtatgataggggaaagagtagaaaagacaggctgggggagggggtagaggttgcaacctgttcccctgggtttactggcctgagctgcctgctgcagggagaaggcagctgagccaggtgacccatcacgtgaaaagcctgagcccaagccccaggggttgcaggtcagtgtgggtgttggcaatgtgcacgccagcatgtatttacccataggctctggtggttgcaggtgtctgtgccccctgtgggcatatgctggcgcatgtggtgctgtgtgtttgtaggtgtgtcctgacatgcatgtgcaggtgtgcatgtgttggtgtgtgtggtgcactgtatgctcacctgtatgctgatggatgtgtgcctgcatctgcagggatgtgtttttatgtgcagcagtgagtgattttgtatgtatggtgtgtgtgtttaggaggagtttggtggcagcaggacagctccatagaaggaagggcaaacattacttcaactcagggctggcagtctgggttctgagtaggtgagcagaaagtcagagatgtcacagatcatgttccagggattctgtggagaagggagacaagaaaacctctgtcggatcatggagaggtgtgagtggcttcttcatagctgcatagacttttcaggttggtctggcctgactcccgtcagaggtctctcatcttccaagtaagcctggggtctttccttttttgtagcttccttcctctcaattctcccgacttagaatgcagacgccactccatcccccacctgcggaaaccctgttcaccccttaattctaagcccagatgtatggggatctcccagacccactgttgacattgttaccagcgttaaagtgacacaagcaggtggaaaattgttgttctcagaattcagggtcaaaaacccaacagaaatagttccctagagtgaggcgttcccagagaagagactcggcacccagatagtttcaggaacatttatctgtcagcggaaaatgtcaacagctccccaaattctcaccagttcttcccacagtgtccatcctatgtattttggctaaaataggcaaacacgcatttaatcttctttttttttcaccctcggaataggttggcaaacaatccaataaggtggttcccaatgacctactttgcccatcaagataaccagatggataatgatttcttttcttttctttttttttttttttttttttgagacagagtttcgcttcgctcttgttgactaggctggagtgcaatggcgcgatctcggctcaccacaacccccatcctgggttcaagtgattctcctgcctcagcctcctgtgtagctgggattacaggcatgtgccaacacacccggctaattttgtatttttagtagagacagggtttctccatgttggtcaggctggtctcaaactcccgacctcaagtgatccacctgcctcggcctcccaaagtgctgggattataggcatgagccaccgcgcccagccatgatttttaatatctgattatttaaaccatctgacaggccacttgacttaccaattgagtaaatatttaccaagcacctctcatgtgccccaaatcaaaccaacagtcctgtagatggttggctcaaccagcccactggccaggccagtgttttgaagatgaaggttaaatgattgagagcccataagcacaagaaagtgtccctggtcccggaaaccatgttctcaatttttgcaaaaaagtctaaatttgtcttcaggctcacttctttatggtctcatctaattttcatcattgtgtggggcctgtgctaatttccatgctgagtttagtagcaaaggaagcatatatggaggataccacccaggacttttgagtcagccaagagagcttcagaaattcctcctttgatttccacagagtcgagagaaatctttgtcttttctcccacttaactatcaacacttcaaagcccagacctacataaaatacacaaatgctttctggacaaaatgaatcttgtccgttttctttgggtttcattgggaacaataaagattgagctacaaaaggttggcaaagaggattatcctttttctttctattaaacatagaggcttcaaaagaacagctcagaaagtcaaataattgtatccaccatcgctcacccatccatctccctgctgacctggcctgtgaaatcccctttaaaaacttagccagctctcacctgcccattgcaaatgctgtattgttaaatgttcccatctataattccatgagggaggctgctataaagcaaactccttccatttcaacggagcatctcgtcgcgcaatagctctttggggtgggagtggtggggtgcctctaattttcaaagctttgtaatctcttgttggaaagggagctacttggaaaccagaaatgggctgtgaatagagttatagccacaagctcttgcagtccaaaggggctggtggggaggacacggagggtaagattttggaagccatttttgatcttcatgacaatttcttgattttgtcggtttggaagacacatctgttagtttttagctatggctgtaaacacaaagtctcagacttgaagctgacctaattcctcgtttggtttcagaccatagcttcctcagaaaattcctgtcagccattttgaagagcacaaaatggctcctatatgccaagagatagagagaatttgctatttagggatttttttttttttaagatagggtctcccttcttcactcaggctggagtgcaggggtgtgatcacagctcactgcagcctcaaaatcctgggctcaagcaatcctaccacttcagcctcctgagccattggaaccataggggtgcaccatcatgcctgactaattttttctttttttgtagatacaggttctcactacgttgccctggctggccttggaaactcctgggctcatataatccaccagccttggcctcccaaagtgctgggattacaggaataagccaccacgctgcctatttagagatttttaaatttggagttgaacatttaatagcagcattcaagtctggaataatttcagttaagaaagaaaagaatagaaatctaaccttctaattggccaaccatctaaaattctatgtggaatgatcaacaaaggaagacagtattgagttgctgggtcctctctacggggtcagaaaagaggcctggttcacctggctctccatgttttccctgtagagtccttcaaacccatggtctttctctaccaactttgaccctgggtcatcatctctctctccaatcaccattccttctctttgaaggttaaaaaaaaaaaaaattcaggccaggcgtggtggcttacacctgtaatctcagaactttgggaggccaaggcaggtggatcacccagggtccggagtttgagatcaacctggccaacatggcaaaaccacatctctactaaaaatacaaaaacaaaaacaaacaaacaacaaaattagctgggcatggtggtgtgctcctgtagtcccagctactcaggaggctgaggcaggagaatcacttgaacctgggatgcaggggttgccgtgagccaagatcacaccattgcactccagcctgggcgacagagcaagactccatctcaaaaaaaaaaaaaaaaaaaaaatcagagggaacatacaataagccatatttaatggagttctgaattatgtaaatttgaaaaaaatgcaatctaaaaattcataaggtctcactatttgtagtattcaagataatgtaagattttacatttattcctatgataaattaatcagaattttgcaaagtctttaggaatgcatctctcatgtaaagtgtagctactttgtagtcagaagtggcctgtggcaatatcgtgattaggctggggtaggggttagggttgctgccacttagttctaccttcagtgataagaagagtagacagcaagccttggcaacatagcaaaaacctgtctcttctaaaatacaaaaaaaataaaaaagagagagagagaaaatattcgctgggcacggtgacacccccctgtagtctcagctatttgggaggttgaggtgggagaaatgcttgagcctgggaggcggaggttgcagtgagccgtgataatactactgcactttagcctggacaacagagtgagagaaaaagaaagggaaggggaggggaggggagggagaggagaaagaaaaaagaaaagaaaagaaaggaaaggaaaagaaaagaaaaaagaaaagaaaagaaaaggaaaagaaaagaaaagaaaagaaaaggaaaagaaaagaaaagaaaagaaaaaagaaagatggcgtctctctgggaagattttctgtgaccagagtgggactgggctggtatgtcagaatctcggaatatttgacctggaaagtattatagataccttcaagttccacttctggattttaccaggggagaaatgatggccctagagaaaaataatacagatcaagaaattgccatgtcataagcaaatggcaaattcagcccttaaacccaggacttttgattcttgctctagctgcctgtcctctgggctgacaagctgccctgttataagtaaatggcacttctggagccagccacttgggtctcaggtgtgtgtgatgagaccctacactagaatgacagccacaaaagagctgcaggtggctgggccaccttagctcatcctgtccatgaaggggatggctttcaactgtttcagggggtacaaaaaggcattggatcatccccacagtgaggaaacccctccccactccccctaccagatagctgtcagtgctgctgagaagcctaccttggtctccctccactccagcttctgcaggccaagtggccattgttgttagattttttgccctgttgtgccataggccctgacctgcctacctgctgggcaacaggctgccggagggcgaggggcagcccttgtgccttgccccacatgagctgcactcatgctgccttccagagaagatgaatttagccttccacctgaatcctttcagaaacgaagcaggcaggcaggcaggcagcaagaaagctatagacacacacatacatgcaaacacatacaagtaaatttagccagagtgcttttagtctataaagatgttcagtaaaagtttaatgagtgaagaagtgacaaaaataccatgtattattagtatatctaggctcagacaatgtcttatggtatgtaaaccgtatctcaacaacaacaacaacaaaaaaagctactcaggaggctgaggcaggaggattgcttgagcccaggagttccaggctgcagtgagctatgatcgtaccactgcagtctaggctgggagacagagcaagaccctgtctctaaaaaaagaaaaagaaaagaaaatgtcagctatgtacaagggaatcttcactcaccagaggaagcccttggaaggttttgtgcaagagaatattataataatatgtaaatataggtagaatatttaaagaaaagctgtcgtgagttatttggtattgtgaggactcttcttctaatctacttgttttcatgagtatgaattttcatttattggatttttgtagaatcgacagatatattaacttttcaaaaatagagacgtgcccagattttcctgtcagttgttcaccaacggatgagatcccaaaaggtaggcaagattgatcgctaggttttagaagggtttccttttaagggctttttaatgaagcccattctaactgagattcccactgccccactgccctagcgtcccgttaatgacaaggagaggcagataaatgcctctttgtgccaataaatcaatggcatctctgcttctctgctgggactgtccaggaccatggtaaccataaggacagtgacccactgatcttaggccttggcaggcaaactagcctttactagtgatggtgtgtcctgttaggctatgccagctggggagagaagactaaaaccaatgagagaaaaacaagtaattttcctgttagtgaagagtgcctgttttaatcatacatttcatcgtcagaaatattatcagagatagaaataaaatgcagaacatagctgcgcattaattcactttcagtgtattttctgagcaagcgattgtatgtcagcggctgcattacagttgctgctctcaaagtactcacatttctagaggagagggatatgcctggctggtaaagtgatagaggggtggtaactgtgaacacttgtgatgccggggcttgttttatgtgctgtttgcatgcattaggtcaactaatcctgtggcacttttattgatgtccccacttgataggtggggaattcagccctgggggtccatggctagcaggagcacagccagcctttggccccaggcagcctgccctcgctccccccgccatgaaccgcattgcctatacacagcaccccgggagcccagaggtaagggtctccctcatatttgcttgtcacggacttggagggaggagttgaggtttgtctgccacatggagaagggaattagcaaagggaagatctggtggtaggaataggattttctgccccagaaaaccaggaaacaccagccgtgactctaatccctaccccatccctgaaactcacttcccaccctcaggctagatgaagaggcgggggtaggggggcatggcctttggcaccctaaccggtagcctacccctcctccaggccaagtcctcacctggccccatggttcctggtcccaggcagaccctgttgccacctgctcagatgtgtcacccttccttgtcccaaggacctggctcctaaacaatgtgttcagtgacgtttataacaaaggcactcttccccaatctatgttcagcaagatgttaatactatactacaaaaaagaaggaatgtacattttggaaacagtagtttaagtgcactgaagaagggttttgtttgttgttgtttaagtgcaggacttttccaaaacttctgtctgcaaatgtgcattacacgttcccagggggaagggaggaagatagagttatgcaatgcaatggaaattaatttcacccctcttggcatctggtttttcagggaacacctattaagtgacctgcagaatacagagtgagggctctaagaggaagttatgagaaagctgggctagaggattaaaggaaagcatcctgggaggtaggatatgaatgggtaggatttaatgcaaaaaggaggaggaggaaactttgcaattcagggcatggcctcagcctgatgacaaatgccccagggggacctcctaaagtcggttcattttgaataccggcaggagcagacgcaagctgccacccctcctgtgtaaggctgaggtagctgttccacagtttcctgggacttgccagcaggatgaagttccagttgttaaccacagtagctaacctcataatgattccttccttcacctgcctcacttcccatgttcttactgtattcatttcctagggctgccttaacaaagtgccacaaactgagtggtttaaggagcaaaaccttattggctcacagttcggaaggctaaaagtccaaagtcaaggtgtctgcagggttggttccttctgagggctgtgcaggagaatctgtcccaagcctctccccagcttctggtggttgctggcaatctttggtgttccttggcttgtagatgcatcatcccaatctctgccttcattttcatgctgcattctccctgtgtgtgcatggctgtctctgtgtccaatttttccctttttataaggacaccagtcatattggattagagcccaccctaatgacctcatcttaacttgataatctgaaagcaccctatttccaaataaggtgacattcagagggtgttaggacttcaatatcttttgaggggacacagttcaacccataacacctaccaacggtttctggaatcatctctcaaaataaactatctaaactctaatctttgtttcagggtcagcttctagcagaactcaatgtaagacacccttttaaagatggtacctcagaggtacagacagattgtgaaccttccctagcacagtgtaacaagtcccttgcaaaaatcctgatttgagtcaacattgtaatttcttgcttaaatctaagaatatgcctcccagcttcttccaagactatctgggggagttgtttctagggtggctcaatttattcgttttgaacgctgatggccgtgcaccacaccgtggctcaatgggtgttgaggacacgttcacatgcaataaatgcacagggctttgccattaggtggcattaggggaaggacacttccagcatactgcagaggcattctcttccagcctggttgcctgtcaagcacctgctgagatgactggcccagacggagattgtggggttgctgagccagccctgtcccctccttatctccggaagatggaataaatgcgtgtcagaagggagggtgcctcccatctcgggagcaccggggttgcccttcagaaaaacattgcctgcacattttgtagtcttgaaatgaatctgagtggcaattcaagcgggcagagcttgttttggattttagacagttctacctgcgtgctcctcttctctgctccagctctgacatctcggctccacatacagtggtctgaagtggcatacggaccctgagaagaggagaggcaaaggtggcagctgtggctggcccctcctgccttctggtctccttggctggtgagggaagaacaaacacagttgtgtttccaggtcacagctgccagggctccacctgtggggtgggtggctccagttctgttctgagttaagaaatgctgcaaatacgttctccttgaaagagacactaggaattgaccatttctttctgcagctttctgtagccaataagcatttgaggaactggagaagggttcagccctgaattgcaagggaaaactgtgtgagtgtgttttagttaagaaaaaagttaattctagtgagacctgcttgtttacaaaacagatgtataggccagacagatgtacagggatgaccttgactttcttttgtcattgcaggaaggtggggtatgtatgacccctggttaagaacaataggaggccgggcgcagtggcttacgcctgtaatcccaacactttgggaggccgaggcgggtggatcacccgaggtcgggagtttgagatcagcctggccaacatggagaaaccccgtctctactgaaaaaaaaaaatacaaaattagccaggtgtggtggcatgcctgtaatcccagctactcgggaggctgaggcaggagaatggcttgaacccgggaggcggaggttgtggtgagccgagatcgcgccattgcactccagcctgggcaacaagtgcaaaactcggtcaaaaaaaaaaaaaaaaaaaagaacaataggatcagaagtcggatggctgggtccatatatacctccacctcttctgactcccagttttccatctctgtttgaaaatgagtctgtaagcctctccccacctctgcctgttcccatcccatagcaccaggctgacttaggcagccacacagctgggatgggaagttggcctcagtgagcagatggtacaggggctgagcttaaaacaaaaatcacaaacatttcttggggggtatcctcaaggccgataaccctcttactagagtgaaatggaaaaccaaatgtgattgagctgcctgagcgataaaagcatttgctgttattctctgatttgctctgttcatggagagtttagtcataggagtttctattctcagaaactcacagcacatcacaatacgtagctcatttgtcctcaaaataattctgccctgcataaatgggtgtgtgtgtaggggggtggtaattataagaaaccatgtgtgttagaattgatgtcagtgctttgagatcatcagataaaagctaataaacaacacatatgtgtacaggccacctctctaattcataccagaagcgctggttgtaatcaaagcatggcatattttattttaaactattttataaataacctttgaaactgtgttgattgtgaaagccaagatctgatgtcctgttaaggaaagatgaaaataggctagaattaaagagcaatatgagtctggataaaagtgacaggaattactgtaggacaataaagcattgggtcatgtcaagttttcatcacatggagaatggaaagctgcatcataaatgctctccacctaatcatcctgagcattctcattttagtgcgatatattgtcctcttggaagcaataatctcatttaggtcatgagtaaaataagttgggttcagcgtttccatttcaagatatagagaatatttctccagtgcagcataagaaatagaatatcacccaggagaagctttacataggcagtgataatgtggaaaaatctaataaagagaacccacgtgccccagggaggagaagccctctgtgtgtgtgattgtcgtcaggtgagcagttatggcacactttgtagttttgccagtgcttttatgtaaattactgtatgcaaaatccacagccctcggggatgtgtgggtatggcctctgttactgacagaggaactgaaacttcaagaaggcaagtgatttgctcaagattggttatcagtagagttggggtttgaattccattcttctggattcaaatctaagctctccctctgcacagttacatgtgggcaggctgtgcatgcatatctgtggcctggaggagtggggtgagtaaccttacaggaaggaataccctgcactaaaatttgcacctaagcttccattattacgcctacttttcattttctgtttgttttcctcttagaggttaaattttccattcctgattttaacccaaagaaaggctaagtaaattccaggggccactcttgaataagggcagctggaaaaaaatacacaatgcccaccatctagcttaacaggatgtggacaccttcagtcgtggctaaatctatacagtttaaccctagcaacaatttcccagaaataaaacagctgctctttctggtatttggttagtctccagctgggtcaagtccctcttgatcgaatgcttggagcagggtttctcgacctctgcactaaaacacctggggccagataattctctgtggctgtgggcaccagggaggctgttctgtgcattgtagaatgttgagcagcatccctgacctctacccactagatgacagtagcactccccaagttgtgacaactcaaaatatctttagacgttgccaaatgtttcctgggggcaaaatctgcccctggttgagaaccagtggattggagtcatcatttgtgttgatcaatcactccgtgatgattaggccatgatggacaattcaggctgactttattttgctcattaggttgaccagaagactggcaattgatccactgctaagcactttcctgacacaagctgaaatgatgtcctcaggagccccgtgtgcgcatggcttttgggatggttcttcccaaggaggacagccgtaaaccatagaaagcaaccacttgactctgggtgtccctggtttgcaagtgcctcttgtgtccgccatctgcatttctgtctagatggtaaggttgagggaggtcagtttcagcagaaagtgcaaagagcatagattcatgaggcaatcagacctaaattaaagttttggctttatcactacccagcaacgtgatcttgggcaggtttcttgacctctctgaaactgtcctcttctttataaaatgaagattacctaccttgcagggtcattgggataattaaagaaaatgtgtgtaatgggcccagatggagctaggcccatagcagcactgtaataaatgttttcgattactgtacattttacaaaaatgaatgactgaatggaaaggtagaaaggccaaatagagtgtctgaggcatctccatggaggagggtgccagtggccatgtctgtctgtggctgatcgcaagactcttttgacagtccctgaagcctgtgctcatcttcagctcccttgaggccctcttcacctggcctcacccagtgtccctgctcttctgactcctgagggacctgagcattccaaaatagatgttaggaagatgtggattcatgtagaaggatgtcaaggcattgttggttttagcagttgatcttacctggggtgtgaaagacaaccgaagtctgtgagctgcagtgatagaaatgtgagcagatgggcaggataggaaggcacttcttgacgggagcagcaacagaagccactgcctttggccaggactgggggagacacaacagaacaggactggcagaagatgccacatcccagggtccccaagatcaagctatggcaccatcacccatctacagcaagatctgtagcaggaaccagggagaaaagtctagaccctcagtaaactatgcggccaggacagtccgaggtcaagccgtaaaggaggcttttttggcatcttttttcttctcccttgggcatcatggaatctgtgctgggtaaagaaccttggagatcccctttgccttgactcctttgtttcactggccctcagaaaggagatggcagaatcacaaagctggtggctgagccagaaagatattgtaggtggaattcaggttatggatagggttaggttccccttgaagagggagtactgagtttgttccttggagagtttgcggttctagctctgtaacccaggactgactctcagcccagtgctttttctgttgtgccaggatgcatcctcatcctatatcttattttaactcatactgtttccaaatcaaataccaacaacatttacaacctttactttcttatttatttaatgctccccctgaaagtagtaatttgccactaagaaagcctcccctgccctagaatactaaagaaaacgagaactaagcgaaagaggacctgtagtcagcgatttcactgtatcctatttaaaatgaaatggactttattttctgctttgagaacctaaaacaccacactttaaaagatgatatttatcatatcttacaaggtcaaagcatacttattggggaaaacttggaaaaggtagaaaagaacaaagagaaaataaaaatcattttaaaaaatgttacccccaagactgattagtatgttggtgactgtccttctaggcttggatatgtgcattttatttcgtaactgttctttttacttagcaataaatcatggtcatctctccctgtcatgaactatgtctctacatcaactttagtgcctgtaaaatgccgtaatttattttaccactgtcttttgtcagacatttagatggcttcatgtttggacgcattatgaatagtgtcactgtggacattcctgtgcatacatctgtgctcacatttgtggctatttctttggaatgcactcctggcagtagggttgctaggtctcccctcactgtcagtgtccccgctctccggggcctgcttttttcatcacccgcctccccgtaaagattcctaagttccagggctatctccttctgatggagatctcctgggtgatgtggtacacgtcacccacacccctgccaccagcctgctgtccaacagatggagctgataaccatttaggaacaatccttaaacttctaaccgactcagtccctgattagacttcttgtcttgccttatccctggttatgtcccttctccctcccaacccttacccacttctcagccccatgcaggaggcccagtttgtcagctggccttgctaatttctctcttggcccagccagagatcgtctaggttccttcttcctactctctgcccgctctgtagagtcagactctccacgggatggagatcttcaaggccagagagatgcaaaacctccttgcttcaggatccggagacaagctgctgcttttaagcagatcccctctcaggcaggttcaagtactgacccaaagagcaggggggttgcagggctctagtgattcttgtgtgaccccagcagactcaagctgttgcagcagtcagacatttggtgatacctacaaaatcatctagtccaacctctcttccaacagaactcacactcgatagcttctttgatgaatggaaatccagacttctgggaacactactgtgatgagcaactcactacctgcctacctacctaccttccttccttctgctttatttcacaattcttgccatacaaaatgaacaaacacacaaacaccactactaatgctattcattcatttatttattcaaccaatatttattatttactatgtagcaggaacagtctaggtgcttttgaaacaatcatgaacaaactgaaccacagcagcaacaaaatcccagatcccatatagaactaacaattaaagacggagacaggcaataaacgaggtaaactgaaaaatatgtagatagttgtgatacatgctgagaagaaaaataacacagagaaggtgcataggaagtgtcaggctggtggtgtggggatacagttttgggtagggtatgcagggatgtccttgctaagatggtggcattctagtcaaacccagaaggaagtgagcgagcaagccaagtgggaacctggagaagacggaggtggcagtgcagactggcatgttcctggaactgcaaagaggtggtatgagtgaggggaaggccagtaggaggtgaggtcagacgtattgagttctgggggtttggggagagagtgacagttgcaaacaagcttgaaggacattttgctctaagtgagatggaggtcaaggacttggagcagaaaagatacatgattatatgttttggcaggatcattctagcagctgggcaaggaacatgctgagggggaataagggcagaagtaggcattacttagtgatgtttgttcaagaaacatctgttgcgctcctatatgccaagcactgcgtaggtgataaggattaaacgggagcacactcttcttccctctggtaggttactgtaacaactactcccacctctgtcatttatgagtatttacccagtcccaagctctggggtgagcacacccgtccattattgcatttattcctctcagcaaactgtaaggtaacaacctcaggatatcctcattttacagttgaagaaactgaggcttagagaggttatgtaacatatcgtagaccacatagatactaggtagacagccaagatttaaacccaggactgtctcactccaaagcctgcaatactaaccacaatgctgttgctggaaactttttcttacgctggactttgaaagttcacctaagacagcatgagaagtgatggtatcttccaggcagccacctgggccaccagccctcccctccttgggatctgccttcccactttgtctgaattgagggtccaacactgctcctcggacacaaggtccaggtggggagagaacagagtccagcccagccaaggcaagtcacctgggctctctggaacccggactgctttgctgtagagtaaagtcctggaataggtctctaaccctctttccagctctgaaatccaaggcgtatgtgatctgaggtaataattgtcttgcctctcatgcttggtttttgatgagaccttcacattctttaataaacatttatctgtatccaggaattataggagatacaaaatgccgaggaatgcactactcatgatcttttttgatccttttaacaatccagagacatgggctgtgtaaagccacagtgcaaaatacaattaggaaaacacagcaataagaccactgtagctttgaatggtgctgttcactaattattacctccccacccattttctcatctgaccctcacagcagccccatgaggtcgacagggtgaggacattatccccatatcacagatgggaaaactgcagcccagggagctatgcgatttctcccgaggtcctcctcacactagtgtagtggagccgctatgtttattgaccccaaatccaagtcaacccaacagtctctagaacgttccaattacctggccagtcctcacttagataacatcggactctttcagaaaagattaaacaaatcctctatttgttgacagaggttttgctgaactggccctggggatccgaattgatcatggccatcaacacaggcagcaaagacccttcagaagtgttttattcactagccacctccatcccccaaccctccatcgtctaaagtgtctcctgactcagttctgtccccctgcttgtcctcttctcctattagctctgggtacccagtccagatcatagccccaagctctgtcatcattttgcagcctccagcctcccccacccaactcagcagagccctgagaagaaagtcctgggtcccggaggctgcacctgcagacacaacagattctgcaatggtgagagcatatagggtattttgggtctccagtggggtttttgctgattctgtgcctgaccagggcacatgcttcctggaattaataattctagataaagaagcctagcagatgcccacctatggaaaatgcttcttaaacaagaagaatctcagaattaggaggccagcttcttgttctcagcactgcccctaactagctgtggggcctcggacacgtcacttgatgcctctggtcctgttttcttctgtacattatggaggatttggacagagatgatcagtaaggtcccttctagccagactgtcaaggttcaaatcctagctctgccacattccagctgtgtgatcttggacaagttactttatctttcctaagcctcagtatccccaactataaaatgggcacaatagcacctatctcatatggctgttgtgaggacccaaagatacaatgaatgtcagccctcagcacagggcctggcacagagacagtactcagctaagtaactgtttctaagttgtttatgatttgaagctaaggatagagatgcttattctgagaagcagaaaggaacagatggagaaggaaaaaggaagatgggacaaaaggagaaaaactggccaggtgcggtggctcacacctgtaatcctagcactttgggaggccgaggctggcggattgcctgaagtcaggagttcgagaccagcctgggtaatatggtgaagccctgtctctactaaaaatgcaaaacttagctgggcatagtggtggacacctgtaatcccaactactcaggaggctgaggcaggagaatagcttgaacccaggaggtggcagttgcagtgagccgagatcgcaccactgcactccagcctgggcgacagagtgacactccatctcaaaaaaaaaaaaaaaaaaaaaaaggagaaaaattagggaaaatagggcaaaattgggggtggagaaaggcaggaaaaatttgagggcagcaactgcaataacttcaacagaaaagtagaacaataagcatacagccccaaaatactcagcccctttcctgcctccccttcctctctgcccctagcagacgccatgatagctcctggaatgatgccttttataactgtggggcatgttcctccaggctgacaccttctgggggggcccctgaacacagcatgggctcagccttcctcccacacacctaccctggtaggcatccctttgagatggatctgagtggtttcccacctccttctaggggtgaactgtcagttcaagaaggaggtttagggggatgggatgtcccctacctggggctaatgggggcttgggaggtggcttacgaatcgaagccgaagacagcagcctacgtgaccagaaaggaaaattcccctccctgctagttaagaagcttcacagaagctcaccagactgttgttttctttctttcttctttttttcctttgtgcacacataagcgtctaaattttgcaaactgttgcctacctttgtctctgagagcagtggaaaatattggactaacaaaaataagcaacagccagtgggagaataatagagaggcaaggtctcctggtctagttggcgccgcaggggttaagcctttctgtcggtgaaacaggctaacagcaccccctgagcctgctgacctcttccggctctcctggtccttcacacatgtcaactgtgagcacgctccaatactgacaagactgtcttacacctgaggcaattttcatagcacaggctgctaatttttcctccaggttttttgtttgtttgtttttttgcctggtaaaatacacataacataaaatttaccctcttagccatgtttaagcgcacagttgagtgatgtcaagtacattcatattgttgcgcaaccatcaccaccatccgtctctagaattcttttcattttgcgaaactgaaactctgtacccatgaaaccataactccccattcccgcctcctcccagcccctgacaaccactattctactttttgtctctatgattttaactactttaagtaactcatataagtgaaatcataccatatttgtctttctgtgtctggcttatttcacttagcctaatgccctcaactttcatccatgtggtagcatgtgtctgaatttccttccttttcaaggccaaataatattccactgtatgcatctgccgcattttgctcatccatccatctgtcaatgagtgcttggttgcgtccaccttttagtgattgtgaacagtgctgctataaacatgggtgcacaaatatctcttaaagaccctgctttcaattcttttgggtatagaaccagaagtagaattgctagaccatatgatagttctattttttttttttttgagatggagtttcgctcttgttgcccaggctggagtgcaatggcatgatcttggctcactgcaacctcccttcctggcttcaagcaattctcttgcctcagcctcccgagtagctgggattacaggcatgggccaccatgtccggctaattttgtatttttagtagagacagggtttctccatgttggtcaggctggtctcgaactcccgacctcaggtgatccactcgccttggcctcccaaagtgctgggattacaggcatg

SEQ ID NO: 8 - DNA, 32306 bp, Homo sapiens
  source 1..32306: mol_type = other DNA
cctaggttcaagcaagtctcctgccttagcctcccaagtagctggaattacaggcatgtgccaccaagcccggcaaatttttgtatttttagtagagacagggtttcaccatgttggccaggctggtctcaaactcctgacctcaggtgatctgcctgcctcggcctcccaaagtgctgggataacaggcgtgagccatcgtgctcggccggttctatgggcattgtggaagtgaccgttaacaagccccaagggtaagtaaaggaaaatgaaaggtgattgattcctcaccacctctccccttttttcctctactctttgcccctgctccccaatctagactctaataaacagaaatgatttttgttgcaagctgaaaactcgttcttgtggttggggctaggggggtggcaagtcaagctttagagttgtctggataataaatgctgtcttcaaggactgtccaacattgtaggaacaagagaacaacccaagtgaacaactaactcctgttcacaaatgctactgcctctctgccatcccctaacctcagcctcacacacttgaatttcgccaatccatttgatgagctcaaatcagtgaattttctcctcgccaaagctcacaaattctgttccccgcttcttgccccacatccaggcattaccaactgcattttccctttgcctgatattctggcatcaagcctccttgccattccaatttatttcaccttcctccagtccttatgatttccttcctgaagagtatttcagcatctctatgcctcaatcttccctgcacaccacaccagctcggcctcatcatcacatcacttggctatgtcccgcctgggttagacagcttcagtggctgaagtccatagatcttattctgtttttcgaggtccacctgactctgaatccagctgatatttctgcccttagcttctacccctctctacttctggttaactatggaccacactctgcttcctcaggaaccacctaccaaggccgtatccatccttcaaggacaatacgtgggcctttcctgatcacatcagctcaacaacttttccctcctacatttcaattgctcttcttaccataatcattagtattcaccccactgtacgtctagaaagaaagtggtcttaaacctaagggaaggcagtctaggtcagaaatttgttgtccgctgttctgagcagtttcttctaggaagtaccaaacatttctgataatagaattgagcaatttcctgatgaagtgagactcagcttgcactgttgaccggctgtccctggatgaacctagttacttttaaccaaatgttcctttcttgaacttgttcctttcttgaacttaatctaccaatgttatctagataactttcctcaaaaaaaaaaaaaaaaaaaaaaaccctctactaggaaagaaatctttgaaccggacttattggaaattacctccttgcagcaggtttgaaacaaaactttgaatttgcctcacaaagaatttgtctgaaactgctttagtatatgctagttatatttgtatgcacatgtggcttcatacatagtggttggacacccatatgtgttatgcactttgttaggtgacgtaagttcacagataaactggacatagggctatcctctagaggcccacagtttgacaccttaacctgagatccttggacctttgaagttcagagtgcccacaaccgctgggtgtggtggctcacacttgtaatcccagtactctgcagggctgaggtgggaggatcgcttgagtccaggagtctgagaccagcctgggcaacatgacaaaaccccatctctacaaaaattacaaaaaaaattagccaggcctggtggtgcatgcctgtggtcccagttacttgagaggctgaagtgggaggatcacttgagtcaaggaggtagaggctgcagtgagttgagatggcaacactgcactccagaaaccccatctcaaacaaaacagagcaaaacaaaacaaactgcccataatgctcctgaaacttcctacggatttatccagagtatgtgctgatgtgcattttttccaaggagggaaaccaaaggttctatcaaattaagaaaagagtttttaccattgtggaagacagtatggcgattcctcaagcatctaaaatcagaaataccatttgacccagcaatcctattactgggtatatacccaaaggaatagaaatcattctactataaagtcacatgcacccgtatgtttattgcagcactatttacaatagcaaaatggaaccaactgaaatgcccatcaatgagactagataaagaaaatgtgatacatatacaccatggaatactatgcagccatgaaaaggaatgagatcatgtccttggcagggacatggatgaagctggaagccattatcctcagcaaactaacacaggaacagaaaaccaaatgccgcatgttctcactcataagtgggagttgaacaatgacagcacatagacatagggaggggaacatcacacgccagggcctgctgcagggtggggggtggggtgtgaggggagggaacttagaagacaggtgagtaggtgcagcagaccaccatggcgcacttatacctatgtaaccaacctgcacgttctacacatgtatcctggaacttaaattaaaaacaaaaaagagtttttaatccaaaaaagattaacagcaactagaagaaagaaagagatataaacagtgaattgcaatgcagcacggcaggtgttatgatggagtagaaaggactggaaaaggcctcctggaggaagggacactcaagtgtctttccatttcacctgtaaactcattaagggcaaaagctttgctacagcttcagtatgagatcctgggcaatccgtgacaaaatgggtctgcttttgcacccccaacttcttctcacatccctgcatcgtgccatgcagcatcaactggaaacctcagcatcagcaaacgacgacagagcgttcatccgtaaggtgaaccagaaaagccagttcaatgacttgtttaaccatggtccatctcagaaccaagagttgggcctcttatttaccagaaaaattgtgggggctttgtgatatggctttaaaaaaatcttgtaattgccaggcgtggtggctcacacctgtaatcccagcactttgggaggccgaggtgggtgaatcgcctaaggtcaggagttcgagaccagcctgaccaacatggtgaaactccgtctctactaaaaatacaaaaactagctggatgtggtgacgcgtgcctgtaatcctagctactcaggaggctgacgcaggagaatcacttgaacctgggaggcagaggttgcagtgagccaagattgtgccattgcgctccaaaaaaaaaaaaaaaaagacattaacataaatttaaatattttataatgacaatccacattaactacttaaagcataagctattttccaggagaggcagcaagtgcattctactcccatgcccaagaagaaaggagcgtgactttggtgggagtactaggagtttctactggagcacttgcccgcagagtgagaaacgttcctagagaggaagttatacctgctgtggaatttaagagaatcttgtcatattttgacaagttttttgagatggaagtctcactctgtcgcccaggctggagtgcagtggcgcaatctcagctcactgcagcctgcacctcctcggttccagctattctcttgtctcagcctcctgagtaactgggattacaggcgcccgccactacgcctggctaatttttgtatttttagtagaaatggggttttaccatgttggccagactggtctcaaactcccgacctcaggtgatctgcctgcctcagcctcccaaagtgctggaattacaggcgtgtgccactgcgcctggctaatttttttttttttttttttttttttttttttttagtagagacggtggtttcaccatgtcatccaggctggtctcaaactcctgacctcaggtgatccacccaccttggtctaccaaagtgctcggattacaggcatgagccaccaggcccagtcaacgtgatgtgttttggaaccctgaattccttggcttgcccggagggttttctttttgttaatatctttgcttgctttctagtatttaaaaaattgtgttttgctctaactatgcaatggctttaagtcttagacaaatttccagggagcaaaacacactcaaccatttcataataatcagaagagagctctgatcaataaataagcaagactgaattttacaaaataatccaaagtttaaaaccaaagcccactttttgcatgatcctttaagagaaagaaatctggaagcaaaacaccttataaaatgacaatgcactttcaggagcccagggcactgtggtgaaatgatgatggctagtacaggttataagccttggggaattatttatgaattctcaggatccttcagttcgccgcatccttctccattatttgaatattggaggctgcctgaccagaatcttgtcaggactttgctccttcatcccaggtggtcccggctgactcctgaggacgttacagccctgaggggaggactcagcttatgaagtgctgggtgagaccactgccaagaagtgcttgctcaccctaccttcaacggcaggggaatctccctctccttttatgggcgtagctgaagaaaggattcataaatgaagttcaatccttctcatcaaccccagcccacacctccagcaattgaacttgaaaaaaaaaacctggtttgaaaaattaccgcaaactatattgtcatcaaaaaaaaaaaaaaaaaaaaacacttcctatatttgagatgagagaagagagtgctaggcagtttcctggctgaacacgccagcccaatacttaaagagagcaactcctgactccgatagagactggatggacccacaagggtgacagcccaggcggaccgatcttcccatcccacatcctccggcgcgatgccaaaaagaggctgacggcaactgggccttctgcagagaaagacctccgcttcactgccccggctggtcccaagggtcaggaagatggattcatacctgctgatgtggggactgctcacgttcatcatggtgcctggctgccaggcaggtaagggcctgtgggtgcccccggaattccgggaaggctgatgggcatccctcttcccagccacagaaccagagggagtccccaggtagatggttccaagaagggagttgaatcttgggttccacctcttgcctgtgacccacggggaccccagtttatgcctcactgttccttggtctgtcaagagagcctgaaatagcattaggttctcctgtccttctcagtccttgacaattaattctgggaagaatagtgtggcatgatatttgggatatttggatgttaacagggtcccgatgagcaggtttctcaagaaattatcttttattcttcaatgaatttccttttattctctgcactcccaccccccaactaagaagctatcaggaagctctttaagttcaggttaatttgctggagacatgagtactcatatatcaaacactgggagaatttaagatctcagaaaagtttagcttcaaggatgtatacactgcttttacgtcttaggaaataacacctaggaatttccttacaagtgcaactttacagaaatttttaaaaatatgcatactcacttttagaacattactacttctcataatcctatgtatatacatgatatgtatatgtatacgtgtttatatatgcaaattttaaatttcattccttgtttcaaatggattttctcgaagattttttttttttgacactgtctctgtcagccagactggagtgcagtggcgcgatattggcttactgcaaccttcgcctcccaggcttagcgatcctcccacctcagcctcctgagtagctgggactacaggcatacgccaccacacctggttaatttatttttttgtagagatggggtttttgccatgttgcccaagctggtcttgaactcctgggctcaagtgatccactcaccctggcctcccatactgctggtactacaggcatgaaccaccgtgcctggccaatatttttttttaaagaaataaaaaaccaccaacttctacctcaaaccactcttcctaattgagtgtgaattttctaataatgtcttccctttgcttactcaggaagacagataagaaagtttatagtgttttttgaaagtctataatggcaacaagtagaatgctaactggctgtagaaagaatttattaagaaatgaaacataatgatgcatttcaggagtctgcttgcttgtttaggggaagtgcccagtcatgctgggctgtgtactggcccagatgtaggagtgtctttaaagagggcattttatttacttattacatttttatttattttttttttactttttgagacagagtctcactctgttgcccaggctggagtgcagtggcgtgatctcagtccactgcaacctccacctcatgggctcaagtaattctcgtgcctcagactcctgagtagctaggtttacaggtgcgtgcaccaccacgaccagctaatttttgtatttttagtagagatggggtttcaccatattggtcaggctggtctcaaactcctgaccttgtggtccaccagccatggcctcccaaagtgctgggattacaggtgtgagccatcgcgcctgacctatgtgttttattttaataggttttggaggacaggtggtgtttggttacatgaataagttctttagtggtgatttgtaagattttggtgcacccatcacctgagcatgtatgctgtacccaatgcgttgtaagattttggtgcacccatcacctgagcatgtatgctgtacccagtgcgtagtctcttttccctcacccacctcgcaccctttcccctgagtcctcagagtccatcgtgccgttcttataaagagggcatacttttaggaaggtttttcaatgtgatttctacatccacagggtctttgctaactcagtctaagccaagaacaggaaaatcttgacaattaatcatgctctgcctctggaagacacatttcaagtaaactcgtgttatttctagctattggtgacttatccaagggtctgagtggtcttggagggagcaggggaactagagtcccataaggactccttcacgccattctcaacatctatccataccccatctgcccctcaacactataggaaatacaaaaatgagggagatatagtcctgacacttaagcaacttataacctggtgaggaggagaaaaacatagatataaatatagtaataccagtcagagtgtggtaagatctacggagcgttgggctatcagagaaggcttcatgaggatgacatgtaaagggagccccaagggaccagtaaatttccatcaatgagattccagggagagagaccccaatgagccaaaacacaggagtgccttagggagcagcaggtggtccaatgtgctcctgccctggacagggagggtggggatatggggaattcaaactggaaagttaggatgaggtaaaatgaatcatctaaggcattaggcatttttagtctgtatattgctgggccctgggatgtggtcagaaagagaattctcctagtgataacttcttaacttcacctagtaggcaataaagaactacaagaaaatttaaaactaccatttataaagtaacttctatgtgccagatatgtgtcctattgtaatcttcatagccactttgcagtatggatatcatgaacccctagatgaagcaagaacccaaaactcagactcgcacaggcagtgagagtgccagggtttgaacctggtctccctggattgttctgtttctgttcattgcaaacagggtcagcgctttatggagatgatactggcatagagtggagagagatcaggtgcacagagacaggtcatgaggctcttaaaatagtccaatcacaaggcaaatgaggacccagtggccaaagacaatgacaggggtcaagtttctggaatcaacaggaatttctaggcatcactccagggtaagaaaccatctcatcacagactttttctccaagaggaacctagtcttgaaacaaatcagactttttattcctcagggtaagagttctctaatgaaatcagactgtcaacagatgcaatttctagctgattcataagggcagattttcattctgaaaatttccaaagccaccatttaacatggcaagggtttatgaggacatattagtcaacttgagtgtttttagtctctatattactgggctctgggatgtggtcagaaagagaattctcctggtattaccaatatggcacgttattgaaggcacctttctcagaactgtggaaattctgagagcttgtatgtcccttgatcatctttctttcattctaggaattctgtgatcaaatctttatcctctagactcaaaaataattctttaaaaaaacagtgtggaaacccattgagataatgtgtttgtgtaattatccattcttgtaaagacaactgcccacaatggaatttcccatcttcacctttgtcagtcaaacacaaaatgtttaacccttcacatttaatctttcaacttcttgagatcccaaagtattattgctactgttacttcattcatcttcaagacatgtcttgaggctgtaagggctttcctaaacattgggagctcttcataagacagttgctactttccttaaaacagcatgccttaaataggcagtttgataaatcacctgtgcatatacttcattttagatgtgagaattgaaaaagaaaaaaattgacttcacaattctgcaccaaaagaatgagtttcacgaatgcgtcagttagaggtgctaatatagaacggttgctgagctctaaaactccactactccaccccactcccctgatagagaagtattcccagactggtttttgcctaaagccttgggtattctgaatacctctttgtgatttttttttctcttcctttttgagacagggtctcactctgtcactcaggctggagtgcagtggcgagatattggctcactgcaacctctgcctcccgggttcaagtgattctcctgcctcagcctccccaagtagctgggattacaggcacctgccaccacgcctggctaatttttgtatttttagtagagacggggtttcaccaagttggccaggctggtctcgaactcctgacctcaggtgatctgcccaccttggcctcccaaagcactgggattacaggcatgagccaccgcacccagtcctctttgtgacttttttgttggagtcattcttctgcgagctatggatatcatcaaggcaggcagtccctcaacgtggcagctgttcccacccttccctgctcctcttcttgcccaaggatgagcagaaactttacagaaaaagagagaattccctcaggaaatgaggcatgttgcttgaggtcacagggaatagaattagcaggaaaataaaccttgcaggaagcagaaagtgggccaaagcactgaggagaggacacggggagggcgtgagtttttgacagatttctctctgtaaaatgaggaggtcacccagggatcttgaaactcccttccagttccttcactctatgtcaaggattccagaaaggaaataaaataagctctgcttaaaaggcattttcaagctggggaactttgggatgaaaaagtttcccaaatgaagacatactggcacggtcagggttgagcgacttttgtgtatcaatgcacccaagcctgatgtctcattgtgggttatttccagctctgaagaagtactgcctggcagccagcatgacccactgcttctgtaattgctgcagttgcaggtgtcaacgcaaaaactccactcactcaattctcaaaatctatccaatatctctcatgccccttttcaatactataggaaatgtaaaaatgagcgacatagtcctgatccttaagagagtcataagttggtgaggaggagaaaggcatagatatagtcatggtaacacaagtcatatgtggtaagatctactgagcatggggctatcagagaaagcttcatgagggtgacatggaaagggagccctgagggactggtaaatttccatcagtgacattccagggagagaggccccaagacacaggggcagcaggtggtccactgtgctcctgccctggacagggagggcggggtcctctgctgtttgtttgcttgtgtaggaattttcccctcaaacaatgtccttgtctcccatgggagaagttttacatgagtgggaacctgtttctcttctctatcccaggccattccctgagagaagagaaaggggaatcttttttttttttggacagtttcgctctgtcgcccaggctagagcacagtggtgcaatctcaactcactgcaaccttcagctcctgggttcaagtgattctcctgcctctgtctcctgagtagctgggattacaggtgtgcaccaccatgcctggctccttttttgtaattttagtagagagggggtttttgctatgttgcgtaggctggtcttgaactccgagcctcaagccatccatctgcatgggccttccaaagtgctgggattacaggcatgagccactgtgcccagctgaaaagagaatcttaattcagttgattacttctaaaagttataagaccttcttcactgaaagagttcacatagaagctgaacttgaatccagccctcttaaatttcagttcaatattccctgcacatctgcacatctttacatgaattctcagtgatattccttcatccatccattcatgcatgaggattacagagatgaagaaagttccagctcttatcctcttgtgctcacagtttaacacgggagatgaaactgctgctgaatggctcccattctgaagtcagccaacagtgtttgccaatagcccccactgaataccccaccatattctcaagtaactcactcatttaggggaaaacaataatataattgtctctagaaattttaggtattctccactctaagattactcagtgttttgactgatctcaaggcgactccgtggtttgcttctgtcaaaaagaaaacacagttcccactgggaaatgtgtggagagtaatggttcaaaacgccagctgtacattagtgtatatccaggatgctttaaaaagtgatggccaacccctactgggaagtgaggagcccctctgcccggccagccgccccgtccgggagggaggtggggggagtcggcccccccgcccggccagccgccccgtccgggaggtggggggcgcctctgcccggccgcccctactgggaagtgaggagcccctctgcccggccaccaccccgtctgggaggtgtgcccaacagctcattgagagcgggccaggatgacaatggcggctttgtggaatggaaaggcgggaaaggtggggaaaagattgagaaatcggatggttgccgtgtctgtgtagagagaggtagacatgggagacttttcattttgttctgcactaagaaaaattcctctgccttgggatcctgttgatctgtgaccttacccccaaccctgtgctctctgaaacatgtgctgtgtccactcaggattaaatggattaagggcggtgaaaaaaaaaaaaaaaaaaaaaaaaaaaaaaaaaaagtgatggccaagactcataagatgaattaagcggactctctggggaacagaaccgaacatcacaataccttcccttccagttccttgaatacttccaaatcgcacttaggattgaaactcaccaaattagagagatggaataatgatttccatggttggtctgattcactaggaacaggtatgctcaagcagtacttctcaacctttaatgcatttagaaatcatccagggatcttgtcacggagcaaattctgactcgggaggtcaggggttaccgcctaagattctggtgcaaacaggttccacaggaggcctatgttgtgggtgcatgaaccccattttgagtaacaaggctatcaagtcagagaataaatacgcattgcttttacccacacagaacttgcgactgaaatgagaaaataggctgagtgtgatagctcatgcctgtaatacctgcactttgggaggccaaggcaggaggatcccatgagcccaggagttcaaaaccagcctgggcaacatagtgagactctgcctcttaaaaaaaaattagaaaaagggaaaaccaatctgggaaagatcaaatattgatcaaacattgcaaataatgccataaatgatcagaatagggagagtaacatgctacctggagaactagaaaaaaaattctcagagaaggtggccctgggacctcctggctctgaatggtggttctgaatatcaggagaggggctggagaatggtgttttctagtgatgctgctggagtcgtgtacatggaccgtgttccatgagtagtgagctgaccaattagagagatgaggggtttgccaggacaactacataatgagccatatttcaacatacatgaagttttgtgtgtgtgtgccaagcaaatgagtctcagattctcggacaaaaacgaatacttttgctcagacagacagcatgtgagtgcttctcccccagcttcaaccctgcagtgtctacaggtgtcattggtccccggtagacgtcctcggttcaatgggtcactgatcacttatcaccagcaccacatgaccctggatggcagccaaagagttcctgggtcttgtttccctacctccacatttttttcctgacccagcccgagccacgtgcacaccacagggctggagtcccagtgcttttgtcctgacagcccattctcccactcccacacccaaccagttccccggggggtttttgaccagcagctccgcctgcagccctcgacccggatccaggcatcctgcttaaggaggggttcaggtggcaggaaactccagggcaacaaagtgtgggtggcatgggtggggttgaccggaaggtcactgtgctgggaggaagaggaacgggtggagggaaggcaagatacggatcaggagtattgacatcctgtgccagtagaggggcctgccccatggtggaaggtgtggccggatgagctcgctggcccatgtccccccgtttcccaggctttttcctcccacgctgtatgcagtgcaagaacaagctggtgcaattggactagcagcaattgagtccttttacccaaaagaagaagaaagagcaatgtttcaaaagaataaaaaggaagctgcagccaggtgcagtggctcattcctgtaatcccagcactttggggggccaagaggggaggatccctggagcccaggagtttgagaccagcctgggcaacatagtgagaccccacctctattttttaaaatgataattaattttaaataaggaagtttgtgaaagccattggtttctttattctaaagagcgtggaaaataggacagtctccaacaggccctgatttcctttggtaagaaaatgagcaaagctatagactaggaaacatgactggttaaagccggcccatgcaccaactccgatactgatagttatgtttcctttcgttcttatttgcttcaagctttgttccagaaaacatttgaacactagctagaaccaccacagtgaatatccattgcctaaatgctatctttcaggtattatgttcagacatagtattactttggttaattctcaaaataaagtaggtcatatgattatccccattttctgcctggtgaactgtggttaaatgggcttacagagattaaatgggttgcctgatgtagcccaacgaggaaggggctaggataggaagcaggcgctctcattccagaacctgccttttttcctcctgtctaaactgctgctaaaggtcaaagacaaaggtgccatgtgagcagggaagttatttttgttttttacaaaggtgaagtaataggctgtgagtaatgcccactgctccttcagccccaggtacatgcagtgaggcggtacccacagagccgcgtggcactcctcaacctgctagagcagccggcctcctgcaggatggcttaaggaccacctggtccttggggtctgggttctccgggtcctgctgttccctgcagccttccttctacccccagagcagcttgggggcatctttagagaaagcggcagtgtcgcacctgccccagcagccacaaaacagaggaaattctgagcacctccgggcgtgagtcaccataccacattcaggaattgccacctctgcccacagaatcacccactcttttctgtttgggaccaaaatagatacctccttcagaagccgctgctttctgtactctcagcaataaaaaaacaaaggctaggataggtgtgtgcctctgaggcgtgagggggggcgcgttttctcccctgggaaggttttcagagagaagttgttgctacagcagtacaggcagctgatgggttgagggtacaggaggaagagggaaagtcagggagattggagacaacctcttgtgtggtaggacttccggctattggtttgttcattcctttgttcattcattcattcagcaaatgttcattgaggaactgctatgtgtcaggcacagggtttgagacacagagatgaataagagcccatccttgacctctgcacagacaattatgacacaggctaagtgctgagatacaagtttaaagaaggagtcgatggaacacacagatcgatgttcattacccctgcaaaacccagcacatctcacatcagggactgcctcattcacatcatgttagatgtgacagagaggacagcagagagcgttgttttgtcatcaactactaaaattattaaaaccaaacaccactaattttgaacttcaggtagacagaggatgtagagttttcctttgctcaagaaattcctttacagagaaaatgggaacatttcaagtgggagtatttcaattcctttaataacaacctgttttctagctcccacattccccaagtattctagaagcacctttcataatggacagctctgaccaactgccccacacacatgcatgtgaaccgttctccaatcctaaaagtctgtgacttctccaaggaaaggaggaaggaatcgtattagtccagtttacattattatatactttgtagcaaagcagcaatgtttccttaaaaaattctatatatatatatatatatatatatagctgggcatgatggctcacgtctgtaatcccagcactttgggaggctgaggcgggcagatcacctgaggtcaggagttcgagaacagctgaccaacatggtgaaaccccgtctctactaaaaatacaaaagttagccaggtgtggtggcacatgcctgtaatcccagctactcagggaggctgaggcaggagaatcccctgaacctggaggctgaggttgcagtgagccgagattgagccactgcactccagcctgggtgacagagggaaactccgtctcaaaaaaaaaaagaaaaaaaatatatatatatatatatatatatttgtgtgtattttctcctcatgaaatgagctccttgaagcaaatcggcccatcttttcagagaaagatcagtttattacgtcaccaagtaaagggcaaaggaatcatttctaaaagtttcgtttgaagtccccggagttaaagttgacgtcagcctcttcctgccctggtgccccgagagtttcccgggagttttggggatgacacccggctgcatgcgtgacatctcgctggctgggggcaggcctggtggcttccccgacaatgaaaccaggcagagcctgcaacccagctgagcaccagtgtggtctctgcttccggtccttgtggtagcacctctgtttacagcctgatttagaacaaaaagaacttttgtaaaggagaaaactccttatattaaatggaaagcaaaacaaacaattaccaaaaaaaaaaaaaagttggaggggtgtttcagaataaaagagccgtctctccagacatctgtctgacgcccaggtggcgctgagaccacagggtgagccttttggctggagatgttgatccctggtttactgcacttcgggctttcactacctttcacccaccggaccatcagtttcactttccctctcccagcgtctctcaagctctgcttctctggccccgttttcaatctagtgcaagaggacctgtagccagggcacccatcctcctcctcctcgtcctgcactgggatcccagctctgagcactcccgggccagggaggccttgctgcgtgctctgcttctgtgatcttacagattcctcatggccaccataggagatggggactgttagcactattcccccaccccaaccaccttttttttgagacagagtctcactctgttgcccagactggagtgcagtggtatgatctcggctcactgcaacctctgcctcccgggttcaagagattctcatgcctcagcctccccagtagctgggattacaggcatgccccatcgccatgcctgactaatttttgtatttttagtagagacaggttagtagagaccatgttggccaggctggtctcaaactcctgacctcaattgatccaccagtctcagcctcccaaagtgctgggattacaggcttgagccaccacgccctgccaccattcccattttacggatgaggaaactgagaccatgaggcagtaattcactctcccaagaaagcaaggagccagtagatctgagcagaacccatgcattcaggccgacacaggaagcaagctgtcaacaggcactgttcatctcctgggctgcctaggcaggccctgaagctctggttttcagtcaccctgttattcatccccaagttaatctcccctgaaagcacctgtcgtgatgccctttcggctgcaagagctccagtcatttccattgcctcaggggcactttgctacctcctctgcctggcacacaacatgctccccagccaagactgttccctcagcacatagcacgccttggccagcaggtctcctcctgtccttcaaagggaacctcctcatttgtgatttcaaagctggactcccaggaacaccctcctcccttactggaacaatcttccccatcttgtctgcctaccggaatttttcccaaccagaaaagcgtcttgagagaatggaaaagtcttatgaatttggaaagttgacagcttgaattcaaatcctagcttgaacttgtgaatttctactttcctttacgaaaacctttgcatgccattctgtgcaggcactagcaaggtggtgggagggagatggatacagtagggtgagtgccgtgtaaagaaaagggagcaaagtctgggactgcagagaaatgccacaaagcacagacagaagagtcccggaggcttcccagagaaagatgacagccaatgggagacctccagggaaagcaggagttaagaaggtagaaatgcattagggaagtgtggcatgagcatgatggtcaatttcaatgtgtccacttgcctgggctaagggatgcccagatagctggctaaacagtctctctgggggtgtctgtgagggtatttccaggagacactagcatttgaattgaatagactgggtaaatttcttgttggtagactgagtaaagaaaatcaccctcaccagtgtggtggggatcatccaaccatagaggaccagaatagaacaagaaggcagaggaaggttgcattctctctgttcaagctgggacatccgtcgtctgctcttggacatttgcactcctggttctccagcttttggactctgggacttacagcagggcccctctgctcctcaggccttcagtcttagactgagccacaccaccagctttcctagttatccagctccagctggcatattgtgggacttctcagcctccataattgagtgagccaactcccattataaatattctcttatttatctacatacatcctattggttttgtttctctagagaaattctgtttccctgaataatacaatgaccaaggcccaagacaaaacacatggtgagctccaagagccacgcagagtcccgcaaggcaggagagctcaggcacatagtggaagcagagattttgaagttgggcatgagctgtgtcttggagtctccccatgtcactgggcttaaactgtgtcctggaaggaacagaacatcccagaggggtctcagtcagggcatggcttgtactttggagagacagcattgggtttatgtgaagaatgaaggaagaagagaaagcactgggtttatgtgaagaatgaagggaagcagaaaggcaaacccatagggcattgagtaacctccatggaagtccatgagggtagggttttaacctcggtgggcctcagcttctgttcgtataaaatgggaataacacagaccctgctgggtgctgtaaggctacataagctagcctgcctagagtgaccagagcacacagtaggtgctcactaaatgtgacttgctgggcttctagtctgcatggccaaaggcccttccctcggacctttccgtgtggatgcctcacaggctgctcagtctacacatccagaactgtcctccctccctggaacctactctttctcctctctttgctatctcaatgaatggcaccccatcctccccatcgcccaggaaatctgggcaaaatcctcatttcttccttgctgtcctccatgcacctaataatccagcctcaatttttggcaattccaacttctaaaatctcttaacgccagcaacccctcttcttgcttacttatcaccacccacctcaggactcccatgacagccttcctgtgatgcatgacgggcacatgacaagggcagttgagggtcaggggccacgggagcaggatgcagccagcacatgagggaggataggcgggaggacaggcagacacttgtgcgtaagagcacgcacttgtcaggctgcctgggtccaagtcctagctccaccactgaggggctgtgtgtctctgggcaagtcactgggcctctctgtgccttagttcctgcctctagagaacggagatgagaagaacacatcacctcataattaattactgcataagtaataattttaattatattgactattatactctattagtgtatgatgtcttttattatatatatttatcatataatatactaattatattaatgagtatactatagtattttatataacataccctaattgcttattatatatctgtataattattttatcatataagataatttcataagctaagatggtattttatatatttcatttattatatatttatatttatatactttattgtatttgaattgtgatgtctaataaaataatagatacgttaattatccttcatattcataataagctccttcattggtgtctgggaagccgagaacatttgtgcgtgtgagctctcctgtgtgttcggcatgcagtgtgttgcgtctcagctgacgtgattgctgtttttggtggcccggaggcacagggtgcatgctacaggcaggaagagctttggagcaggctgactcctgagctctcctctggtgccgtggcttctgaggcccccaggtgacaaccagcctttgccattctagtgtttcctctatgttcatcccgcgttctgagtgtggcacaggccacactcctgaccccaccagcatcaggtgctgtgcacatggtatgaggtcgggatggagtgctgttggcatgggggttgttccttgggctacctgtgtccacatggtctgattaaccccttctgcacatcagcaagggttcagcacctcctcagggttagggcatgcgcagggcatgcagggatttgagggtccttggacaatgcatgttctccatgcaagcatgcagagacatggagagggtctttgtccctttcagtcctaacattctgtggcttcataactacttgtcccacacccgagtaaaaagcaggatcttctcagcactgtgctgaggaagtctttaaaccttccagtaggacatgggatccctgcctgcatcttaagttcccatgtccttaggaaacgtggccaccttggaattcagctctccaagaagcccagtgtgaatgcccattgcaggagcatctgagctcacccccactaaccctgaggtatctgttgaaaagaatagaacccacccactgaaactatcagagatcaaatgttgtgtgttgactaagaatttgtgttaaggcagggatagttgagcaccaatgctttcaaggaactctggagaaatgcaagcctgtatttcctctcagcatggctgcagaagagggccagggcagcagacaggcattttgacccactcacagatgaatacaaagactaaactcagcaaaagccttgaggttgcatttatgtgtctgagaacagtccctggagcacaggcatgggcttttggtaccacgtaacctacaaccgtaagctctctgctctatgctgtccactccatcaatcccacagtgcctgaaatgtcattgtcttacccgatgtctggggtagcatctgtgaacacacggatttgacacttttatgatgctgtttgtctctactgaacccttcttaagcaaaatcacagtagactagcagatcatggtagtatctgaaagcgccaggaccccagaacctcaatcagtgctccccacttcccatggttttctccctgtgtttccacatttggattgtcattgtctgtgaaaaggcatgatccctgtcaacaacacacagtgtatgcaatgtgtgtcttgtgtaaaacggagtgtgaattcttgagagcaggaaggaaaaggggtacaagaggttcccctgcgagatgggtgaggagcagggcagagtgagcctggctgctgggaacccctgaggagagctctggagagggttctggacacgtgggcttggtcagcctgacaattaggatttgtgttaacaggaccggccctcacctgacaccaactggactgaggttcccaggagaaactcttagcatcatgactttggaggttgccatcatttttgatgactttttctttttccttcttttctgtttccctcttgtcaagtttctacctgtaagttatacttacttattctgtgtttctttacagtctcaggggaaattccgttgagttgaaaatttcctcagctcctcgctccagtttctcagaagagtctctctccttgcagaaaagactgtgggtgggagtcaccaatttagcatatttctactaaaaatatgactccatcccaagaaattggttttaaagatttgaggaaaaactgcttttagatttcagagaaagaaaaagagtgtgtttctacttcccccttttaaatctgataacttttattttaatcaggttgacattctgtggtgagcttgtggttaactctttcatgcccggaagcctgttgtcgaccgagaagactcccaggattagaacataaccagcgcccaacatttcatttgcagaagcatctgaggtcccaaaatgtgatggtttagcggcagctgcttgtaggggtatttttggtttcctgcttcttttttcccttctaagtaaagacaaaataactagatccctccaccccaccaccccatcatttcgttgttgtttaacaagtgttgagtaaggcacagttgttctccaaaagggtcttaccctgtgagttactcattttagggttacggaaactctgactatttgacccagtcaaccaggcctcagaaactgtgggtcagataacaaataactcatggtgcttctgtgctaattactgcagagatgtgtccaagtgctcggagatctcctccttgaagccaaggtctcctccttgaaccacaccgccttcttctcctttcccgagggaatcaggcaacttttctttgatgacagcaacttccttaaatcccccaagccctgagacaggcactttacttctcccacatttccttcaacatttgtgcttgctgaatgaaacaaggaaggtgaatgtttgcttgaatgaaccaatcaatgggtaacagcaccagtgctaatgctaatacactcaactactcattcagaacctaccatggcccaaggcttgtgctctgctcttgggtgctgatccagcaaagagtgggtggagggggagtgggaggtgaagtccctataccaccacttatattttgcactgtcttattttttcctctggtcacatgcatacataaattttatatctctgtaattatgccatttaaaaatcttcctactgaggtagaagttgggactcaacccccgaggcagggctcagacatcggacaaaattgaggactagcaaaaacaggattggggcagaagcagctttccataagacatgctcatgagtgtgccatgtcagtttaccattgccatggcaacacccaggcgttcctgctcctttccatagcagtgaccggatgacccaaaagttactaccccttccctagaaatttctgcaaactgccccttaaactgcgtgtaattaaaagcagatataaatatgaatgcaaaactgccctgacctactactctctgcctatgtggtcgccctgctctacagaagcagtcacggagctgtagcacagccggaactataacattgccacttcaataaagctgttttcttttattctaccactggcttgcccctgaattccttcctgggcaaagccaagaacccttgcgggctaagccccacctcggggctcgcttgtcctgcatcactaccaccttctctaggaagatgctctaggcttaatcattgtgttgaattcagctgctgaggactgagaggtgatttggatgattgacttcataaataaaattgggagttagactttccccgctgtggacttgccactgagtttggggagaggggggtttctttctctgataaagttccacatccacagacagaaggaaaaggccctgaactaatcacttcctttggaagaagctttggtaaagaattaacatttgggaggttgtcaagaaaaacctatttgttctataaatatttaaaaattattaaaatttcaaccaatacagaaaaacacaaagattaatataaccaacatgtgcccatcatcaggattcagtaaatgtttacagtttaccttggttgtttcatgtcctttttgtttattttaaatacatgctcttgtcgtgcgtgatggctgatgcctgtaatcccagcaccttgggaggccgaggcaggtagatcacctgaggtcaggagttcaagactagcctggccaacatggtgaaaccccatttctactaaaaatacaaaaattagcctgatgtgatgcaggcgcttgtaatcccagcttcttggggggctgaggcaggagaatcacttaaaccttggaggcagaggttgcagtgagcccagatctcgccattgcactgcagcctgggtgacagagcaagactccatctcaaaaaaaaacaaaacaaacacaaacaaaaacaaataaatacatgctgtagatacagtggagctccctctcggtccaacccaatctcattcttcccctccagtaattgctctcttgaatctgggctgtatctcatgatgcaatatacactactggtttgttttttattttatacataaatgcataatatactgtttctatttgcaactttgcttttttcactcagcagtgtttctgagattcatttttgatgtttattctttttaacaactgtacattattacattgtataataatacagagttcttgataatgccaagtttattaatctaaccttcttttgatggacaattagattgtatctgatttttccctattacaagcaatgctctaataaacagccttgaatgtgtgcacatatgcaagaatttctctaggaatatacctagaagtgagatcactgatcctagaacatggacagcttcaattttactgctatatttaaaatgttctctttgaaatcgctatgctaatttataatcctcatcagcaatttatgaaatttcctgaattcctcacatattcctcatcatcacttaccatagggaaattttaacattttttgccaatctgataatataaaataaaatctcattactatcttaagtttcattgatacctggtgatattgagaatgcttccacatgtttttttggtcatctctgtggcctccttgttcatatcctttgtccctatttttattgggttgttggcctttaacttattgatttgtaagtcttccttatgtattacggatactgagtctttggataagtatagattcttggatcttctaagtagacgttattacttataaatgacaattttattttttgttatccacttcttattcctctaatttcttttgcttgttccattgcattagctggggtgttcaatacagtatagaataaaagcaggaattgtatatgtttccaccattaaatagaaggcttttaaggtatcacgtacatgttattttctgtaagctttcagtgtctactgtctattggttaaataagtttctttttatttctactaccttaaaagttttttttcttttaatcaataatggatattcttttcatcatcattagatgctcttcttcatctattaaaatgattgtgtaggttttcttcctttataccataaaagtatttaagtacattgagagattttctaacgttaaaccattcactattgctgggataaactctgactgatcagaattttttttttttttttttttgcttaatacattgctagcatcaggtgcaaatgtgttaacattctccagtttgactgtagaattaatttctttttgtaattcttcaccttatcaaccatatgtattgttgagtgtatacatgttcaagattatcataacttccagaataatttttatttgtgaaaatgcctttttgatctagtttctcttatgttattgtagctatggagctttcttatatctggctaccttatcaacaatatgtattgttgagtgtttacagggtcaagattattataacttccacagtgatatttatctgtgaaaatacttttttggtctagtttctcctgttactaaagctatcgagctttcttttggttgctaaatatctgcatataatgtgttttccagccctttagttcaaccttcacatgtcactgtattttaaagacgtctcttgacataacaaaaatagaccttgtgcaggactttgggggaaatttttccccctcaatcagaatacctctgtcttttacaagcaagcttaattgatttatatttattgtactacatttggacttatttatgctattttactttgaacttttattaaccattatttttcttttactgttgtatttatcttttcttgtcttctgttaaattgattgagtttcctttattttctcttttccttctactggtttgtgtattatatgttttaattctacttttagaattaagtcaccattggccgggcgcagtggctcccccctgtaatcccagcattttgggaggccgaggcgggtggatcacgaggtcaggagattgagaccatcctggctaacacagtgaaaccccgtctctactaaaaatacaaaaaaattagctgggcgtgatcgtgggcgcctgcagtcccagctactcgggagactgaggcagaatggcctgaacccgagaggcggagcttgcagtgagccaagattgcaccactgcactccagcctgggagacaaagcgagactccgtctcaaaaaaaaaaaaaaaaaaagaattaactcaccctttaagttctcaaagtactcgtgatgttaaaagtccacagttaattaacatctctcccagttccaggatcttctgttcagaacttagcacatttctggcaactaaggattgacaactgttaaaaaatatcatttcccattatatttttggattggttgttgctgatatataaaaaatgttgaattttataaatgaattttctatctggccaccttatgaaactcttaatagttctaatactttaaccgtcttgggttttctaagtacttaattaaattcctcacatagaatgattatttttatcttctctttttccaacaattgtatgccttatctatgttaccagttttatcagatcactaataacgagggttcttttaaagtcaattggagtgagaaaaacccaaagatgccattaagtccttgagtgtctttggggaagaaaaggtaggtttaggaaaagagagacaaaaggcaaagtgaacaactaagacaataccatgtattgcaaatttaggtgtgatttaatgatggctaagaaaaaattctaaattatatataaaagtgtattcgtatataaggtagtttaaataaataacaggtaatgtgggcaggcaacaaccatcaaaaatgactggaaatattcaaattcaaccatggtgatttggttggcagcaatcagagaattgactatgcttcagtagattgaaatccgttataatctattaatgtgaatacaggtgacaggaaacaaccgaatacagcattctcataaatagttcttagaatatgaattcaaaagaaactctatttcctcgttgtgacacaatcaagctttaagcaatgaacgctttaattattgtttttcagcttagaaagtaactatatattttatcattgggttctaaaaacaacaccgctcaagaggtcattgtagcaacaatcaaaatttgtggagcattttacagatttcaatgtcacttatgttatttcaactgatcctcatatttccatccttcataccctcacgattcatgcttcagcaaaacattctacctgtagtctcttgtatcttccatatgatgatgctgttttacacttttacacattctctgccagaatggcctgacgtgcctaaccttccagttgaactcccacccacctttcaggcaccttatagttaagagcagccttcattctgttgcctggctcatgggcaggggcaggattcccagcaaggggtactatgtcaagaggcctaaaaggacatatcagcatggtaaaaggaaatggcttggggtgtatgagaagctcctatggttacatgattgggaggaattcttttcatttcgtgttacagacggagcatctgaaatgagaagggaccacagtgagccagtgagtgaaagaggaggaagattctgacacccagagctttaccttcaacatttccttctgtttattccttatctagacttccattttcctaccccatagatacccactctttgcccctaacttgccttcaactcctctactgccttgtaaatgtcccatgtgtgcttgaaaaaagagtgggctctgctattgttggaatcaaatttgttaattgtggttatcacatttgattgttcttactgattttctgtctgtcctatccatcataaaggaagacactgttaaaatttctcattatgatagtagatttctaaatttatccttgttttgtcaatatttgctttgtgtattttgaagttatgttattgggtgcatagaagtacgtaagtgttatgttttcctgattcttgatttgtatattttgaagtgatgtttttgagtgcatacacatatagcatttttatgttttcctggtgaattgaatctttagcaatacgtactgacttctgtatttctattaatgcttctgccttgaagtctgatgttaatataactccttggattcaattgtccattcttttagtttcaaacttttggagcatttatgctttaagcatgtctcttataaacatcatataactgcatatttttaaaaatatatgcagtccaacattctgtaactgcagggttaatctatccacattgataattattactgatatagtttgatgaatttccactgtcttattttgttctttattttttaccttctctttctatgcatttcccccttcttttgatttaagtaagtttttatcctattttattttcttccaatagcttgtaagttattcactttactctgtcattttagatgtaatttttaacatcttaacatgaatccagactttacaaagcctagatttaaccaatatcatagccttcctctcaaataataaataaccttagaacaaattgcctccacaaaccttttctacacagttccctcctcttcctccttgcttctttgtgcgcatttatgtttgctatttgaaagaatttcctgaaagtaaaagtgagattttttagaaggctgacatgagggcagcaatagtagactttcacgtgactcctgtcccaccaccttgagctgtgtggtttagccaacttacttaatctctctgaacctcaatctcttgctttctaaagtggggatgacagtatctaccttccagagccatggtgagcattgaaggtaatatatgcaaagaagcagcacataggcagggctcagacatcatagttattattatatagataaccccaaatggagggcctgggccctctgacttcttcaaagataataatctgaactaaaccacaacatcacacaaacacctcccagcacccagatagtttagacatagctccacctctttgtaagagataaccttcaacagataggtgaaaagcacgctccttcctgaagattatgattctgggtgttggaaaagcactgaactaggagtcagggaacctggtgatctgttgtaatccaaggcttgccacaaacttgtggaatgaccacaggcccagtgttcttatctataaaatgtagaggttggcctaaatgatctttgagctctcttctagttctcatattctatgataccattaaaagggatgttctacatcccttttgcacccaaaccacttactttctatacggcctaaacttctggtttgggtttcccaaggaagcgagagacaaccttcaatattaggcatatctttggagttcttattaaacagtaaacctcacctcactatcaaagatagcctctggctccagcgtcctctttccagtctcccacagagatttcctctgggagtcaacgagcttaaggtgaaatttggctaggggtctgggaggaattcttttcatttcatattacagacagagcatttgaaatgagaaggggccacaggggggcagtgagcggaacaagaggaagattcgggtctctgacacccagaatctatctgattctgacacagaagatgaaccgcgttagagcaagaaccagatccctctttacccagtggcccaagctcttgatgcgtacctgtgtcccttgtaaagctcagtgaaatcatcacctagacatttcctgtgctccgtgcgttattctaatcttcactgggtacaagtagagcttaaagttctgatatatggacaggaaagagaagagggatgaagcctttatccagggctggctgggctaagagtaaaacaacttcacctggattcacccaaggggcaaagaatcttatctcagactcggtccacatctttatcagcccccttttccctttatctttcatgttgctcacatgggctccaggggccagatgagttttctcttactttgaacatcagagaaaaaaaataacgacaaaaagttactcaaccgacaaacgtttctcacccacacacctttcctccagtcaatgacgtttccactgcaccctgttagacgctgagtgaggaagcatctgggcacaaaccgctgtggatgcagattgagcagcactcactgtgcactggagggatgggagtcctgtcctcacccagggggcagatggagagggggcagagagagcagccggggcttcactccctggaaactctcatactccaagtgcggcctggggctctgcagccccagcatcatgtaggagcttgttagaaatagcctcaatgtccacacctgccctgacaaatcccagtctgcaagatcccgggggctctgcacacacgctgctgcgaacaacctcagcatccgtcctgccggctctagagcagccaaccgggatatccttcttcttcagcagtggtgtccgctctctaaaatgactgtcccccacatctctttccagggttggtatttctggttaactcaagggacacaaaaatagctcagggaaaaattaattttcattctactccaattctaataatgaagtggtaaaaattaaagggacagctttcagattatgggagtgcagctgcgggtatgtggcaaaatcgaagaaagagggctcataattccacgtcagggaagagccgctggcctgcccaggctgctgtttatctttactgtgaatggggcctcttttttgtttgtttgttttgttttgttttcacttaacctcagtggcaagaaaaatggggcctcttgccaggaggagcagagagtgacgcggaaggcaaagaccacctcatttcaccaagttcctcgtcttttatttgaaatccaagacatagaagctgcacatttgtccttctgggaagagtgacgagtgactgagagtggagtcgaacactcgaaaggagagagcaggtgcctgggagtcagaaaagctggcttgaattcctaaatctgtcgccgtttgtcctgggtgaggtcattctggtcatctgtggcaggaggactacaaatgcctggcttttccctttctagcgggcacacaaagagacagagaagttgaatctttagcaatccattgaaggggaaaccccacaatgcaaagagaagcattgtcgagggcccttcacctgcaccctctgttctcccagacctgttccatgatgcattccagaggaggtggctctccgtcactgtcacttgcgattgccaaggcgtcagccagacaaggcaaaagctcccaagggatgttacccttcagctgcatacagaactcctagaggcaacaaatccacaaggaaagatacacccatgatcgtgtatttcagccacaatgatggaggtgaggtggaaaaggacgaggaacaagtgggattctgcccatgtctgtctaatgagcatccacagcaaactccaacggaagatgtgaaacacgctcaacactagaagaactcggtttgactctgtcttggtgtgggtcccaggccaaagccctcggcattttcaggattaatgatcactgctggctgtctctttttccctctgcccacataaaaggcacaaaaactaaagaggaaggccacatcaccgcagagcccgtggttcaggattctcctcctgggagccccgatttcttttcttttctcaccttaacagtgtccagagtacttttattgccagaatagagatatagcctgctcagagcctcatgtgcggcccaccactcaggaacaaagggagagacagatactaagcatgtgtgcaacagagaaaatccagcccacgaaaccgtgtgcaagagcagctccgtcctgcctgaatgtggctttgggaagcccacctagaacttggcctggcgccagtcacccactcagagcctactgccatgaaggcaggccctcactctccatccctgcccacacagggagacccagggcaaacagaaagtcccctagcgggggcccaaggaacccctggctctctcagggtcccattcaagtttctgggctagtcttgcagactaaataccactcagttgcctggtgggagtaggtgctgggcagggtggacaggagccccactgcttctcagccctgtgccaacccctttgtgtgagaccctagtgcagggtggggagccccaatttctaagccttgagtgtttaccaccctacacccccacctccaactcccgaattgatcatctcaaattt

SEQ ID NO: 9 - DNA, 32306 bp, Homo sapiens
  source 1..32306: mol_type = other DNA
gatagtgagtgagttctcacgagttttgatggttgtataagggacccttcccccttcactctgcacttcgctctcctgccagcatgtgaaggtgccttgcttccccttcgccttccaccacgattgtaagtttcctgaggcctccccagccatgtggaaccgtgagtcaattaaacctctttcctttctaaattatccagcctcaggcagttcttcatagcagtgtgagaacgaactaatacagccagcttccctcagggccacaggatggcagtctcaagatggtggccatgagatttctgttctgggtccaggtgtcatatctgggtatcacccgccaggtcccaatctgttctgccagtgtgcagccaatcgctgtgacttgggatttgcaaaagagaaaagattgattcacaagcctgccaagcaaggaggtgggagaacagcgttcaaatctgcctccccaaagacaaggtttagggatatgaatgggttagagaagtggggtgatctaaggtgtggggacaggtgattggcagtggggaagaatgacgtaaccggtttgttctgtacaagcatagttggggttcatggcatttcataggacacatacagaaaacagatgcattggcctgatctgagcgtggtgcttttggtcctccaatatcaaatggccatctcttgggcacatatacggttccccagctgaagggtctcaactggtttcagctggcccaattcctgaaaaacaaccgaagccaccattaccatggtgacctatgaatgttatctctaaagtagccagtgaaggttgtttcagcattcagtggcaaggctttcagctcccttggccttcagcttcatggaaaaaagtctcaaaacccagcgacgaacccttccgtttcatggatgtggcaagggtgggtgaaagagaatcagagattcttctgtccaaaagcctcttctcgcttctcattggccacagctggtcacataaccatacctaacccaacctcggggaagtgagatttacccctgaaccctgaggggctgggctggagccccaggcaagatcagagtcccactcacaggttggggtggggtggggagaagctgttgagagtccagttggttcacatttcgtaaaaactctctggccatattgtcagatgcgggtgcctgactcagccagggtgtcttggttccaggaaatcttcccagatgtgagaggcagctggatccctgagcgtcccctggttatagtgtttagcaatagttggctctgctcacgaggcttcaagctcttggggcagcagggctacagcagcgccaaagctaccacctccaggacccctccccaaccccccctggcagtttagtgtgtggttaatgctgaggctgctggaggcactgcctgggttcaaatctgagtaaatgaggggaggtcactgaaactctctgtgcctcagttttgtcacctgtaaaatggggatgatattaacactatctgggctgttgtgaggattaaaggagtaagtatatgtaaaatactggtacgtaggaggtgctctataaatgttaaaaattattttaaggtagacactttcttatatttattttacagaagaagaaactgtgaagtacagaaaaactgattaatttccttccttcatgtgtcatttgtttacccaatcaataattatggattgattcctattctataggcttggccctgtgctaagcacaggggacagtgtggtgagccaaaaccccacatggtccctgccctcatgcggcttccagtctaataggggagagagacattacacagtcacagcagggcttcagcgtggggtaagtgctggcaagggagaagcaaatttgggagggagaagtggtcagggaaggcctctctgaggaagaggtggttcagctgagacccaaagatgagagacatagcacagctggtggaggggccaatatgggtcaaggccaggaggtaggaggctgctcagcccctcccaggaactgaaagaaagacccagaggccggaaggagccaggttttgcaggacctgggatattcgatcttgtccagtgggccacaccagaaggaagggatggttcctgggaaagaggggaggctcatccaggacctgagctatgacttctcagaggatggctggaggatgggaaacactctctagctctttcggtggttctcaagacagggcagggctccttacaggagtctatgaggaggcttgtgccctcagccccacagccaatggtggctggagccagcttgcaccagagccgactgcatgcatgtcttcccgatcgcaggttcagagacatcacgatgctagcttgaaatgggtcatggtggcaggatatatactgctagaaattggcagatgttgcagtttgccaatttctagttaaagcatttaaaaattcttacaaaacaaagccttcattagaaatgcggcttgggtgggggctgggcccagtgaaggaggcactttgggtgtggccccaggctcacaagttcccctcttctccagggacccctcgccactgcccacccccaaaacctcagccccttaccccacccttgggctcatctctctacctcattctgggccctattggatggttgacggctccaagggaggccccaaccctagcagggccagcttgagtcctgctctgcagatttgggaagcaggaccgcagccaggagctgatggagccgagtgcattaggaggtgcagccagggatcccaagcacagcgggcgccaaggagctggggctaccctggcttttccttcctgcaggtttggttggggctctagccccttgcctccatccccaacatcactttttttttttttttttttactttacctttagttaatagttgagttttcttgctcacaaccagagagcctcaactgaggtgccaaaggtcccagaaatgcaaaccacctacttgagagggagcattaaagcagtaacaggtcatttcaatcctgacaacgtcagccgagcatctgtcccaggagctctggggatccataaatagctcagacaggtcagatccccttgcgggttctcaacccaggggaaagctctgacccgcagtaaaatgagggaaacacagtgtattggggctataaaagaggtttatataaggtgcaagagtagcgagctcaaaggaggagcaaccaactctgcccatatgtgtttgggtcttgaaggctgtgtaggagttttctaggaggaaaggtgaagaagaggattgtgagcagctttctattctgtacaactgcatctataaatcccataaatactttctgagaacaagtcaagcagtctcttctcacgtggagcttgcagtctagcgtaggcaaaggcatgcattgggaatcatgtggcacatttcagggagttataaattgcctcgcatattcaggaagcattccatagttttcatccttcctccagttctgcctggctccctccttccttcagattttcccatcgccttctggtgaggtccatctaccctgactgccttgtttaaaattgtgctcctgattctctgtctcccattttttcttttctgatagcgtttttcatcttttaacctcctatgaaacttacttagtccttatgtttattgtctgtttctcaccccactccccactagactgtaagcgtcacaagggcaggcgtctttgttttgttcactgttgtgtcctaagcctctagaaaaagggtttggcacataggggctcctcaataaatattatttgaatgaaagagcgtgaggccaagcatgacgactcacaactgtaatcccagcactttgagaggccgaggtgggtggatcacctgcggtcaggagtttgagaccagcctggccagcatggtgaaaccctgtctttactaaaaacacaaaaattagccaggcatggtggccgcgcctgtagtcccagctacttgggaggctgagacaggagaattgcttgaacccgggaggcagaggttgcagggagctgagattgtgccactgcactccagcctgggcaacagagcgagcctctgtccccctccccacccaaaaaaaagagcgtgagtcctagaatttgggaggtagtaaaatccaccctcatccataatatcggaagttccacaggctggcaggatcatcccaccacgtgcccagcttccaaccacagaaaggcgctcccagaattttgcaggatttctttggatttgcttcatgtttatctttgagattgcttcatctctttggagatcagcagtaaagtcttaaaggcagcaggaatctgtgtgtttgcggagacaatgaggaaaggaactgagggaagtgtgtgtggcccgtgcagtgaggctgggaggccatgagccacgtcaagcagtgactgcgtggtggagaaccccaaatgtggcacccttgggactgagaggtggccagaaggggcaagtcgactcccatgatgcctccacagagagggatggaggcttgggttctagactgtccacccccaagaggccacgcctgtcattaccccagcccctgggtggcgctgcttcccctgtggcagacacgcatctctccacggctgggggcagcggggacagtgcagagtctgcccccgctgctggcctagcttgtccatccctgtctatgaggagctgttgctcttctcagctgctctcctgttaggtcttgggacctgcaccttctgtgatgtcagctgctaaaacacaccccatctcttctgcaaaacaggtagctcatttcacccccaaccttggcaaacaccaaacttcctgcagctgctttggttttcttcagggcctttctgctgtgttctggccttcctgaaagagtcttattattctgtccatccacatccactgtctgtccatccgtctgtctgttcatcccttcatccatccatccatccatccatccatccatccatccatccattcttccttccttctcccccatccttccttccttctcccccatcattccttctctccctccatcattccctccatgatccccccattcatccttcttctgttcatccatcagtccattttccattcacccattaaccacccttctatccacatcacaatgactaagcacctcctatgctccaggcacataccaggcagcataaagaaatgcagaatgaatgggattcagttcctgcccttggggagcacccactttagttggggagagaaggaaggcctctggcaggtacctaaccccatcagaaagcaagggtaaaggcctgggaggcttcacggaggatgtgaacccggggcctgaaggatgatgagtccgcgggataagaaggcaaaggaaggatgccacatgcagggcagggagggggcaaaccctgggaggagggggaggcatgcaagcgattctgcgttctgtgtggccagaggattggatgcaaagggggatcttcaagagggggcccagttagggacagccttgaagccagactcaggatgcttggatagggtcttgagggcagtggggagcccctgaagagttgtgtgcaaggatgctctggatgcagagtgaaggatgacatggcagcgtcccgctaacaaatatgaacccctctgtaaacagaacccctgtgtaagcaagaaccctctgtaaacagaatccctctgtaaacagaacccctgtgtaagcaagaacccctctgtaaacaagaacccctctgtaaacgtgtaaacaagaacccctctgtaaacagaatccatctgtaagcaagaacccctctgtaaacagaacccctctgtaagcaagaacccctctgaaaacaggaacccctctgtaaacagaacccctctgtaagtaagaacccctctctgtaagcaagaacccctctgtaaacagaatccatctgtaagcaagaacccctctgaaaacaagaatccttctgtaaacagaacccctctgtaagcaagaacccctctgaaaacaagaacccttctgtaaacagaactcctctgtaaacaagaacccctctgtaaacagcatccctctgtaaacaaggacccctctgtaagcgagaacccctctgtaagcaataacccctctgtaaacagcatccctctgtaaacagaacccctctgtaagcaagaacccctctgtaagcagaacccctatataagcagtgcccctctggcactgggcgtggagggaaggagatggagctggggaaatattaggctctggtgataattgttgttccccaactgccttcagccacctagtctgtgccttggctcttgcagctccctctgtcttgggccctttctctcgacccattccggcctatggaacactcatccccatcctcccagagaagaggaagaacccagccttcctcaggtgcctcctgcatgccaggaactgtgggtgatggcttctctgtgtgcacaatctcatgccatccccatgaacctaagaggtggggagtagtcgcatgtttcagatgcagaaacaggctcagagaggtaaaggaacttatccaagatcacacagctaggaagtggccaaggtgggatggggacccagccctcactgcctttgcacaatgacccactgcccacagcaccgatgccacatcctctggagcctcccctatggctccagccaagccacgttctatttttgggtttgtgtttttgtgttttgtcatttgcaggcgtttcaaactgggcacgctctatgcctctcatactcattatgaccacattttctaaagacaaaactgcacagacagcctgaaaaagtattttttctgtcttgttactttctttaataacgtcttagccagtttagaatcaaatcacatgtatttactcttctcacgtgataccaggactgttctgaaagttcaggccaagggtcaccacattgacacgatttccttagttataatcatttgcatacttgtcctacctccataccaagagaaggtactctgagttagagttcaaatcccacacatccttgtcattcccacagtgcccagcatgctgctgccagcaggtgctcagtaaatccttactgaactcatgcaaaggggacagacgccacctcagcccaacaccaagattgtcctccctccctcccgcaccccttcccttcctcccttgcttcccatctttcaataaatgcagtttgaatgcatttgtcacacaaagctttaggctcagagaagctgtgggactccatgcacttcacctgtggccttgaggcaccgcagtccagccaggaagacagacaccaggcaaatcctcccacctatgcccagtcaattaggaatatgagcacctgatccagtgatgggcagggcagcatccactttagatgcacggggtgctgggaaagctgccctgagaaagtgaccgcaggctgaggcctggggcagagagaacgtgggcaggggctcgaggcagggaagagcttggagacttccaggaactgccagacagccaggggcagtggggcaagggagagaagaagccagagaggccgcaggcctgggctttccatggcgaggaggcctggatactgcataagtgcagcaggaaatgttgggtggggtcggattttcactttacaagggcagtcgctggggcagagggcatgggactcagggagggactcaggaggcagagagatggtgggacgagactgagggtgccggctggggctgggggtagaggactgtgagcgcttggcacttggaggcagtcagagctggtctgggcaggggtgctgtcacccagggccgaggaaagggatacaggaggggcagagggtgggtcggaggctgtcctgggaaccctaaacgtgggggccaggagaaggaggtgctcccgtttctgatctgtaccaattaaggggaaggtgagaaattgcttgggtggagtgtccttctctgagtctgggggttaggtgggatgaggggacctcggaggctggaggccagagttcacactcctgccctgctgtgtgacctaaggctgagagctgcctctctctgggcccagatgcatcattacctattcagcaagcagctgggcctgagagcctccgagatcccttgcaggtctgctggtctcggctgcctttccttggtctgccctcatgctgtcttcccagaagccaggggcccagtttgcttcccctgcttgtcctgtgggcacttgcgactcccggggaggcagcagacacggtccagcgtggtcacgctcctggccgcgacttcacctccctcctgcagcacaagtctcgaggaggctggggaggtgcgcgcactgccctctggtggcaacttctgggcccacctgtcattgtgccctgctgttttctaactcaattattcttgaagggatgattgagcgccttttgcatgcaaagtcccgaggtaggcatgaagtagttgggcagtccagatgggggagaaggtgtcgtggatgtggagcccagggtgtcactctacgcgctcccgatccctcccaaggcctaggcgtcctccctccaccgcgggaacggagcaggggtggcacagaactctggtggctgcaatgagggttcaacggttgaatgcgtggagtgcatcttagcttcacttctggcccaggaagcgcaggctccgtgtacaacacacgcagcggtgcagtgccttcaaagggggcgtgtggctgtgtgtgtggatgtgccagttttgggcttcctctcggcaggaggtgggggggcggggaggctctggcgctgagtgccccctttccattgcggtcctttggtgcagttgagggtccccccagtagcaggggcatttccaaggagccaggacttcaagggcctaaggctggaggggccctgtccagtgcaacagagagaccttgagcatgggcggtgaggtcacctctcctggcccagctatcagctgagctggatgtgcatggctcaggtttgcacaaaggaggcccacctgttcacaggagttctggaaagaacacacctgtggcaaagacacacacacgtgcacaccagacaggccatgatctcgctcccctggcgaggaatgacttccttgccgtagggagaattcaggggagtctggccctcttgaggagatgctgtagaggggagttgtggacggttggattcaggatgttttcagctccaagtgtctgctttaggccggtctgcacctgtgtcctccccgcctctgtccttctggcacttgccctgggtaggcctggctcaaagccctcttctcccctcctcccccaaccccacaaacaataataaaattaaattaaattaaattaaattaaatttttaaaaaaagccctcttctccatgaagcctgccctgaagaggcagctgcccaggagcatctgcacatctcatacaaactttagctgtgattatattgcattcgttcccatgcctatttcctgtccctgcagctaaactctgggctccttgagggctgggtggagtcttgggcatcttttgtggctgctataactcccagccctgagccaggataatacagacgcttgacaaacgctccatgctgactggcaggtgttcctaccttccatgcctgggaaaatcatgattcagactctccctggagaggccacggtcagagaaacattaggaaggacagagtgggttctggaaggccagaggcacacggccagagcaggcacagatgcggccagccatagctcaaattctcatggctctcttcttccaaaggtggcggcagcctggtagttaggagcatgtgcttaggagccagatggcccaggcccacatcccatctctgacacttatcacctacctgtgtgcggccctgggcgagttatttaacctccccgtgcctcagttttttcatctgcaaaataggataagagtcctagtctgcaagtgctgttgtcaggattcactgaggtaacatgcggcctctcaaacttctgtgcacactcgagttgcttggggacgtggtgaaaatacagattctggctcgggagggctgggctggggcctgagcatctgcatttctgaccagctctcagggaatgctgatgctcttgaacctgaaactgccttcagcatagtcgggaatcaacatgtgtacttagaatagggcctggtgcgtagtaagggctagaggagttacctagtacctccctgggaaggctatgcttctagaactttctgctctgtagcattctgccctctgcattcctggtggccctctgctctctggagcctcggtgatgctggagcctctggtgtctctgaggtgggctgttcagagcctccttggtggtctgcattctgggcctgagaagggacagacaggaggtgatttcaagcgagagagaaacaggaagaaggcccagtggggagaggtggcttgcccggcccactcacctgaatgtccaggcttggtgtgagcttcacaacttaaaccacacatcttgcagcttccaccatcccccctgcccaccacccccaccacccggcccctcacccctcactcctgccacactgcttcatgcaggaacggcacagatgacttcagccagagtgtcagcagcacctctgggctcaaaccacaggcctgaagccgctgaccacatcctcccgatcaaagcacccccgtgccccctcctcactctgctcccctcctctgaccccttgtgcactggccaggtctcttctgggccctgacgcctggcttagggaggagtgaggctggggttctcgggtacttttggagaggtggaggaggttgggggtgaagtagtggggctgaggggcagggagcgcagagggagaaccctgtgttgacaggctacatggctgactgttgctttccgctctgctctgcaggccgcctctcctcacgcaccctcacgtgtgatacaggtgatggcctctgggctgtttccagaggtcccatgtgcaacccacagaaatggtgtgggaacagcaaagcaccttcgtgcatgagcagcagccatggcactctcggcctgtgctgctggctgccacctgagctccactttggggccgtttctcacagagcagctccacgcactgaggaatgagctggtctcagtttcctccagcctggcccagtaagagggtgacaagcctggggcccttcgtcccctgctgctttcaggtttgctcatgagttcccggaggcccatgaaatcaaggtcacaggctcaagcccatgtgggtggtggagacgagcatccctctgctccactcaggtagcagccccgacccttcctggctttcccccgaatggggggcctcagggcgggggtataaggcatcctcatgccaggagcccctcccaagggctgagaagagtccctcctcccttgagggaggcaaggtgcccagtttggattcctccaatcagaggatgactttcctggaagagcttctgggatcatccaacccaagccagtccttgcggggcatctttggttcctgcttgctcagtaggcatcaggcctcctgcttctagaagcggcccccccatttctctttggggtatctaccttcctgcatttggtgaagtcttggttgaactctcaatcaaagtaccccaccctttccagctggaggatgggcatttgacgtgatcttagccaatgagactctttgaattctaatgaatgaagccagcaaacagctggaggttggtgcctcaaaggggtctctgcagctggaccacactcagctgcaggtgcggctggtctggagtctgcccgtccctgaggtctgggtcttcagctccctccagaacctgagaaccactccagatcctctaactagttctgtttggctcaggttagctacagcctgttcctgcggtctgtagctccagagcagtgatgactgcagggagcgaccctagtgagttggaggcactgctggggctcaaacttaggtctccaaaccctcagtccagtgctcatgcccctggggagaagcctctggcctggggacaggggtgggactgaaatgaagtggaaccgaagggaagaaatgagtgttctcatctcatggtctggtgctggggtgtgggtgttggtggtttggctgctgggcagagtgtgaggaggtgtatagaactaggcaggcaggggtaggatgcggtcaatcagacaggtagcctttggccctcacaatcccctgcctcttccccagtacccctggcatgtggtaggatctctgatctggggtagaggggtggggtgggggtcagggatttggtaaacagagcctgtgcctgacctgattcagactaaccagaatgggtttgtgactccgtgtggtagaggcttcacagtgggactgtctctaactcactttgaaagtgaaaccagccactcaccaaacatgggggcccctgctggttctgaaagctctggcagcctctttattcaccagtcacccgaagtataaaccagttagcaagacagcttcaaggtcagccagccaactggtgtgtgcgtgggcctcaccaggccttccaagtgcgcatttaggtagccttccagcaagtcggtgggctgtgagctcagggtcagctctcccccatgtttgggtcacctaccccaccagtacattaggcagaggaggtttttatcaaagacctcccgtactgtccttgagcatccaatgctcacactggcaccagggcctgtggtacaacactggtcctactccaaggccccgcagacagaaaaccgtgggggcgggcactcttcccctcgctgccctctcactcgatcgccatcacccaacgtggtcagtcattgctgtaattaaagactatttcaaatctgagatctgtactaaatatatgcatgcccctgtacttggctaaggcacctgtgtcagccaggaacttcagttgcaaaaactctactggctactgtcattagaaaaggaatcattcaggatattctgaagctcaggaatctctggcaggacccgcaaatcaggcttggagaagcttggagaagcttggagaggcttggagaattgctgcatcctggacacgatgttccgacaccaccctcgccagcctccaggtcttggagttgaggcttctgattggggaagcctaatcacacgcacacacttcaggactgaggtctggagctgaggcttctgattgggtgagcctggtcacatgatcatattcccagcctgtggtctggaacccaggcttctgactgggggaacctggtcacatgccctcccagtcagcagtctggagctggcttctgattgggggagcctggtcacatgctcactttcccagcctgtggtctggaactgaggcttctgattgggggaacctggtcacatgctctccccgcctgaggtctggagctgaggcttctgattgaaggaacctggtcacatgctcactttcccagcctgaggtctggaatggaggcttctgattgggcagcctggtcacatgcctatgtcccagctgcaagggaggctggaaaagcagtttcagcttactggttagggtaggagactcctaaaaaaggagataactccaacacaagaaagatttgcaaagatgcagcgcacccagatgtaaactaaatatccaggacagcacctgagaagaaacagggctggcagagggtccctttgtcaatgccaccctcagctgcactcctccctctgaaagtggcgcttcctcacatatgggccggctgtggtggtggagttttgttgcatgtttaagttagaattaatggcgagtattttaaatgggaggttttcacgtttttttgttttggtttctttgagatggagtcttgcctgtggcccaggctggagtgcagtggcacgatcttggctcactgcaacctccacctcccggggttcaagcagttctcctgcctcagcctcccaagtagctgggactaaaggcatgtgccaccatgcccagctaattttgtatttttagtagagatggggtttcaccacgttggtcaggctggtctcaaactggtgacctcaggtgatccacctgccttggcctcccaaaatgctgggattacaggtgtgagccaccgcatccagtcttttgtttgtttttttgagactgagtctcgctctgtcacccagtctcgctctgtcaccacgatctcactgcaacctctgcttcccaggctcaagcgatcctcccaccttagcctcccgaatagctgggaattacaggcatgcaccaccatgcccggctaaatttttttttttttttttttggcatagacagggtttcggcatgttggccaggctggtctcgaattcctgagctcaagcaatccacccgccttggcctttcacgtttttaaaaataccaattttagcttcttttgcaaattgagtttcctggtagtatggtgcagacatctccatctgccatttctcctgtagctggagtctccaatgtgccatggtccccacctgtccacctgatgtaaattcacttcctacctggcccccatgggcatctcagttccctgcccaaaccctcccagaaggagagactaggcttccttagtgatcatgctgcgtggtactggttctgttagcccccaccttctcctgtacccactgggcctgcctggagaaagagcgaggccccgctgaatctcatacactccagtgatgctcccttagccacggataagctgccatcccgctgctccccacagggagctgccgtcactgcatcagccaatcagtgtcctggccagtcagacactgggttgtgttgtgttcttaatcagtaagggtgtgcctggttacctctggccatggatgtgaagagtctttgatgttgggaggtcaacagtaggatatattttagctaactgttgattgtccaaaaatgctatttactcaaggaggtctttacaatgcagtggccctcgcaatgtgcccacaaacctgtatacgcagaaatgatatcaagagaatcatgttttaaacttggctgggaacaaggctgggcactatgtgagcacacaggtggagaaggctttgggctgcagatgcagggaccatgggaataacaacgggtggcccccacgccagcacccctgagtgccaaccaaagtggggggtccttctccatgaatccttgtcactggtcaggcacttttgggggctttgccctagtccagaacttcccttctgcaggctcagttcagccccttcgttctggggagggcttcctctgcatcctcactgtggcgtgtttcaggatctgccaaaatgcacctgcctctgtcccctcggcctcaccttgtttagattcttgaagtggtaatggaacacaaatgctaggactccaagaggcagagaccaagaggcagaggttttacagtttctctctatatatgttgtagggccaggtgatacccagatctagcctgccttgctccttcccccttcctgaaatgggcatctttcccgctcggaggagttcagggtcccctgttctcagcgagcccagggtggccgcttctggtgccaggcctgacggagggcatcgtcactcttgagtgagagcatgtttctgaccccattttttgcttttccccaagcagactttgcaggcaacggtgacagccaggcataaccttcttagagggacattttaattttttccgattctttaataaaaacactaagaaaaaaaaaagcaagcaagtgttacttcttgatagagaggaacttctcacagacacttttcaaacatcacacgtcctttttcctcacagcaacacctagattgtcttctgagaaagatcgcagattttggtgaaacctgcccaagaataagaaaaagcaaagagaatgttttcactctacctgtttcccacaggaaaagcggaaaccgttgtgggtactggctttcttgactgtccccatcacaggacactggcctggctgtgaggagacctgggttaggagtccgcctttttgccaaccagcgttgtggctgtggacaaaatgctttcctccctgggtgctctcagcatagtgaagacggcggaggatgggtggttccacgggcctggtgactcagggacagtttcaggtaggaaactggctggaggacttggctgctagcagtgtccagggattcctgcctgagtggctcagtgcaggtagatgctagttcacagacatgctcccacgcaccttcacagacgtatgccacactcatgccttcgaacacagggcttaccgcctttatgtttacctgagcagtggtgcacccacgtgtattctgaacttcgttttttgtctcttctccgtacatcctggagattgctccatatcaggacccagagagcctccccttcctattttttttagacggagtcttgctcagtcacccaggctcgagtgcggttgcgcaatcttggctcactgcaacctctgcctcccgggttcaagtgattcttctgcctcagcctcctgaatagctgggactacggggtgcccaccaccacgcccggctaatttttttttaatttttatttttggtagagacgtggttttgccatgttggccaggctggtctcgatctcctgacctcgtgatccgcccgcctcagcctgccaaagtgctgggattacagacataagccaccgcgcccagccctccccttcctatcttggcatagctacgtggcattccactggatggaagttccctaatttgctcatcacctgtcccctatggatgcgcattttgttggcctcgggtcccgtgactgcaggagttactttgtacacacgtgttcaacatgggtcactctgttgatgagttttaggtgtggaattgctgggtcatgtgtcagagatgttggtagatgtcagattgccttccagggagatgcttgaagcccacgccagcagggtgactcgccttttcaacctgcctttgatggcaaagcttttgaaacttcaaatatgctatcctggtgtttttaatgtgtattctcaggactcttcagggtattagggacttagcacttttgattctttctcttttccttttttccttccttcccctcatattgacactgtccgtatgtcaggtaattttctgggttctggagtggtgtggatgaatcccacactgttcctgttgtccaagagctcctggtgtagactcataagcaaggctctgacatgagatatggtaactggcttgtgcccagaatctgccttggccagaattggagatcagggaaggactggttttaaatcggggagctgattagtctgagtaagtagggtgggaatatggtgaccaactttgtcctaggaatatcactgtaaatcttatgttctggaaacccccctcagtcccaggcacagtgggacagctggtcaccttggcgcgatcctggtaaggaatggcatggtttgcacaagataggagctgctcagggcgctggtgctggtgtggctatgaagcaggcagctcccggatgaacagggaagaacgtggggcttcaggtctggagcctggggggctggcgtggaggtggctgctgtggcaaagcagaagtaagggtgatggcttggaaggagggggtgagggagggaaggagtcagggagaatggccacagtctgtcttggggactggtgggtggggcgcctccagtgaagagcaggtgtcagcagaggggcatgctcggaggacagccctcaggaagtcggccgctagcctgctctttctccttgttctgctctggcccctctgtgggcccctgcagctttgtgatcctcatttgtcatgaggggtaccccacagtctgatggggactgggcggagggtcagacaccaaattatgtataccatagtcctcctgaccagggaccaggactcaaaaccaggtcactggtgaggcctggcactgtgggggcaggactttttttccagctggggactcggctacctgctgactttccagaagacaggtcacagcaccgagcaggggtgggcggaacacaaggacttgagggtgggtgacagtgtcagctggctgagccagggagggcaggaagggaggtggccctggagatactggaggtcttcacaatggcagaggacaggcggtggcagagacagtgaatggcacccccaaacctgctctcccctgcccgtgtgccctctacttatagctggctacatctcccagcctcctctgcagcttggttggctatgtgactcaggcgtagccaatgggatgtacattagaagtggctacggcagctttggggaagggtccttaacaggcagaggcaagccctccctctctccttcctttcccttccatctgcaggaacgcagaggtgatgggtggagcttgaatagctctcttggtctatgaggagatgggagaaggatattgaagagaccctagtagcaagccagatttagctcaggatcctgacatcataaagccaccccacctagcctgggtggcctccctttggacttcttttatgtaagagagaaataaagccctctcttattttaggtcactgttttcttcttctgttctctttggacacacgcagctgaacttcatcctaactgggacactgttgtacagtggaggcggtgatggcagcacgcgggtgagctgaggtggaagttcaaggggtcttggttaacacaggtcagaatgatggcagggtgcaagtgcaggcctgggtgtgggactggtatggagcagttcagagggaacaggaggctggaggctacagtgtggaccctgacatctcctgggaggcggagtggagagggaggctgagcgcaagggccaagcactccagtgagcagagggaggacgggtagggcgtttggcagataccaggactgaggaggatgcagtctggcttggccccaaaaaagacagcccaaggacatcagggctggaagaccccaatatgcccttgtcccatggcccctgtgaaagaggggtctgggtccagtggtgggagggctgtgggactgggtgcatggaaacagagggtatgaggcggtttgttcccagtgggacccatgaagatgcagcgcagagggtcaggtggggatgtggaagacgtaagagaagtagttataggagcaaggcgtggagctcgtggaggaggctgggtccaaaccccagcactcctgagacccaggtctgggtaggcccgagtgggctctgtcagggctctgccatcagagaagcttctggattacatctcaggttgcctcagagagaatcggatgtcctctgggacagacctaggttctaatcctggctctgccacattctggtaggatgactcatgaggcctggtttcctggcctgtaaaatggcaagggaagagcaggctcagcatgcacaaagccctgacagagtgagaaagcacaggaggcagggtcagagatgaagatggggcagtccctgtaggacatgtgggtcaagactgagagtttggattttactgtgaatgcactgggaaggtttgagtgtgtatgatggggagtggctggggggtgaaatgacgtgatttgagtttgggaaaggttgctctggccgctgagtggagaacagaccagagagtcaggagtattaatagaaacagggagaccagggcggaggccaccatgggcctgctgaggtgggagatgacacggctctgagcagggcaggggctgaagaaagcaggtgcacctctctgcacacccctgctggccacccatgcctctgggcttctgctggcgtactgtctcacccctcctcacctggttttatgttcagctgtagcacctgggggacagactgtatgagtcagagaaaacagaaacagcagtccccaggtctcagaggcccaacaatagaggtttatttttcattcaagttatagcccagtgtgaattgtctgaggaggggtgagggcctgtccttcaagggactccagcccctctacccaaaacttcactggattctggcaggggaggggagagagagaaagagatagagggatggccggccgtggtggctaacgtctgtaatcccagcacttagggaggccaaggcaggcagatcacgaggtcaggagatcgagaccgtcctggccaacatggtgaaaccccatctctactgaaaatacaaaaattagctgggtgtggtggtgtgtgcctgtaatcccagctactcgggaagctgaggcaggagaattgtttgaaccggggaggcggagcttgcaatgagccgagatcgcgccactgcactccagcctggctacagagcaagattccgtctaaaaaacaaaaaaaaaaaaagaaaaaaaaagagagatagagggattaagagacacaggagatcataggtgagattttttttaggcctagaagtagaataaatcacttcctcccacatcccattagccagacttggttctggagccccagctcactgcaagggaggctgggaagcagacccgagctgatgccgggaggagggtgcacgccatggcctggggacccattccaccaggtttccctcctacgtgctcccacaggccatgaggtgttacatagaggatgtgaaggagaatcattctctgcgtagccccctccctgcctctccatctgaggtgttctgggctaggacccttgacaaaagaacaaaccggctccatcccctgtctcctggggtgggagactctgtatgtgggggctgcaccatcccccattctgctccccactttcctggctgctgcacatctcaggggcacgagggtggagagtggcctacctccctcccttggtcctaccccgctggcctgggctgcgcgcctcaggcttcctgtgtctgacctggtctttggggcatctggggtgctcagtaaatgtttgttgaataaataaaagaaggaaagcaggatgaggaggaagaagagtcttgggcagtatgtgccaggcagactgatgctgtctcatggatgaaatagctgtacgcagctgctccttgcaagctgggtctgtgacttctggctttggctgcggatgcatttttcttttctttgtatgtgtgtgcttttaaagttttaactgaattcatttatttgggggcaggaaatatgttcatgccatacaaaagggtctatagagaaaagcgtccttcctaacccttcctcacctggctctcctccctgagacaaccggcgttcctaatttcttgcaaagcttctgggaatatttgaggcttaataaatgttggctgttattctcatggttgtggctgttgtgtttagtatcctctcctgccgccagacagccttactctagaggagagggaagggcagtgtggaaggaactggcccttacagcaagcctgcagtgcgtggtcacattaaatgctttatacacaccttcttgtccagttctcccttcatttggtaggtctgcacactgaagcacagggaggcctaagtcacagcctcaatctccagtcctgcccctcctgggcagcagtgctccacgcttcttcttttccagcagcgggctttgcacatgggagttgggcctgattctctttttggagtcttgaattccacttttctcacatttgcagagctgccgtctctgctcttctctctcttcttctcagaagaggtggcaggctgggcgcagtggctcacacctgtaatcccagcactttgggaggccgagggaggtggatcacctgaggtcaggagttcaagatcagcctggccaacatggggaaaccccgtctctactgaaaatacaaaaattagccgggcatggtagcacgtgcctgtaatcccatctactcgggaagccgaggcaggagaatcgcttgaactttgtgggcagaggttgcagtgagccgagattgtgccactgcactccagcctgggtgacagagctagactatgtctcaaaaaaaaaaaaaaaaaaaaaaagaggccacagcttgcaataaaacccctctgaaacagggaatcaggaaagggaagaagcagagctgctgcttctgggctctgaggatggaacttcacagggtctatggggctgtgatgtagcacgtaaatagatgtgcatttttctaaagaccaatccataagtttccatcatttaaaaaaaagagccctgagctgagctcccacgccacccttctcccccgccaaaaaagggggagaagggttaggaaccatgacttgagaggaagaggaaggttctagaatgttcagtaagtgaccatcaaattgttcacaaagacctgatagtcatctctgggaagtgctagctagaacaaacagacaagagaaagaagtaaagggcatccaaattggaaaggaagaagtcaaattatccttgtttgtagacgatattatcttatatttcaaaaagcctaaaaacgccaccacaaaactatgagaactgataaacaaattcagttaagctgcagaatacaaaatcaacatacaaaagttagtagtatttctatgtaccaacagcaaacaatctgaaaaagaaatcaagaaaataactccatttggaatagctacaaataaaataaaatacctaggaataaacgtaaccaaaaaagtgaaagatctctataatgaaaactataaaacgttggtgcaagaaactgaagaggacacaaaaaataaaaagatattctatgtttatggattggaagaatcaatattgttaaaatgtccataccacccaaagaaatatacagactcaatgtaatccctatcaaaatactaatgacattcttcacagaagtagaaaaaataatcgtcaaatttatatggaaccacaaaagatgcagagtagccaaagccatcctaagcaaaaagagtgaaactggacaaatcacattatctgacttcaaattataccacagagctacagtaaccaaaacagcgtggcgctagcgtaaaaacagatgcattgaccaatgaaccattggtcagaatagggaacccagaaacaaatccacacacctacagtgaacttattttcaacaaaattgccaaaaacatacctttggggaaaggccagtcttttcaataaatgttgctgggaaaactggatatccatatgcagaagaatgaaactagaccctcatctctcttcatatacaaaaatcaaatccaaatggattaaagacttaaatctgagacctcaaactatgaaactactaaaaggaacattggggaaactctttaggacatgggactaggcaaagatttcttgagcaataccccaccagcacatgcaaccaaagcagaaatgatagatgagatcacatcaagttaaaaggcttctacacagcaaaggatgcaatcagcaaagtgaagagacaacccacagaatgggagaaaatatttgcaaactatctgacaagggattaataactggactagataaggagctcaaacatctttatggaaaaaaattatagaaatcccatcaaaaattgggcaaatgatctgaatagacagttctcaaaagaaaacatataagtttatgaaaaggtgctcaacatcattgatcatcagagaaatgcatgtcaaaactacaatgagatattatctcacctcagttaaaatgacttttatccaaaaggcaggcaataatgaatgctggtgagggtggggagaaaagggaactctcgtacactgttggtgggaatgtaaattagtacaaccactataaagaacagtttgaagagtcctcaaaacactaaaaatagaactactatatgatccagcaatcccactgctgagtatatacccagaagaaaggaaatcagtatatggaagagacatctgcattcccacatttgttgcagcactgttcacaatacccaggatttggaagcaacctaagtgcccatcaacagatgaatgaataaagaaaatgtggtacatatatgcaatggagtactattcagccatgaaaaagaatgagatcctgtctttgcaaccacatgtttgaaactggaggtcattatgttaagtgaaacaggccaggcacaaaaagacaaagctcacatgttctcacttatttgtgggagctaaaaattaaaacaattgaattcatggaaatgggaagggcagtgatggagatgggaagggcagtgggggttggaggggtggggattgttgttgggtacgaaaacagaattagatcgaatgaataagatctagtatttgatagcataacagggtgactttagtcaacaataatttattgtacatttaaaaataactaaaagagtatacttggattttaacacaaagaaaggataaatacttgaggtgatggataccccatttaccctgatgtgattattatacattgtatgcctgtatcaaaatagctcatgtgcctcatgaatatagacacctaccacatgcccacaaaattaaaaactaaaaaaaacagtcatctctgaatgctaaacggagtaaggggcttcctggaaggctgggtgaaatgggagtctcggaaagatggtgtgttgcaggctgggaggagggtgagacgctggggtcacctagagggacctgcttgtgtgaagcctacgtattagtgggtatgtgtgtgaccggatggaggcgtcagaggtgttgggtagcctgtgtgagttggcgtgggggtgatgtaggaggggagagagggagggcctgcgttcccttggctcctgtgtgcagctaggcccctatttgacaatgtgtgtctgtgtgtgtgtgtgtgtgtgtgtgtgtgtgtgtgtgtgtgtgtgccgcccccagcgtaggaggcagatctttatctggccctgggtgcttgaggagtttcaggctttctcataagcctcgtctccccgcctctccaccccaggccttgcccctctatcctctgcacaggaagtgggctggctctgggcttttagtctttgcggccccagcagccagagctcagcagggccctggagagatggccacggtcccagcaccggggaggactggagagcgcgcgctgccaccgccccatgtctcagccaggtgatgtccccctgcctccctcccggcccctgtggaccagccagagggctgggagtgaaagtcacagagaagactttcagctctgactcagttcccccagcagtttctgcctgaactcccatcccccaactttgtcttagaattcccctagggaggggttgggcccagctctcatggctcctggctggaggaaaaagcggtccccagtcccaggtcagctctccttcccaacccattcaggggaggctagggactgctgtgggatgaggggcttccctgggctctgtgtgctgggcccccaacccggagaggtggagaagggatgggtggatagcctgggtgcccggagagggtacgggatatggggtgggacaggcgggagccccctcctgacctctgtggagtggggcccatataccgggggaggcagccctgtgttgcgtgctcatcaggaggcttgtggagtaagtggggacagaagggtccccttggagagccatctatgaagtgaagttgctgcgaggcctctgggagccccaggctggaggcagagccctgggtttgagtctgggtttgtccctcccttggctgcatgactggacaggtcagaagcatctccggacattggggatgggaccctatctttggggctgcctggagctcatgctctctcctctcctgggggccgaagcctcccactcttaactgtgtttgtagcacctcgggggagggaggtggagtgaggagctgagctatccccaagggtccccttccttcccccacagccagggtcccgcacccagctggagaagcccctgaagttaaatagcaagagacaagcactgtgtatgggagcaggggggtggcaggtggaggtggccagagaggacgaggggacataggccccattgagttcagaggtcaagttggatgaaccatgggctcccgagctcctgctttaccaccctgccccagccaccatatacccctgatggcagcaggggccccgaactgcacctgaccaggttcaaatcccagctctatgcccttctcgcctgggtgagaggttgacccgtttcttagtctcctcatttggcaaaaaggaggcatactagagcctcctcatggggctgtagggagggctaaaagctgaactacccccgagcgctcagggcaggcagtgtaggcagagagagagcaggcaccagtccctgtccccagcagcatctctctgaaaatcagatggctggggtcagccgctccgggcttcagttctggcatggatcaaatggggcagcaagacccaccttgcagggctgtggtggggacgtaggcatccgtgggtgtgaccagtttcagagtcatgaacgacagggcacgtgtgggtgtgcctgaggacccaggagggggtgctcaaactcgaggtcctgggctccacttgctgtcacccgaggctgggcttctcctcaccgtgcaaccttaagtgggttccttggcctccctgaggcacagcttccccattgcagaatgagggtgcagcctcagtgactgccaggcgctccctcagctctgtgcccctctggcacccccacaccatcctcccgtggactctgccctctccgagcatcgctgcacctctgagctccaggcggcgggaagcagactccagatggcgctttctcctcctgcccggctgcctctaggctgggcctgtggtttacccctaaccttcctttgaccttgcacccctttgtccctcagcaccttcctcacacactggggctctctgcagttcctgtcctgggcatggggtcatcctgaggctgggcccgacctggggtgacacccacacaaaacattgatggtccccttgccccagggccaggttggcatgattgagcgtgaaggggcataggggctatgggagtcccctggttgggctatgcactctttggggtcccaaccctgtgccctctgctattgggggatcccaagaaagaggtgagcagcccccaccctggggagctcaccctcctgtggagacggtaggccaggcaaaggcgagcccatggtaccccgaataagcacctgccagtctgtctgtgagctgctgggtcctgactcccaggacagagagggtggcagccttacctctgggaccagtgtctggcagggtgggacagcatgagagagtgagcctggagccctagctccacctcttactgtgtttatgaccctgggcaagttaggggcttggagccttagtttcctcatctgtagaatgggataataaagccattgggcagcttagacgagctggcatatgtgaggcacttggccccaggcctagcatagagtatgtgctcagtacacagaacgagtattattagttcaccagtgcatatttactgtcgtgtgagctgtcgtggtgggaagatgcgtctggggagttccaggaggggaggatccctgcctgggcactgggcagtctgccctgactcagattccagaaaggcctcttatccactgcatgaccttaggcaacgcacttcacctctgtgtgcctccgtttcctcattggaaaatggggataataagagtatccccgccagaggccctgattgctgcatttaaagagttttaaatggagcctggcttacagtaacctgtcagccgatggttttggtgtgactaagggggtggcagagcggcgtggagttgggttctgatcccagctctgtgctgggctcctgatgtaaccttcagcaagtctttcccacttcgggacctcagtgttctcaaatgtagaacaagggactgtgcttgctcagagagcctttgcagctttgacagtgcgtgggcctctgactctcactcctgtgacatctgccttaggagatggaacccatggctcttggggccagggtgtgtgtccctgctccatgctggctctgatgtaagccacgagacaatccagggccacctaaagagctctgcttttttttttttgccgtactgaggcctctgaccacactcattcatttcactacatttattgagcaccgactgtgtgcccggccctgtgctcagcagtccctggagcgagtgtgctggtgggccagcccctggtcaacgccggccctgctgctgccagcctagagggaggagcaaggcgctcagaagaggaagccctcgccctccatcacggaaatcagggaaggcttcatggaaaaaggcatgagggcccgtttgaaatgggcttgaaggatggcaggatctgtaggggacccagggaaaggatagacttgggaaggacctttggagggtgagtgtcggtttcctggtcacctggagccactggcttgtgcaggcaggtaggtgcggtgtgtgtgtgctttggcctgggtacatgcacctgctcctgtgagggcagggtctggaagggtgggctgacctcattccggtcccagaaagcctcgaaaatgggaatttaggctttgattgcaacagggccactgcaggttcctgggcagggatgtgggtggttggaggcgggcttcagggaaggcaggtgagagcctgcagatggggtggtgccgttgtctgggctggaacggcacgaggctggcctggtgagggtggggaagagacaacaagtagtcactggagtgtcctgctgtctgaggagagtcctgactggctaggtatgggggcaaaggtcacctggaataggaagggtacagttgtcccagaactgaaagtcagacaggagaagagctggggatgagctgggtttttgccgggtccaggagcatcccctgtgagggggtcctgcagaccatcgggagggacgtggaggcttggggctgagctggagaaagaccctgggaagctgagggctggagccagtgagaggctcaagagagagaagggaaggggagtgagagctcgagtttggagccgctggagaagggggaacagcgggggaaatgcccaacgctcaggagccttggaagctctcgagatggaagagcaggagggagggaaggatgttccacctcctccacatctcttgactcacctgctcctctcccctctccccctgcccctctgactgaggctaccctcatctttccctggaagctccctcctcatctccctgcctccccctttgcctgctccaaacacctctcctggaagcagtccccttatcttcccgaagtgaccatgtgctcactttctcattcccctccgcacacccagcccctgcagagggtgggggcagctacttttctttcccatttccgctgcgacagttgtggtcttgcctcacgggagcttccacggttggctagaggttccctgagaagcaagtctttgtctcaaaaaaaaaaagtaaataaataaagcactggccagtggccagtgcataataggcactccgggaaaacttttgagcctccctctgacagggctggcttagagatttcatgtctctttcctccctggggtataatctgggtgaccaacctggccctgcctccagggagcttccaggcaggtaggggagtacgagtgtggacaaggtcctccgtaaaggctactgcagccctctgtgcctcagtttcccctgtcctctagggctcttcagtgtttgggtcagagctcctatctgcttttctgcttttccgagttcgcgtcctgatgccttcacaccattgcctcagctccctcgagggacagtgaggggcagaggcaggggctagcctcagagccctctggaagcccctctacctgctgactttagctctgtctatatttattccatcacttgtttgttcaagaattttttttggccagatgcctactatgtgccaggcactgttctaggaattgtggacctagcaatgaacaaaacacaaaaatacctgtccttagtgagcttacgtgactctgtgtgtgtttgtgaagagagatagaggaaaacatgaatcagtcaataaacgatgaataaataatgctatgatgaaaatgtaggggggtggtgagtaacaaagggtgcttcttttatttttttctttgagacagagtcttactctgtcgtccaggctggagtgcagtggtgccatctcggttcactgcaaactccgcctccaggttcaagcaattctcctgtcttagactcccgagtagctgggactacaggcacccaccaccttgcccggctaatttttgtatttttagtagagacagggtttcaccatgttggtcaggctggtctcaaactcttgacctcaggtgattcgtctgcctcgacctcccaaagtgctgggattacaggcatgagccacacgcctggccacgacgggtgcttctttatgtagtctagtcaaagaagacctctcaaaggaggctgcagttgagcagaaacttgaaggaagggtgtgagcatgtgtagggtggtgggattagcaggtgcaaaggccctggggctggactgagctggcaccgaggagtgcagaccatgcacactgagggggcgggcccagcctgcctctaggggcagcgaggcctgggatgtgtatttacgtgtgctctctctttctcctctcccaccagggcttccttcctcggctccaccctgtggatgtaatggcggcccctgctctgtcctggcgtctgcccctcctcatcctcctcctgcccctggctacctcttgggcatctgcagcggtgaatggtgaggaccctgacatctgaacagctctgctttccctatggagagatggctaaggccctgctaaagcctcttcccagaaggggatggggccagagaccaggtgtccacctcctgcctcaccctctgtcctggacccctgccctggcatgcatgcatgaatgcgcgcacacacacacacacacacacacacacacacacacgcctctccaagccacaggcttgtgctactttctgtccctgccttgccttagtttgcctgtaggaacttgcaggctctggagctccatttcctcctctctgaagtgggtccatgaatcccttctccgcttgccagagttacccacctcctcctctggcaggtgtgggtgccaggtgcaggggcagtgggcgtgtcttgggggggctgtgggcacctacagtcagcactcacacctcatcttgcccggcaggcacttcccagttcacatgcttctacaactcgagagccaacatctcctgtgtctggagccaagatggggctctgcaggacacttcctgccaagtccatgcctggccggacagacggtgagtccactggccacactgcagaatcttaggatggtgggatctcatattctggatcctatgcactggactccaaggagtcaacgtttacagaatcttggactaatgggacccacagactttttgaatcacaggagttggggcccaggcctggaatcaagggatcgtttaagagaatttgggagtcagctatttagacttctagaacctgggtggagtccaggctgcagtggaggggctggggatgaactggacagggtagtttggggacactgagctgcaagcctgctatgccaaggctggactccactctagaggcaagcagagaggaagtggggagaaaggagggtccccaagctttttggataagctcggattttgagagaccctgtaggcagctgtggggacagggaacctgggagtggtttgggtgcctggtggatgggagagagatgtcactgtgatgtaaacatccaggcggccagtgagtgagggactgaccccaggtaggcttgatggggtcagggagaagaggaagaccacagagaggtggctgggcccaggtggattccatagggtgtggggtgggggaaaatggggagggagatggaggggtaaggcaggttcgggccctggcttctgggtgggtggtgattccacacggctctgaaggcctaattaaggatctatcttcatcctaagcctcat

SEQ ID NO: 10 - DNA, 11145 bp, Homo sapiens
  source 1..11145: mol_type = other DNA
gtgatccacctgcctccacctctcaaagtgttgggattacaggcgtgagccaccaacctgaaggatatttttaaaaagataatttttcttttctttctttttttttcttttttttttttttttgacaaggtctcactcagtggcccaggctagactgcagtggtgcaatctcacttcactgcaacctctacctcctgggctcaagcaaacctcccacctcagcctcccaagtatctaggatcacaggcgctggccaccacacccggttaatttttgctttttttttttttttttttttttggtggagacagggttttgctatgttgcccaggctggacttaaactcctggtctctagcgatccacccacctcagcctctgaaagtgctgggattacaggcatgagccacagcacccagcataaaagaattttttagagcagttttagattcacagcaaaattgagaggaaggtatggagaattcccatatactgtctgccctcacacatagataaactcccccattgacatcccccaccagagtgggccaggcatggtggctcatgcctgtaatcccaccactttgggaggccgagatgggtggattgcttgagcccaggagttctagaccagcctgggcaacatggtagaacctctatctctccaaaagcaaaataaaacattagcagggtttaggggcgggcgcctgtagtcccagctactctggaggccgaggggagagaatcactcgatctcaggaggtggacgctgcagtaagccatgatgaccccactgcacttcagcctgggcaacagagtgaaactctgtctcaaaacaaaacaaaacaaaacaaaacaacaacaacaacaacaacaacaacaacaaacccaccagaatggtatattcgttagagttgatggacctaccttgaacatcattacccaaagttcatggtttacgttaaagttcactcttgatgttattttatacagtctgtgagtttggacaaagaataacatgtgtccaccattgtagtatcttacagaatattttcactgacctaaaaactttttctgctccacctattcatccttccctctccgacaacccctggcaaccgttgatctttttactgtctccatagtttcgccctttccagaatgtcatatagttggaatcctatagtaggtggccttttctgcatggcttctttcacttagtcatatgtatttaagttttctcccatgtctcttcacggccttaatagctcatttctttttgcaccgaataatatttcactttcaggatgtaccacagtttatccataaacctactgaaagacaccttcgttgcttccaagtttgggcaattatgaatgaaactgctataaacatccgtctgcaggtttttatgtgaacacaagtcttccactcctttgttttttttttttttttttttttttttgagacgaagtttcgctctgtcgcccaggctggagtgcagtggcgcgatctcgactcactgcaagctccacctcccgggttcacgccattctcctgcctcagcctcctgtgtagctgggactacaggcacgcgccaccatgcccggctaatttttgtatttttagtagagacggggtttcaccgtgttagccaggatggtctcgatctcctgacctcgtgatccgcccgtctcagcctcccaaagtgctgggattacaggcgtgagccaccgcgcccggcctccactcctttgttaataccaaggaacatgattgctgaaagagtatgtttagttttataggaaactgccaaaccgtcttccccagtggctgtaccgtaccatttttgcatttcccccagcaacaagtgagagttgttcctgttgctccacatccttgccagcatttggtgtagtccgtgttccggatttgggcaatttttttttttttttttgagacagggtctcactctgtcacccagactggagtgcagtggtgcaatctcagctcactgcaacctccgcctcccaggctcaagcagttctcctgccttagcctctcaagtagctgggattacaggaatgcgccactattgcctggctaatttttgtattttttagtagagacggggtttcaccatgtaggccaggctggtcttgaactcctgacctcaaatgatccactcaccttggccttccaaagtgctgggattacaggcatgagccaccatgcccggcctcttttttttttttttttttttttaaacaagagatgggatcttgctgtgttgcccaggctggtctccaactcctgggctcaagcaacccactcgcctcagcctcccaaagtgctgggatttcaggcaaggtgtgagccaccttgcccagcccattttggctattctaataggcatgtagccctgaaggatttatttattttatttattttattttttaggcagagtcttgctctgtcactctggctggagtgcagtgtgcgatctcagttcactgcaacctctgcctcctgggttcaagtgattcttgtgcctcagccacctgagtagctgggactacaggtgagtgctaccaaacgtggctaatttttgtatttttgtagagatggggttttgccttctgaaggattttagcctacagaaagttaggaagaggctaattccagagaaaggaaatgcgtatgacatatccagtgtggctggaattgatcattggagcttgggctttattctgaaggccatgagtagcttttgaagacttttaaattggagaatgccctctgtagtgggttgaatgtggcctccaaaaagatatgttcacttggaacctgtggatgtcaccttctttgacaaaaggcttttagcagctgtaattaaggcaaagctcctaggagagaacaccctggattagggtgggccataaatccaatgacaggtgtccctgtaagagaagagaaggataaaggagatgcagagaagaagaagaggtaatgcaaagatggaggcagagattggaatgatgtgtctacaagccaaggagcaccaaggatcgacggcagccaccagaagctagaagagaggcatggaacaacttcttcctcagcgcctccagaagggaccaaccctggccacactgatttcggacttctgggctccagaactgtgagaaaataaatgcctttttttcctttttaaaaaatttttatttatttatttatttatttatttgagatggagtctcactctgttgcacaggctagagtgcagtggcgcaattttggctcactgcaacctctgcctcctagattcaagcgattctcatgtttcagcctcccgagtagctgggattacaggtgttgccaccatgtctggctaatttttgtatttttagtagagacggggtttcaccaggttggccaggctggtctcgaactcctcaagcaatccacctgccttggcctcccaaagtgctgggattgcaggcgtgagtcactgcacccagccgagagaataaatttctgttggtttaagccactcagtttggggataacttatggcagccctagcaaactaatacatactaaagatacatactaaatactaagctgggccatatagtccagttttcctgagactcccaggcaagtgctgtttttctttgcttaatatcctacaccactttctgtctggtaaaattacactcattctttaagatgccactgaaatagcacctcttcagcacagccttcactaaactatccccctctccatcttggtaaatttagttacttcctcttctgtgctcacatactttgtagtatctctacatttatgctataggacttgttacactatgttgtattacttgtttatgtcttccccacttttctgtgagtgtctagaaatatgaggatgtcttgttggtctatttccagaacataagcacagtgcctggcacatattaaaaacgtaataaatgtttgctgaataaatagtttctgtaagtggcttctccaatcacctctgtgttttcggggaaggtaaaactggcaacaggatgaagaatggattagagagcagagggcctttagaaagggaggccagttgatggagtctagatagaatcatgactagagctaatgaaagactgatttagcagagtggctgtggtaatggaaaggaggaaaccgttgggagaaacaccacagaagcagagtgggttatattctctgggtgagagagggggagaaattgaagctgattctgaggtttcaagtctgggtgactgagagggtgacgataccattgactgaggtggggaaggcaggaagagaagcagagttgggggaagatgggaagcttgaagctagtattgttgttcctccatttctagaatatttttgtattataagtcacacttcctcgccagtctcaacagggacccagctcaggcagcagctaagggtgggtattctggtttggattagatcagaggaaagacagctgtatatgtgcccacaggagccaagacggtattttccatcctcccaaaacagtagagctttgacagagatttaagggtgaccaagtcaaggaagaggcatggcatagaacggtgatgtcgggggtgggggttcagaacttccattatagaaggtaatgatttagaggagaaggtggttgagaatggtgctagtggtagtgaacagatccttcccaggatctaggtgggctgaggatttttgagtctgtgacactattgtatatccagctttagtttctgtttaccaccttacagcagcacctaatctcctagaggacttagcccgtgtcacacagcacatatttgccacaccctctgtaaagccctggtttataaggttctttccaccggaagctatgacagaggaaacgtgtgggtggggaggggtagtgggtgagggacccaggttcctgacacagacagactacacccagggaatgaagagcaagcgccatgttgaagccatcattaccattcacatccctcttattcctgcagctgcccctgctgggagtggggctgaacacgacaattctgacgcccaatgggaatgaagacaccacagctggtgggaaatctgggactggagggggctggtgagaagggtggctgtgggaaggggccgtacagagatctggtgcctgccactggccattacaatcatgtgggcagaattgaaaagtggagtgggaagggcaagggggagggttccctgcctcacgctacttcttctttctttcttgtttgtttgtttctttctttcttttgaggcagggtctcactatgttgcctaggctggtctcaaactcctggcctctagtgatcctcctgcctcagcctttcaaagcaccaggattacagacatgagccaccgtgcttggcctcctccttctgaccatcatttctctttccctccctgccttcattttctccccaatctagatttcttcctgaccactatgcccactgactccctcagtgtttccactctgcccctcccagaggttcagtgttttgtgttcaatgtcgagtacatgaattgcacttggaacagcagctctgagccccagcctaccaacctcactctgcattattggtatgagaagggacgagggggaggggatgaagaagaggtgggttggatcagagaccaagagagagggtagcaagtctcccaggtaccccactgttttctcctggggtaagtcataagtcggttgaggggagatgaggctaggctctggatatctgcagtacccagattggccccactgttcctcttccttccaacctttctcctctaggtacaagaactcggataatgataaagtccagaagtgcagccactatctattctctgaagaaatcacttctggctgtcagttgcaaaaaaaggagatccacctctaccaaacatttgttgttcagctccaggacccacgggaacccaggagacaggccacacagatgctaaaactgcagaatctgggtaatttggaaagaaagggtcaagagaccagggatactgtgggacattggagtctacagagtagtgttcttttatcataagggtacatgggcagaaaagaggaggtaggggatcatgatgggaagggaggaggtattaggggcactaccttcaggatcctgacttgtctaggccaggggaatgaccacatatgcacacatatctccagtgatcccctgggctccagagaacctaacacttcacaaactgagtgaatcccagctagaactgaactggaacaacagattcttgaaccactgtttggagcacttggtgcagtaccggactgactgggaccacagctggactgtgagtgactagggacgtgaatgtagcagctaaggccaagaaagtagggctaaaggattcaaccagacagatagaaggacctaatatcaagctcctgttctctgcctcccagcttctctgctcaccccctaccctccctcctccaactcctttcccccctattttctccagtgagttttctttttttcttttcttttctttctttctttcttttttttttttttttgagacagagcctcactctgttgcccaggcttgagtgcagtggggcgatcttgggctcactgcgacctctgtctccctggttcaagtgattctcctgcttcagcctcccaagtagctgggagcatgcacaaccatgcctggctaatttttgtatttttagtaaagacagggttttgccatgttggtcaggctggtcttgaactcctgacctcaggtgatctgcccacctcggcctcccaaagtgctgggattacaggcgtgagccaccattcctgacaccagtgagttttcattagggattccctacccatactcttcctgataccagatagacaagtaaacaaaaggaagccattaaggggatccagaggggaggcattagattcaagtcagtgaagggagcagtgtggcttgagtagtcaagagatgagagagaaactgggcagtagcagagatgacactggtgggtgttcaggagtatgttttaattctcccttctctcatagacacccactttccctcatcctctttctcctcaaggaacaatcagtggattatagacataagttctccttgcctagtgtggatgggcagaaacgctacacgtttcgtgttcggagccgctttaacccactctgtggaagtgctcagcattggagtgaatggagccacccaatccactgggggagcaatacttcaaaaggtaaaatgggcccacatgacccaatccatgagcccaacaccccagcctttctaacaccactgtcttttgctccacttccctgtcactaaagcccctaaacttggtgccccatctctccacactgtctaaccccaacctctagaaatcaaggtttttctgtgtagggttgggttagcgtgttgttagagtaggggagtggattgagaaggaggctgaggggtactcaagggggctatagaatgtataggatttccctgaagcattcctagagagcctgcaaggtgaagatggctttggaaccagctggatctaggctgtgccacatactacctctttggccttggccacatccctaaactcttggattctgtttcctaagatgtaagatggaggtaattgttcctgcctcacaggagctgttgtgaggattaaacagagagtatgtctttagcgcggtgcctggcaccagtgcctggcatgtagtaggggcacaacaaatataaggtccactttgcttttcttttttctatagagaatcctttcctgtttgcattggaagccgtggttatctctgttggctccatgggattgattatcagccttctctgtgtgtatttctggctggaacggtgagatttggagaagcccagaaaaatgaggggaacggtagctgacaatagcagaggagggttttgcagggtctttaggagtaaaggatgagacagtaagtaatgagagattacccaagagggtttggtgatggaaggaagccacaggcacagagaacacagaatcactttatttcatatgggacaactgggagaagggtgataaaaaagctttaacctatgtgctcctgctccctctttctcccctgtcaggacgatgccccgaattcccaccctgaagaacctagaggatcttgttactgaataccacgggaacttttcggtgagaacgctgtcataagcatgctgcagtctatcaactgccaactgcctgccagcaagacagacagagtgtgggggtgggggcagagaggagagggaaggaggccctgcactaactgtcaggatgtggccgaccaaatggggcatggactatacagagagagacacacacagaagtgcagattatagattgaatgaggcagatggcaactggtattgggggcccaggagcctgtgtatcccttctgtaatcaattacagtggttgcagacatcatgagtactcctttggcacagagctcggtcttttacttcctgcccctaattgacccctgacctggacatatctgtctttaggcctggagtggtgtgtctaagggactggctgagagtctgcagccagactacagtgaacgactctgcctcgtcagtgagattcccccaaaaggaggggcccttggggaggggcctggggcctccccatgcaaccagcatagcccctactgggcccccccatgttacaccctaaagcctgaaacctgaaccccaatcctctgacagaagaaccccagggtcctgtagccctaagtggtactaactttccttcattcaacccacctgcgtctcatactcacctcaccccactgtggctgatttggaattttgtgcccccatgtaagcaccccttcatttggcattccccacttgagaattacccttttgccccgaacatgtttttcttctccctcagtctggcccttccttttcgcaggattcttcctccctccctctttccctcccttcctctttccatctaccctccgattgttcctgaaccgatgagaaataaagtttctgttgataatcatcaaaaatgtctgtcatgggggtggggagcaggggaagcctgggatgcaatgagggtgggaggggaagaaaaaggagtagtacattcatgctgtggaggcaggagtattaccaatttgctagaaagagtactgttctggaaactgacttttttccaggtgccctgcactcattccagggtttagtttagagttagacttacaatgggctgggcgtggtggctcacgcctgtaatcccagcactttgggaggccaaagcgggtggatcacgaggtcaggagatcgagaccagcctggccaacatggtgaaaccccgtctctactaaaaatacaaaaattagccaggcgtgatggtgtgctcctgtaattccagctacttgggaggctgaggcaggagaatcgcttgaacccaggaggtagaggatgcagtgagccaagattgtgccactgcactccagcctgggcgacagagggagactccatctaaaaaaataaaaaaaataaaaaaagttagacttaccatgttgtcccccctttccaactaatctttaacataccccacccaagtttttctctccttacccttactccactttttggccacacctgccacttctgacctcataatcccttaactgcagtctcaggccccacccctggggatactggttatccacaactatcaacagctctctcctgcctgtccagcctgggcatcctcagcctatgaaatccatctgcctctctagtccaccaaccctataggcctatttgccccacacaaggcctctagaacaccctccttctgcatgactcatcagggcactttgtgaagagtgaggcgatcttgagggcatgttcatcttcatcaaacatggaggtgaggggcagcacagaactagagtctgtggatgggggtggagggatctgagggcagtggagatcacagaaatgggcccatgtgtcccatcctatctcttctcagataatcagcagtttctggtcaataccaactgtgctgtcgtcgtgttgctgtattacatccgcagtaaagtaaagttgcctaaaacaagtgaggagcttagggaggggactaatggccaaaggtcagaaggagttgggtcatctctaacccccaagccctcctcctggcaagaagggagttcctcatatccttagagcttcctccaacccattttgtataggatcactggccccttttgctcccactctctttccttagctgccccaattaccctgtgatctccctccagacaccatcgatttgtgtgaacaaacggggaaaatgaagatgcttttcctgatgaagcccaatcatgcagagtatgccagcaaataccttacagctcgaagcacctactacgtttgtaaggtggaacgtgggccaccaggtagaggctcaggagtgttccccagaaatagaataaggccttcaagatagattatgaaccttatctttaaaacaaggcatccaaggctgggcatagtggctcatgcttgtaatctcagcactttgagaggccaaggcgggagaatcgatagagggcaggagtttgagaccagcctgggcaacataacaagagcctgcctccaaaaccaaaaccaaaaccaaaaacaaccaaaaccaaattaaaacaaaaaacagaacaccctcaaacaacagaacaccctcaaccagatccaaattgaccatctctagtcagcctaaggaagggtactgcctctataataataggttacctcacacatactctcccctaggtccatacctaaggggaagaaatatccaccagctcaaggtaggtttttttttttttttttttgggacagagtttcgctcttgttgtccagttgtccaggctggagtgcaatggcgcgatctcagctcactgcaacctctgcctcccaggttcaagcgattctcctgtcttagcctcctgagtagctgggactacaggcacccgccaccaggcccaactaat

SEQ ID NO: 11 - DNA, 7339 bp, synthetic construct
  misc_feature 1..7339: note = Description of Artificial Sequence: Synthetic Polynucleotide
  source 1..7339: mol_type = other DNA
tcccaggggctgatttctttggttttggatccagctggatgtctgcattgccgaggccaccagggctggctcagcaactgtcggggaatcaccagggtctgagaaatcttgtgcgcatgtgaggggctgtgggagcagagaaccactgggtgggaaattctaatccccaccctgctggaaactctctgggtggccccaacatgctaatcctccggcaaacctctgtttcctcctcaaaaggcaggaggtcggaaagaataaacaatgagagtcacattaaaaacacaaaatcctacggaaatactgaagaatgagtctcagcactaaggaaaagcctccagcagctcctgctttctgagggtgaaggatagacgctgtggctctgcatgactcactagcactctatcacggccatattctggcagggtcagtggctccaactaacatttgtttggtactttacagtttattaaatagatgtttatatggagaagctctcatttctttctcagaagagcctggctaggaaggtggatgaggcaccatattcattttgcaggtgaaattcctgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagattccggatccggagagggcaggggatctctccttacttgtggagacgtcgaggaaaaccctggaccaatggccttaccagtgaccgccttgctcctgccgctggccttgctgctccacgccgcccgcccggaacaaaaactcattagcgaagaggatctcgatattcagatgactcagaccacctcttctttgagcgcaagtttgggggatcgggttacaatatcctgccgcgccagccaagacatcagcaaataccttaattggtaccagcagaaacctgatggcactgtgaaactcctgatctaccataccagcaggttgcacagcggggtaccttcaagatttagcggatcaggaagcggtacagactactcacttacaatcagcaatctcgaacaggaagatatcgccacatacttctgtcagcaaggaaacactctgccctatacgttcggtggcggcacaaaactcgagattaccggaggtggaggctcaggaggaggaggcagtggaggtggtgggtcagaagtgaaactgcaggagtcaggaccgggcttggtcgcaccatcccaatccctttctgtcacatgcactgttagtggagtatccctaccagactacggggtatcttggatacggcagccgcctcgcaaggggctcgaatggctcggagtgatctgggggtctgagactacctattacaattccgctttgaagtcacggttgacgatcataaaagataacagtaaatctcaagtgtttctcaagatgaactcactccaaacagacgatacggccatatattattgcgccaagcactattattacggtggctcctacgcaatggattattgggggcaggggacttctgtaaccgtgtcaagcaccacgacgccagcgccgcgaccaccaacaccggcgcccaccatcgcgtcgcagccactgtcactgcgcccagaagcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagaggcgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgatgccttgcacatgcaagccctgccccctcgccgggctaaacgaagcggatctggggtgaagcaaaccttgaattttgacttgctgaagctcgcgggggatgtggaatctaaccctggtcctatggactggacatggattctgtttctcgtggccgccgccacacgcgtgcacagcagaaaggtgtgcaacggcatcggcatcggcgagtttaaggactctctgagcatcaacgccaccaacatcaagcacttcaagaactgcaccagcatctccggcgacctccacattctccccgtggcctttaggggagactccttcacccacacccctcctctggatcctcaagaactcgacattctgaagaccgtgaaggagatcaccggctttctgctgatccaagcttggcccgagaacagaacagatctccacgccttcgagaatctggagatcattagaggaagaacaaagcagcacggccagtttagcctcgccgtggtctctctgaacatcacatctctgggactgaggtctctgaaagagatcagcgacggcgacgtcatcatctccggcaacaagaatctgtgctacgctaacaccatcaactggaagaagctcttcggcaccagcggccagaagaccaagatcatcagcaatagaggcgagaacagctgcaaggccaccggacaagtctgccacgctctgtgtagccccgagggctgttggggacccgagcccagagactgtgtgagctgcagaaacgtttctagaggaagggagtgcgtggataagtgtaatctgctggagggcgagcctagggagttcgtcgagaactccgagtgtatccaatgccaccccgagtgtctcccccaagccatgaacatcacatgcaccggaagaggccccgacaactgcatccagtgcgcccactacatcgacggaccccactgcgtgaagacatgtcccgccggagtgatgggcgagaacaacacactggtgtggaagtacgccgatgccggacacgtctgtcatctgtgtcaccctaactgcacctatggctgcaccggccccggactggagggatgtcccaccaacggccctaagattccctccattgccaccggcatggtgggagctctgctgctgctgctcgtggtggctctgggaattggactgttcatgcgggccaagcggtctggatccggagccaccaacttcagcctgctgaagcaggccggcgacgtggaggagaaccccggccccattcagaatcctgatcctgcggtgtatcagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacagtattctaaggacgccagaaagctgttgatcggcttcaagcaggggagggacacctaatttgaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtcctcgtgataccaattcggagcctgcttttttgtacaaacttgttgataatggcaattcaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccagatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 12 - DNA, 7756 bp, synthetic construct
  misc_feature 1..7756: note = Description of Artificial Sequence: Synthetic Polynucleotide
  source 1..7756: mol_type = other DNA
tcccaggggctgatttctttggttttggatccagctggatgtctgcattgccgaggccaccagggctggctcagcaactgtcggggaatcaccagggtctgagaaatcttgtgcgcatgtgaggggctgtgggagcagagaaccactgggtgggaaattctaatccccaccctgctggaaactctctgggtggccccaacatgctaatcctccggcaaacctctgtttcctcctcaaaaggcaggaggtcggaaagaataaacaatgagagtcacattaaaaacacaaaatcctacggaaatactgaagaatgagtctcagcactaaggaaaagcctccagcagctcctgctttctgagggtgaaggatagacgctgtggctctgcatgactcactagcactctatcacggccatattctggcagggtcagtggctccaactaacatttgtttggtactttacagtttattaaatagatgtttatatggagaagctctcatttctttctcagaagagcctggctaggaaggtggatgaggcaccatattcattttgcaggtgaaattcctgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatattcaaaatccagacccagcggtatatcaactacgcgattcaaaaagttctgacaagagcgtgtgtctgttcaccgatttcgacagccagacaaatgtatcgcagtcaaaggattctgacgtctacataaccgacaaaactgtgttggacatgagaagtatggactttaagagcaattctgcggttgcttggagcaacaagtccgatttcgcctgcgcaaatgcttttaacaactctattatcccggaagatacctttttcccatcacccgaaagctcctgcgatgtgaagctggtggagaaatcctttgagactgacacgaatctgaacttccagaacctgagtgtgataggattccgaatcttgctcctgaaagtggccggatttaacctcttaatgacccttcggctttggtccagtggatccggagagggcaggggatctctccttacttgtggagacgtcgaggaaaaccctggaccaatggccttaccagtgaccgccttgctcctgccgctggccttgctgctccacgccgcccgcccggaacaaaaactcattagcgaagaggatctcgatattcagatgactcagaccacctcttctttgagcgcaagtttgggggatcgggttacaatatcctgccgcgccagccaagacatcagcaaataccttaattggtaccagcagaaacctgatggcactgtgaaactcctgatctaccataccagcaggttgcacagcggggtaccttcaagatttagcggatcaggaagcggtacagactactcacttacaatcagcaatctcgaacaggaagatatcgccacatacttctgtcagcaaggaaacactctgccctatacgttcggtggcggcacaaaactcgagattaccggaggtggaggctcaggaggaggaggcagtggaggtggtgggtcagaagtgaaactgcaggagtcaggaccgggcttggtcgcaccatcccaatccctttctgtcacatgcactgttagtggagtatccctaccagactacggggtatcttggatacggcagccgcctcgcaaggggctcgaatggctcggagtgatctgggggtctgagactacctattacaattccgctttgaagtcacggttgacgatcataaaagataacagtaaatctcaagtgtttctcaagatgaactcactccaaacagacgatacggccatatattattgcgccaagcactattattacggtggctcctacgcaatggattattgggggcaggggacttctgtaaccgtgtcaagcaccacgacgccagcgccgcgaccaccaacaccggcgcccaccatcgcgtcgcagccactgtcactgcgcccagaagcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagaggcgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgatgccttgcacatgcaagccctgccccctcgccgggctaaacgaagcggatctggggtgaagcaaaccttgaattttgacttgctgaagctcgcgggggatgtggaatctaaccctggtcctatggactggacatggattctgtttctcgtggccgccgccacacgcgtgcacagcagaaaggtgtgcaacggcatcggcatcggcgagtttaaggactctctgagcatcaacgccaccaacatcaagcacttcaagaactgcaccagcatctccggcgacctccacattctccccgtggcctttaggggagactccttcacccacacccctcctctggatcctcaagaactcgacattctgaagaccgtgaaggagatcaccggctttctgctgatccaagcttggcccgagaacagaacagatctccacgccttcgagaatctggagatcattagaggaagaacaaagcagcacggccagtttagcctcgccgtggtctctctgaacatcacatctctgggactgaggtctctgaaagagatcagcgacggcgacgtcatcatctccggcaacaagaatctgtgctacgctaacaccatcaactggaagaagctcttcggcaccagcggccagaagaccaagatcatcagcaatagaggcgagaacagctgcaaggccaccggacaagtctgccacgctctgtgtagccccgagggctgttggggacccgagcccagagactgtgtgagctgcagaaacgtttctagaggaagggagtgcgtggataagtgtaatctgctggagggcgagcctagggagttcgtcgagaactccgagtgtatccaatgccaccccgagtgtctcccccaagccatgaacatcacatgcaccggaagaggccccgacaactgcatccagtgcgcccactacatcgacggaccccactgcgtgaagacatgtcccgccggagtgatgggcgagaacaacacactggtgtggaagtacgccgatgccggacacgtctgtcatctgtgtcaccctaactgcacctatggctgcaccggccccggactggagggatgtcccaccaacggccctaagattccctccattgccaccggcatggtgggagctctgctgctgctgctcgtggtggctctgggaattggactgttcatgcgggccaagcggtctggatccggagccaccaacttcagcctgctgaagcaggccggcgacgtggaggagaaccccggccccattcagaatcctgatcctgcggtgtatcagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacagtattctaaggacgccagaaagctgttgatcggcttcaagcaggggagggacacctaatttgaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtcctcgtgataccaattcggagcctgcttttttgtacaaacttgttgataatggcaattcaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccagatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 13 - DNA, 7756 bp, synthetic construct
  misc_feature 1..7756: note = Description of Artificial Sequence: Synthetic Polynucleotide
  source 1..7756: mol_type = other DNA
tcccaggggctgatttctttggttttggatccagctggatgtctgcattgccgaggccaccagggctggctcagcaactgtcggggaatcaccagggtctgagaaatcttgtgcgcatgtgaggggctgtgggagcagagaaccactgggtgggaaattctaatccccaccctgctggaaactctctgggtggccccaacatgctaatcctccggcaaacctctgtttcctcctcaaaaggcaggaggtcggaaagaataaacaatgagagtcacattaaaaacacaaaatcctacggaaatactgaagaatgagtctcagcactaaggaaaagcctccagcagctcctgctttctgagggtgaaggatagacgctgtggctctgcatgactcactagcactctatcacggccatattctggcagggtcagtggctccaactaacatttgtttggtactttacagtttattaaatagatgtttatatggagaagctctcatttctttctcagaagagcctggctaggaaggtggatgaggcaccatattcattttgcaggtgaaattcctgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaatcccgaccctgcggtttatcagctacgcgactccaaatccagcgacaagtctgtgtgcctgttcacggatttcgattctcagacaaacgttagccagtcaaaagattctgacgtgtatatcactgacaaaaccgtcctggatatgaggagtatggattttaagtccaatagcgctgtcgcctggtctaacaagagcgactttgcttgtgcaaacgcctttaacaactcaattattccagaggatacttttttcccaagtcccgaatcctcctgcgacgtgaagctggtggagaagtcgtttgaaacagacaccaatttgaatttccaaaacttgtcagtgatcgggttcagaatactccttctgaaagtagccggcttcaatctgttaatgacccttcggctctggagcagtggatccggagagggcaggggatctctccttacttgtggagacgtcgaggaaaaccctggaccaatggccttaccagtgaccgccttgctcctgccgctggccttgctgctccacgccgcccgcccggaacaaaaactcattagcgaagaggatctcgatattcagatgactcagaccacctcttctttgagcgcaagtttgggggatcgggttacaatatcctgccgcgccagccaagacatcagcaaataccttaattggtaccagcagaaacctgatggcactgtgaaactcctgatctaccataccagcaggttgcacagcggggtaccttcaagatttagcggatcaggaagcggtacagactactcacttacaatcagcaatctcgaacaggaagatatcgccacatacttctgtcagcaaggaaacactctgccctatacgttcggtggcggcacaaaactcgagattaccggaggtggaggctcaggaggaggaggcagtggaggtggtgggtcagaagtgaaactgcaggagtcaggaccgggcttggtcgcaccatcccaatccctttctgtcacatgcactgttagtggagtatccctaccagactacggggtatcttggatacggcagccgcctcgcaaggggctcgaatggctcggagtgatctgggggtctgagactacctattacaattccgctttgaagtcacggttgacgatcataaaagataacagtaaatctcaagtgtttctcaagatgaactcactccaaacagacgatacggccatatattattgcgccaagcactattattacggtggctcctacgcaatggattattgggggcaggggacttctgtaaccgtgtcaagcaccacgacgccagcgccgcgaccaccaacaccggcgcccaccatcgcgtcgcagccactgtcactgcgcccagaagcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagaggcgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgatgccttgcacatgcaagccctgccccctcgccgggctaaacgaagcggatctggggtgaagcaaaccttgaattttgacttgctgaagctcgcgggggatgtggaatctaaccctggtcctatggactggacatggattctgtttctcgtggccgccgccacacgcgtgcacagcagaaaggtgtgcaacggcatcggcatcggcgagtttaaggactctctgagcatcaacgccaccaacatcaagcacttcaagaactgcaccagcatctccggcgacctccacattctccccgtggcctttaggggagactccttcacccacacccctcctctggatcctcaagaactcgacattctgaagaccgtgaaggagatcaccggctttctgctgatccaagcttggcccgagaacagaacagatctccacgccttcgagaatctggagatcattagaggaagaacaaagcagcacggccagtttagcctcgccgtggtctctctgaacatcacatctctgggactgaggtctctgaaagagatcagcgacggcgacgtcatcatctccggcaacaagaatctgtgctacgctaacaccatcaactggaagaagctcttcggcaccagcggccagaagaccaagatcatcagcaatagaggcgagaacagctgcaaggccaccggacaagtctgccacgctctgtgtagccccgagggctgttggggacccgagcccagagactgtgtgagctgcagaaacgtttctagaggaagggagtgcgtggataagtgtaatctgctggagggcgagcctagggagttcgtcgagaactccgagtgtatccaatgccaccccgagtgtctcccccaagccatgaacatcacatgcaccggaagaggccccgacaactgcatccagtgcgcccactacatcgacggaccccactgcgtgaagacatgtcccgccggagtgatgggcgagaacaacacactggtgtggaagtacgccgatgccggacacgtctgtcatctgtgtcaccctaactgcacctatggctgcaccggccccggactggagggatgtcccaccaacggccctaagattccctccattgccaccggcatggtgggagctctgctgctgctgctcgtggtggctctgggaattggactgttcatgcgggccaagcggtctggatccggagccaccaacttcagcctgctgaagcaggccggcgacgtggaggagaaccccggccccattcagaatcctgatcctgcggtgtatcagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacagtattctaaggacgccagaaagctgttgatcggcttcaagcaggggagggacacctaatttgaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtcctcgtgataccaattcggagcctgcttttttgtacaaacttgttgataatggcaattcaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccagatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 14 - DNA, 66 bp, synthetic construct
  misc_feature 1..66: note = Description of Artificial Sequence: Synthetic Polynucleotide
  source 1..66: mol_type = other DNA
tccggatccggagagggcaggggatctctccttacttgtggagacgtcgaggaaaaccctggacca

SEQ ID NO: 15 - DNA, 90 bp, synthetic construct
  misc_feature 1..90: note = Description of Artificial Sequence: Synthetic Polynucleotide
  source 1..90: mol_type = other DNA
cgggctaaacgaagcggatctggggtgaagcaaaccttgaattttgacttgctgaagctcgcgggggatgtggaatctaaccctggtcct

SEQ ID NO: 16 - DNA, 1059 bp, synthetic construct
  misc_feature 1..1059: note = Description of Artificial Sequence: Synthetic Polynucleotide
  source 1..1059: mol_type = other DNA
atggactggacatggattctgtttctcgtggccgccgccacacgcgtgcacagcagaaaggtgtgcaacggcatcggcatcggcgagtttaaggactctctgagcatcaacgccaccaacatcaagcacttcaagaactgcaccagcatctccggcgacctccacattctccccgtggcctttaggggagactccttcacccacacccctcctctggatcctcaagaactcgacattctgaagaccgtgaaggagatcaccggctttctgctgatccaagcttggcccgagaacagaacagatctccacgccttcgagaatctggagatcattagaggaagaacaaagcagcacggccagtttagcctcgccgtggtctctctgaacatcacatctctgggactgaggtctctgaaagagatcagcgacggcgacgtcatcatctccggcaacaagaatctgtgctacgctaacaccatcaactggaagaagctcttcggcaccagcggccagaagaccaagatcatcagcaatagaggcgagaacagctgcaaggccaccggacaagtctgccacgctctgtgtagccccgagggctgttggggacccgagcccagagactgtgtgagctgcagaaacgtttctagaggaagggagtgcgtggataagtgtaatctgctggagggcgagcctagggagttcgtcgagaactccgagtgtatccaatgccaccccgagtgtctcccccaagccatgaacatcacatgcaccggaagaggccccgacaactgcatccagtgcgcccactacatcgacggaccccactgcgtgaagacatgtcccgccggagtgatgggcgagaacaacacactggtgtggaagtacgccgatgccggacacgtctgtcatctgtgtcaccctaactgcacctatggctgcaccggccccggactggagggatgtcccaccaacggccctaagattccctccattgccaccggcatggtgggagctctgctgctgctgctcgtggtggctctgggaattggactgttcatg

SEQ ID NO: 17 - DNA, 1935 bp, Homo sapiens
  source 1..1935: mol_type = other DNA
tataaaacccggcggcgcaacgcgcagccactgtcgagtcgcgtccacccgcgagcacagcttctttgcagctccttcgttgccggtccacacccgccaccagttcgccatggatgacgatatcgctgcgctggtcgtcgacaacggctccggcatgtgcaaagccggcttcgcgggcgacgatgctccccgggctgtattcccctccatcgtgggccgccctaggcaccagggtgtgatggtgggaatgggtcagaaggactcctatgtgggtgacgaggcccagagcaagagaggtatcctgaccctgaagtaccccattgaacatggcattgttaccaactgggacgacatggagaagatctggcaccacaccttctacaatgagctgcgtgtggcccctgaggagcaccctgtgctgctcaccgaggcccccctgaaccctaaggccaaccgtgaaaagatgacccagatcatgtttgagaccttcaacaccccagccatgtacgtagccatccaggctgtgctgtccctgtatgcctctggtcgtaccacaggcattgtgatggactccggagacggggtcacccacactgtgcccatctacgagggctatgctctccctcacgccatcctgcgtctggacctggctggccgggacctgacagactacctcatgaagatcctgaccgagcgtggctacagcttcaccaccacagctgagagggaaatcgtgcgtgacatcaaagagaagctgtgctatgttgctctagacttcgagcaggagatggccactgccgcatcctcttcctccctggagaagagctatgagctgcctgacggccaggtcatcactattggcaacgagcggttccgatgccctgaggctcttttccagccttccttcttgggtatggaatcctgtggcatccatgaaactacattcaattccatcatgaagtgtgacgttgacatccgtaaagacctctatgccaacacagtgctgtctggtggtaccaccatgtacccaggcattgctgacaggatgcagaaggagattactgctctggctcctagcaccatgaagatcaagatcattgctcctcctgagcgcaagtactctgtgtggatcggtggctccatcctggcctcactgtccaccttccagcagatgtggatcagcaagcaggagtacgatgagtccggcccctccatcgtgcaccgcaagtgcttctaggcggactgttactgagctgcgttttacaccctttctttgacaaaacctaacttgcgcagaaaaaaaaaaaataagagacaacattggcatggctttgtttttttaaattttttttaaagtttttttttttttttttttttttttttttaagtttttttgttttgttttggcgcttttgactcaggatttaaaaactggaacggtgaaggcgacagcagttggttggagcaaacatcccccaaagttctacaaatgtggctgaggactttgtacattgttttgtttttttttttttttggttttgtctttttttaatagtcattccaagtatccatgaaataagtggttacaggaagtccctcaccctcccaaaagccacccccactcctaagaggaggatggtcgcgtccatgccctgagtccaccccggggaaggtgacagcattgcttctgtgtaaattatgtactgcaaaaatttttttaaatcttccgccttaatacttcatttttgtttttaatttctgaatggcccaggtctgaggcctcccttttttttgtccccccaacttgatgtatgaaggctttggtctccctgggagggggttgaggtgttgaggcagccagggctggcctgtacactgacttgagaccaataaaagtgcacaccttaccttacacaaacaaaaaaaaaaaaaaaa

SEQ ID NO: 18 - DNA, 1641 bp, Homo sapiens
  source 1..1641: mol_type = other DNA
gggacagccaagccaataagtgaggtcagctactccagcctcggtctggacgccagcagagcagaaaccttcgttgggggccgcactaggatccttgaaaaaccacagagaacctacccagcagcgaccactcaggctggagaccccgagcagaagggaaccatctacggtggggagagcaaagttataagacccagactcgacgagagaccctgactcggcgccggagggaccgcacacgtggtgtacctctgcgcaaccgcccctctccgagggaattcgctctacttctcgcgagaattgtgtctacccttccagcctgcttttccgtctcctgtccccgcagcgggattttgtttgtttccctccctcagccccacccattttttctcctcttctcgcgatagtggggagggggcggggaaaaagccgagaacttccgagaaaaagcgagctgatctcttctcgcgagatgtgcgagcggccatcttgctcctgcccctgacaggctcgggtcggggtcacagggacacggagcccgctgtcccgctcttcgcagacaatgctgtcccgggtggtgctttctgctgccgccacagcggccccgtgtctgaagaacgcggccgccctaggtccaggggtattacaggcaacaagggcctttcacacaggacagcctcgccttgcccctctaccacctcttcctgaatatggaggaaaagtgcgtcttgggctgattcctgaggaatttttccagttcctttaccctaagactggtgtaacaggaccttatgtgcttggaactggacttagcttgtattttctatccaaagaaatatatgtgattaccccagagaccttctctaccatatcagtagtagggttgatagtctatgtgattaagaaatatggcgcctcttttggagaatttattgacaaacttaatgaggaaaaaattgctcaactagaagaagtaaagcagtcgagcatgaaacaaatccaggatgcaatcgacatggagaaggcacagcaggcactggttcagaagcgccattacctcttcgatgtgcagaggaataacattgccctggccttggaggtcacttaccgggaacggctacataaagcatataaggaggtaaagaatcgcctggactaccacatctctgtacagaacatgatgcgtcgcaaggaggaagaacacatgatagactgggtagaaaagcatgtggtgaagagcatttctgtacagcaggaaaaggagaccattgccaagtgcattgaagatctaaagctgcttgcaaagaaggctcaagctcagccaattatgtgaatatgtctttctcagttgtgatagccagagagagttaaatgggaactagtatatgtgaagaactctttctgtattgctatctattgaaataaaatgatcaaggtccacttagtggcttaaccctattggccagtaaggtatttctgatccttgctctgtatgcggagttatctgatcataatttgaataagcaacttgcagcaacttgctgcccaactgaaattaccaagttataatttaaacttgtaattaactaaagcatcttgcaataaaatgtttgaaacagaatttataagtttcatactctg

SEQ ID NO: 19 - DNA, 858 bp, Homo sapiens
  source 1..858: mol_type = other DNA
tttcagtggctgctactcggcgcttcagtcgcggtcgcttcagtcgtcagcatggctcgctcggtgaccctggtctttctggtgcttgtctcactgaccggcctgtatgctatccagaaaacccctcaaattcaagtatactcacgccacccaccggagaatgggaagccgaacatactgaactgctacgtaacacagttccacccgcctcacattgaaatccaaatgctgaagaacgggaaaaaaattcctaaagtagagatgtcagatatgtccttcagcaaggactggtctttctatatcctggctcacactgaattcacccccactgagactgatacatacgcctgcagagttaagcatgccagtatggccgagcccaagaccgtctactgggatcgagacatgtgatcaagcatcatgatgctctgaagattcatttgaacctgcttaattacaaatccagtttctaatatgctatacaatttatgcacgcagaaagaaatagcaatgtacacatcaccttctttatatcttactttaaatattttatgcatgttttcaaaaaattggaaatatcctagatagctgagcaataaatcttcaataagtattttgatcagaataataaatataattttaagaacaatagttgatcatatgccaaaccctctgtacttctcattacttggatgcagttactcatctttggtctatcacaacataagtgacatactttccttttggtaaagcaaagaggcctaattgaagtctgtcactgtgcccaatgcttagcaattctcacccccaaccctgtggctacttctgcttttgttacttttactaaaaataaaaaac

SEQ ID NO: 20 - DNA, 1296 bp, Homo sapiens
  source 1..1296: mol_type = other DNA
aacccttaagagggatgctgcccttaccccggggtcccagcttaggttcatcaggtaaactcaggagagtgtttcctcgtcccgtagacaaaatggtgaaggtcggtgtgaacggatttggccgtattgggcgcctggtcaccagggctgccatttgcagtggcaaagtggagattgttgccatcaacgaccccttcattgacctcaactacatggtctacatgttccagtatgactccactcacggcaaattcaacggcacagtcaaggccgagaatgggaagcttgtcatcaacgggaagcccatcaccatcttccaggagcgagaccccactaacatcaaatggggtgaggccggtgctgagtatgtcgtggagtctactggtgtcttcaccaccatggagaaggccggggcccacttgaagggtggagccaaaagggtcatcatctccgccccttctgccgatgcccccatgtttgtgatgggtgtgaaccacgagaaatatgacaactcactcaagattgtcagcaatgcatcctgcaccaccaactgcttagcccccctggccaaggtcatccatgacaactttggcattgtggaagggctcatgaccacagtccatgccatcactgccacccagaagactgtggatggcccctctggaaagctgtggcgtgatggccgtggggctgcccagaacatcatccctgcatccactggtgctgccaaggctgtgggcaaggtcatcccagagctgaacgggaagctcactggcatggccttccgtgttcctacccccaatgtgtccgtcgtggatctgacgtgccgcctggagaaacctgccaagtatgatgacatcaagaaggtggtgaagcaggcatctgagggcccactgaagggcatcttgggctacactgaggaccaggttgtctcctgcgacttcaacagcaactcccactcttccaccttcgatgccggggctggcattgctctcaatgacaactttgtcaagctcatttcctggtatgacaatgaatacggctacagcaacagggtggtggacctcatggcctacatggcctccaaggagtaagaaaccctggaccacccaccccagcaaggacactgagcaagagaggccctatcccaactcggcccccaacactgagcatctccctcacaatttccatcccagacccccataataacaggaggggcctagggagccctccctactctcttgaataccatcaataaagttcgctgcacccacaaaaaaaaaaaaaaaaaaaaaa

SEQ ID NO: 21 - DNA, 2694 bp, Homo sapiens
  source 1..2694: mol_type = other DNA
gtcaggcatatggggaaacgatccaggacttcaagcgacttcctctttctgcagagtccagccttggggcctctagatagccttgagccagcagctgtacaagcgcttgccctcaaccaagttggcgcaggcggcttctctcgagaacatgtgactcttccaggtcccgcctctgccctcccagagaggaggctaaaaagcccagccgctggtcggtgggcagcctggccagtatgtccctaaaatggagtgcgtgttgggtcgcgctgggccagctgctgtgcagctgcgcgctggctctgaagggcgggatgctgttcccgaaggagagcccgtcgcgggagctcaaggcgctggacggactgtggcacttccgcgccgacctctcgaacaaccggctgcagggtttcgagcagcaatggtaccggcagccgctacgggagtcgggcccagtcttggacatgcctgtcccttctagcttcaatgacatcacccaagaagcagcccttcgggactttattggctgggtgtggtatgaacgggaagcaatcctgccacggcgatggacccaagataccgacatgagagtggtgttgaggatcaacagtgcccattattatgcagttgtgtgggtgaatgggattcatgtggtggaacatgagggaggtcacctcccctttgaggctgacattagcaagctggtccagagtgggcccctgaccacctgccggatcacgattgccatcaacaacacactgacccctcatacccttccgccggggaccatcgtctacaagactgacacctccatgtatcccaagggttactttgtccaggacacaagctttgacttcttcaactatgcgggactgcatcgatctgtggtcctctataccacccctaccacttacatcgatgatatcactgtgatcactaatgtggagcaagacatcgggctggtgacctactggatttctgtgcagggcagtgaacatttccagctagaagtgcaacttttggatgagggtggcaaagtcgtggcccatgggacagggaaccagggtcaacttcaggttcccagtgccaacctctggtggccttacctgatgcatgagcatccagcctacatgtactccttggaggtgaaggtgacaacaactgagtctgtgactgactactacacccttcctatcgggattcgaacagtggctgtcacaaagagcaagttcctcataaacgggaagcccttctatttccaaggggtcaataagcacgaggattcagatatccgagggaaaggcttcgactggccgctgctggtaaaggatttcaacctgctccgttggctcggggcaaattcctttcgtaccagccactatccctactcagaggaggtacttcagctctgtgaccgatacgggattgtggtcatcgatgagtgtcccggtgtgggcattgtgctacctcagagttttggcaacgagtcacttcggcaccacctagaggtgatggaggagctggttcgccgggacaaaaatcaccctgcggttgtgatgtggtctgtggccaatgagccttcctctgctctgaaacccgccgcatattactttaagacgctgatcacccacaccaaagccctggacctcacccgtcccgtgacctttgtgagcaacgccaaatatgatgcagacctgggggccccgtacgtggatgttatctgtgtaaacagctacttttcttggtatcatgactatgggcatttggaggtgattcagccacagctgaatagccagtttgagaactggtataagacgcatcagaagccgattatccagagcgagtatggagcagacgcaatcccagggatccacgaggacccgcctcgcatgttcagtgaggagtaccagaaggctgttctggagaattaccattcagttctggatcagaaacgtaaagaatacgtggtcggagagctcatctggaatttcgccgacttcatgacgaaccagtcaccgctgagagtaatcggaaacaagaaggggatcttcactcgccagagacagcccaaaacttcggcctttattttgcgagagagatactggaggattgccaacgaaaccggaggtcacggttcagggccgagaacccagtgtttcggaagcagaccgttcacgttctaaagttacaactacctcactgcagactcaaaggtgtccccagacacttcctgacactagtgtttctatttgggattttgtgaactgatacaaggggactttagaagtgacatcaaagcttttcttccgtggggataataacttacagtgccttaggctcagtaatatgtcctgctgagaggtgtccaagataatttgttagttttttggccttggctttgtgaactcttgaaagcctgctgtgtgaacattctcactaaattaagagctactggtgagaggctggagtgaaggctggctcacaatttaagagcactgactgttctactggaggttccagattcaattcctagcacccacctggtggctaactatgagctggtcctccatttcccaggtgatccaaatgcccttttggcccctgcgggtaccacatgtatgtggtatgtggatctacatgtggggaaaaaagtcatacatgcaaaataaaaatgaatctaaaaaaaaaaaaaaaaaa

SEQ ID NO: 22 - DNA, 1349 bp, Homo sapiens
  source 1..1349: mol_type = other DNA
ggagcctggccggcagcgtttctgagccattgctgaggcggcgagggagagcgttgggcttacctcactgctttccggagcggtagcacctcctccgccggcttcctcctcagaccgctttttgccgcgagccgaccggtcccgtcatgccgacccgcagtcccagcgtcgtgattagcgatgatgaaccaggttatgacctagatttgttttgtatacctaatcattatgccgaggatttggaaaaagtgtttattcctcatggactgattatggacaggactgaaagacttgctcgagatgtcatgaaggagatgggaggccatcacattgtggccctctgtgtgctcaaggggggctataagttctttgctgacctgctggattacattaaagcactgaatagaaatagtgatagatccattcctatgactgtagattttatcagactgaagagctactgtaatgatcagtcaacgggggacataaaagttattggtggagatgatctctcaactttaactggaaagaatgtcttgattgttgaagatataattgacactggtaaaacaatgcaaactttgctttccctggttaagcagtacagccccaaaatggttaaggttgcaagcttgctggtgaaaaggacctctcgaagtgttggatacaggccagactttgttggatttgaaattccagacaagtttgttgttggatatgcccttgactataatgagtacttcagggatttgaatcacgtttgtgtcattagtgaaactggaaaagccaaatacaaagcctaagatgagcgcaagttgaatctgcaaatacgaggagtcctgttgatgttgccagtaaaattagcaggtgttctagtcctgtggccatctgcctagtaaagctttttgcatgaaccttctatgaatgttactgttttatttttagaaatgtcagttgctgcgtccccagacttttgatttgcactatgagcctataggccagcctaccctctggtagattgtcgcttatcttgtaagaaaaacaaatctcttaaattaccacttttaaataataatactgagattgtatctgtaagaaggatttaaagagaagctatattagttttttaattggtattttaatttttatatattcaggagagaaagatgtgattgatattgttaatttagacgagtctgaagctctcgatttcctatcagtaacagcatctaagaggttttgctcagtggaataaacatgtttcagcagtgttggctgtattttcccactttcagtaaatcgttgtcaacagttccttttaaatgcaaataaataaattctaaaaattc

SEQ ID NO: 23 - DNA, 1840 bp, Homo sapiens
  source 1..1840: mol_type = other DNA
gggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctcacggtgttgccaaaatgtcgctttccaacaagctgactttggacaagctggacgtgaaggggaagcgggtcgtgatgagggtggacttcaacgttcctatgaagaacaaccagataacaaacaaccaaaggatcaaggctgctgttccaagcatcaaattctgcttggacaatggagccaagtccgttgtccttatgagccacctgggccggcctgatggtgttcccatgcctgacaagtactccttagagccagttgctgctgaactcaaatctctgctgggcaaggatgttctgttcttgaaggattgtgtgggcccagaagtcgagaatgcctgtgccaacccagcggctgggactgtcatcctgctggaaaacctccgctttcatgtagaggaagaagggaagggaaaagatgcttctggaaacaaggttaaagctgagccggccaaaattgatgctttccgagcctcactgtccaaactaggagatgtctatgtcaatgatgcttttgggactgcacaccgagcccatagctccatggtgggtgtgaatctgccacagaaggctggtggatttttgatgaagaaggagctgaactactttgccaaggctttggagagtccagagcgacccttcctggctatcttgggaggcgctaaagttgcagacaagatccagctgatcaataatatgctagacaaagtcaatgagatgatcattggtggtggaatggcctttaccttccttaaggtgctcaacaacatggagattggcacatctctgtatgatgaagaaggagccaagattgtcaaagatctcatgtccaaagctgagaaaaatggtgtgaagattaccttgcctgttgactttgtcactgctgacaaatttgatgagaatgccaagactggccaagctactgtggcctctggtatacctgctggctggatgggcttggactgtggtactgagagcagcaagaaatatgccgaggctgtgggtcgagctaagcagattgtttggaatggtcctgttggggtatttgaatgggaagcctttgccaggggaaccaagtcactcatggatgaggtggtgaaagccacttctaggggttgcatcactatcataggtggtggagacactgccacttgctgtgccaaatggaacacagaggataaagtcagccatgtgagcactgggggcggtgccagtctagagctcctggaaggtaaagtccttcctggggtggatgctctcagcaatgtttagtattttctttcctgcctttggttcctgtgctcctaagtcaacctagtgttttccacatctccatttggtgttagcgcaagattcagctagtggctgagatgtggcaccaggaacccttaaacagttgcacagcatctcagctcgtctttactgcatcaggatttgtctgtgttcttcaagatcccatttaaattccttagtgactaaaaccattgtgcattgtagagggcgtctatttatattctgcctgagaaaggaagtgagctgtaaaggctgagctctctctctgacgtatgtagcctctggttagcttcgtcactcactgttcttgactcagcatggcaatctgatgaaatcccagctgtaagtctgcagaaattgatgatctattaaacaataaagatgtccactaaaatggaaaaaaaaaaaaaaaaa

SEQ ID NO: 24 - DNA, 736 bp, Homo sapiens
  source 1..736: mol_type = other DNA
tttgcagacgccactgtcgcttttcgccgcttgctgcagccatggtcaaccccaccgtgttcttcgacatcacggccgatgacgagcccttgggccgcgtctccttcgagctgtttgcagacaaagttccaaagacagcagaaaactttcgagctctgagcactggagagaaaggatttggctataagggttcctcctttcacagaattattccaggattcatgtgccagggtggtgactttacacgccataatggcactggcggcaggtccatctacggagagaaatttgaggatgagaacttcatcctaaagcatacaggtcctggcatcttgtccatggcaaatgctggaccaaacacaaacggttcccagttttttatctgcactgccaagactgaatggctggatggcaagcatgtggtctttgggaaggtgaaagaaggcatgaacattgtggaagccatggagcgttttgggtccaggaatggcaagaccagcaagaagatcaccatttccgactgtggacagctctaatttcttttgacttgcgggcattttacccatcaaaccattccttctgtagctcaggagagcgtccctaccccatctgctcgcaatgtcctgtaatctctgctctcactgaagttctttgggttccatattttcctcattccccttcaagtctagctggattgcaaagttaagtttatgattatgaataaaaactaaataag

SEQ ID NO: 25 - DNA, 524 bp, Homo sapiens
  source 1..524: mol_type = other DNA
gccgccgtctgtgccgccgccatgtctctagtgatccctgagaagttccagcacattttgcgagtactcaacaccaacatcgatgggcggcggaaaatagccttcgccatcactgccattaagggcgtggggcggagatatgctcatgtggtgttgaggaaagcagacatcgacctcaccaagagggctggagaactcacggaggatgaggtggagcgagtgatcaccatcatgcagaacccacgacagtacaagatcccagactggttcctgaacagacagaaggatgtgaaggatgggaagtacagccaggttctggccaacggtctagacaacaagctgcgtgaggacctggagaggctgaagaaaattcgagcccatagagggctgcgccacttttggggccttcgtgtccgcggtcagcacaccaagaccactggccgcaggggccgaactgtgggtgtatccaagaagaaatgagtctctgggcctttgctgttaataaatagtttatatacctatga

SEQ ID NO: 26 - DNA, 1842 bp, Homo sapiens
  source 1..1842: mol_type = other DNA
aagagcgcaactggcggaagtgacggcatcagatgtgcgtcaggcgttcggtggatcgagtccggtagcggtggcgggtatctgctggcggtttggctaggtttctgcggtcgcgtcattttctccgcagtgcccagcatcactatttcatggtgtgtgaagataacccagaacattgaagacgtttctaaggagatattcagaggatgctctagggaagatctgagtactgaagaaagggagaatcatggaccagaacaacagccttccaccttatgctcagggcttggcctccccacagggcgccatgactcctggaattcccatctttagtccaatgatgccttacggcacaggacttactccacagcctattcagaacaccaacagtctctctattttggaagagcaacaaagacagcagcagcaacagcaacagcagcagcaacaacagcaggcagtagcaactgcagcagcctcagtacagcaatcaacatctcagcaacccacacagggtgcctcaggccagaccccacaactcttccattctcaaactctgaccactgcaccgttgccaggcaccacccccttgtacccttcaccaatgactcctatgacccctatcactcctgccacaccagcttctgagagctctggaattgtaccgcagcttcaaaatattgtatctaccgtgaatcttggctgtaaacttgacctaaagaccattgcacttcgtgcaagaaatgctgaatataatcccaagcgatttgctgcagtcatcatgagaataagagagccacggacaactgcgttgattttcagttctggaaaaatggtgtgcacaggagccaagagtgaagaacaatccagactagcagcaagaaaatatgctagagttgtgcagaagttgggcttcccagctaagttcttagacttcaagatccagaacatggtggggagctgtgatgtgaagttccctataaggctggaaggccttgtgctgacccaccagcagttcagtagctatgagccagaattatttcctggattaatctacagaatgatcaaacccagaattgttctccttatttttgtttctggaaaagttgtattaacaggtgctaaagttagagcagagatttatgaagcatttgaaaacatctaccccatcttaaagggattcaggaagaccacatagttgtctgccatgttctcctgccttccctatccacgtgttttttaaaaccagtcagttttggtaccactgatggtacagttggtgaggacactcagttacaggtggcagcatgaagtgacactgtgtgtcctactgcaggatactagaaaggttccccctctgcactgaaatcaccctgcagcactactgtgagttgcttgctctgtgctgctacttgggcggcactgcccatttatttatatttagattttaaacactgctgttggtgattgttggtttaagggacagaactttaagtgttcaagccacctgtacaattggacttttcattttaatctttcccacacaagccagtttttatatttctaccagaaaagtaaaaatcttttttaaaagtgttgtttttctagtttgtaactcttaggagttatttttgtgccagatacattccgccttcccagtattgcaggactgaatagttgtattaatcaaaacaatggctgtacatacttttctttcttcagagtactctgcacaaaaacgcagcttgtaaattgttagatttttgttataaatgataccttgtaagtcatgtgatcatactgtcaaagaaatttattttagatataatgcctgagaccatt

SEQ ID NO: 27 - DNA, 5008 bp, Homo sapiens
  source 1..5008: mol_type = other DNA
tgcgggcggggaggcggaggggtgtcgcggagtccggggctgaggagcgcgggttgcaggtgcagcgcggtgggtgtggggagccgctgtaccctgcgcccctcgggtcctccgggccttcgcaggccagtgctaggccgcgggttcgagagtcaccacgctgaggcgcaggcttgttccgccgggagcacgtggtggcggctggaggaagtcgccccagggaacggctgtcggggtactcccttgtagcagctgagaatgatggatcaagccagatcagcattctctaacttgtttggtggggaaccattgtcatacacccggtttagccttgctcggcaagtagatggagataacagtcatgtggagatgaaactggctgcagatgaagaagaaaatgccgacaataacatgaaggctagtgtcagaaaacccaagaggtttaatggaagactctgctttgcagctattgcactagtcattttcttcttgattggattcatgagtggctacctgggctattgtaagcgtgtagaacaaaaagaggagtgtgtgaaactggctgaaacggaggagacagacaagtcagaaaccatggaaacagaggatgttcctacatcatctcgcttatattgggcagacctcaaaacactgttgtcagagaagttgaactccatagagtttgctgacaccatcaagcagctgagccagaatacatacactcctcgtgaggctggatctcaaaaagatgaaagtcttgcctattatattgaaaatcagttccatgaatttaaattcagcaaagtctggcgagatgaacactatgtgaagattcaagtgaaaagcagcattggtcaaaacatggtgaccatagtgcagtcaaatggtaacttagacccagtggagtctcccgagggttatgtggcattcagtaaacctacagaagtttctggtaaactggtccatgctaattttggcactaaaaaggactttgaagaactaagttattctgtgaatggatctttagtgattgttagagcaggggaaattacttttgcagaaaaggttgcaaatgcccaaagctttaatgcaattggtgtcctcatatacatggacaagaataaattccccgttgttgaggcagaccttgcactctttggacatgctcatctaggaactggtgatccatacacacctggctttccttctttcaatcatactcagtttccgccatctcagtcatcagggttgcctaatatacctgtgcaaacaatctcaagagctgctgcagaaaagctatttggaaaaatggaaggaagctgtcctgctagatggaacatagattcttcatgtaagctggaactttcacagaatcaaaatgtgaagctcattgtgaaaaacgtactgaaagaaagaagaatacttaacatctttggagttattaaaggttatgaggaaccagaccgttatgttgtagtaggagcccagagagacgctttgggtgctggtgttgcggcgaagtccagtgtgggaacaggtcttctgttgaaacttgcccaagtattctcagatatgatttcaaaagatggatttagacccagcagaagtataatctttgccagctggactgcaggcgactttggagctgttggtgccactgagtggttggagggatacctttcatctttgcatttaaaagctttcacttatattaatttggataaagttgtccttggtactagtaacttcaaagtttctgccagccccttattatatacacttatgggaaagataatgcaagatgtaaagcatccagttgatggaaaatctctatatagagacagcaattggattagcaaagttgagaaactttcctttgacaatgctgcatatcctttccttgcatattctggaatcccagcagtttctttttgtttttgtgaggatgcagactatccttatttgggcactagattggatacctatgaggcattgactcagaaagttcctcagctcaaccaaatggttcgtacagcagcggaagtggctggtcagctcattattaaacttacccatgacgttgaattgaacctggactatgagatgtataacagcaaactactgtcatttatgaaggatctgaaccagttcaaaacagatatcagggatatgggtctaagtctacagtggctgtattccgctcgtggagactacttccgtgctacttctagactaacaactgattttcataatgctgagaaaacaaacagatttgtcatgagggaaatcaatgatcgtattatgaaagtggagtatcacttcctgtcgccctatgtatctccaagagagtctcctttccgacatatcttctggggctctggctctcacactctctcagctttagtggagaacttgaagcttcgtcaaaaaaatattactgcttttaatgaaaccctcttcagaaaccagttggccctggctacttggactattcagggagtcgcaaatgccctctctggtgacatttggaatattgacaatgagttttaaatgtaacatgcataattaaataagagcagggtagtctgtttctagacttgtgttggttgtgctaaattttcattagagctctaaaatctaatgttaaaattctacccaatcatctaatgtcttaggcagcagcttttagtgcggggttggacctacacttcaagtacaatggataacactttccatgtttgtgatatcttctcagattatctttaaaatttttgagtcctttgtaataacttcctctttgcttcatggtcatgaaaatgtcagaaccagttgtaagaacaatgctatatgtcctgagggcattaactagtgtcttttgaggtaggagaaagagtgtgcttgtggagggtcaacgtggtagttgggtagttggagattgccccgtctccatctgatcttccattgagctgtatgcccttaaggatgtagctggtttgcatttccctaaattagacagtaactttcagaagagatggaacttgttttcttgccagcaaggttgaaaataggtccttagctggaccaatttgtctgtttacaggaataagtaaggccttactggttaaccttggtgttctttatgatgagaccagaagccaaagacctcaagttttctctcctcttggcatctgccctatagtctttagttctttgtttttattttgtttttctttttttcccaacattttctgaaaaagaacaagttttagactcaatttgtcagacttgaaaagaacacactgccaagttttggccaaagtgttagtcttcaggaaagctttctatcattttggcactgagatatttattgtttatttatcagtgacagagttcactataaatagtgttgttttttttttatatatagaagataattatcggaagcagtgccttccataattatgacagttatactgtcgttttcttttaataaaagcagcatctgctaatgagacccacagatactggaagttttgcacttatggtcagcacttgcaactttaggaaggaagaatgccacaatccaaataatatgagctagaagaggattgggttaaataagagattcctaattgagttggggaaaaaaatgatagttttcctaagtgcagtgagttgtggccaagttaaatgtcatttaaaggctatggtagtacttacatctacaaaattttacagctcaatttattcaagatgtaactcaaaatcaattttgcaaaatttccagtacctttgtcacaaacttaactcacattatcgggagcagtgtcttccataatgcataaagaacaaggtagtttttgcctaccacagtgtctatatcggagacagtgatctccatatgttacactaagggtgtacgtaattatcgggaacagtgtttcccataattttcttcatgcaatgacatcttcagagcttgaagatcgttagtatctaacatgaattcctaacttcctgtagctccttagttttagttgcaaaaaacatttgtggtcattgagcatttggtgggtaaaatcaactgctgtaaaatcattacttccaagatccttttttaaaatgtgggtattttgttatctctggtttatggaataaaagcatacgtttataatgtttgctggtgacaaaggaatttaactatctccctttatttgcatgttacatgttatttcctaaatgtagttcttagaagtttgaattcatttttggtttttgggagggtagaaagtagagaataatgggtgttgggaagacaaagcttgcaaaaggatctttatgtacctgctcatcactatggtggctatgaattatgtaggtaatgagcaagatcacattaacaaagactagttaaccatcattacgcatctaatcagttttgccatggggtcagttcaaagctgccacctgagaatgtcactaggctctcagggtcttggcaccactcgcccaagttatatccaccagattattttgagcctcactgagttgttttgctactcagtcccttcagtcttcacagtgtttctcttgtaccagtatgagtatcaccgtgcataatgggattccaagtcttgaatgaaggcatgcattttacatagacgtctctgtgaaataacctttgttgtaagtggtgtttccagtcaaagcagtaagttggtaaggtttagtcttgggtgaaatgtggatctgagtaataagtagcttctgctacttatcaggttattgttggttaaaatgtagattttgaagataaaaatagatcttttgtcatgactcactgctagaatttgcatgacctttactgtgttagcttttgaatgccttttggtttggacttgccaacctatactgtctttgctgaaatgcccttataaatgtatacagctggtatgtaacaatgtgaagattccttacctgacttaataaaatacttcgataatactga

SEQ ID NO: 28 - DNA, 3801 bp, Homo sapiens
  source 1..3801: mol_type = other DNA
ggaggagcctcgctcctcctccagctagagccctaggaacggccacgctccctaaccttgcttccaggacctaaaagggtcggtcactctcttacaatgcgctaattaggaacaatgtgtctcccaatgaaagctctacagagggtaaagtgcagcccacgacaacagttcggtgtgttctagcctgtttccccgcctcgccccacgtcaggcttctacgatcacgtccaacccgctagcccaaccctttccccgctcctcccagcgaccacccattgtccccgcggctgtcacggtgtggacgccgagctcgcgactggaaaggcagggcgtcattcagcctcgcgcttttcccagccttaaaaggtctaaggccgcttcctgcagccagaagcgtttgacgtcatcgtgcgtgtggtgcccctgctgccggggctagtgattggaggaaaccccgtgtctgcggagcggctgtagcctgtgagcagccagatcagggacagagtctcagcctcgccgccaccgctgctgccgccgccgcccagagaccgccgagccgcgtccgtgtgccgccacccactccggacacagaatatccagttatggataaaaatgagctggtgcagaaggccaagctggccgagcaggcagagcgatatgatgacatggcagcctgcatgaagtctgtcactgagcagggagctgagctgtcgaatgaggagagaaaccttctctctgttgcttataaaaacgttgtaggagcccgtaggtcatcgtggagggtcgtctcaagtattgagcagaagacggaaggtgctgagaaaaagcagcagatggctcgagaatacagagagaagatcgagacggagctgcgtgacatctgcaacgatgtactgtctcttttggaaaagttcttgatccccaatgcttcgcaaccagaaagcaaagtcttctatttgaaaatgaagggtgactactaccgttacttggccgaggttgctgctggtgatgacaagaaaggaattgtggaccagtcacagcaagcataccaagaagcatttgaaatcagcaaaaaggagatgcagccgacacaccccatcagactgggtctggccctcaacttctctgtgttctattacgagatcctgaactccccagagaaagcctgctctcttgcaaaaacagctttcgatgaagccattgctgaacttgatacattaagtgaagagtcgtacaaagacagcacgctaataatgcagttactgagagacaacttaacattgtggacatcggatacccaaggagatgaagcagaagcaggagaaggaggggaaaattaaccggccttccaacctttgtctgcctcattctaaaatttacacagtagaccatttgtcatccatgctgtcccacagatagtttttttgtttacgatttatgacaggtttatgttacttctatttgaatttctatatttcccatgtggtttttatgttttaatattaggggagtagagccagttaactttagggagttactcattttcatcttgaggtggccaatatgggatgtggaatttttacatgagttacacatgtttggcatagtacttttggtacattgtggcttcagaagggccagtgttaaaactgcttccatgtctaagcaaagaaaactgcctacatattggtgtgtgctggcggggaataatcaggataatgggttccagtcatgagtgtagtctttgtgggtactgtaaggcctggagcacttgtgaggctgtgacacaaacaccctgtggatgcacgctaggatcgtgtgtgtctgcgtgcacactcttgaccacagctccagaagttgtctctagacaaagtcgtgacccaatttactctggtaagggcagaaacggttcacattccattatttgtaaagttacctgctgtttgctttcattatttttgctacacattttatttgtatttaaatgttttaggcaatctaagaacaaatgtaaaagtaaagatgcagtaaaaacgagttgcttggtgtgcccggctccatacgtatcaagcacagcggtaaacaaaatcccatgtatttaactttttctttctttttttttttttttaaagttttttgttttgttttgtttttgcttttgtgattttttttttttctttttgatacttgcctaacatgcatgtgctgtaaaaatagttaacagggaaataacttgagatgacggctagctttgttttaatgtcttaagaaattttcatgaacaatccaagcataattgttaagaacacatgtattaaagttcatgtaagtggaataaaagttttatgaatggacttttcaactacttttctctacagcttttcatgtaaattagtcttttggttttgagacttctccaaaggaaattgtctccttattccctcttggcagctaatgggcttttaccaattttgaatgcagagtttatcataaaaaaaaaaaaaccgctaatgtagcttccatcctcagccacctcccacattttccccttactcctcaacaattgaagtggacttttatggggtggggggtgattggcaaaaggtaatgtgttccatttataattttggtctatggagttttctaacttaggaagccacaatgttcttggcccatgtgactgacactgggcagcattaactgttaagttctgtgcttccaagtcgctgttggttttaagaatttcctgatgctcttacagtctgcctgggattctattcctctatttccatgttgggcacaggctcacttcccttttactgtcttgtcaccaaccattccaacttggtggccatgtatttgggaaaagggccgcatgatctttctggctccactcagtgtctaagatacccgcttcccctgcttgcctcccacagcctcccctcatcctctctacagcccagctcgcctcagttgagcttgtgtttatctccctggaagtcctgccctaaatggtctgtcaccgtctccctttaaaatccttcctcctcctcttcctcctcctcctcctcctcacataatgatggggctaagtcacacccaaagctcacaccctaccgagtatttcctcagtactttacagaaaacaccaaacaaaaatgccattttacaagaggtgtatttttttttcttttagaatgtaagctcgtctagagcagggacaatgttttctgtatgttctattgtgcctagtacactgtaaatgctcaataaatattgatgatgggaggcatgagtctcagtggtgagggaggggagagaaatcccaagcatgttttctggcttgcttcatgatgatctgaatctgaaatcccactccttttggacagattatcacaactactgcattcaggtggggtggggtggggtggggtgggggtgttttctggagaaacgacatcacacacgtggtgccatgacccatcttcctttacaactcaattccttgattttgcctcagctcccagactgaggaagattaagcaatggacagcaggatgtcatctcatttgagtaaattacaaatgatacctggaacctgtcacatttgcctaattggcttattcacatgcattagacattgctaaactagtttgattttagttattgatgtggtttttttttttttttatgaatagtgcccagtagatggagaaagatttgcaacctcaaattttccaccccctggaatttgaataataaatttgtttttaacttacttccctaattgtcccaaaaaaa

SEQ ID NO: 29 - DNA, 2223 bp, Homo sapiens
  source 1..2223: mol_type = other DNA
aaatctatcgccttgagccgttggccttcagataggctgatttggttggtgtcttgctctttctgtgggaaggctgcggctcacttccttctgacttcttgataattttgcattagacatttaactcttctttctatgatctttccttctagacactgagttttttggttgttgcctaaaaccttttcagaaatcccttccctcgccatcacactgacatgagtgtgggtcttcctggtccccacagtttgcctagttctgaggaagcatcgaattctgggaacgcctcatcaatgcctgcagtttttcatcccgagaactattcttgcttacaagggtctgctactgagatgctctgcacagaggctgcctctcctcgcccttcctctgaagacctgcctcttcaaggcagccctgattcttctaccagtcccaaacaaaagctctcaagtcctgaggctgacaagggccctgaggaggaggagaacaaggtccttgccaggaagcagaagatgcggactgtgttctctcaggcccagctgtgtgcactcaaggacaggtttcagaagcagaagtacctcagcctccagcagatgcaagaactctcctccattctgaacctgagctataagcaggttaagacctggtttcaaaaccaaaggatgaagtgcaagcggtggcagaaaaaccagtggttgaagactagcaatggtctgattcagaagggctcagcaccagtggagtatcccagcatccattgcagctatccccagggctatctggtgaacgcatctggaagcctttccatgtggggcagccagacttggaccaacccaacttggagcagccagacctggaccaacccaacttggaacaaccagacctggaccaacccaacttggagcagccaggcctggaccgctcagtcctggaacggccagccttggaatgctgctccgctccataacttcggggaggactttctgcagccttacgtacagttgcagcaaaacttctctgccagtgatttggaggtgaatttggaagccactagggaaagccatgcgcattttagcaccccacaagccttggaattattcctgaactactctgtgactccaccaggtgaaatatgagacttacgcaacatctgggcttaaagtcagggcaaagccaggttccttccttcttccaaatattttcatattttttttaaagatttatttattcattatatgtaagtacactgtagctgtcttcagacactccagaagagggcgtcagatcttgttacgtatggttgtgagccaccatgtggttgctgggatttgaactcctgaccttcggaagagcagtcgggtgctcttatccactgagccatctcaccagcccctggtttatttttttaattattatttgctttttgtttatcaagacagggtttctctgcatagctctaattgtctttgaactagctctgcagaccagcctggccttgaactcagagatctgcccacttatctttgcctcctgaatgctgggaccaaaggtggcataccaccacacctggcatatatattgtttatttctatttctatttttattggtgccagagcaaacctaggacttagaacatgctgggcaccaactcaacttctgagctctatttacaacttggtgtgttagtgtatttgtcttagttctgaatttgtcctttttttagtgttaactctaggctttggagacagtgaggtgcatatactctctccttcccaagaataagtgcttgaacacccttacccacgcccacccacccatgctagtcttttttcttagaagcgtgggtcttggtatacactgtgtcattttgaggggtgaggtttaaaagtatatacaaagtataacgatatggtggctactctcgaggatgagacagaaggaccaggagtttgagggtagctcagatatgcaataagttcaaggccaacctgtactatgtttaaatagtaagacagcatctcgataaaataataaaactaaagtctcaacaaaataaaagctttcacctattaaggtgcttgcttgtccttggagtcccccaagagtaactgctatgttaatatctgtagaaagatgtttatatttgactgtaccatgatgaaccgatgccagctggactagtttaaacaaaataaaacactaattttacctttaaaaaaaaaa

SEQ ID NO: 30 - DNA, 1816 bp, Homo sapiens
  source 1..1816: mol_type = other DNA
ccggaacagagttcgtccatctaaaaagggaggggtccaaggagctgaactcctagccgcctagatttccactgtggctctgggtacgagtggcagtttcttcttgggatttcagaaaggaaaccaaggaggaaataggtagagcgcatcgcatcgctgtgggcattagagaaatggactaagagctgggacacgtggcaaaagaagatagtcttcttctctcaatagagtgagtgtgcagtgcagccagcagctcctgcacacagaagaaagcaggatcgcctcactgtgctgcctccaagtgttgtccccaaataccactgaccaaaaagcaggtgtttgcggatcagtgccccctggaagtgagtcataggcattgttcaagaaggaagcagctaagacaacatgaatgaacaaaaaatgaatgaacaaatgaagaaaacggcaaagacaagtggccagaaagggccgggcggaagagccctcgacagactgaccctaaagcaagacgaggcaaggccagtccagaataccagagtggaagctccccgtgtaacatacaccatccgggatgaaagtgagattagccccgagactgaggaagatggcttccctgacggatacctagagtgcatcatacgaggtgagttttccgaacccattctggaagaggatttcctttttaaatccttcgaaagcttggaggaagtggagcaaaacctttctcgccaggttctggaagcgagttcccttctcgagtcttctttggagtacatgacaaaggggacgaagcaagagaagagagaggtcacgcaagagacgcctccgctgagggttggggcgagctcattacttgcaggcggccctgcagagaagccagagggcggtgtgtactgtggtgtcttatcgatgctggagtgtcctcaagccgggtgcaagaagaagctgagggataaaaccgccctgaggaagcacatgcttgtccacgggccccggcggcacgtgtgtgcagagtgtggcaaagccttcacggagagctcgaaactaaagcgacattttctggtgcacaccggagagaagccgtatcagtgcacgttcgaaggctgtgggaagcgcttctccctggatttcaacttgcgcacccatatccgcatccacaccggggagaggcgctttgtgtgtccttttgatggctgcgagaagagctttattcagtcaaataaccagaagattcacatcctaacccacgcaaaggccgggaagaaatgctgaaggtggagacagattgtcctcaggctgggtagtcttaatgacagaggcactggggatacacgcattatgtacagattgttatttgtagggatagattctatggctaaaataattttgcaactcttcattatacagttttaaggcctcttttggtattccatggtatagttccaacaggaaagtcaatggtgaaagcagtttcaaaagcataacccttactatatgtgctattgaaggggatggggacttttgcatacgtcggaaaatcagttatttttaactatgcagttcctaattgcctagcttttacctttagaaatatgcttcatggtaaatttgataatcctaaagaatggccaactttggcagtagtcaacaaatgaatacatatttctaaattttatgttttgatttttgtttttctcattgtaccaaactatattaatcatagttaaatgtgaatagttacaaaaaagttgatccatctgaagattttagatgtttcaaaataaaaggaaataaaacactaaaaaaaaaaaaaaaa

SEQ ID NO: 31 - DNA, 1353 bp, Homo sapiens
  source 1..1353: mol_type = other DNA
gaggtgaaaccgtccctaggtgagccgtctttccaccaggcccccggctcggggtgcccaccttccccatggctggacacctggcttcagacttcgccttctcacccccaccaggtgggggtgatgggtcagcagggctggagccgggctgggtggatcctcgaacctggctaagcttccaagggcctccaggtgggcctggaatcggaccaggctcagaggtattggggatctccccatgtccgcccgcatacgagttctgcggagggatggcatactgtggacctcaggttggactgggcctagtcccccaagttggcgtggagactttgcagcctgagggccaggcaggagcacgagtggaaagcaactcagagggaacctcctctgagccctgtgccgaccgccccaatgccgtgaagttggagaaggtggaaccaactcccgaggagtcccaggacatgaaagccctgcagaaggagctagaacagtttgccaagctgctgaagcagaagaggatcaccttggggtacacccaggccgacgtggggctcaccctgggcgttctctttggaaaggtgttcagccagaccaccatctgtcgcttcgaggccttgcagctcagccttaagaacatgtgtaagctgcggcccctgctggagaagtgggtggaggaagccgacaacaatgagaaccttcaggagatatgcaaatcggagaccctggtgcaggcccggaagagaaagcgaactagcattgagaaccgtgtgaggtggagtctggagaccatgtttctgaagtgcccgaagccctccctacagcagatcactcacatcgccaatcagcttgggctagagaaggatgtggttcgagtatggttctgtaaccggcgccagaagggcaaaagatcaagtattgagtattcccaacgagaagagtatgaggctacagggacacctttcccagggggggctgtatcctttcctctgcccccaggtccccactttggcaccccaggctatggaagcccccacttcaccacactctactcagtcccttttcctgagggcgaggcctttccctctgttcccgtcactgctctgggctctcccatgcattcaaactgaggcaccagccctccctggggatgctgtgagccaaggcaagggaggtagacaagagaacctggagctttggggttaaattcttttactgaggagggattaaaagcacaacaggggtggggggtgggatggggaaagaagctcagtgatgctgttgatcaggagcctggcctgtctgtcactcatcattttgttcttaaataaagactgggacacacagtagatagct

SEQ ID NO: 32 - DNA, 20 bp, synthetic construct
  misc_feature 1..20: note = Description of Artificial Sequence: Synthetic Polynucleotide
  source 1..20: mol_type = other DNA
gtgtgtatttctggctggaa

SEQ ID NO: 33 - DNA, 8775 bp, synthetic construct
  misc_feature 1..8775: note = Description of Artificial Sequence: Synthetic Polynucleotide
  source 1..8775: mol_type = other DNA
gtgtgtatttctggctggaacgggcgtgttgttagagtaggggagtggattgagaaggaggctgaggggtactcaagggggctatagaatgtataggatttccctgaagcattcctagagagcctgcaaggtgaagatggctttggaaccagctggatctaggctgtgccacatactacctctttggccttggccacatccctaaactcttggattctgtttcctaagatgtaagatggaggtaattgttcctgcctcacaggagctgttgtgaggattaaacagagagtatgtctttagcgcggtgcctggcaccagtgcctggcatgtagtaggggcacaacaaatataaggtccactttgcttttcttttttctatagagaatcctttcctgtttgcattggaagccgtggttatctctgttggctccatgggattgattatcagccttctctgtgtgtatttctggttagagcgaacgatgccccgaattcccaccctgaagaacctagaggatcttgttactgaataccacgggaacttttcggcctggagtggtgtgtctaagggactggctgagagtctgcagccagactacagtgaacgactctgcctcgtcagtgagattcccccaaaaggaggggcccttggggaggggcctggggcctccccatgcaaccagcatagcccctactgggcccccccatgttacaccctaaagcctgaaacctgataatctagatttatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaagcggaaacacagaaaaaagcccgcacctgacagtgcgggctttttttttcgaccaaaggctcgagataagcttgatatcgaattcggagcactgtcctccgaacgtcggagcactgtcctccgaacgtcggagcactgtcctccgaacgtcggagcactgtcctccgaacggagcatgtcctccgaacgtcggagcactgtcctccgaacgactagtctagagggtatataatgggggccactagtctactaccagagttcatcgctagcgctaccggatccgccaccatggccctgccagtaacggctctgctgctgccacttgctctgctcctccatgcagccaggcctgactacaaagacgatgacgacaagcaagtccagctccagcagtcgggcccagagttggagaagcctggggcgagcgtgaagatctcatgcaaagcctcaggctactcctttactggatacacgatgaattgggtgaaacagtcgcatggaaagtcactggaatggatcggtctgattacgccctacaacggcgcctccagctacaaccagaagttcaggggaaaggcgacccttactgtcgacaagtcgtcaagcaccgcctacatggacctcctgtccctgacctccgaagatagcgcggtctacttttgtgcacgcggaggttacgatggacggggattcgactactggggccagggaaccactgtcaccgtgtcgagcggaggcggagggagcggaggaggaggcagcggaggtggagggtcggatatcgaactcactcagtccccagcaatcatgtccgcttcaccgggagaaaaggtgaccatgacttgctcggcctcctcgtccgtgtcatacatgcactggtaccaacaaaaatcggggacctcccctaagagatggatctacgataccagcaaactggcttcaggcgtgccgggacgcttctcgggttcggggagcggaaattcgtattcgttgaccatttcgtccgtggaagccgaggacgacgcaacttattactgccaacagtggtcaggctacccgctcactttcggagccggcactaagctggagatcaaggcggcagcaaccacgacgccagcgccgcgaccaccaacaccggcgcctaccatcgcgtcgcagccactgtcactgcgcccagaagcgtgccggccagcggcgggtggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaagaggacggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagaggcgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgatgccttgcacatgcaagccctgccccctcgctaacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggatccttgacttgcggccgcaactcccacctgcaacatgcgtgactgactgaggccgcgactctagagtcgaccggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacacctgcagcccaagcttaccatggccttaccagtgaccgccttgctcctgccgctggccttgctgctccacgccgccaggcctgaacaaaaactcattagcgaagaggatctcgacatacagatgacacagagccctagcagtctgagcgccagtgtgggcgatagagttactatcacttgtagagcatccgagaacatatacagttacgtggcctggtatcagcaaaaacctggcaaagctcccaagttattgatttacaatgctaagagcttggcctctggggtgccatcgaggttcagcggtagcgggagcgggaccgacttcactctgaccatctcgagtctccagccggaggactttgcgacatactattgtcaacaccattacgtatcaccctggaccttcggcggcgggactaagttagagatcaagggtggaggaggatcaggcggcggtggatcaggaggaggagggtcacaagtgcagttacaggaatcagggcccggcctggtgaagccaagtgaaaccctgagtctgacgtgcacggtttcaggatttagcctcacttcctacggtgtctcttggattcggcagccagccggcaaagggctcgagtggattggggtgatctgggaagatggctcaacaaactatcattctgcactaatctctcgcgtgacaatgtcggtggacacgtccaagaatcaattttcccttaaactgtcctccgtgaccgcagccgatacagcggtatattattgcgcgcgacctcactacggatctagctatgtcggcgcgatggagtattggggcgctggcacaaccgtcaccgtttcttccgcaaccacgacgccagcgccgcgaccaccaacaccggcgcccaccatcgcttcccagcccttgagcctcagacccgaggcctgcttcatgtacgtcgcggcggccgcctttgtccttctcttcttcgtcggctgcggggtcctcctctccaagaggaaacggaagcacaagctgctgagcagcatcgagcaggcctgtgacatctgccggctgaagaaactgaagtgcagcaaagaaaagcccaagtgcgccaagtgcctgaagaacaactgggagtgccggtacagccccaagaccaagagaagccccctgaccagagcccacctgaccgaggtggaaagccggctggaaagactggaacagctgtttctgctgatcttcccacgcgaggacctggacatgatcctgaagatggacagcctgcaggacatcaaggccctgctgaccggcctgttcgtgcaggacaacgtgaacaaggacgccgtgaccgacagactggccagcgtggaaaccgacatgcccctgaccctgcggcagcacagaatcagcgccaccagcagcagcgaggaaagcagcaacaagggccagcggcagctgacagtgtctgctgctgcaggcggaagcggaggctctggcggatctgatgccctggacgacttcgacctggatatgctgggcagcgacgccctggatgattttgatctggacatgctgggatctgacgctctggacgatttcgatctcgacatgttgggatcagatgcactggatgactttgacctggacatgctcggatcataaaggacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagaaacggggtttcaccatattggccaggctgatctccaactcctaatctcaggtgatctacccaccttggcctcccaaattgctgggattacaggcgtgaaccactgctcccttccctgtccttcggatccgaacggtgagatttggagaagcccagaaaaatgaggggaacggtagctgacaatagcagaggagggttttgcagggtctttaggagtaaaggatgagacagtaagtaatgagagattacccaagagggtttggtgatggaaggaagccacaggcacagagaacacagaatcactttatttcatatgggacaactgggagaagggtgataaaaaagctttaacctatgtgctcctgctccctctttctcccctgtcaggacgatgccccgaattcccaccctgaagaacctagaggatcttgttactgaataccacgggaacttttcggtgagaacgctgtcataagcatgctgcagtctatcaactgccaactgcctgccagcaagacagacagagtgtgggggtgggggcagagaggagagggaaggaggccctgcactaactgtcaggccgttccagccagaaatacacacatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaaacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttttgtctcgcgcgtttcggtgatgacggtgaaaacctctgacacatgcagctcccggagactgtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggat

(19) United States
(12) Patent Application Publication
(10) Pub. No.: US 2023/0365997 A1
(43) Pub. Date: Nov. 16, 2023

(54) COMPOSITIONS AND METHODS OF GENOMIC MODIFICATION OF CELLS AND USES THEREOF

(71) Applicant: Arsenal Biosciences, Inc., South San Francisco, CA (US)

(72) Inventors: Aaron Cooper, Berkeley, CA (US); Angela Boroughs, San Mateo, CA (US); Pei-Ken Hsu, San Francisco, CA (US)

(21) Appl. No.: 18/308,481

(22) Filed: Apr. 27, 2023

Related U.S. Application Data
(63) Continuation of application No. PCT/US2021/057457, filed on Oct. 29, 2021.
(60) Provisional application No. 63/107,401, filed on Oct. 29, 2020.

(51) Int. Cl. C12N 15/90; C12N 15/11; C12N 9/22; A61P 35/00; A61K 39/00

(52) U.S. Cl. CPC C12N 15/907; A61K 39/4611; A61K 39/4631; A61P 35/00; C12N 9/22; C12N 15/11; C12N 2310/20; C12N 2800/80

(57) ABSTRACT

Provided herein are compositions and methods for producing genomically edited cells expressing an exogenous transgene and restored or continued expression of an endogenous gene. Methods of using the genomically edited cells for treating or preventing a disease in a subject are also provided.

### CROSS-REFERENCE TO RELATED APPLICATIONS

This application is a continuation of International Application No. PCT/US2021/057457, filed Oct. 29, 2021, which claims the benefit of and priority to U.S. Provisional Pat. Application No. 63/107,401, filed Oct. 29, 2020, the disclosure of which is hereby incorporated by reference in its entirety for all purposes.

### SEQUENCE LISTING

The instant application contains a Sequence Listing which has been submitted electronically in XML format and is hereby incorporated by reference in its entirety. Said XML copy, created on Apr. 27, 2023, is named ANB-204WOCl_SL.xml and is 313,191 bytes in size.

### FIELD OF THE INVENTION

This invention generally relates to compositions and methods for transgene insertion into a cell for application in adoptive cell therapies.

### BACKGROUND

Genetically-engineered immune cell therapies have been in development for decades and have proven effective in treating certain cancers. The evolution from randomly integrating viral gene modification methods to targeted non-viral integrations holds great promise for further unlocking the potential of cellular immunotherapies. However, crucial engineering challenges unique to targeted transgene integrations remain. Efficiency of transgene incorporation is invariably less than 100% and current methods for selecting and/or enriching cells having integrated transgenes are largely based on the expression of transgene products allowing affinity purification or conferring antibiotic resistance.

For example, transgenes can be engineered to include a gene coding for a cell surface protein that is accessible to antibody reagents, which can be fluorescently labeled to enable fluorescence-activated cell sorting (FACS), or linked to magnetic beads to enable magnet-based enrichment. Alternatively, cells can be engineered to express a fluorescent protein (e.g., green fluorescent protein) to enable FACS. In another example, an antibiotic resistance marker (e.g., a puromycin resistance gene) can be incorporated into and expressed from the transgene, such that cells having successful integration of the transgene are antibioticresistant, while the cells not having successful integration are sensitive to antibiotic treatment. While these standard methods are effective, they require expression of relatively long, foreign proteins, unless a selection reagent can be produced for the transgene itself, assuming it is a cell surface protein.

In another approach, a transgene can be integrated into a locus such as hypoxanthine phosphoribosyltransferase (HPRT). HPRT catalyzes the conversion of 2-thioguanine into a cytotoxic metabolite. Insertion of a transgene into the HPRT locus, disrupts the expression of HPRT and integrated cells can be selected for by treating cells with 2-thioguanine. This and other methods of site-specific transgene insertion are often made into a gene that is essential for survival or function of the host cell and insertion typically inactivates the gene at the insertion site. Therefore, methods simultaneously achieving transgene insertion whilst correcting for gene disruption to promote cell survival and function can be beneficial in the development and application of adoptive immune cell therapies.

### BRIEF SUMMARY OF THE INVENTION

The present disclosure is directed to compositions and methods for site-specific transgene insertion in the genome of a cell while maintaining expression of the locus gene product to benefit the health and survival of the cell.

Provided herein is a composition for targeted insertion of a nucleic acid comprising a sequence of equivalent coding potential to a 3′ portion of an endogenous gene of a cell and an exogenous transgene. In some embodiments, the composition comprises: a guide RNA (gRNA) targeting the endogenous gene; an RNA guided nuclease complexed with the gRNA; and a nucleic acid complexed with the RNA-guided nuclease and comprising a sequence coding for one or more region(s) of homology to the endogenous gene, the sequence of equivalent coding potential to the 3′ portion of the endogenous gene and the transgene. In some embodiments, the RNA-guided nuclease specifically cleaves the endogenous gene in the cell to create an insertion site, wherein the sequence of equivalent coding potential to the 3′ portion of the endogenous gene and the transgene of the nucleic acid are inserted into the insertion site, and wherein insertion of the sequence of equivalent coding potential to the 3′ portion of the endogenous gene and the transgene of the nucleic acid results in restored or continued expression of the endogenous gene and expression of the transgene in the cell.

In other embodiments, the composition comprises: a gRNA targeting the endogenous gene; an RNA guided nuclease complexed with the gRNA; and a nucleic acid complexed with the RNA-guided nuclease and comprising a sequence coding for one or more region(s) of homology to the endogenous gene, an exogenous transgene, and a sequence of equivalent coding potential to a 5′ portion of an endogenous gene. In some embodiments, the RNA-guided nuclease specifically cleaves the endogenous gene in the cell to create an insertion site, wherein the transgene and the sequence of equivalent coding potential to the 5′ portion of the endogenous gene of the nucleic acid are inserted into the insertion site, and wherein insertion of the transgene and the sequence of equivalent coding potential to the 5′ portion of the endogenous gene of the nucleic acid results in restored or continued expression of the endogenous gene and expression of the transgene in the cell.

Also provided herein is a cell comprising a nucleic acid comprising from 5′ to 3′: (1) a sequence encoding a 5′ portion of an endogenous gene of the cell, (2) a sequence of equivalent coding potential to a 3′ portion of the endogenous gene of the cell, (3) a sequence encoding an exogenous transgene, and (4) a sequence encoding the 3′ portion of the endogenous gene of the cell, wherein the cell expresses each of the endogenous gene encoded by (1) and (2) and the transgene encoded by (3).

In other embodiments provided herein is a cell comprising a nucleic acid comprising from 5′ to 3′: (1) a sequence of equivalent coding potential to a 5′ portion of an endogenous gene of the cell, (2) a sequence encoding an exogenous transgene, (3) a sequence encoding the 5′ portion of the endogenous gene of the cell, and (4) a sequence encoding a 3′ portion of the endogenous gene of the cell, wherein the cell expresses each of the transgene encoded by (2) and the endogenous gene encoded by (3) and (4).

In another aspect provided herein is a method for editing the genome of a cell comprising: introducing into the cell an gRNA targeting an endogenous gene in the cell, an RNA-guided nuclease complexed with the gRNA, and a nucleic acid complexed with the RNA-guided nuclease and comprising a sequence coding for one or more region(s) of homology to the endogenous gene, a sequence of equivalent coding potential to a 3′ portion of the endogenous gene and an exogenous transgene. In some embodiments, the RNA guided nuclease specifically cleaves the endogenous gene in the cell to create an insertion site, wherein the sequence of equivalent coding potential to the 3′ portion of the endogenous gene and the exogenous transgene of the nucleic acid are inserted into the insertion site, and wherein insertion of the sequence of equivalent coding potential to the 3′ portion of the endogenous gene and the exogenous transgene of the nucleic acid results in restored or continued expression of the endogenous gene and expression of the transgene in the cell.

In yet another aspect, provided herein is a method for editing the genome of a cell comprising: introducing into the cell an gRNA targeting an endogenous gene in the cell, an RNA-guided nuclease complexed with the gRNA, and a nucleic acid complexed with the RNA-guided nuclease and comprising a sequence coding for one or more region(s) of homology to the endogenous gene, an exogenous transgene, and a sequence of equivalent coding potential to a 5′ portion of the endogenous gene. In some embodiments, the RNA guided nuclease specifically cleaves the endogenous gene in the cell to create an insertion site, wherein the exogenous transgene and the sequence of equivalent coding potential to the 5′ portion of the endogenous gene of the nucleic acid are inserted into the insertion site, and wherein insertion of the exogenous transgene and the sequence of equivalent coding potential to the 5′ portion of the endogenous gene of the nucleic acid results in restored or continued expression of the endogenous gene and expression of the transgene in the cell.

In some embodiments, the gRNA, RNA-guided nuclease, and nucleic acid are introduced into the cell via non-viral delivery. For example, in some embodiments, the gRNA, RNA-guided nuclease, and nucleic acid are introduced into the cell via electroporation. In some embodiments, the gRNA, RNA-guided nuclease, and/or nucleic acid are introduced into the cell via viral delivery. For example, in some embodiments, the gRNA, RNA-guided nuclease, and/or nucleic acid are introduced into the cell via an adeno-associated virus (e.g., AAV6).

In some embodiments, the endogenous gene is selected from the from the group consisting of: T-cell receptor alpha chain constant (TRAC), T-cell receptor beta chain constant (TRBC), CD3γ chain, CD3δ chain, CD3ε chain, CD3ξ chain, IL-2Rα chain, IL-2Rβ chain, and IL-2Rγ chain (IL2RG). For example, in some embodiments, the endogenous gene is TRAC. For example, in other embodiments, the endogenous gene is IL2RG.

In some embodiments, the endogenous gene is one or more of beta actin (Actb), ATP synthase H⁺ transporting, mitochondrial F0 complex subunit B1 (Atp5f1), beta-2 microglobulin (B2m), glyceraldehyde-3-phosphate dehydrogenase (Gapdh), glucuronidase beta (Gusb), hypoxanthine guanine phosphoribosyl transferase (Hprt), phosphoglycerate kinase I (Pgk1), peptidylprolyl isomerase A (Ppia), ribosomal protein S18 (Rps18), TATA box binding protein (Tbp), transferrin receptor (Tfrc), tyrosine 3-monooxygenase/tryptophan 5-monooxygenase activation protein zeta polypeptide (Ywhaz), Nanog homeobox (Nanog), zinc finger protein 42 (Rex1), and POU domain class 5 transcription factor 1 (Oct4). In some embodiments, the endogenous gene is Gapdh.

In some embodiments, the transgene comprises a chimeric antigen receptor (CAR).

In some embodiments, the cell is an immune cell, optionally a T-cell. For example, in some embodiments, the T-cell is a CD4+ or a CD8+ T-cell. In some embodiments, the cell is an induced pluripotent stem cell (iPSC). In some embodiments, the cell is an iPSC-derived natural killer cell (iNK). In some embodiments, the immune cell is an immune cell progenitor cell such as a pluripotent stem cell.

In some embodiments, the RNA-guided nuclease is Cas9.

In some embodiments, the gRNAis a single guide RNA (sgRNA) or a crRNA:trans-activating RNA (tracrRNA).

In another aspect, a method of treating a disease in a subject is provided, comprising: obtaining a cell comprising a nucleic acid as described herein, and administering the cell to the subject. In some embodiments, the disease is a cancer. In some embodiments, the cell is obtained from the subject. For example, in certain embodiments, the cell is a T-cell, optionally a CD4+ or a CD8+ T-cell.

### BRIEF DESCRIPTION OF THE DRAWINGS

FIG. 1 is a conceptual drawing illustrating an exemplary introduction of a guide RNA (gRNA), an RNA-guided nuclease (e.g., Cas9), and a nucleic acid encoding an exogenous transgene (e.g., a chimeric antigen receptor (CAR)), and a sequence of equivalent coding potential to a 5′ portion or a 3′ portion of an endogenous gene of cell (e.g., T-cell receptor alpha chain constant (TRAC)), into a cell (e.g., a T-cell) resulting in expression of both the exogenous transgene and endogenous gene.

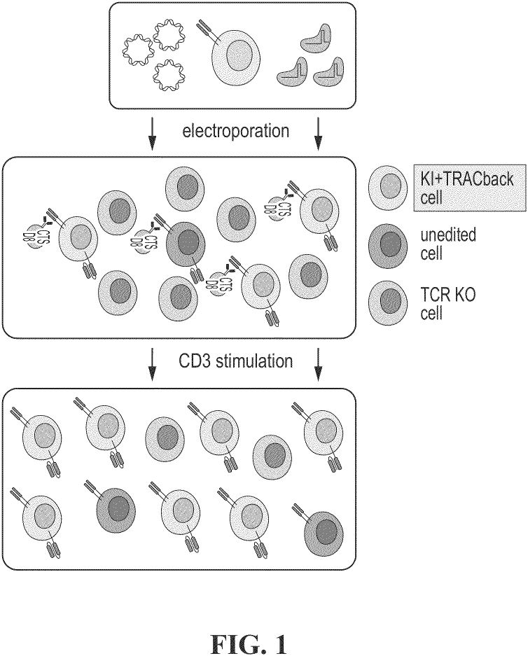

FIG. 2A is a conceptual drawing illustrating an exemplary insertion of a sequence of equivalent coding potential to a 3′ portion of an endogenous gene and an exogenous transgene into a double stranded break in the endogenous gene in the cell cleaved by an RNA-guided nuclease. FIG. 2B is a conceptual drawing illustrating exemplary outcomes of editing T-cells with non-viral targeting of IL2RG with and without gene circuit insertion.

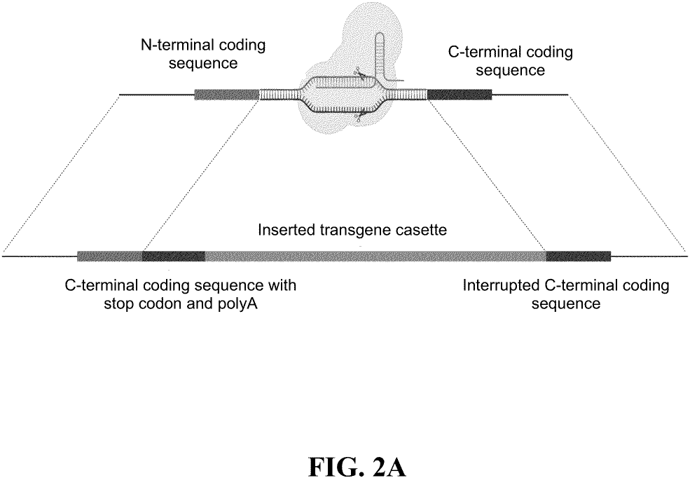

FIG. 3 shows flow cytometry dot plots of T-cells electroporated with a CRISPR ribonucleoprotein (RNP) targeting the TRAC locus with a plasmid repair template. FIG. 3A shows a flow cytometry dot plot of T-cells electroporated with a CRISPR RNP and a plasmid repair template encoding a CAR and truncated EGFR transgene. FIG. 3B shows a flow cytometry dot plot of T-cells electroporated with a CRISPR RNP and a plasmid repair template encoding a CAR, a truncated EGFR transgene, and all of the coding sequence of TRAC after the CRISPR target cut site. FIG. 3C shows a flow cytometry dot plot of the EGFR positive cells of FIG. 3B.

FIG. 4 is a graph showing the fold increase in the percentage of cells expressing CAR in T-cells electroporated with a CRISPR RNP targeting the TRAC locus with a plasmid repair template and stimulated with CD3/CD28 beads.

FIG. 5 shows flow cytometry dot plots of T-cells obtained from two donors electroporated with a CRISPR ribonucleoprotein (RNP) targeting the IL2RG locus with a plasmid repair template for expressing an exogenous transgene encoding a circuit with a Prime and CAR receptor and Myc-tag, at days 9 and 14 post-electroporation.

FIG. 6A is a graph showing the percentage of cells expressing both IL2RG and an exogenous transgene in T-cells obtained from four donors and electroporated with a CRISPR ribonucleoprotein (RNP) targeting the IL2RG locus with a plasmid repair template for expressing an exogenous transgene encoding a circuit with a Prime and CAR receptor and Myc-tag (pS6651), at days 9 and 14 post-electroporation.

FIG. 6B is a graph showing the percentage of cells having IL2RG knocked out and without integration of the transgene in T-cells obtained from four donors and electroporated with a CRISPR ribonucleoprotein (RNP) targeting the IL2RG locus with a plasmid repair template for expressing an exogenous transgene encoding a circuit with a Prime and CAR receptor and Myc-tag (pS6651), at days 9 and 14 post-electroporation.

### DETAILED DESCRIPTION

The present invention provides compositions and methods for the targeted insertion of a nucleic acid at a target site within an endogenous gene of a cell, wherein the nucleic acid comprises an exogenous transgene and a portion of the endogenous gene, and insertion of the nucleic acid allows for the expression of the exogenous transgene and the restored or continued expression of the endogenous gene in the cell. The restored or continued expression of the endogenous gene of the cell can be beneficial to the health and survival of the cell and/or advantageous for therapeutic cell manufacturing.

To facilitate an understanding of the present invention, a number of terms and phrases are defined below.

The terms “a” and “an” as used herein mean “one or more” and include the plural unless the context is inappropriate.

The term “nucleic acid”, “nucleotide”, or “oligonucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The term “gene” can refer to the segment of DNA involved in producing or encoding a polypeptide chain. It may include regions preceding and following the coding region (leader and trailer) as well as intervening sequences (introns) between individual coding segments (exons). Alternatively, the term “gene” can refer to the segment of DNA involved in producing or encoding a non-translated RNA, such as an rRNA, tRNA, guide RNA (gRNA), short-interfering RNA (siRNA), or micro RNA (miRNA).

As used herein, the term “endogenous” with reference to a nucleic acid, for example, a gene, or a protein in a cell is a nucleic acid or protein that occurs in that particular cell as it is found in nature, for example, at its natural genomic location or locus. Moreover, a cell “endogenously expressing” a nucleic acid or protein expresses that nucleic acid or protein as it is found in nature.

A “promoter” is defined as one or more nucleic acid control sequence(s) that direct transcription of a nucleic acid. As used herein, a promoter includes nucleic acid sequences near the start site of transcription, such as, in the case of a polymerase II type promoter, a TATA element. A promoter also optionally includes distal enhancer or repressor elements, which can be located as much as several thousand base pairs from the start site of transcription.

A nucleic acid is “operably linked” when it is placed into a functional relationship with another nucleic acid sequence. For example, a promoter or enhancer is operably linked to a coding sequence if it affects the transcription of the sequence; or a ribosome binding site is operably linked to a coding sequence if it is positioned so as to facilitate translation.

As used herein, the term “sequence of equivalent coding potential” refers to a nucleic acid sequence having functional equivalence to another reference nucleic acid. A sequence of equivalent coding potential may or may not have the same primary nucleotide sequence. For example, for a reference nucleic acid coding for an expressed polypeptide, a sequence of equivalent coding potential is functionally able to code for the same expressed polypeptide and may comprise an identical primary nucleotide sequence as the reference nucleic acid, or may comprise one or more alternative codon(s) as compared to the reference nucleic acid. For example, an endogenous nucleic acid sequence encoding a polypeptide may be altered via codon optimization to result in a sequence that codes for an identical polypeptide. A codon optimized sequence may be one in which codons in a polynucleotide encoding a polypeptide have been substituted in order to modify the activity, expression, and/or stability of the polynucleotide. For example, codon optimization can be used to vary the degree of sequence similarity of a sequence of equivalent coding potential as compared to an endogenous gene sequence, while preserving the potential to encode the protein product of the endogenous gene.

“Polypeptide,” “peptide,” and “protein” are used interchangeably herein to refer to a polymer of amino acid residues. As used herein, the terms encompass amino acid chains of any length, including full-length proteins, wherein the amino acid residues are linked by covalent peptide bonds.

As used herein, the term “complementary” or “complementarity” refers to specific base pairing between nucleotides or nucleic acids. Complementary nucleotides are, generally, A and T (or A and U), and G and C. The guide RNAs (gRNAs) described herein can comprise sequences, for example, DNA targeting sequences that are perfectly complementary or substantially complementary (e.g., having 1-4 mismatches) to a genomic sequence.

As used herein, the term “targeted nuclease” refers to an endonuclease that recognizes and binds to a specific sequence of DNA to introduce a single or double-stranded cut at a specific cut site. Target nucleases include, but are not limited to, RNA-guided nucleases, transcription activator-like effector nucleases (TALENs), zinc finger nucleases (ZFNs) and megaTALs.

As used herein, the term “RNA-guided nuclease” refers to an endonuclease that can be used to perform targeted genome editing that complexes with a guide RNA (e.g., sgRNA or crRNA:tracrRNA).

As used herein, the term “target cut site” refers to a genomic site at which an endoclease specifically cleaves resulting in a single-stranded or double-stranded break.

The “CRISPR/Cas” system refers to a widespread class of bacterial systems for defense against foreign nucleic acid. CRISPR/Cas systems are found in a wide range of eubacterial and archaeal organisms. CRISPR/Cas systems include type I, II, and III sub-types. Wild-type type II CRISPR/Cas systems utilize an RNA-guided nuclease, for example, Cas9, in complex with guide and activating RNA to recognize and cleave foreign nucleic acid. Guide RNAs having the activity of both a guide RNA and an activating RNA are also known in the art. In some cases, such dual activity guide RNAs are referred to as a single guide RNA (sgRNA).

Cas9 homologs are found in a wide variety of eubacteria, including, but not limited to bacteria of the following taxonomic groups: Actinobacteria, Aquificae, Bacteroidetes-Chlorobi, Chlamydiae-Verrucomicrobia, Chlroflexi, Cyanobacteria, Firmicutes, Proteobacteria, Spirochaetes, and Thermotogae. An exemplary Cas9 protein is the Streptococcus pyogenes Cas9 protein. Additional Cas9 proteins and homologs thereof are described in, e.g., Chylinksi, et al., RNA Biol. 2013 May 1; 10(5): 726-737 ; Nat. Rev. Microbiol. 2011 June; 9(6): 467-477; Hou, et al., Proc Natl Acad Sci U S A. 2013 Sep 24;110(39):15644-9; Sampson et al., Nature. 2013 May 9;497(7448):254-7; and Jinek, et al., Science. 2012 Aug 17;337(6096):816-21. Variants of any of the Cas9 nucleases provided herein can be optimized for efficient activity or enhanced stability in the host cell. Thus, engineered Cas9 nucleases are also contemplated. See, for example, Slaymaker et al., Rationally engineered Cas9 nucleases with improved specificity, Science 351 (6268): 84-88 (2016)).

As used herein, the term “Cas9” refers to an RNA-guided nuclease (e.g., of bacterial or archeal orgin, or derived therefrom). Exemplary RNA-guided nucleases include the foregoing Cas9 proteins and homologs thereof. Other RNA-guided nucleases include Cpf1 (See, e.g., Zetsche et al., Cell, Volume 163, Issue 3, p759-771, 22 Oct. 2015) and homologs thereof.

As used herein, the term “ribonucleoprotein” and the like refers to a complex of a targeted nuclease, for example, the Cas9 protein and a sgRNA, the Cas9 protein and a crRNA, the Cas9 protein and a trans-activating crRNA (tracrRNA), the Cas9 protein and a guide RNA, or a combination thereof (e.g., the Cas9 protein, a tracrRNA, and a crRNA guide RNA are complexed together). It is understood that in any of the embodiments described herein, a Cas9 nuclease can be subsittuted with a Cpf1 nuclease or any other guided nuclease.

As used herein, the term “complexed” refers to two or more molecules that are physically associated via non-covalent interactions. For example, in the case of an RNA-guided nuclease complexed with an gRNA, the nuclease functionally associates with the gRNA via non-covalent interactions which can facilitate the recruitment of the nuclease to the genomic locus targeted by the gRNA. Similarly, in the case of an RNA-guided nuclease complexed with a nucleic acid, the nuclease functionally associates with the nucleic acid via non-covalent interactions which can facilitate the recruitment of the nucleic acid to a targeted genomic locus where it can serve as template for e.g., homology directed repair (HDR).

As used herein, the terms “editing” or “modifying” in the context of editing or modifying a genome of a cell refers to inducing a structural change in the sequence of the genome at a target genomic region. For example, editing or modifying can take the form of inserting a nucleotide sequence into the genome of the cell. For example, an exogenous transgene encoding a polypeptide can be inserted into the genomic sequence of the T-Cell receptor (TCR) locus of a T-cell. As used throughout a “TCRlocus” is a location in the genome where the gene encoding a TCRa subunit, a TCRβ subunit, a TCRγ subunit, or a TCRδ subunit is located. Such editing modifying can be performed, for example, by inducing a double stranded break within a target genomic region, or a pair of single stranded nicks on opposite strands and flanking the target genomic region. Methods for inducing single or double stranded breaks at or within a target genomic region include the use of a Cas9 nuclease domain, or a derivative thereof, and a guide RNA (e.g., sgRNA or crRNA:tracrRNA), or pair of guide RNAs, directed to the target genomic region.

As used herein, the term “introducing” in the context of introducing a nucleic acid or a complex comprising a nucleic acid, for example, an RNP-DNA template complex, refers to the translocation of the nucleic acid sequence or the RNP-DNA template complex from outside a cell to inside the cell. In some cases, introducing refers to translocation of the nucleic acid or the complex from outside the cell to inside the nucleus of the cell. Various methods of such translocation are contemplated, including but not limited to, electroporation, contact with nanowires or nanotubes, receptor mediated internalization, translocation via cell penetrating peptides, liposome-mediated translocation, and the like.

As used herein the term “exogenous” refers to what is not normally found in nature. For example, the term “exogenous gene” refers to a gene not normally found in a given cell in nature.

As used herein, the term “transgene” refers to an exogenous gene artificially introduced into the genome of a cell, or an endogenous gene artificially introduced into a non-natural locus in the genome of a cell. A transgene can refer to a segment of DNA involved in producing or encoding a polypeptide chain. Transgenes may include regions preceding and following the coding region (leader and trailer) as well as intervening sequences (introns) between individual coding segments (exons). Alternatively, transgenes can refer to the segment of DNA involved in producing or encoding a non-translated RNA, such as an rRNA, tRNA, gRNA, siRNA, or miRNA.

As used herein, the term “housekeeping gene” refers to genes required for basic cellular functions and are constitutively and stably expressed in varying physiological and experimental conditions. An exemplary housekeeping gene is Gapdh.

As used herein, a “cell” can be a eukaryotic cell, a prokaryotic cell, an animal cell, a plant cell, a fungal cell, and the like. Optionally, the cell is a mammalian cell, for example, a human cell. In some cases, the cell is an immune cell. For example, in some embodiments, the cell is a human T-cell (e.g., a CD4+ or a CD8+ T-cell) or a cell capable of differentiating into a T-cell that expresses a TCR receptor molecule. These include hematopoietic stem cells and cells derived from hematopoietic stem cells. In some embodiments, the cell is an induced progenitor stem cell (iPSC). In some embodiments, the cell is an iPSC-derived natural killer cell (iNK).

As used herein, the term “selectable marker” refers to a gene which allows selection of a host cell, for example, a T-cell, comprising a marker. The selectable markers may include, but are not limited to: fluorescent markers, luminescent markers and drug selectable markers, cell surface receptors, and the like. In some embodiments, the selection can be positive selection; that is, the cells expressing the marker are isolated from a population, e.g., to create an enriched population of cells expressing the selectable marker. Separation can be by any convenient separation technique appropriate for the selectable marker used. For example, if a fluorescent marker is used, cells can be separated by fluorescence activated cell sorting (FACS), whereas if a cell surface marker has been inserted, cells can be separated from the heterogeneous population by affinity separation techniques, e.g., magnetic separation, affinity chromatography, “panning” with an affinity reagent attached to a solid matrix, FACS or other convenient technique.

As used herein, the term “hematopoietic stem cell” refers to a type of stem cell that can give rise to a blood cell. Hematopoietic stem cells can give rise to cells of the myeloid or lymphoid lineages, or a combination thereof. Hematopoietic stem cells are predominantly found in the bone marrow, although they can be isolated from peripheral blood, or a fraction thereof. Various cell surface markers can be used to identify, sort, or purify hematopoietic stem cells. In some cases, hematopoietic stem cells are identified as c-kit⁺ and lin⁻. In some cases, human hematopoietic stem cells are identified as CD34⁺, CD59⁺, Thy1/CD90⁺, CD38^{lo/-}, C-kit/CD117⁺, lin-. In some cases, human hematopoietic stem cells are identified as CD34-, CD59⁺, Thy ⅟CD90⁺, CD38^{lo/-}, C-kit/CD117⁺, lin⁻. In some cases, human hematopoietic stem cells are identified as CD133⁺, CD59⁺, Thyl/CD90⁺, CD38^{lo/-}, C-kit/CD117⁺, lin⁻. In some cases, mouse hematopoietic stem cells are identified as CD34^{lo/-}, SCA-1⁺, Thy1^{+/lo}, CD38⁺, C-kit⁺, lin-. In some cases, the hematopoietic stem cells are CD150⁺CD48-CD244-.

As used herein, the phrase “hematopoietic cell” refers to a cell derived from a hematopoietic stem cell. The hematopoietic cell may be obtained or provided by isolation from an organism, system, organ, or tissue (e.g., blood, or a fraction thereof). Altematively, a hematopoietic stem cell can be isolated and the hematopoietic cell obtained or provided by differentiating the stem cell. Hematopoietic cells include cells with limited potential to differentiate into further cell types. Such hematopoietic cells include, but are not limited to, multipotent progenitor cells, lineage-restricted progenitor cells, common myeloid progenitor cells, granulocyte-macrophage progenitor cells, or megakaryocyte-erythroid progenitor cells. Hematopoietic cells include cells of the lymphoid and myeloid lineages, such as lymphocytes, erythrocytes, granulocytes, monocytes, and thrombocytes. In some embodiments, the hematopoietic cell is an immune cell, such as a T-cell, B-cell, macrophage, a natural killer (NK) cell or dendritic cell. In some embodiments the cell is an innate immune cell.

As used herein, the term “T-cell” refers to a lymphoid cell that expresses a TCR molecule. T-cells include human alpha beta (αβ) T-cells and human gamma delta (γδ) T-cells. T-cells include, but are not limited to, naïve T-cells, stimulated or activated T-cells, primary T-cells (e.g., uncultured), cultured T-cells, immortalized T-cells, helper T-cells, cytotoxic T-cells, memory T-cells, regulatory T-cells (Tregs), natural killer T-cells, combinations thereof, or sub-populations thereof. T-cells can be CD4⁺, CD8⁺, or CD4⁺ and CD8⁺. T-cells can also be CD4⁻, CD8⁻, or CD4⁻ and CD8⁻ T-cells can be helper cells, for example helper cells of type T_{H}1, T_{H}2, T_{H}3, T_{H}9, T_{H}17, or T_{FH}. T-cells can be cytotoxic T-cells. Tregs can be FOXP3⁺ or FOXP3⁻. T-cells can be alpha/beta T-cells or gamma/delta T-cells. In some cases, the T-cell is a CD4⁺CD25^{hi}CD127^{lo} Treg. In some cases, the T cell is a Treg selected from the group consisting of type 1 regulatory (Tr1), T_{H}3, CD8+CD28-, Treg17, and Qa-1 restricted T cells, or a combination or sub-population thereof. In some cases, the T-cell is a FOXP3⁺ T cell. In some cases, the T-cell is a CD4⁺CD25^{lo}CD127^{hi} effector T-cell. In some cases, the T-cell is a CD4⁺CD25^{lo}CD127^{hi}CD45RA^{hi}CD45RO⁻ naïve T-cell. A T-cell can be a recombinant T-cell that has been genetically manipulated.

As used herein, the term “primary” in the context of a primary cell is a cell that has not been transformed or immortalized. Such primary cells can be cultured, sub-cultured, or passaged a limited number of times (e.g., cultured 0, 1, 2, 3, 4, 5, 6, 7, 8, 9, 10, 11, 12, 13, 14, 15, 16, 17, 18, 19, or 20 times). In some cases, the primary cells are adapted to in vitro culture conditions. In some cases, the primary cells are isolated from an organism, system, organ, or tissue, optionally sorted, and utilized directly without culturing or sub-culturing. In some cases, the primary cells are stimulated, activated, or differentiated. For example, primary T-cells can be activated by contact with (e.g., culturing in the presence of) CD3, CD28 agonists, IL-2, IFN-γ, or a combination thereof.

As used herein, the term “homology directed repair” or HDR refers to a cellular process in which cut or nicked ends of a DNA strand are repaired by polymerization from a homologous template nucleic acid. Thus, the original sequence is replaced with the sequence of the template. In some cases, an exogenous template nucleic acid, for example, a DNA template, can be introduced to obtain a specific HDR-induced change of the sequence at a target site. In this way, specific mutations can be introduced at a cut site, for example, a cut site created by a targeted nuclease. A single-stranded DNA template or a double-stranded DNA template can be used by a cell as a template for editing or modifying the genome of a cell, for example, by HDR. Generally, the single-stranded DNA template or a double-stranded DNA template has at least one region of homology to a target site. In some cases, the single-stranded DNA template or double-stranded DNA template has two homologous regions, for example, a 5′ end and a 3′ end, flanking a region that contains the DNA template to be inserted at a target cut or insertion site.

As used herein, the term “targeted insertion” refers to the integration of a molecule (e.g., a nucleic acid) to a specific site within a cell. In the case of a nucleic acid, targeted insertion can refer to the integration of a nucleic acid into a single-stranded or double-stranded break at a specific location in the genomic DNA of a cell, for example, via HDR, resulting in a contiguous genomic DNA strand.

The term “substantial identity” or “substantially identical,” as used in the context of polynucleotide or polypeptide sequences, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 90%, 91%, 92%, 93%, 94%, 95%, 96%, 97%, 98%, or 99%, as compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or altemative parameters can be designated. The sequence comparison algorithm then calculates the percent sequence identities for the test sequences relative to the reference sequence, based on the program parameters.

Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=-2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat′l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

As used herein, the term “cancer-specific antigen” refers to an antigen that is unique to cancer cells or is expressed more abundantly in cancer cells than in in non-cancerous cells. In some embodiments, the cancer-specific antigen is a tumor-specific antigen.

As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably include humans.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

### I. Compositions

Provided herein is a composition for the targeted insertion of a nucleic acid comprising a sequence of equivalent coding potential to a 3′ portion or a 5′ portion of an endogenous gene of a cell and an exogenous transgene. In some embodiments, the composition comprises: (A) a guide RNA (gRNA); (B) a targeted nuclease; and (C) a nucleic acid (e.g., template for DNA repair). In other embodiments, the composition comprises: (A) a targeted nuclease; and (B) a nucleic acid (e.g., template for DNA repair).

### A. Guide RNA

As used herein, a guide RNA (gRNA) is a nucleic acid that interacts with a site-specific or targeted nuclease and specifically binds to or hybridizes to a target nucleic acid within the genome of a cell, such that the gRNA, and the nuclease complexed therewith, co-localize to the target nucleic acid in the genome of the cell. In some embodiments, an gRNA includes a DNA targeting sequence or protospacer sequence of about 10 to about 50 nucleotides in length that specifically binds to or hybridizes to a target DNA sequence in the genome. For example, in some embodiments, the DNA targeting sequence is about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments the gRNA comprises a single guide RNA (sgRNA). In some embodiments, the gRNA comprises a crRNA sequence and a transactivating crRNA tracrRNA sequence (crRNA:tracrRNA). In some embodiments, the gRNA does not comprise a tracrRNA sequence.

In some embodiments, the DNA targeting sequence is designed to complement (e.g., perfectly complement) or substantially complement the target DNA sequence. In some cases, the DNA targeting sequence can incorporate wobble or degenerate bases to bind multiple genetic elements. In some cases, the 19 nucleotides at the 3′ or 5′ end of the binding region are perfectly complementary to the target genetic element or elements. In some cases, the binding region can be altered to increase stability. For example, non-natural nucleotides, can be incorporated to increase RNA resistance to degradation. In some cases, the binding region can be altered or designed to avoid or reduce secondary structure formation in the binding region. In some cases, the binding region can be designed to optimize G-C content. In some cases, G-C content is preferably between about 40% and about 60% (e.g., 40%, 45%, 50%, 55%, 60%).

In some embodiments, the DNA targeting sequence is complementary or substantially complementary to an endogenous gene of a cell. For example, in some embodiments, the DNA targeting sequence is complementary or substantially complementary to an endogenous gene encoding T-cell receptor alpha chain constant (TRAC), T-cell receptor beta chain constant (TRBC), CD3γ chain, CD3δ chain, CD3ε chain. IL-2Rα chain, IL-2Rβ chain, or IL-2Rγ chain (IL2RG). In certain embodiments, the DNA targeting sequence is complementary or substantially complementary to the endogenous I TRAC and comprises the sequence AAGTCTCTCAGCTGGTACA (SEQ ID NO:1).

### B. Targeted Nuclease

In some embodiments, the composition comprises a targeted nuclease including, but not limited to, an RNA-guided nuclease, a transcription activator-like effector nuclease (TALEN), a zinc finger nuclease (ZFN), or a megaTAL. For example, in some embodiments the targeted nuclease is an RNA-guided nuclease that is complexed with the gRNA and is guided by the gRNA to a target region in the genome of the cell, where it introduces a single-stranded or double stranded break in the genomic DNA. For example, in certain embodiments, the targeted nuclease is a RNA-guided nuclease. In some embodiments, the RNA-guided nuclease is a Cas9 nuclease.

In certain embodiments, the Cas9 protein can be in an active endonuclease form, such that when bound to target nucleic acid as part of a complex with a gRNA and/or part of a complex with a nucleic acid (e.g., DNA template), a double strand break is introduced into the target nucleic acid. In the compositions and methods provided herein, a Cas9 polypeptide or a nucleic acid encoding a Cas9 polypeptide can be introduced into the cell. The double strand break can be repaired by HDR to insert the DNA template into the genome of the cell. Various Cas9 nucleases can be utilized in the methods described herein. For example, a Cas9 nuclease that requires an NGG protospacer adjacent motif (PAM) immediately 3′ of the region targeted by the guide RNA can be utilized. Such Cas9 nucleases can be targeted to, for example, a region in exon 1 of TRAC or exon 1 of TRAB that contains an NGG sequence. As another example, Cas9 proteins with orthogonal PAM motif requirements can be used to target sequences that do not have an adjacent NGG PAM sequence. Exemplary Cas9 proteins with orthogonal PAM sequence specificities include, but are not limited to those described in Esvelt et al., Nature Methods 10: 1116-1121 (2013).

In some cases, the Cas9 protein is a nickase, such that when bound to target nucleic acid as part of a complex with a agRNA, a single strand break or nick is introduced into the target nucleic acid. A pair of Cas9 nickases, each bound to a structurally different gRNA, can be targeted to two proximal sites of a target genomic region and thus introduce a pair of proximal single stranded breaks into the target genomic region, for example exon 1 of a TRAC gene or exon 1 of a TRBC gene. Nickase pairs can provide enhanced specificity because off-target effects are likely to result in single nicks, which are generally repaired without lesion by base-excision repair mechanisms. Exemplary Cas9 nickases include Cas9 nucleases having a D10A or H840A mutation (See, for example, Ran et al. “Double nicking by RNA-guided CRISPR Cas9 for enhanced genome editing specificity,” Cell 154(6): 1380-1389 (2013)).

In other embodiments, the targeted nuclease can be a TALEN, a ZFN, or a megaTAL (See, for example, Merkert and Martin “Site-Specific Genome Engineering in Human Pluripotent Stem Cells,” Int. J. Mol. Sci. 18(7): 1000 (2016)).

### C. Nucleic Acids

In some embodiments, the composition further comprises a nucleic acid complexed with the RNA-guided nuclease, wherein the nucleic acid comprises one or more region(s) of homology to an endogenous gene of the cell, a sequence of equivalent coding potential to a 5′ portion or 3′ portion of the endogenous gene, and an exogenous transgene. In some embodiments the nucleic acid functions as a template for DNA repair mechanisms such as HDR. For example, in some embodiments, a nucleic acid provided herein comprises: one or more portions of homology to at least one region flanking a target cut site in an endogenous gene of a cell; a sequence of equivalent coding potential to the 5′ coding portion or 3′ coding portion of the endogenous gene; and an exogenous transgene, wherein the sequence of equivalent coding potential to the 5′ coding portion or 3′ coding portion of the endogenous gene and the exogenous transgene are inserted into the target cut site within the endogenous gene of the cell.

For example, in one embodiment, a nucleic acid comprises in order from 5′ to 3′: (i) a 5′ homology arm having sequence homology or substantial sequence homology to a 5′ portion of an endogenous gene in the cell; (ii) a sequence of equivalent coding potential to a 3′ portion of the endogenous gene in the cell having a stop codon and polyadenylation sequence that codes for a carboxy-terminal portion of the protein product of the endogenous gene; (iii) an exogenous transgene; and (iv) a 3′ homology arm having sequence homology or substantial sequence homology to a 3′ portion of the endogenous gene in the cell. When introduced into the cell, the 5′ and 3′ homology arms align the nucleic acid to the target endogenous gene, and the sequence of equivalent coding potential to the 3′ portion of the endogenous gene in the cell that codes for the carboxy-terminal portion of the protein product of the endogenous gene and the exogenous transgene are inserted into a target cut site (e.g., introduced by the targeted nuclease) within the endogenous gene via DNA repair mechanisms (e.g., homology directed repair (HDR)). Insertion of the sequence of equivalent coding potential to the 3′ portion of the endogenous gene and the exogenous transgene results in restored or continued expression of the endogenous gene product and expression of the exogenous transgene.

In another embodiment, a nucleic acid comprises in order from 5′ to 3′: (i) a 5′ homology arm having sequence homology or substantial sequence homology to a 5′ portion of an endogenous gene in the cell; (ii) an exogenous transgene; (iii) a sequence of equivalent coding potential to a 5′ portion of the endogenous gene in the cell that codes for an amino-terminal portion of the protein product of the endogenous gene; and (iv) a 3′ homology arm having sequence homology or substantial sequence homology to a3′ portion of the endogenous gene in the cell. When introduced into the cell, the 5′ and 3′ homology arms align the nucleic acid to the target endogenous gene, and the exogenous transgene and sequence of equivalent coding potential to the 5′ portion of the endogenous gene in the cell that codes for the amino-terminal portion of the protein product of the endogenous gene are inserted into a target cut site (e.g., introduced by the targeted nuclease) within the endogenous gene via DNA repair mechanisms (e.g., HDR). Insertion of the exogenous transgene and sequence of equivalent coding potential to the 5′ portion of the endogenous gene results in expression of the exogenous transgene and restored or continued expression of the endogenous gene product.

The concept of using a targeted nuclease to deliver a cut site within a gene encoding a protein involved with cell survival or expansion (e.g., Gapdh, IL2RG or TRAC) and then introducing the sequence of equivalent coding potential to the 3′ portion or 5′ portion of the survival gene together with a desired exogenous transgene (e.g., a CAR or gene circuit) can be generalized to all proteins involved with cell survival. In certain aspects, the cells that undergo target nuclease activity will either integrate or not integrate with the desired transgene to restore critical protein expression. The set of cells that do not receive the insert will lack the corresponding protein (e.g., IL2RG or other housekeeping gene), and will not be able to survive. Cells without an integration will generally be depleted in the culture over time. In contrast, cells that receive the desired transgene will also have the expression of the corresponding protein restored and will generally be enriched in the culture during culture and manufacturing. Using this method, cells with successful integration of the exogenous transgene (e.g., a CAR or gene circuit) will generally have preferential survival and enrichment. In some embodiments, the transgene can be a CAR, gene circuit, or any other payload to add desired functionality to the cell of interest. The target gene can encode any protein involved with a cell’s survival or expansion, e.g., during manufacturing. In the case of T cells, this can include one or more genes that make up the TCR signaling complex, cytokine receptors and their downstream signaling molecules, and/or any housekeeping genes involved with T cell survival or expansion, e.g., TRAC, IL2RG, or Gapdh.

In some embodiments described herein, the length of each of the one or more region(s) of homology to an endogenous gene is at least about 50, 100, 150, 200, 250, 300, 350, 400 or 450 nucleotides. In some embodiments, the one or more region(s) of homology to an endogenous gene is at least 80%, 90%, 95%, 99% or 100% complementary to the endogenous gene. In some embodiments, the one or more region(s) of homology are homologous to genomic sequences in a human immune cell, for example, a T-cell. In some embodiments, the one or more region(s) of homology are homologous to TRAC, TRBC, CD3γ chain, CD3δ chain, CD3ε chain, CD3ξ chain IL-2Rα chain, IL-2Rβ chain, or IL-2Rγ chain (IL2RG).

For example, in some embodiments, a region of homology of an endogenous gene may be at least about 50, 100, 150, 200, 250, 300, 350, 400 or 450 nucleotides in length and having at least 80%, 90%, 95%, 99% or 100% complementary to any endogenous gene sequence in Table 1 over the length of the region of homology.

TABLE 1

Endogenous Genes

| | | |
|---|---|---|
| SEQ ID NO: | Gene | NCBI Reference Seqeunce |
| SEQ ID NO:2 | TRAC | NG_001332.3 |
| SEQ ID NO:3 | TRBC | NG_001333.2 |
| SEQ ID NO:4 | CD3γ chain | NG_007566.1 |
| SEQ ID NO:5 | CD3δ chain | NG_009891.1 |
| SEQ ID NO:6 | CD3ε chain | NG_007383.1 |
| SEQ ID NO:7 | CD3ξ chain | NG_007384.1 |
| SEQ ID NO:8 | IL-2Rα chain | NG_007403.1 |
| SEQ ID NO:9 | IL-2Rβ chain | NC_000022.11:c37175118-37125838 |
| SEQ ID NO:10 | IL-2Rγ chain (IL2RG) | NG_009088.1 |

In some embodiments, the one or more region(s) of homology are homologous to genomic sequences of one or more endogenous housekeeping genes. In some embodiments, the one or more region(s) of homology are homologous to beta actin (Actb), ATP synthase H⁺ transporting, mitochondrial F0 complex subunit B1 (Atp5f1), beta-2 microglobulin (B2m), glyceraldehyde-3-phosphate dehydrogenase (Gapdh), glucuronidase beta (Gusb), hypoxanthine guanine phosphoribosyl transferase (Hprt), phosphoglycerate kinase I (Pgk1), peptidylprolyl isomerase A (Ppia), ribosomal protein S18 (Rps18), TATA box binding protein (Tbp), transferrin receptor (Tfrc), tyrosine 3-monooxygenase/tryptophan 5-monooxygenase activation protein zeta polypeptide (Ywhaz), Nanog homeobox (Nanog), zinc finger protein 42 (Rex1), or POU domain class 5 transcription factor 1 (Oct4).

For example, in some embodiments, a region of homology of an endogenous housekeeping gene may be at least about 50, 100, 150, 200, 250, 300, 350, 400 or 450 nucleotides in length and having at least 80%, 90%, 95%, 99% or 100% complementary to any endogenous gene sequence in Table 2 over the length of the region of homology.

TABLE 2

Endogenous Housekeeping Genes

| | | |
|---|---|---|
| SEQ ID NO: | Gene | NCBI Reference Seqeunce |
| SEQ ID NO:17 | ActB | NM_007393.5 |
| SEQ ID NO:18 | Atp5f1 | NM_009725.4 |
| SEQ ID NO:19 | B2m | NM_009735.3 |
| SEQ ID NO:20 | Gapdh | NM_001289726.1 |
| SEQ ID NO:21 | Gusb | NM_010368.2 |
| SEQ ID NO:22 | Hprt | NM_013556.2 |
| SEQ ID NO:23 | Pgk1 | NM_008828.3 |
| SEQ ID NO:24 | Ppia | NM_008907.1 |
| SEQ ID NO:25 | Rps18 | NM_011296.2 |
| SEQ ID NO:26 | Tbp | NM_013684.3 |
| SEQ ID NO:27 | Tfrc | NM_001357298.1 |
| SEQ ID NO:28 | Ywhaz | NM_011740.3 |
| SEQ ID NO:29 | Nanog | NM_028016.3 |
| SEQ ID NO:30 | Rex1 | NM_009556.3 |
| SEQ ID NO:31 | Oct4 | NM_013633.3 |

In some embodiments, the nucleic acid comprises a homology directed repair (HDR) template and one or more RNA-guided nuclease target sequence(s). In some embodiments, the nucleic acid comprises one RNA-guided nuclease target sequence and one or more protospacer adjacent motif(s) (PAM). The complex containing the RNA-guided nuclease, gRNA, and nucleic acid can shuttle the HDR template, without cleavage of the RNA-guided nuclease target sequence, to the desired intracellular location (e.g., the nucleus) such that the HDR template can integrate into the cleaved target site in the endogenous gene. In some embodiments, the RNA-guided nuclease target sequence and the PAM are located at the 5′ terminus of the HDR template. Particularly, in some embodiments, the PAM can be located at the 5′ terminus of the RNA-guided nuclease target sequence. In other embodiments, the PAM can be located at the 3′ terminus of the RNA-guided nuclease target sequence. In some embodiments, the RNA-guided nuclease target sequence and the PAM are located at the 3′ terminus of the HDR template. Particularly, in some embodiments, the PAM can be located at the 5′ terminus of the RNA-guided nuclease target sequence. In other embodiments, the PAM is located at the 3′ terminus of the RNA-guided nuclease target sequence. In some embodiments, the nucleic acid comprises two RNA-guided nuclease target sequences and two PAMs. Particularly, in some embodiments, a first RNA-guided nuclease target sequence and a first PAM are located at the 5′ terminus of the HDR template and a second RNA-guided nuclease target sequence and a second PAM are located at the 3′ terminus of the HDR template. In some embodiments, the first PAM is located at the 5′ terminus of the first RNA-guided nuclease target sequence and the second PAM is located at the 5′ of the second RNA-guided nuclease target sequence. In other embodiments, the first PAM is located at the 5′ terminus of the first RNA-guided nuclease target sequence and the second PAM is located at the 3′ of the second RNA-guided nuclease sequence. In yet other embodiments, the first PAM is located at the 3′ terminus of the first RNA-guided nuclease target sequence and the second PAM is located at the 5′ of the second RNA-guided nuclease target sequence. In yet other embodiments, the first PAM is located at the 3′ terminus of the first RNA-guided nuclease target sequence and the second PAM is located at the 3′ of the second RNA-guided nuclease target sequence.

In some embodiments, a nucleic acid described herein comprises a sequence of equivalent coding potential to the 3′ portion of an endogenous gene in the cell. In certain embodiments, the sequence of equivalent coding potential to the 3′ portion codes for a carboxy-terminal portion of the protein product of the endogenous gene. In some embodiments, the sequence of equivalent coding potential to the 3′ portion of the endogenous gene includes a stop codon and polyadenylation sequence. In some embodiment the sequence of equivalent coding potential to the 3′ portion of the endogenous gene comprises all of the coding sequence 3′ of the target cut site. For example, when inserted into the target cut site in the endogenous gene, the inserted sequence of equivalent coding potential to the 3′ portion forms a contiguous open reading frame with the 5′ portion of the endogenous gene located immediately 5′ of the target cut site and allows restored or continued expression of the protein product encoded by the endogenous gene and under the control of the endogenous promoter. In some embodiments, the sequence of equivalent coding potential to the 3′ portion of the endogenous gene comprises a sequence that is identical to the 3′ portion of the endogenous gene located immediately 3′ of the target cut site. In some embodiments, the sequence of equivalent coding potential to the 3′ portion of the endogenous gene comprises a sequence that is not identical to the 3′ portion of the endogenous gene located immediately 3′ of the target cut site and comprises one or more alternative codon(s).

In some embodiments, the length of the sequence of equivalent coding potential to the 3′ portion of the endogenous gene is about 1- 2500 nucleotides in length. For example, the length of the sequence of equivalent coding potential to the 3′ portion of the endogenous gene is about 1-100, 1-200, 1-300, 1-400, 1-500, 1-600, 1-700, 1-800, 1-900, 1-1000, 100-2500, 200-2500, 300-2500, 400-2500, 500-2500, 600-2500, 700-2500, 800-2500, 900-2500, 1000-2500, 1100-2500, 1200-2500, 1300-2500, 1400-2500, 1500-2500, 1600-2500, 1700-2500, 1800-2500, 1900-2500, 2000-2500, 2100-2500, 2200-2500, 2300-2500, 2500-2500, 100-2000, 200-2000, 300-2000, 400-2000, 500-2000, 600-2000, 700-2000, 800-2000, 900-2000, 1000-2000, 1100-2000, 1200-2000, 1300-2000, 1400-2000, 1500-2000, 1600-2000, 1700-2000, 1800-2000, 1900-2000, 100-1500, 200-1500, 300-1500, 400-1500, 500-1500, 600-1500, 700-1500, 800-1500, 900-1500, 1000-1500, 1100-1500, 1200-1500, 1300-1500, 1400-1500, 100-1250, 200-1250, 300-1250, 400-1250, 500-1250, 600-1250, 700-1250, 800-1250, 900-1250, 1000-1250, 1100-1250, 1200-1250, 100-1000, 200-1000, 300-1000, 400-1000, 500-1000, 600-1000, 700-1000, 800-1000, or 900-1000 nucleotides in length.

In some embodiments, the sequence of equivalent coding potential to the 3′ portion is about 80%, 85%, 90%, 91%, 92%, 93%, 94%, 95%, 96%, 97%, 98%, 99%, or 100% identical to the endogenous gene over the length of the 3′ portion.

In some embodiments, the sequence of equivalent coding potential to the 3′ portion of the endogenous gene can be a 3′ portion of TRAC, TRBC, CD3γ chain, CD3δ chain, CD3_{ε} chain, CD3ξ chain, IL-2Rα chain, IL-2Rβ chain, or IL-2Rγ chain (IL2RG). For example, the sequence of equivalent coding potential to the 3′ portion can have a nucleotide sequence having about 80%, 85%, 90%, 91%, 92%, 93%, 94%, 95%, 96%, 97%, 98%, 99%, or 100% sequence identity to a 3′ portion of any of the sequences described in Table 1.

In some embodiments, the sequence of equivalent coding potential to the 3′ portion of the endogenous gene can be a 3′ portion of Actb, Atp5f1, B2m, Gapdh, Gusb, Hprt, Pgk1, Ppia, Rps18, Tbp, Tfrc, Ywhaz, Nanog, Rex1, or Oct4. For example, the sequence of equivalent coding potential to the 3′ portion can have a nucleotide sequence having about 80%, 85%, 90%, 91%, 92%, 93%, 94%, 95%, 96%, 97%, 98%, 99%, or 100% sequence identity to a 3′ portion of any of the sequences described in Table 2.

In other embodiments, a nucleic acid described herein comprises a sequence of equivalent coding potential to a 5′ portion of an endogenous gene in the cell. In certain embodiments, the sequence of equivalent coding potential to the 5′ portion codes for an amino-terminal portion of the protein product of the endogenous gene. In some embodiments, the sequence of equivalent coding potential to the 5′ portion of the endogenous gene comprises all of the coding sequence 5′ of the target cut site. For example, when inserted into the target cut site in the endogenous gene, the inserted sequence of equivalent coding potential to the 5′ portion forms a contiguous open reading frame with the 3′ portion of the endogenous gene located immediately 3′ of the target cut site and allows restored or continued expression of the protein product encoded by the endogenous gene. In some embodiments, restored or continued expression of the protein product encoded by the endogenous gene is under the control of the endogenous promoter. In other embodiments, an exogenous promoter is inserted into the target cut site and operably linked with the sequence of equivalent coding potential to the 5′ portion of the endogenous gene to drive expression of the protein product of the endogenous gene in the cell. In some embodiments, the sequence of equivalent coding potential to the 5′ portion of the endogenous gene comprises a sequence that is identical to the 5′ portion of the endogenous gene located immediately 5′ of the target cut site. In some embodiments, the sequence of equivalent coding potential to the 5′ portion of the endogenous gene comprises a sequence that is not identical to the 5′ portion of the endogenous gene located immediately 5′ of the target cut site and comprises one or more alternative codon(s).

In some embodiments, the length of the sequence of equivalent coding potential to the 5′ portion of the endogenous gene is about 1- 2500 nucleotides in length. For example, the length of the sequence of equivalent coding potential to the 5′ portion of the endogenous gene is about 1-100, 1-200, 1-300, 1-400, 1-500, 1-600, 1-700, 1-800, 1-900, 1-1000, 100-2500, 200-2500, 300-2500, 400-2500, 500-2500, 600-2500, 700-2500, 800-2500, 900-2500, 1000-2500, 1100-2500, 1200-2500, 1300-2500, 1400-2500, 1500-2500, 1600-2500, 1700-2500, 1800-2500, 1900-2500, 2000-2500, 2100-2500, 2200-2500, 2300-2500, 2500-2500, 100-2000, 200-2000, 300-2000, 400-2000, 500-2000, 600-2000, 700-2000, 800-2000, 900-2000, 1000-2000, 1100-2000, 1200-2000, 1300-2000, 1400-2000, 1500-2000, 1600-2000, 1700-2000, 1800-2000, 1900-2000, 100-1500, 200-1500, 300-1500, 400-1500, 500-1500, 600-1500, 700-1500, 800-1500, 900-1500, 1000-1500, 1100-1500, 1200-1500, 1300-1500, 1400-1500, 100-1250, 200-1250, 300-1250, 400-1250, 500-1250, 600-1250, 700-1250, 800-1250, 900-1250, 1000-1250, 1100-1250, 1200-1250, 100-1000, 200-1000, 300-1000, 400-1000, 500-1000, 600-1000, 700-1000, 800-1000, or 900-1000 nucleotides in length.

In some embodiments, the sequence of equivalent coding potential to the 5′ portion is about 80%, 85%, 90%, 91%, 92%, 93%, 94%, 95%, 96%, 97%, 98%, 99%, or 100% identical to the endogenous gene over the length of the 5′ portion.

In some embodiments, the sequence of equivalent coding potential to the 5′ portion of the endogenous gene can be a 5′ portion of TRAC, TRBC, CD3γ chain, CD3δ chain, CD3_{ε} chain, CD3ξ chain, IL-2Rα chain, IL-2Rβ chain, or IL-2Rγ chain (IL2RG). For example, the sequence of equivalent coding potential to the 5′ portion can have a nucleotide sequence having about 80%, 85%, 90%, 91%, 92%, 93%, 94%, 95%, 96%, 97%, 98%, 99%, or 100% sequence identity to a 5′ portion of any of the sequences described in Table 1.

In some embodiments, the sequence of equivalent coding potential to the 3′ portion of the endogenous gene can be a 5′ portion of Actb, Atp5f1, B2m, Gapdh, Gusb, Hprt, Pgk1, Ppia, Rps18, Tbp, Tfrc, Ywhaz, Nanog, Rex1, or Oct4. For example, the sequence of equivalent coding potential to the 5′ portion can have a nucleotide sequence having about 80%, 85%, 90%, 91%, 92%, 93%, 94%, 95%, 96%, 97%, 98%, 99%, or 100% sequence identity to a 3′ portion of any of the sequences described in Table 2.

Nucleic acids described herein further comprise an exogenous transgene. In some embodiments, the exogenous transgene is inserted into a target cut site in an endogenous gene in the cell resulting in the expression of the transgene. In some embodiments, an exogenous promoter is inserted into the target cut site and operably linked with the exogenous transgene to drive expression of the transgene in the cell.

In some embodiments, the exogenous transgene comprises a sequence encoding one or more polypeptide that is expressed in the cell. For example, in some embodiments, the exogenous transgene comprises a sequence encoding one or more protein expressed on the surface of the cell membrane. In some embodiments, the exogenous transgene comprises a sequence encoding a transmembrane protein, or fragment thereof. For example, in some embodiments, the exogenous transgene comprises one or more sequence encoding a chimeric receptor, CD28, CD45, CD2, CD4, CD5, CD7, CD8, CD9, CD16, CD22, CD27, CD28, CD30, CD33, CD37, CD40, CD64, CD80, CD83, CD86, CD127, CD134, CD137, CD154, CIITA, 4-1BBL. PD-1, PD-1L, LIGHT, DAP10, DAP12, ICAM-1, LFA-1, LCK, TNFR2, ICOS, NKG2C, HLA-E, B7-H3, or beta 2-microglobulin. In some embodiments, the exogenous transgene comprises a sequence encoding a cell surface marker that can be used as a selection marker for cells having successful transgene insertion into the genome of the cell. For example, in some embodiments the exogenous transgene comprises a sequence encoding an epidermal growth factor receptor (EGFR), or truncated fragment thereof, which can be readily detected using an anti-EGFR antibody and flow cytometry. For example, in some embodiments, the exogenous transgene comprises a sequence encoding a truncated EGFR having a nucleotide sequence according to SEQ ID NO:16. in Table 3.

TABLE 3

Surface Markers

| | |
|---|---|
| SEQ ID NO: 16 | ATGGACTGGACATGGATTCTGTTTCTCGTGGCCGCCGCCA |
| | CACGCGTGCACAGCAGAAAGGTGTGCAACGGCATCGGCA |
| | TCGGCGAGTTTAAGGACTCTCTGAGCATCAACGCCACCAA |
| | CATCAAGCACTTCAAGAACTGCACCAGCATCTCCGGCGAC |
| | CTCCACATTCTCCCCGTGGCCTTTAGGGGAGACTCCTTCAC |
| | CCACACCCCTCCTCTGGATCCTCAAGAACTCGACATTCTG |
| | AAGACCGTGAAGGAGATCACCGGCTTTCTGCTGATCCAAG |
| | CTTGGCCCGAGAACAGAACAGATCTCCACGCCTTCGAGAA |
| | TCTGGAGATCATTAGAGGAAGAACAAAGCAGCACGGCCA |
| | GTTTAGCCTCGCCGTGGTCTCTCTGAACATCACATCTCTGG |
| | GACTGAGGTCTCTGAAAGAGATCAGCGACGGCGACGTCA |
| | TCATCTCCGGCAACAAGAATCTGTGCTACGCTAACACCAT |
| | CAACTGGAAGAAGCTCTTCGGCACCAGCGGCCAGAAGAC |
| | CAAGATCATCAGCAATAGAGGCGAGAACAGCTGCAAGGC |
| | CACCGGACAAGTCTGCCACGCTCTGTGTAGCCCCGAGGGC |
| | TGTTGGGGACCCGAGCCCAGAGACTGTGTGAGCTGCAGA |
| | AACGTTTCTAGAGGAAGGGAGTGCGTGGATAAGTGTAATC |
| | TGCTGGAGGGCGAGCCTAGGGAGTTCGTCGAGAACTCCG |
| | AGTGTATCCAATGCCACCCCGAGTGTCTCCCCCAAGCCAT |
| | GAACATCACATGCACCGGAAGAGGCCCCGACAACTGCAT |
| | CCAGTGCGCCCACTACATCGACGGACCCCACTGCGTGAAG |
| | ACATGTCCCGCCGGAGTGATGGGCGAGAACAACACACTG |
| | GTGTGGAAGTACGCCGATGCCGGACACGTCTGTCATCTGT |
| | GTCACCCTAACTGCACCTATGGCTGCACCGGCCCCGGACT |
| | GGAGGGATGTCCCACCAACGGCCCTAAGATTCCCTCCATT |
| | GCCACCGGCATGGTGGGAGCTCTGCTGCTGCTGCTCGTGG |
| | TGGCTCTGGGAATTGGACTGTTCATG |

In some embodiments, the exogenous transgene comprises a sequence encoding a fluorescent protein (e.g., GFP or mCherry) that can be used as a selection marker for cells having successful transgene insertion into the genome of the cell.

In some embodiments, the exogenous transgene comprises a sequence encoding a synthetic antigen receptor, wherein the synthetic antigen receptor is a chimeric antigen receptor (CAR) or a SynNotch receptor. See, for example, Sadelain et al., Cancer Discov. 3(4): 388-398 (2013)); Srivastava Trends Immunol. 36(8): 494-502 (2015)); Toda et al. Science 361(6398): 156-162 (2018); and Cho et al. Scientific Reports 8: 3846 (2018) regarding CAR and SynNotch design and uses). In certain embodiments the exogenous transgene comprises a sequence encoding a chimeric antigen receptor (CAR). In some embodiments, the exogenous transgene comprises a CAR specifically recognizing cancer cell-associated targets such as CD19, BCMA, CD20, CD22, CD30, CD33, CD123, CD133, CEA, EGFR, EGFRvIII, EphA2, ErbB family, GPC3, HER2, FAP, FRα, FD2, Igχ, IL-13α2, Mesothelin, Muc1, PSMA, ROR1, VEGFR2, B7-H3, B7H6, CD5, CD23, CD70, CSPG4, EpCAM, GD3, HLA-A1+MAGE, IL-11Rα, Lewis-Y, Muc16, NKG2D ligands, PSCA, or TAG72. For example, in some embodiments, the exogenous transgene comprises a sequence encoding a CD19-CD28-CD3ξ CAR, a CD19-4-1BB-CD3ξ CAR, a MSLN-CD28-CD3ξ CAR, or a MSLN-4-1BB-CD3ξ CAR.

In some embodiments, the exogenous transgene encodes one or more protein that alters the functionality of the cell. For example, in the case of an exogenous transgene encoding a CAR inserted into the genome of a T-cell, the expression of the CAR can alter the specificity and functionality of the T-cell.

In other embodiments, the exogenous transgene encodes one or more cytoplasmic protein, intracellular protein, or soluble protein. In some embodiments, the exogenous transgene encodes a therapeutic protein. In some embodiments, the exogenous transgene encodes a cytokine or a functional fragment thereof. In some embodiments, the exogenous transgene encodes a transcription factor. In some embodiments, the exogenous transgene encodes an immune checkpoint inhibitor.

In other embodiments, exogenous transgenes can comprise sequences encoding non-translated RNA, such as rRNA, tRNA, gRNA, siRNA, or miRNA.

In some embodiments, the nucleic acid is introduced into a cell as a linear DNA template. In some embodiments, the nucleic acid is introduced into the cell as a double-stranded DNA template. In other embodiments, the DNA template is a single-stranded DNA template. In some embodiments, the DNA template is a double-stranded or single-stranded plasmid.

In some embodiments, the nucleic acid comprises one or more 2A sequence(s) to facilitate co-translation of two or more protein products. For example, in some embodiments, the one or more 2A sequence(s) may be a sequence according to SEQ ID NO:14 or SEQ ID NO:15 in Table 4.

TABLE 4

2A Sequences

| | |
|---|---|
| SEQ ID NO: 14 | TCCGGATCCGGAGAGGGCAGGGGATCTCTCCTTACTTG TGGAGACGTCGAGGAAAACCCTGGACCA |
| SEQ ID NO: 15 | CGGGCTAAACGAAGCGGATCTGGGGTGAAGCAAACCT TGAATTTTGACTTGCTGAAGCTCGCGGGGGATGTGGAA TCTAACCCTGGTCCT |

For example, in some embodiments, the nucleic acid can be a plasmid having a sequence according to SEQ ID NO: 12, SEQ ID NO:13, or SEQ ID NO:33 in Table 5.

TABLE 5

Plasmids

| | |
|---|---|
| TRAC-2A-CD19-CD8A-4-1BB-CD3z-EGFRt-2A-TRAC (SEQ ID NO:11) | TCCCAGGGGCTGATTTCTTTGGTTTTGGATCCAGCTGG ATGTCTGCATTGCCGAGGCCACCAGGGCTGGCTCAGCA ACTGTCGGGGAATCACCAGGGTCTGAGAAATCTTGTGC GCATGTGAGGGGCTGTGGGAGCAGAGAACCACTGGGT GGGAAATTCTAATCCCCACCCTGCTGGAAACTCTCTGG GTGGCCCCAACATGCTAATCCTCCGGCAAACCTCTGTT TCCTCCTCAAAAGGCAGGAGGTCGGAAAGAATAAACA ATGAGAGTCACATTAAAAACACAAAATCCTACGGAAA TACTGAAGAATGAGTCTCAGCACTAAGGAAAAGCCTC CAGCAGCTCCTGCTTTCTGAGGGTGAAGGATAGACGCT GTGGCTCTGCATGACTCACTAGCACTCTATCACGGCCA TATTCTGGCAGGGTCAGTGGCTCCAACTAACATTTGTT TGGTACTTTACAGTTTATTAAATAGATGTTTATATGGA GAAGCTCTCATTTCTTTCTCAGAAGAGCCTGGCTAGGA AGGTGGATGAGGCACCATATTCATTTTGCAGGTGAAAT TCCTGAGATGTAAGGAGCTGCTGTGACTTGCTCAAGGC CTTATATCGAGTAAACGGTAGTGCTGGGGCTTAGACGC AGGTGTTCTGATTTATAGTTCAAAACCTCTATCAATGA GAGAGCAATCTCCTGGTAATGTGATAGATTTCCCAACT TAATGCCAACATACCATAAACCTCCCATTCTGCTAATG CCCAGCCTAAGTTGGGGAGACCACTCCAGATTCCAAG ATGTACAGTTTGCTTTGCTGGGCCTTTTTCCCATGCCTG CCTTTACTCTGCCAGAGTTATATTGCTGGGGTTTTGAA GAAGATCCTATTAAATAAAAGAATAAGCAGTATTATT AAGTAGCCCTGCATTTCAGGTTTCCTTGAGTGGCAGGC CAGGCCTGGCCGTGAACGTTCACTGAAATCATGGCCTC TTGGCCAAGATTGATAGCTTGTGCCTGTCCCTGAGTCC CAGTCCATCACGAGCAGCTGGTTTCTAAGATGCTATTT CCCGTATAAAGCATGAGACCGTGACTTGCCAGCCCCAC |
| | AGAGCCCCGCCCTTGTCCATCACTGGCATCTGGACTCC |
| | AGCCTGGGTTGGGGCAAAGAGGGAAATGAGATCATGT |
| | CCTAACCCTGATCCTCTTGTCCCACAGATTCCGGATCC |
| | GGAGAGGGCAGGGGATCTCTCCTTACTTGTGGAGACG |
| | TCGAGGAAAACCCTGGACCAATGGCCTTACCAGTGAC |
| | CGCCTTGCTCCTGCCGCTGGCCTTGCTGCTCCACGCCG |
| | CCCGCCCGGAACAAAAACTCATTAGCGAAGAGGATCT |
| | CGATATTCAGATGACTCAGACCACCTCTTCTTTGAGCG |
| | CAAGTTTGGGGGATCGGGTTACAATATCCTGCCGCGCC |
| | AGCCAAGACATCAGCAAATACCTTAATTGGTACCAGC |
| | AGAAACCTGATGGCACTGTGAAACTCCTGATCTACCAT |
| | ACCAGCAGGTTGCACAGCGGGGTACCTTCAAGATTTA |
| | GCGGATCAGGAAGCGGTACAGACTACTCACTTACAAT |
| | CAGCAATCTCGAACAGGAAGATATCGCCACATACTTCT |
| | GTCAGCAAGGAAACACTCTGCCCTATACGTTCGGTGGC |
| | GGCACAAAACTCGAGATTACCGGAGGTGGAGGCTCAG |
| | GAGGAGGAGGCAGTGGAGGTGGTGGGTCAGAAGTGA |
| | AACTGCAGGAGTCAGGACCGGGCTTGGTCGCACCATC |
| | CCAATCCCTTTCTGTCACATGCACTGTTAGTGGAGTAT |
| | CCCTACCAGACTACGGGGTATCTTGGATACGGCAGCCG |
| | CCTCGCAAGGGGCTCGAATGGCTCGGAGTGATCTGGG |
| | GGTCTGAGACTACCTATTACAATTCCGCTTTGAAGTCA |
| | CGGTTGACGATCATAAAAGATAACAGTAAATCTCAAG |
| | TGTTTCTCAAGATGAACTCACTCCAAACAGACGATACG |
| | GCCATATATTATTGCGCCAAGCACTATTATTACGGTGG |
| | CTCCTACGCAATGGATTATTGGGGGCAGGGGACTTCTG |
| | TAACCGTGTCAAGCACCACGACGCCAGCGCCGCGACC |
| | ACCAACACCGGCGCCCACCATCGCGTCGCAGCCACTGT |
| | CACTGCGCCCAGAAGCGTGCCGGCCAGCGGCGGGGGG |
| | CGCAGTGCACACGAGGGGGCTGGACTTCGCCTGTGAT |
| | ATCTACATCTGGGCGCCCTTGGCCGGGACTTGTGGGGT |
| | CCTTCTCCTGTCACTGGTTATCACCCTTTACTGCAAACG |
| | GGGCAGAAAGAAACTCCTGTATATATTCAAACAACCA |
| | TTTATGAGACCAGTACAAACTACTCAAGAGGAAGATG |
| | GCTGTAGCTGCCGATTTCCAGAAGAAGAAGAAGGAGG |
| | ATGTGAACTGAGAGTGAAGTTCAGCAGGAGCGCAGAC |
| | GCCCCCGCGTACAAGCAGGGCCAGAACCAGCTCTATA |
| | ACGAGCTCAATCTAGGACGAAGAGAGGAGTACGATGT |
| | TTTGGACAAGAGGCGTGGCCGGGACCCTGAGATGGGG |
| | GGAAAGCCGAGAAGGAAGAACCCTCAGGAAGGCCTGT |
| | ACAATGAACTGCAGAAAGATAAGATGGCGGAGGCCTA |
| | CAGTGAGATTGGGATGAAAGGCGAGCGCCGGAGGGGC |
| | AAGGGGCACGATGGCCTTTACCAGGGTCTCAGTACAG |
| | CCACCAAGGACACCTACGATGCCTTGCACATGCAAGC |
| | CCTGCCCCCTCGCCGGGCTAAACGAAGCGGATCTGGG |
| | GTGAAGCAAACCTTGAATTTTGACTTGCTGAAGCTCGC |
| | GGGGGATGTGGAATCTAACCCTGGTCCTATGGACTGG |
| | ACATGGATTCTGTTTCTCGTGGCCGCCGCCACACGCGT |
| | GCACAGCAGAAAGGTGTGCAACGGCATCGGCATCGGC |
| | GAGTTTAAGGACTCTCTGAGCATCAACGCCACCAACAT |
| | CAAGCACTTCAAGAACTGCACCAGCATCTCCGGCGAC |
| | CTCCACATTCTCCCCGTGGCCTTTAGGGGAGACTCCTT |
| | CACCCACACCCCTCCTCTGGATCCTCAAGAACTCGACA |
| | TTCTGAAGACCGTGAAGGAGATCACCGGCTTTCTGCTG |
| | ATCCAAGCTTGGCCCGAGAACAGAACAGATCTCCACG |
| | CCTTCGAGAATCTGGAGATCATTAGAGGAAGAACAAA |
| | GCAGCACGGCCAGTTTAGCCTCGCCGTGGTCTCTCTGA |
| | ACATCACATCTCTGGGACTGAGGTCTCTGAAAGAGATC |
| | AGCGACGGCGACGTCATCATCTCCGGCAACAAGAATC |
| | TGTGCTACGCTAACACCATCAACTGGAAGAAGCTCTTC |
| | GGCACCAGCGGCCAGAAGACCAAGATCATCAGCAATA |
| | GAGGCGAGAACAGCTGCAAGGCCACCGGACAAGTCTG |
| | CCACGCTCTGTGTAGCCCCGAGGGCTGTTGGGGACCCG |
| | AGCCCAGAGACTGTGTGAGCTGCAGAAACGTTTCTAG |
| | AGGAAGGGAGTGCGTGGATAAGTGTAATCTGCTGGAG |
| | GGCGAGCCTAGGGAGTTCGTCGAGAACTCCGAGTGTA |
| | TCCAATGCCACCCCGAGTGTCTCCCCCAAGCCATGAAC |
| | ATCACATGCACCGGAAGAGGCCCCGACAACTGCATCC |
| | AGTGCGCCCACTACATCGACGGACCCCACTGCGTGAA |
| | GACATGTCCCGCCGGAGTGATGGGCGAGAACAACACA |
| | CTGGTGTGGAAGTACGCCGATGCCGGACACGTCTGTCA |
| | TCTGTGTCACCCTAACTGCACCTATGGCTGCACCGGCC |
| | CCGGACTGGAGGGATGTCCCACCAACGGCCCTAAGAT |
| | TCCCTCCATTGCCACCGGCATGGTGGGAGCTCTGCTGC |
| | TGCTGCTCGTGGTGGCTCTGGGAATTGGACTGTTCATG |
| | CGGGCCAAGCGGTCTGGATCCGGAGCCACCAACTTCA |
| | GCCTGCTGAAGCAGGCCGGCGACGTGGAGGAGAACCC |
| | CGGCCCCATTCAGAATCCTGATCCTGCGGTGTATCAGC |
| | TGAGAGACTCTAAATCCAGTGACAAGTCTGTCTGCCTA |
| | TTCACCGATTTTGATTCTCAAACAAATGTGTCACAAAG |
| | TAAGGATTCTGATGTGTATATCACAGACAAAACTGTGC |
| | TAGACATGAGGTCTATGGACTTCAAGAGCAACAGTGC |
| | TGTGGCCTGGAGCAACAAATCTGACTTTGCATGTGCAA |
| | ACGCCTTCAACAACAGCATTATTCCAGAAGACACCTTC |
| | TTCCCCAGCCCAGGTAAGGGCAGCTTTGGTGCCTTCGC |
| | AGGCTGTTTCCTTGCTTCAGGAATGGCCAGGTTCTGCC |
| | CAGAGCTCTGGTCAATGATGTCTAAAACTCCTCTGATT |
| | GGTGGTCTCGGCCTTATCCATTGCCACCAAAACCCTCT |
| | TTTTACTAAGAAACAGTGAGCCTTGTTCTGGCAGTCCA |
| | GAGAATGACACGGGAAAAAAGCAGATGAAGAGAAGG |
| | TGGCAGGAGAGGGCACGTGGCCCAGCCTCAGTCTCTC |
| | CAACTGAGTTCCTGCCTGCCTGCCTTTGCTCAGACTGTT |
| | TGCCCCTTACTGCTCTTCTAGGCCTCATTCTAAGCCCCT |
| | TCTCCAAGTTGCCTCTCCTTATTTCTCCCTGTCTGCCAA |
| | AAAATCTTTCCCAGCTCACTAAGTCAGTCTCACGCAGT |
| | CACTCATTAACCCACCAATCACTGATTGTGCCGGCACA |
| | TGAATGCACCAGGTGTTGAAGTGGAGGAATTAAAAAG |
| | TCAGATGAGGGGTGTGCCCAGAGGAAGCACCATTCTA |
| | GTTGGGGGAGCCCATCTGTCAGCTGGGAAAAGTCCAA |
| | ATAACTTCAGATTGGAATGTGTTTTAACTCAGGGTTGA |
| | GAAAACAGCTACCTTCAGGACAAAAGTCAGGGAAGGG |
| | CTCTCTGAAGAAATGCTACTTGAAGATACCAGCCCTAC |
| | CAAGGGCAGGGAGAGGACCCTATAGAGGCCTGGGACA |
| | GGAGCTCAATGAGAAAGGAGAAGAGCAGCAGGCATG |
| | AGTTGAATGAAGGAGGCAGGGCCGGGTCACAGGGCCT |
| | TCTAGGCCATGAGAGGGTAGACAGTATTCTAAGGACG |
| | CCAGAAAGCTGTTGATCGGCTTCAAGCAGGGGAGGGA |
| | CACCTAATTTGAGGCTAGGTGGAGGCTCAGTGATGATA |
| | AGTCTGCGATGGTGGATGCATGTGTCATGGTCATAGCT |
| | GTTTCCTGTGTGAAATTGTTATCCGCTCAGAGGGCACA |
| | ATCCTATTCCGCGCTATCCGACAATCTCCAAGACATTA |
| | GGTGGAGTTCAGTTCGGCGTATGGCATATGTCGCTGGA |
| | AAGAACATGTGAGCAAAAGGCCAGCAAAAGGCCAGG |
| | AACCGTAAAAAGGCCGCGTTGCTGGCGTTTTTCCATAG |
| | GCTCCGCCCCCCTGACGAGCATCACAAAAATCGACGCT |
| | CAAGTCAGAGGTGGCGAAACCCGACAGGACTATAAAG |
| | ATACCAGGCGTTTCCCCCTGGAAGCTCCCTCGTGCGCT |
| | CTCCTGTTCCGACCCTGCCGCTTACCGGATACCTGTCC |
| | GCCTTTCTCCCTTCGGGAAGCGTGGCGCTTTCTCATAG |
| | CTCACGCTGTAGGTATCTCAGTTCGGTGTAGGTCGTTC |
| | GCTCCAAGCTGGGCTGTGTGCACGAACCCCCCGTTCAG |
| | CCCGACCGCTGCGCCTTATCCGGTAACTATCGTCTTGA |
| | GTCCAACCCGGTAAGACACGACTTATCGCCACTGGCA |
| | GCAGCCACTGGTAACAGGATTAGCAGAGCGAGGTATG |
| | TAGGCGGTGCTACAGAGTTCTTGAAGTGGTGGCCTAAC |
| | TACGGCTACACTAGAAGAACAGTATTTGGTATCTGCGC |
| | TCTGCTGAAGCCAGTTACCTTCGGAAAAAGAGTTGGTA |
| | GCTCTTGATCCGGCAAACAAACCACCGCTGGTAGCGGT |
| | GGTTTTTTTGTTTGCAAGCAGCAGATTACGCGCAGAAA |
| | AAAAGGATCTCAAGAAGATCCTTTGATCTTTTCTACGG |
| | GGTCTGACGCTCTATTCAACAAAGCCGCCGTCCCGTCA |
| | AGTCAGCGTAAATGGGTAGGGGGCTTCAAATCGTCCTC |
| | GTGATACCAATTCGGAGCCTGCTTTTTTGTACAAACTT |
| | GTTGATAATGGCAATTCAAGGATCTTCACCTAGATCCT |
| | TTTAAATTAAAAATGAAGTTTTAAATCAATCTAAAGTA |
| | TATATGAGTAAACTTGGTCTGACAGTTACCAATGCTTA |
| | ATCAGTGAGGCACCTATCTCAGCGATCTGTCTATTTCG |
| | TTCATCCATAGTTGCCTGACTCCCCGTCGTGTAGATAA |
| | CTACGATACGGGAGGGCTTACCATCTGGCCCCAGTGCT |
| | GCAATGATACCGCGAGAGCCACGCTCACCGGCTCCAG |
| | ATTTATCAGCAATAAACCAGCCAGCCGGAAGGGCCGA |
| | GCGCAGAAGTGGTCCTGCAACTTTATCCGCCTCCATCC |
| | AGTCTATTAATTGTTGCCGGGAAGCTAGAGTAAGTAGT |
| | TCGCCAGTTAATAGTTTGCGCAACGTTGTTGCCATTGC |
| | TACAGGCATCGTGGTGTCACGCTCGTCGTTTGGTATGG |
| | CTTCATTCAGCTCCGGTTCCCAACGATCAAGGCGAGTT |
| | ACATGATCCCCCATGTTGTGCAAAAAAGCGGTTAGCTC |
| | CTTCGGTCCTCCGATCGTTGTCAGAAGTAAGTTGGCCG |
| | CAGTGTTATCACTCATGGTTATGGCAGCACTGCATAAT |
| | TCTCTTACTGTCATGCCATCCGTAAGATGCTTTTCTGTG |
| | ACTGGTGAGTACTCAACCAAGTCATTCTGAGAATAGTG |
| | TATGCGGCGACCGAGTTGCTCTTGCCCGGCGTCAATAC |
| | GGGATAATACCGCGCCACATAGCAGAACTTTAAAAGT |
| | GCTCATCATTGGAAAACGTTCTTCGGGGCGAAAACTCT |
| | CAAGGATCTTACCGCTGTTGAGATCCAGTTCGATGTAA |
| | CCCACTCGTGCACCCAACTGATCTTCAGCATCTTTTACT |
| | TTCACCAGCGTTTCTGGGTGAGCAAAAACAGGAAGGC |
| | AAAATGCCGCAAAAAAGGGAATAAGGGCGACACGGA |
| | AATGTTGAATACTCATACTCTTCCTTTTTCAATATTATT |
| | GAAGCATTTATCAGGGTTATTGTCTCATGAGCGGATAC |
| | ATATTTGAATGTATTTAGAAAAATAAACAAATAGGGG |
| | TTCCGCGCACATTTCCCCGAAAAGTGCCAGATACCTGA |
| | AACAAAACCCATCGTACGGCCAAGGAAGTCTCCAATA |
| | ACTGTGATCCACCACAAGCGCCAGGGTTTTCCCAGTCA |
| | CGACGTTGTAAAACGACGGCCAGTCATGCATAATCCG |
| | CACGCATCTGGAATAAGGAAGTGCCATTCCGCCTGACC |
| | T |
| TRACback _v1-2A-CD19-CD8A-4-1BB-CD3z-EGFRt-2A-TRAC (SEQ ID NO:12) | TCCCAGGGGCTGATTTCTTTGGTTTTGGATCCAGCTGG |
| | ATGTCTGCATTGCCGAGGCCACCAGGGCTGGCTCAGCA |
| | ACTGTCGGGGAATCACCAGGGTCTGAGAAATCTTGTGC |
| | GCATGTGAGGGGCTGTGGGAGCAGAGAACCACTGGGT |
| | GGGAAATTCTAATCCCCACCCTGCTGGAAACTCTCTGG |
| | GTGGCCCCAACATGCTAATCCTCCGGCAAACCTCTGTT |
| | TCCTCCTCAAAAGGCAGGAGGTCGGAAAGAATAAACA |
| | ATGAGAGTCACATTAAAAACACAAAATCCTACGGAAA |
| | TACTGAAGAATGAGTCTCAGCACTAAGGAAAAGCCTC |
| | CAGCAGCTCCTGCTTTCTGAGGGTGAAGGATAGACGCT |
| | GTGGCTCTGCATGACTCACTAGCACTCTATCACGGCCA |
| | TATTCTGGCAGGGTCAGTGGCTCCAACTAACATTTGTT |
| | TGGTACTTTACAGTTTATTAAATAGATGTTTATATGGA |
| | GAAGCTCTCATTTCTTTCTCAGAAGAGCCTGGCTAGGA |
| | AGGTGGATGAGGCACCATATTCATTTTGCAGGTGAAAT |
| | TCCTGAGATGTAAGGAGCTGCTGTGACTTGCTCAAGGC |
| | CTTATATCGAGTAAACGGTAGTGCTGGGGCTTAGACGC |
| | AGGTGTTCTGATTTATAGTTCAAAACCTCTATCAATGA |
| | GAGAGCAATCTCCTGGTAATGTGATAGATTTCCCAACT |
| | TAATGCCAACATACCATAAACCTCCCATTCTGCTAATG |
| | CCCAGCCTAAGTTGGGGAGACCACTCCAGATTCCAAG |
| | ATGTACAGTTTGCTTTGCTGGGCCTTTTTCCCATGCCTG |
| | CCTTTACTCTGCCAGAGTTATATTGCTGGGGTTTTGAA |
| | GAAGATCCTATTAAATAAAAGAATAAGCAGTATTATT |
| | AAGTAGCCCTGCATTTCAGGTTTCCTTGAGTGGCAGGC |
| | CAGGCCTGGCCGTGAACGTTCACTGAAATCATGGCCTC |
| | TTGGCCAAGATTGATAGCTTGTGCCTGTCCCTGAGTCC |
| | CAGTCCATCACGAGCAGCTGGTTTCTAAGATGCTATTT |
| | CCCGTATAAAGCATGAGACCGTGACTTGCCAGCCCCAC |
| | AGAGCCCCGCCCTTGTCCATCACTGGCATCTGGACTCC |
| | AGCCTGGGTTGGGGCAAAGAGGGAAATGAGATCATGT |
| | CCTAACCCTGATCCTCTTGTCCCACAGATATTCAAAAT |
| | CCAGACCCAGCGGTATATCAACTACGCGATTCAAAAA |
| | GTTCTGACAAGAGCGTGTGTCTGTTCACCGATTTCGAC |
| | AGCCAGACAAATGTATCGCAGTCAAAGGATTCTGACG |
| | TCTACATAACCGACAAAACTGTGTTGGACATGAGAAG |
| | TATGGACTTTAAGAGCAATTCTGCGGTTGCTTGGAGCA |
| | ACAAGTCCGATTTCGCCTGCGCAAATGCTTTTAACAAC |
| | TCTATTATCCCGGAAGATACCTTTTTCCCATCACCCGA |
| | AAGCTCCTGCGATGTGAAGCTGGTGGAGAAATCCTTTG |
| | AGACTGACACGAATCTGAACTTCCAGAACCTGAGTGT |
| | GATAGGATTCCGAATCTTGCTCCTGAAAGTGGCCGGAT |
| | TTAACCTCTTAATGACCCTTCGGCTTTGGTCCAGTGGA |
| | TCCGGAGAGGGCAGGGGATCTCTCCTTACTTGTGGAGA |
| | CGTCGAGGAAAACCCTGGACCAATGGCCTTACCAGTG |
| | ACCGCCTTGCTCCTGCCGCTGGCCTTGCTGCTCCACGC |
| | CGCCCGCCCGGAACAAAAACTCATTAGCGAAGAGGAT |
| | CTCGATATTCAGATGACTCAGACCACCTCTTCTTTGAG |
| | CGCAAGTTTGGGGGATCGGGTTACAATATCCTGCCGCG |
| | CCAGCCAAGACATCAGCAAATACCTTAATTGGTACCA |
| | GCAGAAACCTGATGGCACTGTGAAACTCCTGATCTACC |
| | ATACCAGCAGGTTGCACAGCGGGGTACCTTCAAGATTT |
| | AGCGGATCAGGAAGCGGTACAGACTACTCACTTACAA |
| | TCAGCAATCTCGAACAGGAAGATATCGCCACATACTTC |
| | TGTCAGCAAGGAAACACTCTGCCCTATACGTTCGGTGG |
| | CGGCACAAAACTCGAGATTACCGGAGGTGGAGGCTCA |
| | GGAGGAGGAGGCAGTGGAGGTGGTGGGTCAGAAGTG |
| | AAACTGCAGGAGTCAGGACCGGGCTTGGTCGCACCAT |
| | CCCAATCCCTTTCTGTCACATGCACTGTTAGTGGAGTA |
| | TCCCTACCAGACTACGGGGTATCTTGGATACGGCAGCC |
| | GCCTCGCAAGGGGCTCGAATGGCTCGGAGTGATCTGG |
| | GGGTCTGAGACTACCTATTACAATTCCGCTTTGAAGTC |
| | ACGGTTGACGATCATAAAAGATAACAGTAAATCTCAA |
| | GTGTTTCTCAAGATGAACTCACTCCAAACAGACGATAC |
| | GGCCATATATTATTGCGCCAAGCACTATTATTACGGTG |
| | GCTCCTACGCAATGGATTATTGGGGGCAGGGGACTTCT |
| | GTAACCGTGTCAAGCACCACGACGCCAGCGCCGCGAC |
| | CACCAACACCGGCGCCCACCATCGCGTCGCAGCCACT |
| | GTCACTGCGCCCAGAAGCGTGCCGGCCAGCGGCGGGG |
| | GGCGCAGTGCACACGAGGGGGCTGGACTTCGCCTGTG |
| | ATATCTACATCTGGGCGCCCTTGGCCGGGACTTGTGGG |
| | GTCCTTCTCCTGTCACTGGTTATCACCCTTTACTGCAAA |
| | CGGGGCAGAAAGAAACTCCTGTATATATTCAAACAAC |
| | CATTTATGAGACCAGTACAAACTACTCAAGAGGAAGA |
| | TGGCTGTAGCTGCCGATTTCCAGAAGAAGAAGAAGGA |
| | GGATGTGAACTGAGAGTGAAGTTCAGCAGGAGCGCAG |
| | ACGCCCCCGCGTACAAGCAGGGCCAGAACCAGCTCTA |
| | TAACGAGCTCAATCTAGGACGAAGAGAGGAGTACGAT |
| | GTTTTGGACAAGAGGCGTGGCCGGGACCCTGAGATGG |
| | GGGGAAAGCCGAGAAGGAAGAACCCTCAGGAAGGCC |
| | TGTACAATGAACTGCAGAAAGATAAGATGGCGGAGGC |
| | CTACAGTGAGATTGGGATGAAAGGCGAGCGCCGGAGG |
| | GGCAAGGGGCACGATGGCCTTTACCAGGGTCTCAGTA |
| | CAGCCACCAAGGACACCTACGATGCCTTGCACATGCA |
| | AGCCCTGCCCCCTCGCCGGGCTAAACGAAGCGGATCT |
| | GGGGTGAAGCAAACCTTGAATTTTGACTTGCTGAAGCT |
| | CGCGGGGGATGTGGAATCTAACCCTGGTCCTATGGACT |
| | GGACATGGATTCTGTTTCTCGTGGCCGCCGCCACACGC |
| | GTGCACAGCAGAAAGGTGTGCAACGGCATCGGCATCG |
| | GCGAGTTTAAGGACTCTCTGAGCATCAACGCCACCAAC |
| | ATCAAGCACTTCAAGAACTGCACCAGCATCTCCGGCG |
| | ACCTCCACATTCTCCCCGTGGCCTTTAGGGGAGACTCC |
| | TTCACCCACACCCCTCCTCTGGATCCTCAAGAACTCGA |
| | CATTCTGAAGACCGTGAAGGAGATCACCGGCTTTCTGC |
| | TGATCCAAGCTTGGCCCGAGAACAGAACAGATCTCCA |
| | CGCCTTCGAGAATCTGGAGATCATTAGAGGAAGAACA |
| | AAGCAGCACGGCCAGTTTAGCCTCGCCGTGGTCTCTCT |
| | GAACATCACATCTCTGGGACTGAGGTCTCTGAAAGAG |
| | ATCAGCGACGGCGACGTCATCATCTCCGGCAACAAGA |
| | ATCTGTGCTACGCTAACACCATCAACTGGAAGAAGCTC |
| | TTCGGCACCAGCGGCCAGAAGACCAAGATCATCAGCA |
| | ATAGAGGCGAGAACAGCTGCAAGGCCACCGGACAAGT |
| | CTGCCACGCTCTGTGTAGCCCCGAGGGCTGTTGGGGAC |
| | CCGAGCCCAGAGACTGTGTGAGCTGCAGAAACGTTTCT |
| | AGAGGAAGGGAGTGCGTGGATAAGTGTAATCTGCTGG |
| | AGGGCGAGCCTAGGGAGTTCGTCGAGAACTCCGAGTG |
| | TATCCAATGCCACCCCGAGTGTCTCCCCCAAGCCATGA |
| | ACATCACATGCACCGGAAGAGGCCCCGACAACTGCAT |
| | CCAGTGCGCCCACTACATCGACGGACCCCACTGCGTGA |
| | AGACATGTCCCGCCGGAGTGATGGGCGAGAACAACAC |
| | ACTGGTGTGGAAGTACGCCGATGCCGGACACGTCTGTC |
| | ATCTGTGTCACCCTAACTGCACCTATGGCTGCACCGGC |
| | CCCGGACTGGAGGGATGTCCCACCAACGGCCCTAAGA |
| | TTCCCTCCATTGCCACCGGCATGGTGGGAGCTCTGCTG |
| | CTGCTGCTCGTGGTGGCTCTGGGAATTGGACTGTTCAT |
| | GCGGGCCAAGCGGTCTGGATCCGGAGCCACCAACTTC |
| | AGCCTGCTGAAGCAGGCCGGCGACGTGGAGGAGAACC |
| | CCGGCCCCATTCAGAATCCTGATCCTGCGGTGTATCAG |
| | CTGAGAGACTCTAAATCCAGTGACAAGTCTGTCTGCCT |
| | ATTCACCGATTTTGATTCTCAAACAAATGTGTCACAAA |
| | GTAAGGATTCTGATGTGTATATCACAGACAAAACTGTG |
| | CTAGACATGAGGTCTATGGACTTCAAGAGCAACAGTG |
| | CTGTGGCCTGGAGCAACAAATCTGACTTTGCATGTGCA |
| | AACGCCTTCAACAACAGCATTATTCCAGAAGACACCTT |
| | CTTCCCCAGCCCAGGTAAGGGCAGCTTTGGTGCCTTCG |
| | CAGGCTGTTTCCTTGCTTCAGGAATGGCCAGGTTCTGC |
| | CCAGAGCTCTGGTCAATGATGTCTAAAACTCCTCTGAT |
| | TGGTGGTCTCGGCCTTATCCATTGCCACCAAAACCCTC |
| | TTTTTACTAAGAAACAGTGAGCCTTGTTCTGGCAGTCC |
| | AGAGAATGACACGGGAAAAAAGCAGATGAAGAGAAG |
| | GTGGCAGGAGAGGGCACGTGGCCCAGCCTCAGTCTCT |
| | CCAACTGAGTTCCTGCCTGCCTGCCTTTGCTCAGACTG |
| | TTTGCCCCTTACTGCTCTTCTAGGCCTCATTCTAAGCCC |
| | CTTCTCCAAGTTGCCTCTCCTTATTTCTCCCTGTCTGCC |
| | AAAAAATCTTTCCCAGCTCACTAAGTCAGTCTCACGCA |
| | GTCACTCATTAACCCACCAATCACTGATTGTGCCGGCA |
| | CATGAATGCACCAGGTGTTGAAGTGGAGGAATTAAAA |
| | AGTCAGATGAGGGGTGTGCCCAGAGGAAGCACCATTC |
| | TAGTTGGGGGAGCCCATCTGTCAGCTGGGAAAAGTCC |
| | AAATAACTTCAGATTGGAATGTGTTTTAACTCAGGGTT |
| | GAGAAAACAGCTACCTTCAGGACAAAAGTCAGGGAAG |
| | GGCTCTCTGAAGAAATGCTACTTGAAGATACCAGCCCT |
| | ACCAAGGGCAGGGAGAGGACCCTATAGAGGCCTGGGA |
| | CAGGAGCTCAATGAGAAAGGAGAAGAGCAGCAGGCA |
| | TGAGTTGAATGAAGGAGGCAGGGCCGGGTCACAGGGC |
| | CTTCTAGGCCATGAGAGGGTAGACAGTATTCTAAGGA |
| | CGCCAGAAAGCTGTTGATCGGCTTCAAGCAGGGGAGG |
| | GACACCTAATTTGAGGCTAGGTGGAGGCTCAGTGATG |
| | ATAAGTCTGCGATGGTGGATGCATGTGTCATGGTCATA |
| | GCTGTTTCCTGTGTGAAATTGTTATCCGCTCAGAGGGC |
| | ACAATCCTATTCCGCGCTATCCGACAATCTCCAAGACA |
| | TTAGGTGGAGTTCAGTTCGGCGTATGGCATATGTCGCT |
| | GGAAAGAACATGTGAGCAAAAGGCCAGCAAAAGGCC |
| | AGGAACCGTAAAAAGGCCGCGTTGCTGGCGTTTTTCCA |
| | TAGGCTCCGCCCCCCTGACGAGCATCACAAAAATCGA |
| | CGCTCAAGTCAGAGGTGGCGAAACCCGACAGGACTAT |
| | AAAGATACCAGGCGTTTCCCCCTGGAAGCTCCCTCGTG |
| | CGCTCTCCTGTTCCGACCCTGCCGCTTACCGGATACCT |
| | GTCCGCCTTTCTCCCTTCGGGAAGCGTGGCGCTTTCTC |
| | ATAGCTCACGCTGTAGGTATCTCAGTTCGGTGTAGGTC |
| | GTTCGCTCCAAGCTGGGCTGTGTGCACGAACCCCCCGT |
| | TCAGCCCGACCGCTGCGCCTTATCCGGTAACTATCGTC |
| | TTGAGTCCAACCCGGTAAGACACGACTTATCGCCACTG |
| | GCAGCAGCCACTGGTAACAGGATTAGCAGAGCGAGGT |
| | ATGTAGGCGGTGCTACAGAGTTCTTGAAGTGGTGGCCT |
| | AACTACGGCTACACTAGAAGAACAGTATTTGGTATCTG |
| | CGCTCTGCTGAAGCCAGTTACCTTCGGAAAAAGAGTTG |
| | GTAGCTCTTGATCCGGCAAACAAACCACCGCTGGTAGC |
| | GGTGGTTTTTTTGTTTGCAAGCAGCAGATTACGCGCAG |
| | AAAAAAAGGATCTCAAGAAGATCCTTTGATCTTTTCTA |
| | CGGGGTCTGACGCTCTATTCAACAAAGCCGCCGTCCCG |
| | TCAAGTCAGCGTAAATGGGTAGGGGGCTTCAAATCGT |
| | CCTCGTGATACCAATTCGGAGCCTGCTTTTTTGTACAA |
| | ACTTGTTGATAATGGCAATTCAAGGATCTTCACCTAGA |
| | TCCTTTTAAATTAAAAATGAAGTTTTAAATCAATCTAA |
| | AGTATATATGAGTAAACTTGGTCTGACAGTTACCAATG |
| | CTTAATCAGTGAGGCACCTATCTCAGCGATCTGTCTAT |
| | TTCGTTCATCCATAGTTGCCTGACTCCCCGTCGTGTAG |
| | ATAACTACGATACGGGAGGGCTTACCATCTGGCCCCA |
| | GTGCTGCAATGATACCGCGAGAGCCACGCTCACCGGC |
| | TCCAGATTTATCAGCAATAAACCAGCCAGCCGGAAGG |
| | GCCGAGCGCAGAAGTGGTCCTGCAACTTTATCCGCCTC |
| | CATCCAGTCTATTAATTGTTGCCGGGAAGCTAGAGTAA |
| | GTAGTTCGCCAGTTAATAGTTTGCGCAACGTTGTTGCC |
| | ATTGCTACAGGCATCGTGGTGTCACGCTCGTCGTTTGG |
| | TATGGCTTCATTCAGCTCCGGTTCCCAACGATCAAGGC |
| | GAGTTACATGATCCCCCATGTTGTGCAAAAAAGCGGTT |
| | AGCTCCTTCGGTCCTCCGATCGTTGTCAGAAGTAAGTT |
| | GGCCGCAGTGTTATCACTCATGGTTATGGCAGCACTGC |
| | ATAATTCTCTTACTGTCATGCCATCCGTAAGATGCTTTT |
| | CTGTGACTGGTGAGTACTCAACCAAGTCATTCTGAGAA |
| | TAGTGTATGCGGCGACCGAGTTGCTCTTGCCCGGCGTC |
| | AATACGGGATAATACCGCGCCACATAGCAGAACTTTA |
| | AAAGTGCTCATCATTGGAAAACGTTCTTCGGGGCGAA |
| | AACTCTCAAGGATCTTACCGCTGTTGAGATCCAGTTCG |
| | ATGTAACCCACTCGTGCACCCAACTGATCTTCAGCATC |
| | TTTTACTTTCACCAGCGTTTCTGGGTGAGCAAAAACAG |
| | GAAGGCAAAATGCCGCAAAAAAGGGAATAAGGGCGA |
| | CACGGAAATGTTGAATACTCATACTCTTCCTTTTTCAAT |
| | ATTATTGAAGCATTTATCAGGGTTATTGTCTCATGAGC |
| | GGATACATATTTGAATGTATTTAGAAAAATAAACAAAT |
| | AGGGGTTCCGCGCACATTTCCCCGAAAAGTGCCAGAT |
| | ACCTGAAACAAAACCCATCGTACGGCCAAGGAAGTCT |
| | CCAATAACTGTGATCCACCACAAGCGCCAGGGTTTTCC |
| | CAGTCACGACGTTGTAAAACGACGGCCAGTCATGCAT |
| | AATCCGCACGCATCTGGAATAAGGAAGTGCCATTCCG |
| | CCTGACCT |
| TRACback _v2-2A-CD19-CD8A-4-1BB-CD3z-EGFRt-2A-TRAC (SEQ ID NO:13) | TCCCAGGGGCTGATTTCTTTGGTTTTGGATCCAGCTGG |
| | ATGTCTGCATTGCCGAGGCCACCAGGGCTGGCTCAGCA |
| | ACTGTCGGGGAATCACCAGGGTCTGAGAAATCTTGTGC |
| | GCATGTGAGGGGCTGTGGGAGCAGAGAACCACTGGGT |
| | GGGAAATTCTAATCCCCACCCTGCTGGAAACTCTCTGG |
| | GTGGCCCCAACATGCTAATCCTCCGGCAAACCTCTGTT |
| | TCCTCCTCAAAAGGCAGGAGGTCGGAAAGAATAAACA |
| | ATGAGAGTCACATTAAAAACACAAAATCCTACGGAAA |
| | TACTGAAGAATGAGTCTCAGCACTAAGGAAAAGCCTC |
| | CAGCAGCTCCTGCTTTCTGAGGGTGAAGGATAGACGCT |
| | GTGGCTCTGCATGACTCACTAGCACTCTATCACGGCCA |
| | TATTCTGGCAGGGTCAGTGGCTCCAACTAACATTTGTT |
| | TGGTACTTTACAGTTTATTAAATAGATGTTTATATGGA |
| | GAAGCTCTCATTTCTTTCTCAGAAGAGCCTGGCTAGGA |
| | AGGTGGATGAGGCACCATATTCATTTTGCAGGTGAAAT |
| | TCCTGAGATGTAAGGAGCTGCTGTGACTTGCTCAAGGC |
| | CTTATATCGAGTAAACGGTAGTGCTGGGGCTTAGACGC |
| | AGGTGTTCTGATTTATAGTTCAAAACCTCTATCAATGA |
| | GAGAGCAATCTCCTGGTAATGTGATAGATTTCCCAACT |
| | TAATGCCAACATACCATAAACCTCCCATTCTGCTAATG |
| | CCCAGCCTAAGTTGGGGAGACCACTCCAGATTCCAAG |
| | ATGTACAGTTTGCTTTGCTGGGCCTTTTTCCCATGCCTG |
| | CCTTTACTCTGCCAGAGTTATATTGCTGGGGTTTTGAA |
| | GAAGATCCTATTAAATAAAAGAATAAGCAGTATTATT |
| | AAGTAGCCCTGCATTTCAGGTTTCCTTGAGTGGCAGGC |
| | CAGGCCTGGCCGTGAACGTTCACTGAAATCATGGCCTC |
| | TTGGCCAAGATTGATAGCTTGTGCCTGTCCCTGAGTCC |
| | CAGTCCATCACGAGCAGCTGGTTTCTAAGATGCTATTT |
| | CCCGTATAAAGCATGAGACCGTGACTTGCCAGCCCCAC |
| | AGAGCCCCGCCCTTGTCCATCACTGGCATCTGGACTCC |
| | AGCCTGGGTTGGGGCAAAGAGGGAAATGAGATCATGT |
| | CCTAACCCTGATCCTCTTGTCCCACAGATATCCAGAAT |
| | CCCGACCCTGCGGTTTATCAGCTACGCGACTCCAAATC |
| | CAGCGACAAGTCTGTGTGCCTGTTCACGGATTTCGATT |
| | CTCAGACAAACGTTAGCCAGTCAAAAGATTCTGACGT |
| | GTATATCACTGACAAAACCGTCCTGGATATGAGGAGT |
| | ATGGATTTTAAGTCCAATAGCGCTGTCGCCTGGTCTAA |
| | CAAGAGCGACTTTGCTTGTGCAAACGCCTTTAACAACT |
| | CAATTATTCCAGAGGATACTTTTTTCCCAAGTCCCGAA |
| | TCCTCCTGCGACGTGAAGCTGGTGGAGAAGTCGTTTGA |
| | AACAGACACCAATTTGAATTTCCAAAACTTGTCAGTGA |
| | TCGGGTTCAGAATACTCCTTCTGAAAGTAGCCGGCTTC |
| | AATCTGTTAATGACCCTTCGGCTCTGGAGCAGTGGATC |
| | CGGAGAGGGCAGGGGATCTCTCCTTACTTGTGGAGAC |
| | GTCGAGGAAAACCCTGGACCAATGGCCTTACCAGTGA |
| | CCGCCTTGCTCCTGCCGCTGGCCTTGCTGCTCCACGCC |
| | GCCCGCCCGGAACAAAAACTCATTAGCGAAGAGGATC |
| | TCGATATTCAGATGACTCAGACCACCTCTTCTTTGAGC |
| | GCAAGTTTGGGGGATCGGGTTACAATATCCTGCCGCGC |
| | CAGCCAAGACATCAGCAAATACCTTAATTGGTACCAG |
| | CAGAAACCTGATGGCACTGTGAAACTCCTGATCTACCA |
| | TACCAGCAGGTTGCACAGCGGGGTACCTTCAAGATTTA |
| | GCGGATCAGGAAGCGGTACAGACTACTCACTTACAAT |
| | CAGCAATCTCGAACAGGAAGATATCGCCACATACTTCT |
| | GTCAGCAAGGAAACACTCTGCCCTATACGTTCGGTGGC |
| | GGCACAAAACTCGAGATTACCGGAGGTGGAGGCTCAG |
| | GAGGAGGAGGCAGTGGAGGTGGTGGGTCAGAAGTGA |
| | AACTGCAGGAGTCAGGACCGGGCTTGGTCGCACCATC |
| | CCAATCCCTTTCTGTCACATGCACTGTTAGTGGAGTAT |
| | CCCTACCAGACTACGGGGTATCTTGGATACGGCAGCCG |
| | CCTCGCAAGGGGCTCGAATGGCTCGGAGTGATCTGGG |
| | GGTCTGAGACTACCTATTACAATTCCGCTTTGAAGTCA |
| | CGGTTGACGATCATAAAAGATAACAGTAAATCTCAAG |
| | TGTTTCTCAAGATGAACTCACTCCAAACAGACGATACG |
| | GCCATATATTATTGCGCCAAGCACTATTATTACGGTGG |
| | CTCCTACGCAATGGATTATTGGGGGCAGGGGACTTCTG |
| | TAACCGTGTCAAGCACCACGACGCCAGCGCCGCGACC |
| | ACCAACACCGGCGCCCACCATCGCGTCGCAGCCACTGT |
| | CACTGCGCCCAGAAGCGTGCCGGCCAGCGGCGGGGGG |
| | CGCAGTGCACACGAGGGGGCTGGACTTCGCCTGTGAT |
| | ATCTACATCTGGGCGCCCTTGGCCGGGACTTGTGGGGT |
| | CCTTCTCCTGTCACTGGTTATCACCCTTTACTGCAAACG |
| | GGGCAGAAAGAAACTCCTGTATATATTCAAACAACCA |
| | TTTATGAGACCAGTACAAACTACTCAAGAGGAAGATG |
| | GCTGTAGCTGCCGATTTCCAGAAGAAGAAGAAGGAGG |
| | ATGTGAACTGAGAGTGAAGTTCAGCAGGAGCGCAGAC |
| | GCCCCCGCGTACAAGCAGGGCCAGAACCAGCTCTATA |
| | ACGAGCTCAATCTAGGACGAAGAGAGGAGTACGATGT |
| | TTTGGACAAGAGGCGTGGCCGGGACCCTGAGATGGGG |
| | GGAAAGCCGAGAAGGAAGAACCCTCAGGAAGGCCTGT |
| | ACAATGAACTGCAGAAAGATAAGATGGCGGAGGCCTA |
| | CAGTGAGATTGGGATGAAAGGCGAGCGCCGGAGGGGC |
| | AAGGGGCACGATGGCCTTTACCAGGGTCTCAGTACAG |
| | CCACCAAGGACACCTACGATGCCTTGCACATGCAAGC |
| | CCTGCCCCCTCGCCGGGCTAAACGAAGCGGATCTGGG |
| | GTGAAGCAAACCTTGAATTTTGACTTGCTGAAGCTCGC |
| | GGGGGATGTGGAATCTAACCCTGGTCCTATGGACTGG |
| | ACATGGATTCTGTTTCTCGTGGCCGCCGCCACACGCGT |
| | GCACAGCAGAAAGGTGTGCAACGGCATCGGCATCGGC |
| | GAGTTTAAGGACTCTCTGAGCATCAACGCCACCAACAT |
| | CAAGCACTTCAAGAACTGCACCAGCATCTCCGGCGAC |
| | CTCCACATTCTCCCCGTGGCCTTTAGGGGAGACTCCTT |
| | CACCCACACCCCTCCTCTGGATCCTCAAGAACTCGACA |
| | TTCTGAAGACCGTGAAGGAGATCACCGGCTTTCTGCTG |
| | ATCCAAGCTTGGCCCGAGAACAGAACAGATCTCCACG |
| | CCTTCGAGAATCTGGAGATCATTAGAGGAAGAACAAA |
| | GCAGCACGGCCAGTTTAGCCTCGCCGTGGTCTCTCTGA |
| | ACATCACATCTCTGGGACTGAGGTCTCTGAAAGAGATC |
| | AGCGACGGCGACGTCATCATCTCCGGCAACAAGAATC |
| | TGTGCTACGCTAACACCATCAACTGGAAGAAGCTCTTC |
| | GGCACCAGCGGCCAGAAGACCAAGATCATCAGCAATA |
| | GAGGCGAGAACAGCTGCAAGGCCACCGGACAAGTCTG |
| | CCACGCTCTGTGTAGCCCCGAGGGCTGTTGGGGACCCG |
| | AGCCCAGAGACTGTGTGAGCTGCAGAAACGTTTCTAG |
| | AGGAAGGGAGTGCGTGGATAAGTGTAATCTGCTGGAG |
| | GGCGAGCCTAGGGAGTTCGTCGAGAACTCCGAGTGTA |
| | TCCAATGCCACCCCGAGTGTCTCCCCCAAGCCATGAAC |
| | ATCACATGCACCGGAAGAGGCCCCGACAACTGCATCC |
| | AGTGCGCCCACTACATCGACGGACCCCACTGCGTGAA |
| | GACATGTCCCGCCGGAGTGATGGGCGAGAACAACACA |
| | CTGGTGTGGAAGTACGCCGATGCCGGACACGTCTGTCA |
| | TCTGTGTCACCCTAACTGCACCTATGGCTGCACCGGCC |
| | CCGGACTGGAGGGATGTCCCACCAACGGCCCTAAGAT |
| | TCCCTCCATTGCCACCGGCATGGTGGGAGCTCTGCTGC |
| | TGCTGCTCGTGGTGGCTCTGGGAATTGGACTGTTCATG |
| | CGGGCCAAGCGGTCTGGATCCGGAGCCACCAACTTCA |
| | GCCTGCTGAAGCAGGCCGGCGACGTGGAGGAGAACCC |
| | CGGCCCCATTCAGAATCCTGATCCTGCGGTGTATCAGC |
| | TGAGAGACTCTAAATCCAGTGACAAGTCTGTCTGCCTA |
| | TTCACCGATTTTGATTCTCAAACAAATGTGTCACAAAG |
| | TAAGGATTCTGATGTGTATATCACAGACAAAACTGTGC |
| | TAGACATGAGGTCTATGGACTTCAAGAGCAACAGTGC |
| | TGTGGCCTGGAGCAACAAATCTGACTTTGCATGTGCAA |
| | ACGCCTTCAACAACAGCATTATTCCAGAAGACACCTTC |
| | TTCCCCAGCCCAGGTAAGGGCAGCTTTGGTGCCTTCGC |
| | AGGCTGTTTCCTTGCTTCAGGAATGGCCAGGTTCTGCC |
| | CAGAGCTCTGGTCAATGATGTCTAAAACTCCTCTGATT |
| | GGTGGTCTCGGCCTTATCCATTGCCACCAAAACCCTCT |
| | TTTTACTAAGAAACAGTGAGCCTTGTTCTGGCAGTCCA |
| | GAGAATGACACGGGAAAAAAGCAGATGAAGAGAAGG |
| | TGGCAGGAGAGGGCACGTGGCCCAGCCTCAGTCTCTC |
| | CAACTGAGTTCCTGCCTGCCTGCCTTTGCTCAGACTGTT |
| | TGCCCCTTACTGCTCTTCTAGGCCTCATTCTAAGCCCCT |
| | TCTCCAAGTTGCCTCTCCTTATTTCTCCCTGTCTGCCAA |
| | AAAATCTTTCCCAGCTCACTAAGTCAGTCTCACGCAGT |
| | CACTCATTAACCCACCAATCACTGATTGTGCCGGCACA |
| | TGAATGCACCAGGTGTTGAAGTGGAGGAATTAAAAAG |
| | TCAGATGAGGGGTGTGCCCAGAGGAAGCACCATTCTA |
| | GTTGGGGGAGCCCATCTGTCAGCTGGGAAAAGTCCAA |
| | ATAACTTCAGATTGGAATGTGTTTTAACTCAGGGTTGA |
| | GAAAACAGCTACCTTCAGGACAAAAGTCAGGGAAGGG |
| | CTCTCTGAAGAAATGCTACTTGAAGATACCAGCCCTAC |
| | CAAGGGCAGGGAGAGGACCCTATAGAGGCCTGGGACA |
| | GGAGCTCAATGAGAAAGGAGAAGAGCAGCAGGCATG |
| | AGTTGAATGAAGGAGGCAGGGCCGGGTCACAGGGCCT |
| | TCTAGGCCATGAGAGGGTAGACAGTATTCTAAGGACG |
| | CCAGAAAGCTGTTGATCGGCTTCAAGCAGGGGAGGGA |
| | CACCTAATTTGAGGCTAGGTGGAGGCTCAGTGATGATA |
| | AGTCTGCGATGGTGGATGCATGTGTCATGGTCATAGCT |
| | GTTTCCTGTGTGAAATTGTTATCCGCTCAGAGGGCACA |
| | ATCCTATTCCGCGCTATCCGACAATCTCCAAGACATTA |
| | GGTGGAGTTCAGTTCGGCGTATGGCATATGTCGCTGGA |
| | AAGAACATGTGAGCAAAAGGCCAGCAAAAGGCCAGG |
| | AACCGTAAAAAGGCCGCGTTGCTGGCGTTTTTCCATAG |
| | GCTCCGCCCCCCTGACGAGCATCACAAAAATCGACGCT |
| | CAAGTCAGAGGTGGCGAAACCCGACAGGACTATAAAG |
| | ATACCAGGCGTTTCCCCCTGGAAGCTCCCTCGTGCGCT |
| | CTCCTGTTCCGACCCTGCCGCTTACCGGATACCTGTCC |
| | GCCTTTCTCCCTTCGGGAAGCGTGGCGCTTTCTCATAG |
| | CTCACGCTGTAGGTATCTCAGTTCGGTGTAGGTCGTTC |
| | GCTCCAAGCTGGGCTGTGTGCACGAACCCCCCGTTCAG |
| | CCCGACCGCTGCGCCTTATCCGGTAACTATCGTCTTGA |
| | GTCCAACCCGGTAAGACACGACTTATCGCCACTGGCA |
| | GCAGCCACTGGTAACAGGATTAGCAGAGCGAGGTATG |
| | TAGGCGGTGCTACAGAGTTCTTGAAGTGGTGGCCTAAC |
| | TACGGCTACACTAGAAGAACAGTATTTGGTATCTGCGC |
| | TCTGCTGAAGCCAGTTACCTTCGGAAAAAGAGTTGGTA |
| | GCTCTTGATCCGGCAAACAAACCACCGCTGGTAGCGGT |
| | GGTTTTTTTGTTTGCAAGCAGCAGATTACGCGCAGAAA |
| | AAAAGGATCTCAAGAAGATCCTTTGATCTTTTCTACGG |
| | GGTCTGACGCTCTATTCAACAAAGCCGCCGTCCCGTCA |
| | AGTCAGCGTAAATGGGTAGGGGGCTTCAAATCGTCCTC |
| | GTGATACCAATTCGGAGCCTGCTTTTTTGTACAAACTT |
| | GTTGATAATGGCAATTCAAGGATCTTCACCTAGATCCT |
| | TTTAAATTAAAAATGAAGTTTTAAATCAATCTAAAGTA |
| | TATATGAGTAAACTTGGTCTGACAGTTACCAATGCTTA |
| | ATCAGTGAGGCACCTATCTCAGCGATCTGTCTATTTCG |
| | TTCATCCATAGTTGCCTGACTCCCCGTCGTGTAGATAA |
| | CTACGATACGGGAGGGCTTACCATCTGGCCCCAGTGCT |
| | GCAATGATACCGCGAGAGCCACGCTCACCGGCTCCAG |
| | ATTTATCAGCAATAAACCAGCCAGCCGGAAGGGCCGA |
| | GCGCAGAAGTGGTCCTGCAACTTTATCCGCCTCCATCC |
| | AGTCTATTAATTGTTGCCGGGAAGCTAGAGTAAGTAGT |
| | TCGCCAGTTAATAGTTTGCGCAACGTTGTTGCCATTGC |
| | TACAGGCATCGTGGTGTCACGCTCGTCGTTTGGTATGG |
| | CTTCATTCAGCTCCGGTTCCCAACGATCAAGGCGAGTT |
| | ACATGATCCCCCATGTTGTGCAAAAAAGCGGTTAGCTC |
| | CTTCGGTCCTCCGATCGTTGTCAGAAGTAAGTTGGCCG |
| | CAGTGTTATCACTCATGGTTATGGCAGCACTGCATAAT |
| | TCTCTTACTGTCATGCCATCCGTAAGATGCTTTTCTGTG |
| | ACTGGTGAGTACTCAACCAAGTCATTCTGAGAATAGTG |
| | TATGCGGCGACCGAGTTGCTCTTGCCCGGCGTCAATAC |
| | GGGATAATACCGCGCCACATAGCAGAACTTTAAAAGT |
| | GCTCATCATTGGAAAACGTTCTTCGGGGCGAAAACTCT |
| | CAAGGATCTTACCGCTGTTGAGATCCAGTTCGATGTAA |
| | CCCACTCGTGCACCCAACTGATCTTCAGCATCTTTTACT |
| | TTCACCAGCGTTTCTGGGTGAGCAAAAACAGGAAGGC |
| | AAAATGCCGCAAAAAAGGGAATAAGGGCGACACGGA |
| | AATGTTGAATACTCATACTCTTCCTTTTTCAATATTATT |
| | GAAGCATTTATCAGGGTTATTGTCTCATGAGCGGATAC |
| | ATATTTGAATGTATTTAGAAAAATAAACAAATAGGGG |
| | TTCCGCGCACATTTCCCCGAAAAGTGCCAGATACCTGA |
| | AACAAAACCCATCGTACGGCCAAGGAAGTCTCCAATA |
| | ACTGTGATCCACCACAAGCGCCAGGGTTTTCCCAGTCA |
| | CGACGTTGTAAAACGACGGCCAGTCATGCATAATCCG |
| | CACGCATCTGGAATAAGGAAGTGCCATTCCGCCTGACC |
| | T |
| pS6651 (SEQ ID NO:33) | GTGTGTATTTCTGGCTGGAACGGGCGTGTTGTTAGAGT |
| | AGGGGAGTGGATTGAGAAGGAGGCTGAGGGGTACTCA |
| | AGGGGGCTATAGAATGTATAGGATTTCCCTGAAGCATT |
| | CCTAGAGAGCCTGCAAGGTGAAGATGGCTTTGGAACC |
| | AGCTGGATCTAGGCTGTGCCACATACTACCTCTTTGGC |
| | CTTGGCCACATCCCTAAACTCTTGGATTCTGTTTCCTAA |
| | GATGTAAGATGGAGGTAATTGTTCCTGCCTCACAGGAG |
| | CTGTTGTGAGGATTAAACAGAGAGTATGTCTTTAGCGC |
| | GGTGCCTGGCACCAGTGCCTGGCATGTAGTAGGGGCA |
| | CAACAAATATAAGGTCCACTTTGCTTTTCTTTTTTCTAT |
| | AGAGAATCCTTTCCTGTTTGCATTGGAAGCCGTGGTTA |
| | TCTCTGTTGGCTCCATGGGATTGATTATCAGCCTTCTCT |
| | GTGTGTATTTCTGGTTAGAGCGAACGATGCCCCGAATT |
| | CCCACCCTGAAGAACCTAGAGGATCTTGTTACTGAATA |
| | CCACGGGAACTTTTCGGCCTGGAGTGGTGTGTCTAAGG |
| | GACTGGCTGAGAGTCTGCAGCCAGACTACAGTGAACG |
| | ACTCTGCCTCGTCAGTGAGATTCCCCCAAAAGGAGGG |
| | GCCCTTGGGGAGGGGCCTGGGGCCTCCCCATGCAACC |
| | AGCATAGCCCCTACTGGGCCCCCCCATGTTACACCCTA |
| | AAGCCTGAAACCTGATAATCTAGATTTATTTGTGAAAT |
| | TTGTGATGCTATTGCTTTATTTGTAACCATCTAGCTTTA |
| | TTTGTGAAATTTGTGATGCTATTGCTTTATTTGTAACCA |
| | TTATAAGCTGCAATAAACAAGTTAACAACAACAATTG |
| | CATTCATTTTATGTTTCAGGTTCAGGGGGAGATGTGGG |
| | AGGTTTTTTAAAGCGGAAACACAGAAAAAAGCCCGCA |
| | CCTGACAGTGCGGGCTTTTTTTTTCGACCAAAGGCTCG |
| | AGATAAGCTTGATATCGAATTCGGAGCACTGTCCTCCG |
| | AACGTCGGAGCACTGTCCTCCGAACGTCGGAGCACTGT |
| | CCTCCGAACGTCGGAGCACTGTCCTCCGAACGGAGCAT |
| | GTCCTCCGAACGTCGGAGCACTGTCCTCCGAACGACTA |
| | GTCTAGAGGGTATATAATGGGGGCCACTAGTCTACTAC |
| | CAGAGTTCATCGCTAGCGCTACCGGATCCGCCACCATG |
| | GCCCTGCCAGTAACGGCTCTGCTGCTGCCACTTGCTCT |
| | GCTCCTCCATGCAGCCAGGCCTGACTACAAAGACGAT |
| | GACGACAAGCAAGTCCAGCTCCAGCAGTCGGGCCCAG |
| | AGTTGGAGAAGCCTGGGGCGAGCGTGAAGATCTCATG |
| | CAAAGCCTCAGGCTACTCCTTTACTGGATACACGATGA |
| | ATTGGGTGAAACAGTCGCATGGAAAGTCACTGGAATG |
| | GATCGGTCTGATTACGCCCTACAACGGCGCCTCCAGCT |
| | ACAACCAGAAGTTCAGGGGAAAGGCGACCCTTACTGT |
| | CGACAAGTCGTCAAGCACCGCCTACATGGACCTCCTGT |
| | CCCTGACCTCCGAAGATAGCGCGGTCTACTTTTGTGCA |
| | CGCGGAGGTTACGATGGACGGGGATTCGACTACTGGG |
| | GCCAGGGAACCACTGTCACCGTGTCGAGCGGAGGCGG |
| | AGGGAGCGGAGGAGGAGGCAGCGGAGGTGGAGGGTC |
| | GGATATCGAACTCACTCAGTCCCCAGCAATCATGTCCG |
| | CTTCACCGGGAGAAAAGGTGACCATGACTTGCTCGGC |
| | CTCCTCGTCCGTGTCATACATGCACTGGTACCAACAAA |
| | AATCGGGGACCTCCCCTAAGAGATGGATCTACGATAC |
| | CAGCAAACTGGCTTCAGGCGTGCCGGGACGCTTCTCGG |
| | GTTCGGGGAGCGGAAATTCGTATTCGTTGACCATTTCG |
| | TCCGTGGAAGCCGAGGACGACGCAACTTATTACTGCC |
| | AACAGTGGTCAGGCTACCCGCTCACTTTCGGAGCCGGC |
| | ACTAAGCTGGAGATCAAGGCGGCAGCAACCACGACGC |
| | CAGCGCCGCGACCACCAACACCGGCGCCTACCATCGC |
| | GTCGCAGCCACTGTCACTGCGCCCAGAAGCGTGCCGG |
| | CCAGCGGCGGGTGGCGCAGTGCACACGAGGGGGCTGG |
| | ACTTCGCCTGTGATATCTACATCTGGGCGCCCTTGGCC |
| | GGGACTTGTGGGGTCCTTCTCCTGTCACTGGTTATCAC |
| | CCTTTACTGCAAACGGGGCAGAAAGAAACTCCTGTAT |
| | ATATTCAAACAACCATTTATGAGACCAGTACAAACTAC |
| | TCAAGAAGAGGACGGCTGTAGCTGCCGATTTCCAGAA |
| | GAAGAAGAAGGAGGATGTGAACTGAGAGTGAAGTTCA |
| | GCAGGAGCGCAGACGCCCCCGCGTACCAGCAGGGCCA |
| | GAACCAGCTCTATAACGAGCTCAATCTAGGACGAAGA |
| | GAGGAGTACGATGTTTTGGACAAGAGGCGTGGCCGGG |
| | ACCCTGAGATGGGGGGAAAGCCGAGAAGGAAGAACC |
| | CTCAGGAAGGCCTGTACAATGAACTGCAGAAAGATAA |
| | GATGGCGGAGGCCTACAGTGAGATTGGGATGAAAGGC |
| | GAGCGCCGGAGGGGCAAGGGGCACGATGGCCTTTACC |
| | AGGGTCTCAGTACAGCCACCAAGGACACCTACGATGC |
| | CTTGCACATGCAAGCCCTGCCCCCTCGCTAACGACTGT |
| | GCCTTCTAGTTGCCAGCCATCTGTTGTTTGCCCCTCCCC |
| | CGTGCCTTCCTTGACCCTGGAAGGTGCCACTCCCACTG |
| | TCCTTTCCTAATAAAATGAGGAAATTGCATCGCATTGT |
| | CTGAGTAGGTGTCATTCTATTCTGGGGGGTGGGGTGGG |
| | GCAGGACAGCAAGGGGGAGGATTGGGAAGACAATAG |
| | CAGGCATGCTGGGGATGCGGTGGGCTCTATGGGATCCT |
| | TGACTTGCGGCCGCAACTCCCACCTGCAACATGCGTGA |
| | CTGACTGAGGCCGCGACTCTAGAGTCGACCGGATCTGC |
| | GATCGCTCCGGTGCCCGTCAGTGGGCAGAGCGCACAT |
| | CGCCCACAGTCCCCGAGAAGTTGGGGGGAGGGGTCGG |
| | CAATTGAACGGGTGCCTAGAGAAGGTGGCGCGGGGTA |
| | AACTGGGAAAGTGATGTCGTGTACTGGCTCCGCCTTTT |
| | TCCCGAGGGTGGGGGAGAACCGTATATAAGTGCAGTA |
| | GTCGCCGTGAACGTTCTTTTTCGCAACGGGTTTGCCGC |
| | CAGAACACAGCTGAAGCTTCGAGGGGCTCGCATCTCTC |
| | CTTCACGCGCCCGCCGCCCTACCTGAGGCCGCCATCCA |
| | CGCCGGTTGAGTCGCGTTCTGCCGCCTCCCGCCTGTGG |
| | TGCCTCCTGAACTGCGTCCGCCGTCTAGGTAAGTTTAA |
| | AGCTCAGGTCGAGACCGGGCCTTTGTCCGGCGCTCCCT |
| | TGGAGCCTACCTAGACTCAGCCGGCTCTCCACGCTTTG |
| | CCTGACCCTGCTTGCTCAACTCTACGTCTTTGTTTCGTT |
| | TTCTGTTCTGCGCCGTTACAGATCCAAGCTGTGACCGG |
| | CGCCTACACCTGCAGCCCAAGCTTACCATGGCCTTACC |
| | AGTGACCGCCTTGCTCCTGCCGCTGGCCTTGCTGCTCC |
| | ACGCCGCCAGGCCTGAACAAAAACTCATTAGCGAAGA |
| | GGATCTCGACATACAGATGACACAGAGCCCTAGCAGT |
| | CTGAGCGCCAGTGTGGGCGATAGAGTTACTATCACTTG |
| | TAGAGCATCCGAGAACATATACAGTTACGTGGCCTGGT |
| | ATCAGCAAAAACCTGGCAAAGCTCCCAAGTTATTGATT |
| | TACAATGCTAAGAGCTTGGCCTCTGGGGTGCCATCGAG |
| | GTTCAGCGGTAGCGGGAGCGGGACCGACTTCACTCTG |
| | ACCATCTCGAGTCTCCAGCCGGAGGACTTTGCGACATA |
| | CTATTGTCAACACCATTACGTATCACCCTGGACCTTCG |
| | GCGGCGGGACTAAGTTAGAGATCAAGGGTGGAGGAGG |
| | ATCAGGCGGCGGTGGATCAGGAGGAGGAGGGTCACAA |
| | GTGCAGTTACAGGAATCAGGGCCCGGCCTGGTGAAGC |
| | CAAGTGAAACCCTGAGTCTGACGTGCACGGTTTCAGG |
| | ATTTAGCCTCACTTCCTACGGTGTCTCTTGGATTCGGCA |
| | GCCAGCCGGCAAAGGGCTCGAGTGGATTGGGGTGATC |
| | TGGGAAGATGGCTCAACAAACTATCATTCTGCACTAAT |
| | CTCTCGCGTGACAATGTCGGTGGACACGTCCAAGAATC |
| | AATTTTCCCTTAAACTGTCCTCCGTGACCGCAGCCGAT |
| | ACAGCGGTATATTATTGCGCGCGACCTCACTACGGATC |
| | TAGCTATGTCGGCGCGATGGAGTATTGGGGCGCTGGC |
| | ACAACCGTCACCGTTTCTTCCGCAACCACGACGCCAGC |
| | GCCGCGACCACCAACACCGGCGCCCACCATCGCTTCCC |
| | AGCCCTTGAGCCTCAGACCCGAGGCCTGCTTCATGTAC |
| | GTCGCGGCGGCCGCCTTTGTCCTTCTCTTCTTCGTCGGC |
| | TGCGGGGTCCTCCTCTCCAAGAGGAAACGGAAGCACA |
| | AGCTGCTGAGCAGCATCGAGCAGGCCTGTGACATCTG |
| | CCGGCTGAAGAAACTGAAGTGCAGCAAAGAAAAGCCC |
| | AAGTGCGCCAAGTGCCTGAAGAACAACTGGGAGTGCC |
| | GGTACAGCCCCAAGACCAAGAGAAGCCCCCTGACCAG |
| | AGCCCACCTGACCGAGGTGGAAAGCCGGCTGGAAAGA |
| | CTGGAACAGCTGTTTCTGCTGATCTTCCCACGCGAGGA |
| | CCTGGACATGATCCTGAAGATGGACAGCCTGCAGGAC |
| | ATCAAGGCCCTGCTGACCGGCCTGTTCGTGCAGGACAA |
| | CGTGAACAAGGACGCCGTGACCGACAGACTGGCCAGC |
| | GTGGAAACCGACATGCCCCTGACCCTGCGGCAGCACA |
| | GAATCAGCGCCACCAGCAGCAGCGAGGAAAGCAGCAA |
| | CAAGGGCCAGCGGCAGCTGACAGTGTCTGCTGCTGCA |
| | GGCGGAAGCGGAGGCTCTGGCGGATCTGATGCCCTGG |
| | ACGACTTCGACCTGGATATGCTGGGCAGCGACGCCCTG |
| | GATGATTTTGATCTGGACATGCTGGGATCTGACGCTCT |
| | GGACGATTTCGATCTCGACATGTTGGGATCAGATGCAC |
| | TGGATGACTTTGACCTGGACATGCTCGGATCATAAAGG |
| | ACGGGTGGCATCCCTGTGACCCCTCCCCAGTGCCTCTC |
| | CTGGCCCTGGAAGTTGCCACTCCAGTGCCCACCAGCCT |
| | TGTCCTAATAAAATTAAGTTGCATCATTTTGTCTGACT |
| | AGGTGTCCTTCTATAATATTATGGGGTGGAGGGGGGTG |
| | GTATGGAGCAAGGGGCAAGTTGGGAAGACAACCTGTA |
| | GGGCCTGCGGGGTCTATTGGGAACCAAGCTGGAGTGC |
| | AGTGGCACAATCTTGGCTCACTGCAATCTCCGCCTCCT |
| | GGGTTCAAGCGATTCTCCTGCCTCAGCCTCCCGAGTTG |
| | TTGGGATTCCAGGCATGCATGACCAGGCTCAGCTAATT |
| | TTTGTTTTTTTGGTAGAAACGGGGTTTCACCATATTGGC |
| | CAGGCTGATCTCCAACTCCTAATCTCAGGTGATCTACC |
| | CACCTTGGCCTCCCAAATTGCTGGGATTACAGGCGTGA |
| | ACCACTGCTCCCTTCCCTGTCCTTCGGATCCGAACGGT |
| | GAGATTTGGAGAAGCCCAGAAAAATGAGGGGAACGGT |
| | AGCTGACAATAGCAGAGGAGGGTTTTGCAGGGTCTTT |
| | AGGAGTAAAGGATGAGACAGTAAGTAATGAGAGATTA |
| | CCCAAGAGGGTTTGGTGATGGAAGGAAGCCACAGGCA |
| | CAGAGAACACAGAATCACTTTATTTCATATGGGACAAC |
| | TGGGAGAAGGGTGATAAAAAAGCTTTAACCTATGTGC |
| | TCCTGCTCCCTCTTTCTCCCCTGTCAGGACGATGCCCCG |
| | AATTCCCACCCTGAAGAACCTAGAGGATCTTGTTACTG |
| | AATACCACGGGAACTTTTCGGTGAGAACGCTGTCATAA |
| | GCATGCTGCAGTCTATCAACTGCCAACTGCCTGCCAGC |
| | AAGACAGACAGAGTGTGGGGGTGGGGGCAGAGAGGA |
| | GAGGGAAGGAGGCCCTGCACTAACTGTCAGGCCGTTC |
| | CAGCCAGAAATACACACATCCCAATGGCGCGCCGAGC |
| | TTGGCTCGAGCATGGTCATAGCTGTTTCCTGTGTGAAA |
| | TTGTTATCCGCTCACAATTCCACACAACATACGAGCCG |
| | GAAGCATAAAGTGTAAAGCCTGGGGTGCCTAATGAGT |
| | GAGCTAACTCACATTAATTGCGTTGCGCTCACTGCCCG |
| | CTTTCCAGTCGGGAAACCTGTCGTGCCAGCTGCATTAA |
| | TGAATCGGCCAACGCGCGGGGAGAGGCGGTTTGCGTA |
| | TTGGGCGCTGTTCCGCTTCCTCGCTCACTGACTCGCTGC |
| | GCTCGGTCGTTCGGCTGCGGCGAGCGGTATCAGCTCAC |
| | TCAAAGGCGGTAATACGGTTATCCACAGAATCAGGGG |
| | ATAACGCAGGAAAGAACATGTGAGCAAAAGGCCAGCA |
| | AAAGGCCAGGAACCGTAAAAAGGCCGCGTTGCTGGCG |
| | TTTTTCCATAGGCTCCGCCCCCCTGACGAGCATCACAA |
| | AAATCGACGCTCAAGTCAGAGGTGGCGAAACCCGACA |
| | GGACTATAAAGATACCAGGCGTTTCCCCCTGGAAGCTC |
| | CCTCGTGCGCTCTCCTGTTCCGACCCTGCCGCTTACCG |
| | GATACCTGTCCGCCTTTCTCCCTTCGGGAAGCGTGGCG |
| | CTTTCTCATAGCTCACGCTGTAGGTATCTCAGTTCGGT |
| | GTAGGTCGTTCGCTCCAAGCTGGGCTGTGTGCACGAAC |
| | CCCCCGTTCAGCCCGACCGCTGCGCCTTATCCGGTAAC |
| | TATCGTCTTGAGTCCAACCCGGTAAGACACGACTTATC |
| | GCCACTGGCAGCAGCCACTGGTAACAGGATTAGCAGA |
| | GCGAGGTATGTAGGCGGTGCTACAGAGTTCTTGAAGT |
| | GGTGGCCTAACTACGGCTACACTAGAAGAACAGTATTT |
| | GGTATCTGCGCTCTGCTGAAGCCAGTTACCTTCGGAAA |
| | AAGAGTTGGTAGCTCTTGATCCGGCAAACAAACCACC |
| | GCTGGTAGCGGTGGTTTTTTTGTTTGCAAGCAGCAGAT |
| | TACGCGCAGAAAAAAAGGATCTCAAGAAGATCCTTTG |
| | ATCTTTTCTACGGGGTCTGACGCTCAGTGGAACGAAAA |
| | CTCACGTTAAGGGATTTTGGTCATGAGATTATCAAAAA |
| | GGATCTTCACCTAGATCCTTTTAAATTAAAAATGAAGT |
| | TTTAAATCAATCTAAAGTATATATGAGTAAACTTGGTC |
| | TGACAGTTAGAAAAACTCATCGAGCATCAAATGAAAC |
| | TGCAATTTATTCATATCAGGATTATCAATACCATATTTT |
| | TGAAAAAGCCGTTTCTGTAATGAAGGAGAAAACTCAC |
| | CGAGGCAGTTCCATAGGATGGCAAGATCCTGGTATCG |
| | GTCTGCGATTCCGACTCGTCCAACATCAATACAACCTA |
| | TTAATTTCCCCTCGTCAAAAATAAGGTTATCAAGTGAG |
| | AAATCACCATGAGTGACGACTGAATCCGGTGAGAATG |
| | GCAAAAGTTTATGCATTTCTTTCCAGACTTGTTCAACA |
| | GGCCAGCCATTACGCTCGTCATCAAAATCACTCGCATC |
| | AACCAAACCGTTATTCATTCGTGATTGCGCCTGAGCGA |
| | AACGAAATACGCGATCGCTGTTAAAAGGACAATTACA |
| | AACAGGAATCGAATGCAACCGGCGCAGGAACACTGCC |
| | AGCGCATCAACAATATTTTCACCTGAATCAGGATATTC |
| | TTCTAATACCTGGAATGCTGTTTTCCCAGGGATCGCAG |
| | TGGTGAGTAACCATGCATCATCAGGAGTACGGATAAA |
| | ATGCTTGATGGTCGGAAGAGGCATAAATTCCGTCAGCC |
| | AGTTTAGTCTGACCATCTCATCTGTAACATCATTGGCA |
| | ACGCTACCTTTGCCATGTTTCAGAAACAACTCTGGCGC |
| | ATCGGGCTTCCCATACAATCGATAGATTGTCGCACCTG |
| | ATTGCCCGACATTATCGCGAGCCCATTTATACCCATAT |
| | AAATCAGCATCCATGTTGGAATTTAATCGCGGCCTAGA |
| | GCAAGACGTTTCCCGTTGAATATGGCTCATACTCTTCC |
| | TTTTTCAATATTATTGAAGCATTTATCAGGGTTATTGTC |
| | TCATGAGCGGATACATATTTGAATGTATTTAGAAAAAT |
| | AAACAAATAGGGGTTCCGCGCACATTTCCCCGAAAAG |
| | TGCCACCTGACGTCTAAGAAACCATTATTATCATGACA |
| | TTAACCTATAAAAATAGGCGTATCACGAGGCCCTTTTG |
| | TCTCGCGCGTTTCGGTGATGACGGTGAAAACCTCTGAC |
| | ACATGCAGCTCCCGGAGACTGTCACAGCTTGTCTGTAA |
| | GCGGATGCCGGGAGCAGACAAGCCCGTCAGGGCGCGT |
| | CAGCGGGTGTTGGCGGGTGTCGGGGCTGGCTTAACTAT |
| | GCGGCATCAGAGCAGATTGTACTGAGAGTGCACCATA |
| | TGCGGTGTGAAATACCGCACAGATGCGTAAGGAGAAA |
| | ATACCGCATCAGGCGCCATTCGCCATTCAGGCTGCGCA |
| | ACTGTTGGGAAGGGCGATCGGTGCGGGCCTCTTCGCTA |
| | TTACGCCAGCTGGCGAAAGGGGGATGTGCTGCAAGGC |
| | GATTAAGTTGGGTAACGCCAGGGTTTTCCCAGTCACGA |
| | CGTTGTAAAACGACGGCCAGTGAATTGACGCGTATTG |
| | GGAT |

### II. Cells

The present disclosure also provides a cell comprising a nucleic acid comprising: a 5′ portion of an endogenous gene of the cell; a 3′ portion of the endogenous gene; an exogenous sequence of equivalent coding potential to the 5′ portion of the endogenous gene or the 3′ portion of the endogenous gene; and an exogenous transgene, wherein the cell expresses each of the endogenous gene, and the exogenous transgene. In some embodiments, a cell disclosed herein is produced by introducing a composition as previous described comprising a gRNA, a targeted nuclease; and a nucleic acid, into the cell.

In some embodiments, a cell disclosed herein comprises a nucleic acid comprising, from 5′ to 3′: (1) a sequence encoding a 5′ portion of an endogenous gene of the cell; (2) a sequence of equivalent coding potential to a 3′ portion of the endogenous gene of the cell; (3) a sequence encoding an exogenous transgene; and (4) a sequence encoding the 3′ portion of the endogenous gene of the cell, and wherein the cell expresses each of (a) the endogenous gene encoded by (1) and (2) and (b) the exogenous transgene encoded by (3).

In other embodiments, a cell disclosed herein comprises a nucleic acid comprising from 5′ to 3′: (a) sequence encoding a 5′ portion of an endogenous gene of the cell; (2) a sequence encoding an exogenous transgene; (3) a sequence of equivalent coding potential to the 5′ portion of the endogenous gene of the cell; and (4) a sequence encoding a 3′ portion of the endogenous gene of the cell, and wherein the cell expresses each of (a) the exogenous transgene encoded by (2) and (b) the endogenous gene encoded by (3) and (4).

In certain embodiments, the sequence of equivalent coding potential to the 3′ portion codes for a carboxy-terminal portion of the protein product of the endogenous gene. In some embodiments, the sequence of equivalent coding potential to the 3′ portion of the endogenous gene comprises all of the coding sequence 3′ of the target cut site. For example, when the sequence of equivalent coding potential to the 3′ portion is contiguous and operably linked with a 5′ portion of the endogenous gene, the cell expresses the protein product encoded by the endogenous gene under the control of the endogenous promoter. In some embodiments, the sequence of equivalent coding potential to the 3′ portion of the endogenous gene comprises a sequence that is identical to the 3′ portion of the endogenous gene located immediately 3′ of a target cut site. In some embodiments, the sequence of equivalent coding potential to the 3′ portion of the endogenous gene comprises a sequence that is not identical to the 3′ portion of the endogenous gene located immediately 3′ of the target cut site and comprises one or more alternative codon(s).

In some embodiments, the length of the sequence of equivalent coding potential to the 3′ portion of the endogenous gene is about 1- 2500 nucleotides in length. For example, the length of the sequence of equivalent coding potential to the 3′ portion of the endogenous gene is about 1-100, 1-200, 1-300, 1-400, 1-500, 1-600, 1-700, 1-800, 1-900, 1-1000, 100-2500, 200-2500, 300-2500, 400-2500, 500-2500, 600-2500, 700-2500, 800-2500, 900-2500, 1000-2500, 1100-2500, 1200-2500, 1300-2500, 1400-2500, 1500-2500, 1600-2500, 1700-2500, 1800-2500, 1900-2500, 2000-2500, 2100-2500, 2200-2500, 2300-2500, 2500-2500, 100-2000, 200-2000, 300-2000, 400-2000, 500-2000, 600-2000, 700-2000, 800-2000, 900-2000, 1000-2000, 1100-2000, 1200-2000, 1300-2000, 1400-2000, 1500-2000, 1600-2000, 1700-2000, 1800-2000, 1900-2000, 100-1500, 200-1500, 300-1500, 400-1500, 500-1500, 600-1500, 700-1500, 800-1500, 900-1500, 1000-1500, 1100-1500, 1200-1500, 1300-1500, 1400-1500, 100-1250, 200-1250, 300-1250, 400-1250, 500-1250, 600-1250, 700-1250, 800-1250, 900-1250, 1000-1250, 1100-1250, 1200-1250, 100-1000, 200-1000, 300-1000, 400-1000, 500-1000, 600-1000, 700-1000, 800-1000, or 900-1000 nucleotides in length.

In some embodiments, the sequence of equivalent coding potential to the 3′ portion is about 80%, 85%, 90%, 91%, 92%, 93%, 94%, 95%, 96%, 97%, 98%, 99%, or 100% identical to the endogenous gene over the length of the 3′ portion.

In some embodiments, the sequence of equivalent coding potential to the 3′ portion of the endogenous gene can be a 3′ portion of TRAC, TRBC, CD3γ chain, CD3δ chain, CD3_{ε} chain, CD3ξ chain, IL-2Rα chain, IL-2Rβ chain, or IL-2Rγ chain (IL2RG). For example, the sequence of equivalent coding potential to the 3′ portion can have a nucleotide sequence having about 80%, 85%, 90%, 91%, 92%, 93%, 94%, 95%, 96%, 97%, 98%, 99%, or 100% sequence identity to a 3′ portion of any of the sequences described in Table 1.

In some embodiments, the sequence of equivalent coding potential to the 3′ portion of the endogenous gene can be a 3′ portion of Actb, Atp5f1, B2m, Gapdh, Gusb, Hprt, Pgk1, Ppia, Rps18, Tbp, Tfrc, Ywhaz, Nanog, Rex1, or Oct4. For example, the sequence of equivalent coding potential to the 3′ portion can have a nucleotide sequence having about 80%, 85%, 90%, 91%, 92%, 93%, 94%, 95%, 96%, 97%, 98%, 99%, or 100% sequence identity to a 3′ portion of any of the sequences described in Table 2.

In certain embodiments, the sequence of equivalent coding potential to a 5′ portion codes for an amino-terminal portion of the protein product of the endogenous gene. In some embodiments, the sequence of equivalent coding potential to the 5′ portion of the endogenous gene comprises all of the coding sequence 5′ of the target cut site. For example, when the sequence of equivalent coding potential to the 5′ portion is contiguous and operably linked with a 3′ portion of the endogenous gene, the cell expresses the protein product encoded by the endogenous gene under the control of the endogenous promoter. In other embodiments, expression of the protein product of the endogenous gene is under the regulation of an exogenously introduced promoter. In some embodiments, the sequence of equivalent coding potential to the 5′ portion of the endogenous gene comprises a sequence that is identical to the 5′ portion of the endogenous gene located immediately 5′ of the target cut site. In some embodiments, the sequence of equivalent coding potential to the 5′ portion of the endogenous gene comprises a sequence that is not identical to the 5′ portion of the endogenous gene located immediately 5′ of the target cut site and comprises one or more alternative codon(s).

In some embodiments, the length of the sequence of equivalent coding potential to the 5′ portion of the endogenous gene is about 1- 2500 nucleotides in length. For example, the length of the sequence of equivalent coding potential to the 5′ portion of the endogenous gene is about 1-100, 1-200, 1-300, 1-400, 1-500, 1-600, 1-700, 1-800, 1-900, 1-1000, 100-2500, 200-2500, 300-2500, 400-2500, 500-2500, 600-2500, 700-2500, 800-2500, 900-2500, 1000-2500, 1100-2500, 1200-2500, 1300-2500, 1400-2500, 1500-2500, 1600-2500, 1700-2500, 1800-2500, 1900-2500, 2000-2500, 2100-2500, 2200-2500, 2300-2500, 2500-2500, 100-2000, 200-2000, 300-2000, 400-2000, 500-2000, 600-2000, 700-2000, 800-2000, 900-2000, 1000-2000, 1100-2000, 1200-2000, 1300-2000, 1400-2000, 1500-2000, 1600-2000, 1700-2000, 1800-2000, 1900-2000, 100-1500, 200-1500, 300-1500, 400-1500, 500-1500, 600-1500, 700-1500, 800-1500, 900-1500, 1000-1500, 1100-1500, 1200-1500, 1300-1500, 1400-1500, 100-1250, 200-1250, 300-1250, 400-1250, 500-1250, 600-1250, 700-1250, 800-1250, 900-1250, 1000-1250, 1100-1250, 1200-1250, 100-1000, 200-1000, 300-1000, 400-1000, 500-1000, 600-1000, 700-1000, 800-1000, or 900-1000 nucleotides in length.

In some embodiments, the sequence of equivalent coding potential to the 5′ portion is about 80%, 85%, 90%, 91%, 92%, 93%, 94%, 95%, 96%, 97%, 98%, 99%, or 100% identical to the endogenous gene over the length of the 5′ portion.

In some embodiments, the sequence of equivalent coding potential to the 5′ portion of the endogenous gene can be a 5′ portion of TRAC, TRBC, CD3γ chain, CD3δ chain, CD3_{ε} chain, CD3ξ chain, IL-2Rα chain, IL-2Rβ chain, or IL-2Rγ chain (IL2RG). For example, the sequence of equivalent coding potential to the 5′ portion can have a nucleotide sequence having about 80%, 85%, 90%, 91%, 92%, 93%, 94%, 95%, 96%, 97%, 98%, 99%, or 100% sequence identity to a 5′ portion of any of the sequences described in Table 1.

In some embodiments, the sequence of equivalent coding potential to the 3′ portion of the endogenous gene can be a 5′ portion of Actb, Atp5f1, B2m, Gapdh, Gusb, Hprt, Pgk1, Ppia, Rps18, Tbp, Tfrc, Ywhaz, Nanog, Rex1, or Oct4. For example, the sequence of equivalent coding potential to the 5′ portion can have a nucleotide sequence having about 80%, 85%, 90%, 91%, 92%, 93%, 94%, 95%, 96%, 97%, 98%, 99%, or 100% sequence identity to a 3′ portion of any of the sequences described in Table 2.

A cell disclosed herein further comprises an exogenous transgene. In some embodiments, expression of the exogenous transgene is under the control of an endogenous promoter. In other embodiments, the expression of the exogenous transgene is under the regulation of an exogenously introduced and operably linked promoter.

In some embodiments, the exogenous transgene comprises a sequence encoding one or more polypeptide that is expressed in the cell. For example, in some embodiments, the exogenous transgene comprises a sequence encoding one or more protein expressed on the surface of the cell membrane. In some embodiments, the exogenous transgene comprises a sequence encoding a transmembrane protein, or fragment thereof. For example, in some embodiments, the exogenous transgene comprises one or more sequence encoding CD28, CD45, CD2, CD4, CD5, CD7, CD8, CD9, CD16, CD22, CD27, CD28, CD30, CD33, CD37, CD40, CD64, CD80, CD83, CD86, CD127, CD134, CD137, CD154, CIITA, 4-1BBL. PD-1, PD-1L, LIGHT, DAP10, DAP12, ICAM-1, LFA-1, LCK, TNFR2, ICOS, NKG2C, HLA-E, B7-H3, or beta 2-microglobulin. In some embodiments, the exogenous transgene comprises a sequence encoding a cell surface marker that can be used as a selection marker for cells having successful transgene insertion into the genome of the cell. For example, in some embodiments the exogenous transgene comprises a sequence encoding an epidermal growth factor receptor (EGFR), or truncated fragment thereof, which can be readily detected using an anti-EGFR antibody and flow cytometry.

In some embodiments, the exogenous transgene comprises a sequence encoding a synthetic antigen receptor, wherein the synthetic antigen receptor is a chimeric antigen receptor (CAR) or a SynNotch receptor. See, for example, Sadelain et al., Cancer Discov. 3(4): 388-398 (2013)); Srivastava Trends Immunol. 36(8): 494-502 (2015)); Toda et al. Science 361(6398): 156-162 (2018); and Cho et al. Scientific Reports 8: 3846 (2018) regarding CAR and SynNotch design and uses). In certain embodiments the exogenous transgene comprises a sequence encoding a chimeric antigen receptor (CAR). In some embodiments, the exogenous transgene comprises a CAR specifically recognizing cancer cell-associated targets such as CD19, BCMA, CD20, CD22, CD30, CD33, CD123, CD133, CEA, EGFR, EGFRvIII, EphA2, ErbB family, GPC3, HER2, FAP, FRα, FD2, Igχ, IL-13α2, Mesothelin, Muc1, PSMA, ROR1, VEGFR2, B7-H3, B7H6, CD5, CD23, CD70, CSPG4, EpCAM, GD3, HLA-A1+MAGE, IL-11Rα, Lewis-Y, Muc16, NKG2D ligands, PSCA, or TAG72. For example, in some embodiments, the exogenous transgene comprises a sequence encoding a CD19-CD28-CD3ξ CAR, a CD19-4-1BB-CD3ξ CAR, a MSLN-CD28-CD3ξ CAR, or a MSLN-4-1BB-CD3ξ CAR.

In some embodiments, the exogenous transgene encodes one or more protein that alters the functionality of the cell. For example, in the case of an exogenous transgene encoding a CAR inserted into the genome of a T-cell, the expression of the CAR can alter the specificity and functionality of the T-cell.

In other embodiments, the exogenous transgene encodes one or more cytoplasmic protein, intracellular protein, or soluble protein. In some embodiments, the exogenous transgene encodes a therapeutic protein. In some embodiments, the exogenous transgene encodes a cytokine or a functional fragment thereof. In some embodiments, the exogenous transgene encodes a transcription factor. In some embodiments, the exogenous transgene encodes an immune checkpoint inhibitor.

In other embodiments, exogenous transgenes can comprise sequences encoding non-translated RNA, such as rRNA, tRNA, gRNA, siRNA, or miRNA.

In some embodiments, a cell described herein is a mammalian cell. For example, in some embodiments, the mammalian cell is a human cell. In some embodiments, the human cells are pluripotent stem cells or induced pluripotent stem cells (iPSCs). In some embodiments, the human cells are T-cells, B-cells, natural killer (NK) cells, myeloid cells, macrophages, dendritic cells, hematopoietic stem cells, or other immune cells.. In some embodiments, the T-cells are regulatory T-cells, effector T-cells or naive T-cells. In some embodiments, the effector T-cells are CD8+ T-cells or CD4+ T-cells. In some embodiments, the effector T-cells are CD8+ CD4+ T cells. In some embodiments, the T-cell is a T-cell that expresses a TCR receptor or differentiates into a T-cell that expresses a TCR receptor. In some embodiments, the human cells are iPSC-derived NK cells. In some embodiments, the cells are primary cells. In some embodiments the cell is obtained from a subject. For example, in some embodiments the cell is obtained from a subject and modified ex vivo by introducing a composition as described herein.

### III. Methods of Genome Editing

Also disclosed herein are methods of editing the genome of a cell comprising introducing into the cell a composition for the targeted insertion of a nucleic acid comprising a sequence coding for a 3′ portion or a 5′ portion of an endogenous gene of a cell and an exogenous transgene. In some embodiments, a method of editing the genome of a cell comprises introducing a composition into the cell that comprises: (A) a guide RNA (gRNA); (B) a targeted nuclease; and (C) a nucleic acid (e.g. template for DNA repair). In other embodiments, a method of editing the genome of a cell comprises introducing a composition into the cell that comprising: (A) a targeted nuclease; and (B) a nucleic acid (e.g., template for DNA repair).

In some embodiments, a method of editing the genome of a cell disclosed herein comprises: introducing into the cell a gRNA targeting an endogenous gene in the cell, an RNA guided nuclease complexed with the gRNA, and a nucleic acid complexed with the RNA guided nuclease and comprising one or more region(s) of homology to the endogenous gene, a sequence of equivalent coding potential to a 3′ portion of the endogenous gene, and an exogenous transgene. In some embodiments, the RNA-guided nuclease specifically cleaves the endogenous gene in the cell to create an insertion site into which the sequence of equivalent coding potential to the 3′ portion of the endogenous gene and the exogenous transgene are inserted resulting in the restored or continued expression of the endogenous gene and the expression of the exogenous transgene in the cell.

In other embodiments, a method of editing the genome of a cell disclosed herein comprises: introducing into the cell a gRNA targeting an endogenous gene in the cell, an RNA guided nuclease complexed with the gRNA, and a nucleic acid complexed with the RNA guided nuclease and comprising one or more region(s) of homology to the endogenous gene, an exogenous transgene, and a sequence of equivalent coding potential to the 5′ portion of the endogenous gene. In some embodiments, the RNA-guided nuclease specifically cleaves the endogenous gene in the cell to create an insertion site into which the exogenous transgene and the sequence of equivalent coding potential to the 5′ portion of the endogenous gene are inserted resulting in the restored or continued expression of the endogenous gene and the expression of the exogenous transgene in the cell.

In some embodiments, the gRNA, RNA-guided nuclease, and nucleic acid are introduced into the cell via non-viral delivery. For example, in some embodiments, the gRNA, RNA-guided nuclease, and nucleic acid are introduced into the cell via electroporation. In some embodiments, the gRNA, RNA-guided nuclease, and/or nucleic acid are introduced into the cell via viral delivery. For example, in some embodiments, the gRNA, RNA-guided nuclease, and/or nucleic acid are introduced into the cell via viral transduction (e.g., a retrovirus, adenovirus, lentivirus, or adeno-associated virus). In some embodiments, the gRNA, RNA-guided nuclease, and/or nucleic acid are introduced into the cell via an adeno-associated virus (e.g., AAV1, AAV2, AAV3, AAV4, AAV5, AAV6, AAV7, AAV8, AAV9, AAV10, AAV11, AAV12, or AAV13).

For example, in some embodiments, the gRNA, targeted nuclease (e.g., RNA-guided nuclease), and nucleic acid sequence are introduced into the cell as a ribonucleoprotein complex (RNP)-DNA complex, wherein the RNP-DNA complex comprises:(i) the RNP, wherein the RNP comprises the the RNA-guided nuclease (e.g., Cas9) and the gRNA; and (ii) the nucleic acid that functions as a DNA template.

In some embodiments, the molar ratio of RNP to nucleic acid can be from about 3:1 to about 100:1. For example, the molar ratio can be from about 5:1 to 10:1, from about 5:1 to about 15:1, 5:1 to about 20:1; 5:1 to about 25:1; from about 8:1 to about 12:1; from about 8:1 to about 15:1, from about 8:1 to about 20:1, or from about 8:1 to about 25:1.

In some embodiments, the nucleic acid in the RNP-DNA template complex is at a concentration of about 2.5 pM to about 25 pM. In some embodiments, the amount of nucleic acid is about 1 µg to about 10 µg.

In some embodiments, the RNP-DNA complex is formed by incubating the RNP with the nucleic acid for less than about one minute to about thirty minutes, at a temperature of about 20° C. to about 25° C. In some embodiments, the RNP-DNA complex and the cell are mixed prior to introducing the RNP-DNA complex into the cell.

In some embodiments the nucleic acid sequence or the RNP-DNA complex is introduced into the cells by electroporation. Methods, compositions, and devices for electroporating cells to introduce a RNP-DNA complex can include those described in the examples herein. Additional or alternative methods, compositions, and devices for electroporating cells to introduce a RNP-DNA complex can include those described in WO/2006/001614 or Kim, J.A. et al. Biosens. Bioelectron. 23, 1353-1360 (2008). Additional or alternative methods, compositions, and devices for electroporating cells to introduce a RNP-DNA complex can include those described in U.S. Pat. Appl. Pub. Nos. 2006/0094095; 2005/0064596; or 2006/0087522. Additional or alternative methods, compositions, and devices for electroporating cells to introduce a RNP-DNA complex can include those described in Li, L.H. et al. Cancer Res. Treat. 1, 341-350 (2002); U.S. Pat. Nos.: 6,773,669; 7,186,559; 7,771,984; 7,991,559; 6485961; 7029916; and U.S. Pat. Appl. Pub. Nos: 2014/0017213; and 2012/0088842. Additional or alternative methods, compositions, and devices for electroporating cells to introduce a RNP-DNA complex can include those described in Geng, T. et al.. J. Control Release 144, 91-100 (2010); and Wang, J., et al. Lab. Chip 10, 2057-2061 (2010). In some embodiments, the RNP is delivered to the cells in the presence of an anionic polymer. In some embodiments, the anionic polymer is an anionic polypeptide or an anionic polysaccharide. In some embodiments, the anionic polymer is an anionic polypeptide (e.g., a polyglutamic acid (PGA), a polyaspartic acid, or polycarboxyglutamic acid). In some embodiments, the anionic polymer is an anionic polysaccharide (e.g., hyaluronic acid (HA), heparin, heparin sulfate, or glycosaminoglycan). In some embodiments, the anionic polymer is poly(acrylic acid) (PAA), poly(methacrylic acid) (PMAA), poly(styrene sulfonate), or polyphosphate. In some embodiments, the anionic polymer has a molecular weight of at least 15 kDa (e.g., between 15 kDa and 50 kDa). In some embodiments, the anionic polymer and the RNA-guided nuclease are in a molar ratio of between 10:1 and 120:1, respectively (e.g., 10:1, 20:1, 30:1, 40:1, 50:1, 60:1, 70:1, 80:1, 90:1, 100:1, 110:1, or, 120:1). In some embodiments, the molar ratio of gRNA:RNA-guided nuclease is between 0.25:1 and 4:1 (e.g., 0.25:1, 0.5:1, 1:1, 1.2:1, 1.4:1, 1.6:1, 1.8:1, 2:1, 2.2:1, 2.4:1, 2.6:1, 2.8:1, 3:1, 3.2:1, 3.4:1, 3.6:1, 3.8:1, or 4:1).

In some embodiments, the nucleic acid or RNP-DNA complex are introduced into about 1 × 10⁵ to about 100 × 10⁶ cells (e.g.,T-cells). For example, the nucleic acid or RNP-DNA complex can be introduced into about 1 × 10⁵ cells to about 5 × 10⁵ cells, about 1 × 10⁵ cells to about 1 × 10⁶ cells, 1 × 10⁵ cells to about 1.5 × 10⁶ cells, 1 × 10⁵ cells to about 2 × 10⁶ cells, about 1 × 10⁶ cells to about 1.5 × 10⁶ cells or about 1 × 10⁶ cells to about 2 × 10⁶ cells.

In some embodiments of a method disclosed herein, upon introduction into the cell, the RNP-DNA complex translocates to the locus of the endogenous gene in the cell, where the targeted nuclease (e.g., RNA-guided nuclease 9) is guided by the DNA-targeting sequence of the gRNA and introduces a double stranded break in the genomic DNA at a target cut site. In certain embodiments, one or more region(s) of homology to the endogenous gene of the cell align(s) the nucleic acid to the endogenous gene of the cell and, via HDR, a sequence of equivalent coding potential to a 3′ portion of the endogenous gene in the cell that codes for a carboxy-terminal portion of the protein product of the endogenous gene and an exogenous transgene are inserted into the target cut site within the endogenous gene. In some embodiments, the inserted sequence of equivalent coding potential to the 3′ portion forms a contiguous open reading frame with the 5′ portion of the endogenous gene located immediately 5′ of the target cut site and allows restored or continued expression of the protein product encoded by the endogenous gene and under the control of the endogenous promoter. In some embodiments, insertion of the exogenous transgene results in expression of a protein product encoded by the transgene (e.g., a CAR). In some embodiments, expression of the exogenous transgene in the cell is under the control of an endogenous promoter. In some embodiments, an exogenous promoter is operably linked with the exogenous transgene and is inserted into the target cut site with the exogenous transgene to drive expression of the transgene in the cell.

In other embodiments of a method disclosed herein, upon introduction into the cell, the RNP-DNA complex translocates to the locus of the endogenous gene in the cell, where the targeted nuclease (e.g., RNA-guided nuclease) is guided by the DNA-targeting sequence of the gRNA and introduces a double stranded break in the genomic DNA at a target cut site. In certain embodiments, one or more region(s) of homology to the endogenous gene of the cell align(s) the nucleic acid to the endogenous gene of the cell and, via HDR, an exogenous transgene and a sequence of equivalent coding potential to the 5′ portion of the endogenous gene in the cell that codes for an amino-terminal portion of the protein product of the endogenous gene are inserted into the target cut site within the endogenous gene. In some embodiments, the inserted sequence of equivalent coding potential to the 5′ portion forms a contiguous open reading frame with the 3′ portion of the endogenous gene located immediately 3′ of the target cut site and allows restored or continued expression of the protein product encoded by the endogenous gene. In some embodiments, insertion of the exogenous transgene results in expression of a protein product encoded by the transgene (e.g., a CAR). In some embodiments, expression of the exogenous transgene is under the control of the endogenous promoter of the endogenous gene in the cell. In other embodiments, an exogenous promoter is operably linked with the exogenous transgene and is inserted into the target cut site with the exogenous transgene to drive expression of the transgene in the cell. In some embodiments, expression of the endogenous gene in the cell is under the control of an endogenous promoter. In other embodiments, an exogenous promoter is operably linked with the sequence of equivalent coding potential to the 5′ portion of the endogenous gene and is inserted into the target cut site with the sequence of equivalent coding potential to the 5′ portion of the endogenous gene to drive expression of the endogenous gene in the cell.

In some embodiments, a method of editing the genome of a cell comprises introducing a composition disclosed herein into a mammalian cell. For example, in some embodiments, the mammalian cell is a human cell, e.g. an immune cell. In certain embodiments, the immune cell is a T-cell, e.g., a CD4+ or a CD8+ T-cell. In some embodiments, the method of editing the genome of a cell comprises inserting an exogenous transgene into the genomic locus of TRAC, TRBC, CD3γ chain, CD3δ chain, CD3ε chain. IL-2Rα chain, IL-2Rβ chain, or IL-2Rγ chain (IL2RG). For example, in certain embodiments, the exogenous transgene is inserted into a target cut site within TRAC. In some embodiments, the method of editing the genome of the cell comprises restoring or continuing the expression of an endogenous gene whose expression is interrupted by the insertion of the exogenous transgene. For example, methods disclosed herein can restore or continue the expression of TRAC, TRBC, CD3γ chain, CD3δ chain, CD3ε chain. IL-2Rα chain, IL-2Rβ chain, or IL-2Rγ chain.

In some embodiments, the method of editing the genome of a cell comprises inserting an exogenous transgene into the genomic locus of at least one of Actb, Atp5f1, B2m, Gapdh, Gusb, Hprt, Pgk1, Ppia, Rps18, Tbp, Tfrc, Ywhaz, Nanog, Rex1, or Oct4. In some embodiments, the method of editing the genome of the cell comprises restoring or continuing the expression of an endogenous gene whose expression is interrupted by the insertion of the exogenous transgene. For example, methods disclosed herein can restore or continue the expression of Actb, Atp5f1, B2m, Gapdh, Gusb, Hprt, Pgk1, Ppia, Rps18, Tbp, Tfrc, Ywhaz, Nanog, Rex1, or Oct4.

In alternative embodiments, a method of editing the genome of a cell disclosed herein comprises: introducing into the cell a targeted nuclease selected from a TALEN, ZFN, or megaTAL, and a nucleic acid complexed with the targeted nuclease and comprising one or more region(s) of homology to the endogenous gene, a sequence of equivalent coding potential to a 3′ portion of the endogenous gene, and an exogenous transgene. In some embodiments, the targeted nuclease specifically cleaves the endogenous gene in the cell to create an insertion site into which the sequence of equivalent coding potential to the 3′ portion of the endogenous gene and the exogenous transgene are inserted resulting in the restored or continued expression of the endogenous gene and the expression of the exogenous transgene in the cell.

In yet another embodiment, a method of editing the genome of a cell disclosed herein comprises: introducing into the cell a targeted nuclease selected from a TALEN, ZFN, or megaTAL, and a nucleic acid complexed with the targeted nuclease and comprising one or more region(s) of homology to the endogenous gene, an exogenous transgene, and a sequence of equivalent coding potential to the 5′ portion of the endogenous gene. In some embodiments, the targeted nuclease specifically cleaves the endogenous gene in the cell to create an insertion site into which the exogenous transgene and the sequence of equivalent coding potential to the 5′ portion of the endogenous gene are inserted resulting in the restored or continued expression of the endogenous gene and the expression of the exogenous transgene in the cell.

### IV. Methods of Treatment

Also provided in this disclosure are methods of treating or preventing a disease in a subject comprising editing the genome of a cell by a method as disclosed herein and/or administering a cell as disclosed herein to the subject.

For example, in some embodiments, a method of treating or preventing a disease in a subject comprises: obtaining a cell comprising a nucleic acid comprising: a 5′ portion of an endogenous gene of the cell; a 3′ portion of the endogenous gene; a sequence of equivalent coding potential to the 5′ portion or 3′ portion of the endogenous gene; and an exogenous transgene, wherein the cell expresses each of the endogenous gene and the exogenous transgene, and administering the cell to the subject.

In some embodiments, the methods and compositions described herein can be used to edit the genome of immune cells, e.g., T-cells. In some embodiments, the immune cells (e.g., T-cells) are obtained from the subject having the disease or at risk of having the disease. For example, in some embodiments, immune cells (e.g., T-cells) having edited genomes using the methods and compositions described herein can be administered to the subject to treat or prevent a disease such as cancer, an infectious disease, an autoimmune disease, transplantation rejection, graft vs. host disease, or other inflammatory disorder in the subject. In some embodiments, expression of the exogenous transgene alters the specificity and/or functionality of the cell such that the cell treats and or prevents the disease in the subject. For example, in some embodiments, a T-cell (e.g., a CD4+ or CD8+ T-cell) is obtained from the subject and its genome edited to express a CAR, and wherein the T-cell expressing the CAR is administered to the subject for the treatment of a cancer. In certain examples, a method disclosed herein is for the treatment or prevention of a cancer in a subject and the CAR recognizes a cancer-specific antigen (e.g. a tumor specific antigen or neoantigen). In certain examples, a method disclosed herein is for the treatment or prevention of an autoimmune disease in a subject and the CAR recognizes an antigen associated with the autoimmune disorder.

In certain embodiments, a method disclosed herein can be used for the treatment or prevention of a cancer in a subject wherein the cancer is bladder cancer, breast cancer, cervical cancer, colorectal cancer, esophageal cancer, gastric cancer, head and neck cancer, hepatocellular cancer, leukemia, lung cancer, lymphoma, mesothelioma, melanoma, myeloma, ovarian cancer, endometrial cancer, prostate cancer, pancreatic cancer, renal cell cancer, non-small cell lung cancer, small cell lung cancer, brain cancer, sarcoma, neuroblastoma, or squamous cell carcinoma of the head and neck.

In some embodiments a method disclosed herein can be used for the treatment or prevention of an autoimmune disease in a subject. In certain embodiments, the autoimmune disorder is selected from the group consisting of multiple sclerosis, diabetes mellitus Type I, rheumatoid arthritis, systemic lupus erythematosus, inflammatory bowel disease, celiac disease, Graves’ disease, Hashimoto’s autoimmune thyroiditis, vitiligo, rheumatic fever, pernicious anemia/atrophic gastritis, alopecia areata, immune thrombocytopenic purpura, temporal arteritis, ulcerative colitis, Crohn’s disease, scleroderma, antiphospholipid syndrome, autoimmune hepatitis type 1, primary biliary cirrhosis, Sjogren’s syndrome, Addison’s disease, dermatitis herpetiformis, Kawasaki disease, sympathetic ophthalmia, HLA-B27 associated acute anterior uveitis, primary sclerosing cholangitis, discoid lupus erythematosus, polyarteritis nodosa, CREST Syndrome, myasthenia gravis, polymyositis/dermatomyositis, Still’s disease, autoimmune hepatitis type 2, Wegener’s granulomatosis, mixed Connective tissue disease, microscopic polyangiitis, autoimmune polyglandular syndrome, Felty’s syndrome, autoimmune hemolytic anemia, chronic inflammatory demyelinating polyneuropathy, Guillain-Barre Syndrome, Behcet disease, autoimmune neutropenia, bullous pemphigoid, essential mixed cryoglobulinemia, linear morphea, autoimmune polyglandular syndrome 1 (APECED), acquired hemophilia A, Batten disease/neuronal ceroid lipofuscinoses, autoimmune pancreatitis, Hashimoto’s encephalopathy, Goodpasture’s disease, pemphigus vulgaris, autoimmune disseminated encephalomyelitis, relapsing polychondritis, Takayasu arteritis, Churg-Strauss syndrome, epidermolysis bullosa acquisita, cicatricial pemphigoid, pemphigus foliaceus, autoimmune hypoparathyroidism, autoimmune hypophysitis, autoimmune inner ear disease, autoimmune lymphoproliferative syndrome, autoimmune oophoritis, autoimmune orchitis, autoimmune polyglandular syndrome, Cogan’s syndrome, encephalitis lethartica, erythema elevatum diutinum, Evans syndrome, immunodysregulation polyendocrinopathy enteropathy X-linked (IPEX), Issac’s syndrome/acquired neuromyotonia, Miller Fisher syndrome, Morvan’s syndrome, PANDAS, POEMS syndrome, Rasmussen’s encephalitis, stiff-person syndrome, Vogt-Koyanagi-Harada syndrome, neuromyelitis optica, graft vs host disease, and autoimmune uveitis.

In some embodiments, cells are obtained from a subject, the genomes of the cells are edited to express an exogenous transgene and endogenous gene, and expanded ex vivo prior to administration to the subject for the treatment or prevention of the disease. For example, in some embodiments, tumor infiltrating lymphocytes, a heterogeneous and cancer-specific T-cell population, are obtained from a cancer subject and expanded ex vivo. In certain embodiments, the characteristics of the subject’s cancer determine a set of tailored cellular modifications (e.g. the exogenous transgene to be inserted into the cell), and these modifications are applied to the tumor infiltrating lymphocytes using any of the methods described herein.

The description above describes multiple aspects and embodiments of the invention. The patent application specifically contemplates all combinations and permutations of the aspects and embodiments.

### EXAMPLES

The invention now being generally described, will be more readily understood by reference to the following examples, which are included merely for purposes of illustration of certain aspects and embodiments of the present invention, and is not intended to limit the invention.

### EXAMPLE 1 - CAR Transgene Insertion Into TRAC

Described herein is a non-viral genome editing method of inserting an exogenous transgene (e.g., encoding a CAR) into a targeted site within the TRAC gene of a T-cell. Cells having successful insertion of the exogenous transgene and sequence of equivalent coding potential to the 3′ portion of TRAC express both the exogenously introduced CAR and a functional TCR complex resulting from the restored or continued expression of the TCRα chain.

### T-Cell Isolation and Activation

T-cells were enriched from peripheral blood mononuclear cells (PBMCs) prepared using Lymphoprep (STEMCELL Technologies) from normal donor Leukopaks (STEMCELL Technologies) using the EasySep Human T-Cell Isolation Kit (STEMCELL Technologies). T-cells were subsequently activated with T-Cell TransAct, human (Miltenyi, 130-111-160) in TexMACS medium (Miltenyi 130-197-196) supplemented with 3% human AB serum (Gemini Bio) and 12.5 ng/ml human IL-7 and IL-15 (Miltenyi premium grade) and grown at 37° C., 5% CO₂ for 48 hours before electroporation.

### T-Cell Gene Editing

CRISPR RNP were prepared by combining 120 µM sgRNA (Synthego) targeting DNA sequence AAGTCTCTCAGCTGGTACA (SEQ ID NO:1), 62.5 µM sNLS-SpCas9-sNLS (Aldevron), 100 ng/ml poly-L-glutamic acid (Sigma P4761-25MG) and P3 buffer (Lonza) at a ratio of 5:1:3:6. 5 µg of plasmid DNA (i.e. plasmids having sequences according to SEQ ID NO:11, SEQ ID NO:12, or SEQ ID NO:13) was mixed with 17.5 µl of RNP. T-cells were counted, centrifuged at 90 X G for 10 minutes and resuspended at 5 × 10⁶ cells/94 µl of P3 with supplement added (Lonza). 94 µl of T-cell suspension was added to the DNA/RNP mixture, transferred to a Lonza electroporation cuvette, and pulsed in a Lonza X-unit with code EH-115. Cells were allowed to rest for 10 minutes at room temperature before transfer to 24-well G-Rex plates (Wilson Worf) in TexMACS medium supplemented with 12.5 ng/ml human IL-7 and IL-15 (Miltenyi premium grade). For some conditions, cells were recovered with a 1:1 ratio of CTS Dynabeads (CD3/CD28) (Thermo Fisher) mixed into the aforementioned medium formulation.

### Flow Cytometry

Transgene expression was detected by staining with anti-EGFR antibody (BioLegend clone AY13) and analysis on an Attune NxT Flow Cytometer. TCR alpha/beta complex expression was detected with CD3E antibody (BD clone UCHT1) and TCRalpha/beta antibody (BioLegend clone IP26).

### Genomic Editing of T-Cells to Express CAR and TCRα Chain

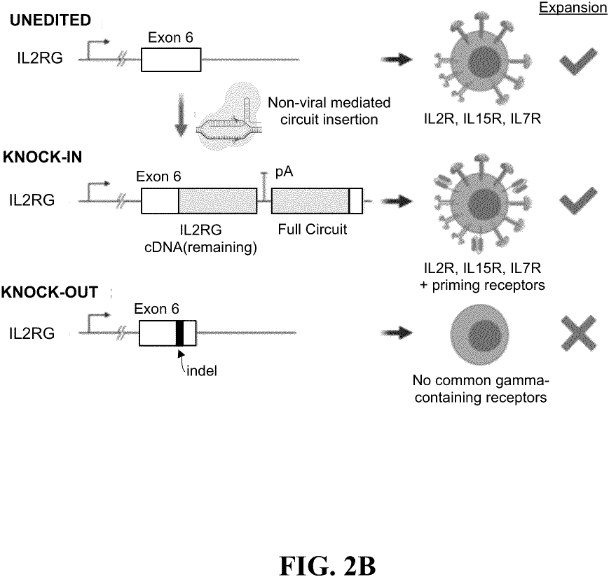

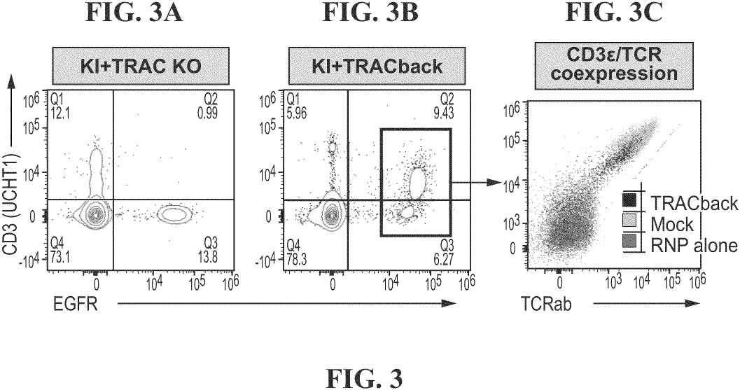

T-cells were genomically edited via electroporation of CRISPR RNP targeting the TRAC locus with a plasmid repair template to express an exogenous transgene encoding CD19-4-1BB-CD3ξ-CAR 2A-linked to a truncated EGFR surface marker gene. As shown in FIG. 3A, the specific target cut site in the TRAC locus disrupts the coding sequence of TRAC, such that cells electroporated with a plasmid having a sequence according to SEQ ID NO:11 and expressing the exogenous CAR transgene, no longer express TCRα chain protein, evidenced by a loss of TCR complex surface expression as indicated by the absence of CD3ε and TCRα/β (data not shown) detection by flow cytometry. FIGS. 3A and 3B show that the exogenous transgene is readily detected in electroporated cells stained with EGFR antibody and analyzed by flow cytometry. As shown in FIGS. 3B and 3C, cells electroporated with plasmid having a sequence according to SEQ ID NO:12, wherein the plasmid repair template includes the 3′ coding sequence that comes after the CRISPR target cut site in TRAC, along with a 2A sequence to yield co-translation of the CAR and EGFR transgenes, results in a TRAC locus with a full TCRα chain coding sequence in addition to the transgene. These cells have detectable TCR complex expression on the cell surface as indicated by the presence of CD3 (FIG. 3B) and TCRα/β (FIG. 3C). As shown in FIG. 4, T-cells electroporated with plasmids comprising the 3′ coding sequence that comes after the CRISPR target cut site in TRAC (i.e. plasmids having the sequences according to SEQ ID NO:12 and SEQ ID NO:13), have TCR complex expression and respond to TCR stimulation with CD3/CD28 Dynabeads as compared to T-cells electroporated with a plasmid lacking the 3′ coding sequence that comes after the CRISPR target cut site in TRAC (i.e., a plasmid having the sequence according to SEQ ID NO:11).

### EXAMPLE 2 - CAR Transgene Insertion Into IL2RG

Described herein is a non-viral genome editing method of inserting an exogenous gene circuit (e.g., encoding a CAR) into a targeted site within the IL2RG gene of a T-cell. Cells having successful insertion of the exogenous transgene and sequence of equivalent coding potential to the 3′ portion of IL2RG express both the exogenously introduced CAR and a functional IL2RG complex, resulting in restored or continued expression of the IL-2 receptor γ chain.

### T-Cell Isolation and Activation

T-cell enrichment from PMBCs and activation with T-Cell TransAct was performed as described in EXAMPLE 1.

### T-Cell Gene Editing

CRISPR RNP were prepared by combining 36 µM sgRNA (Synthego) targeting DNA sequence GTGTGTATTTCTGGCTGGAA (SEQ ID NO:32) and 62.5 µM sNLS-SpCas9-sNLS (Aldevron) at a ratio of 16.5:1. 0.25 µg of plasmid DNA (i.e. a plasmid having a sequence according to SEQ ID NO:33) was mixed with 3.5 µl of RNP. T-cells were counted, centrifuged at 90 X G for 10 minutes and resuspended at 1 × 10⁶ cells/14.5 µl of P3 with supplement added (Lonza). 20 µl of T-cell suspension was added to the DNA/RNP mixture, transferred to a Lonza 384-well electroporation plate, and pulsed in a Lonza HT with code EH-115 AA. Cells were allowed to rest for 15 minutes at room temperature before transfer to 96-well plates (Sarstedt) in TexMACS medium supplemented with 12.5 ng/ml human IL-7 and IL-15 (Miltenyi premium grade).

### Flow Cytometry

Transgene expression was detected by staining with anti-Myc antibody (Cell Signaling Technology clone 9B11) and analysis on an Intellicyt iQue3 instrument. IL2RG expression was detected with CD132 antibody (Biolegend clone TUGh4).

### Genomic Editing of T-Cells to Express Circuit and IL2RG Chain

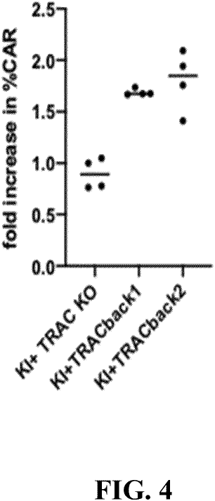

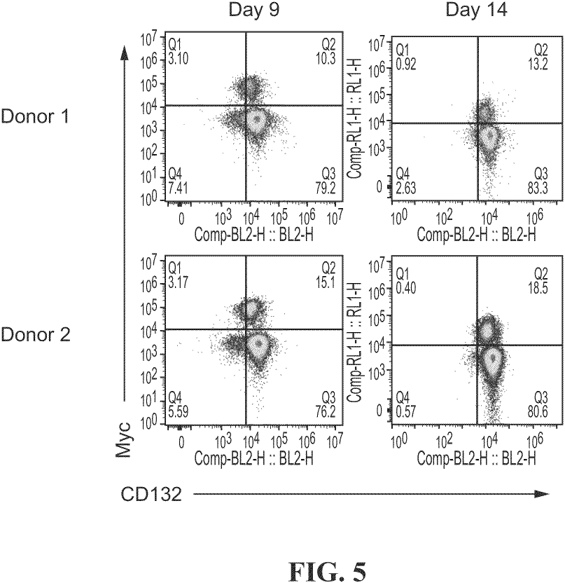

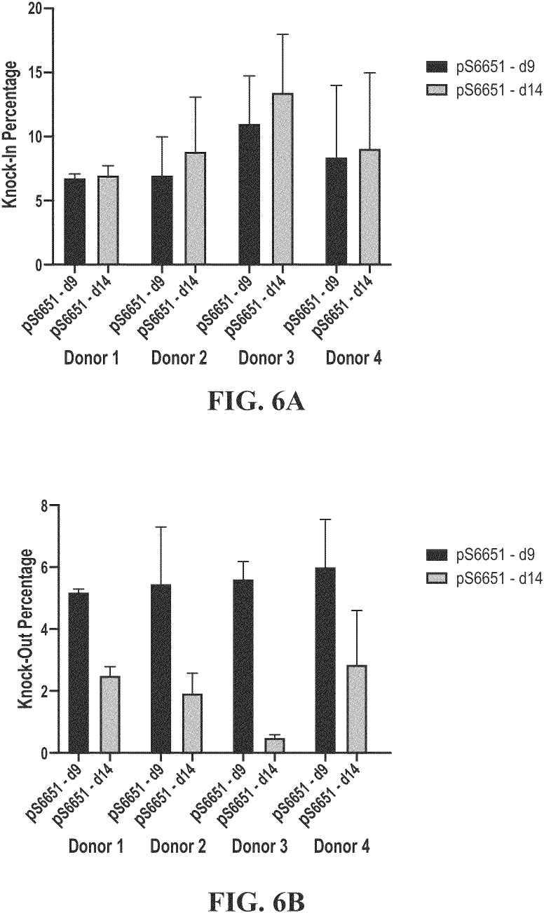

T-cells were genomically edited via electroporation of CRISPR RNP targeting the IL2RG locus with a plasmid repair template to express an exogenous transgene encoding a circuit with a Prime and CAR receptor and Myc-tag. FIG. 5 shows that the exogenous transgene (Myc-tagged prime receptor) is readily detected in electroporated cells stained with Anti-Myc antibody and analyzed by flow cytometry. As shown in FIG. 5, cells electroporated with plasmid having a sequence according to SEQ ID NO:33, in which the plasmid repair template includes the 3′ coding sequence that follows the CRISPR target cut site in IL2RG, along with a Prime and CAR receptor containing circuit, results in an IL2RG locus that expresses a full-length IL-2 receptor γ chain coding sequence in addition to the transgene. These cells have detectable IL2RG complex expression on the cell surface as indicated by the presence of CD132 (FIG. 5). As shown in FIG. 6A, cells from 4 donors electroporated with ps6651, IL2RG sgRNA, and CAS9, and assayed via flow cytometry demonstrate an increase in percentage of cells expressing both IL2RG and the exogenous transgene from day 9 post-electroporation to day 14 post-electroporation. Additionally, as shown in FIG. 6B, the population of cells with the IL2RG gene knocked out and that did not integrate the transgene, showed depletion over time due to a lack of IL2RG expression.

### Example 3

### In Vivo Treatment of Solid Tumors in Xenograft Mouse Model

T-cells expressing tumor antigen specific CAR are produced via a genome editing method described herein. Primary human solid tumor cells are grown in immune compromised mice. Exemplary solid cancer cells include solid tumor cell lines, such as provided in The Cancer Genome Atlas (TCGA) and/or the Broad Cancer Cell Line Encyclopedia (CCLE, see Barretina et al., Nature 483:603 (2012)). Exemplary solid cancer cells include primary tumor cells isolated from lung cancer, ovarian cancer, melanoma, colon cancer, gastric cancer, renal cell carcinoma, esophageal carcinoma, glioma, urothelial cancer, retinoblastoma, breast cancer, Non-Hodgkin lymphoma, pancreatic carcinoma, Hodgkin’s lymphoma, myeloma, hepatocellular carcinoma, leukemia, cervical carcinoma, cholangiocarcinoma, oral cancer, head and neck cancer, or mesothelioma. These mice are used to test the efficacy of T-cells expressing the exogenous CAR transgene and the functional TCR complex in the human tumor xenograft models. Following a subcutaneous implant or injection of 1×10⁵-1×10⁷ tumor cells, tumors are allowed to grow to 200-500 mm³ prior to initiation of treatment. T-cells genomically edited to express the exogenous CAR transgene and the functional TCR complex are then introduced into the mice. Tumor shrinkage in response to treatment with T-cells genomically edited to express the exogenous CAR transgene and the functional TCR complex can be either assessed by caliper measurement of tumor size or by following the intensity of a luciferase protein (ffluc) signal emitted by ffluc-expressing tumor cells.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A composition for targeted insertion of a nucleic acid comprising a sequence of equivalent coding potential to a 3′ portion or a 5′ portion of an endogenous gene of a cell and an exogenous transgene, the composition comprising:
a guide RNA (gRNA) targeting the endogenous gene;
an RNA-guided nuclease complexed with the gRNA; and
a nucleic acid complexed with the RNA-guided nuclease and comprising a sequence coding for one or more region(s) of homology to the endogenous gene, the sequence of equivalent coding potential to the 3′ portion or the 5′ portion of the endogenous gene and the transgene,
wherein the RNA-guided nuclease specifically cleaves the endogenous gene in the cell to create an insertion site, wherein the sequence of equivalent coding potential to the 3′ portion or the 5′ portion of the endogenous gene and the transgene of the nucleic acid are inserted into the insertion site, and wherein the insertion of the sequence of equivalent coding potential to the 3′ portion or the 5′ portion of the endogenous gene and the transgene of the nucleic acid results in restored or continued expression of the endogenous gene and expression of the transgene in the cell.

2-11. (canceled)

12. A cell comprising:
(i) a nucleic acid comprising, from 5′ to 3′:
(1) a sequence encoding a 5′ portion of an endogenous gene of the cell,
(2) a sequence of equivalent coding potential to a 3′ portion of the endogenous gene of the cell,
(3) a sequence encoding an exogenous transgene, and
(4) a sequence encoding the 3′ portion of the endogenous gene of the cell; and
wherein the cell expresses each of: (a) the endogenous gene encoded by (1) and (2) and (b) the transgene encoded by (3); or
(ii) a nucleic acid comprising from 5′ to 3′:
(1) a sequence encoding a 5′ portion of an endogenous gene of the cell,
(2) a sequence encoding an exogenous transgene,
(3) a sequence having equivalent coding potential to the 5′ portion of the endogenous gene of the cell, and
(4) a sequence encoding a 3′ portion of the endogenous gene of the cell; and
wherein the cell expresses each of (a) the transgene encoded by (2) and (b) the endogenous gene encoded by (3) and (4).

13. (canceled)

14. The cell of claim 12, wherein the endogenous gene is selected from the group consisting of: T-cell receptor alpha chain constant (TRAC), T-cell receptor beta chain constant (TRBC), CD3γ chain, CD3δ chain, CD3ε chain, CD3ξ chain, IL-2Rα chain, IL-2Rβ chain, and IL-2Rγ chain (IL2RG).

15. The cell of claim 14, wherein the endogenous gene is TRAC.

16. The cell of claim 14, wherein the endogenous gene is IL2RG.

17. The cell of claim 12, wherein the endogenous gene comprises a gene selected from the group consisting of: beta actin (Actb), ATP synthase H+ transporting, mitochondrial F0 complex subunit B1 (Atp5f1), beta-2 microglobulin (B2m), glyceraldehyde-3-phosphate dehydrogenase (Gapdh), glucuronidase beta (Gusb), hypoxanthine guanine phosphoribosyl transferase (Hprt), phosphoglycerate kinase I (Pgk1), peptidylprolyl isomerase A (Ppia), ribosomal protein S18 (Rps18), TATA box binding protein (Tbp), transferrin receptor (Tfrc), tyrosine 3-monooxygenase/tryptophan 5-monooxygenase activation protein zeta polypeptide (Ywhaz), Nanog homeobox (Nanog), zinc finger protein 42 (Rex1), and POU domain class 5 transcription factor 1 (Oct4).

18. The cell of claim 12, wherein the transgene comprises a chimeric antigen receptor (CAR).

19. The cell of claim 12, wherein the cell is an immune cell.

20. The cell of claim 39, wherein the T-cell is a CD4+ T-cell or a CD8+ T-cell.

21. A method of editing the genome of a cell comprising:
introducing into the cell a guide RNA (gRNA) targeting an endogenous gene in the cell, an RNA-guided nuclease complexed with the gRNA, and a nucleic acid complexed with the RNA-guided nuclease and comprising a sequence coding for one or more region(s) of homology to the endogenous gene, a sequence of equivalent coding potential to a 3′ portion or a 5′ portion of the endogenous gene, and an exogenous transgene,
wherein the RNA-guided nuclease specifically cleaves the endogenous gene in the cell to create an insertion site, wherein the sequence of equivalent coding potential to the 3′ portion or the 5′ portion of the endogenous gene and the exogenous transgene of the nucleic acid are inserted into the insertion site, and wherein insertion of the sequence of equivalent coding potential to the 3′ portion or the 5′ portion of the endogenous gene and the exogenous transgene of the nucleic acid results in restored or continued expression of the endogenous gene and expression of the transgene in the cell.

22-33. (canceled)

34. A method of treating or preventing a disease in a subject, comprising:
obtaining the cell of claim 12, and
administering the cell to the subject.

35. The method of claim 34, wherein the disease is cancer.

36. The method of claim 35, wherein the cell is obtained from the subject.

37. The method of claim 36, wherein the cell is a T-cell.

38. The method of claim 37, wherein the T cell is a CD4+ T-cell or a CD8+ T-cell.

39. The cell of claim 19, wherein the immune cell is a T Cell.
